(19) United States
(12) Patent Application Publication
(10) Pub. No.: US 2026/0167632 A1
(43) Pub. Date: Jun. 18, 2026

(54) NICOTINAMIDE DERIVATIVES FOR USE IN TREATING DISORDERS ASSOCIATED WITH KCNK13 ACTIVITY

(71) Applicant: Cerevance, Inc., Boston, MA (US)

(72) Inventors: Roland Bürli, Cambridge (GB); Kevin Doyle, Cambridge (GB); Martin Teall, Cambridge (GB)

(21) Appl. No.: 19/126,688

(22) PCT Filed: Nov. 2, 2023
(86) PCT No.: PCT/GB2023/052860

§ 371 Date: May 2, 2025

(30) Foreign Application Priority Data
Nov. 2, 2022 (GB) 2216324.0

(51) Int. Cl. C07D 413/12; A61K 31/4545; A61K 31/455; A61K 31/4725; A61K 31/553; A61P 29/00; C07D 213/71; C07D 213/82; C07D 267/14; C07D 401/06; C07D 401/12; C07D 405/12; C07D 409/12

(52) U.S. Cl. CPC C07D 413/12; A61K 31/4545; A61K 31/455; A61K 31/4725; A61K 31/553; A61P 29/00; C07D 213/71; C07D 213/82; C07D 267/14; C07D 401/06; C07D 401/12; C07D 405/12; C07D 409/12

(57) ABSTRACT

The present invention provides compounds of formula (I) and pharmaceutically acceptable salts, N-oxides, solvates and prodrugs thereof: Formula (I) wherein Z, X, R1, R2, R4, R1 R5, R6 and R7 are as defined in the specification, processes for their preparation, pharmaceutical compositions containing them and their use in therapy, particularly for use in treating disorders associated with KCNK13 activity.

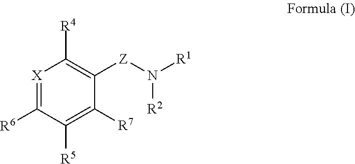

### FIELD OF THE INVENTION

The present invention relates to aryl and heteroaryl amides and sulfonamides, processes for their preparation, pharmaceutical compositions containing them and their use in therapy, particularly for use in treating disorders associated with KCNK13 activity.

### BACKGROUND OF THE INVENTION

### Inflammation & Neuroinflammation

Inflammation is part of the complex biological response of the body's tissue systems to harmful stimuli, such as invading pathogens or irritants and cellular damage. This is a generally protective response involving the cells of the immune system, blood vessels, and a diverse range of molecular mediators that function to eliminate the initial cause of irritation and cellular injury, clear out necrotic cells and tissues damaged from the original insult and initiate tissue repair. However, if inflammation becomes chronic or uncontrolled, then it can become causative or involved in the long-term progression of a range of diseases throughout the body, for example, arthritis, autoimmune disease, inflammatory bowel disorders, coeliac disease, hepatitis, asthma etc.

In the central nervous system (CNS) inflammation or neuroinflammation is a common underlying pathological feature of most neurological disorders and chronic neuroinflammation is evident in most if not all progressive neurodegenerative diseases such as Alzheimer's (AD) and Parkinson's disease (PD) (Heneka et al, 2014, Nat Rev Immunol, 14, 463-477), autoimmune disorders such as multiple sclerosis (Barclay & Shinohara, 2017, Brain Pathol, 27(2), 213-219) and can mediate ongoing damage to the CNS following brain injuries such as stroke (Jayaraj et al, 2019, J Neuroinflam, 16, 142-166) or traumatic brain injury (Simon et al, 2017, Nat Rev Neurol, 13(3), 171-191). Neuroinflammation has even been shown to be present and to play a role in psychiatric illnesses such as depression (Najjar et al, 2013, J Neuroinflammation, 10, 43-67; Wohleb et al, 2016, Nat Rev Neurosci, 17(8), 497-511) where overt tissue damage is less evident. The importance of neuroinflammation in disease is further underlined by findings that suggest that genes for immune receptors, such as TREM2 and CD33 are risk factors for, and afford selective vulnerability to a variety of neurodegenerative diseases including AD and PD (Jay et al, 2017, Mol Neurodegener, 12, 56-89). Many of these genes, including TREM2 and CD33, are exclusively expressed in brain microglia (MG) pointing to a key role of this cell type in neuroinflammation and pathogenic disease processes (Colonna & Butovsky, 2017, Annu Rev Immunol, 35, 441-468; Ransohoff, 2016, Science, 353, 777-783).

### Microglia

Microglia (MG) are generally considered to be the brain's resident macrophages playing a central role in the development, homeostasis and ultimately diseases of the CNS. MG arise solely from yolk sac erythromyeloid precursors and interact with almost all CNS components during embryonic and postnatal development. Adult MG have a sentinel type role surveying their environment and interacting with essentially all CNS components and thus have a marked impact on normal brain functioning and maintenance of tissue integrity. In order to achieve this, MG have the ability to rapidly adapt to their environment, increasing their cell number and modifying their cellular function and activation states (of which they have a broad spectrum), mediating and responding to damage, infection and inflammation. Specifically, during these challenged environments MG change their morphology, from the ramified sentinel phenotype to more amoeboid, which is accompanied by higher levels of phagocytic activity; increased proliferation and a cascade of cellular biochemistry results in cytokine release and an orchestrated inflammatory response process to ultimately resolve the adverse event/challenge (Li & Barres, 2018, Nat Rev Immunol, 18, 225-242). This microglial activation is a salient feature of all neurodegenerative diseases and can alter disease processes and progression. Although microglial activation is an initially favourable response to environment, there is clear evidence that this becomes dysfunctional and ultimately plays a role in driving inflammation, cell damage and loss, progressing the neurodegenerative disease process. The biochemical processes involved are complex, but a number of pathways have been identified as being key to the disease processes and potential intervention points for therapeutic approaches; one such process is that involving the nod-like receptor family pyrin domain containing 3 (NLRP3) cascades (Heneka et al, 2018, Nat Revs Neurosci, 19, 610-621).

### NLRP3

NLRP3 is a component of the innate immune system that functions as a pattern recognition receptor (PRR) that recognises pathogen-associated molecular patterns (PAMPs) and damage-associated molecular patterns (DAMPs) which are generated by endogenous stress and trigger downstream inflammatory pathways to eliminate microbial infection and repair damaged tissues (Kelley et al, 2019, Int J Mol Sci, 20, 3328-3352). The activation of the NLRP3 inflammasome requires a two-step process, comprising priming and then activation. Priming usually occurs through the stimulation of toll-like receptors (TLRs) (Toma et al, 2010, J Immunol, 184, 5287-5297; Qiao et al, 2012, FEBS Lett, 586, 1022-1026), which mediates upregulation of the nuclear factor-kappa B (NF-κB) pathway to increase the expression of NLRP3, caspase-1, and prointerleukin-1p (pro-IL-1). The secondary step is then required to trigger the formation of the inflammasome complex comprising NLRP3 together with the adaptor ASC protein PYCARD and caspase-1. This activated NLRP3 inflammasome leads to activation of caspase-1 which in turn activates the inflammatory cytokine, IL-1p. The NLRP3 inflammasome appears to be activated by changes in intracellular potassium (K⁺), and K⁺ efflux in itself is capable of activating NLRP3, while high extracellular K⁺ blocks the activation of the NLRP3 inflammasome but not the other inflammasomes (Pétrilli et al, 2007, Cell Death Differ, 14, 1583-1589; Munoz-Planillo et al, 2013, Immunity, 38, 1142-1153). Thus, a decrease of intracellular K⁺ has been considered to be the common trigger for NLRP3 inflammasome activation.

Genetic gain of function (GoF) mutations in the NLRP3 gene have been associated with a spectrum of dominantly inherited autoinflammatory diseases called cryopyrin-associated periodic syndrome (CAPS). These include familial cold autoinflammatory syndrome (FCAS), Muckle-Wells syndrome (MWS), chronic infantile neurological cutaneous and articular (CINCA) syndrome and neonatal onset multisystem inflammatory disease (NOMID). These diseases produce a diversity of immune-mediated organ changes and permanent central nervous system damage resulting in intellectual abnormalities (Izawa et al, 2012, DNA Research, 19(2), 143-152). In addition, exome sequencing data for genetic variation of NLRP3 in Parkinson's populations identified multiple single-nucleotide polymorphisms (SNPs) including rs7525979 that was associated with a significantly reduced risk of developing PD. Mechanistic studies indicated that the synonymous SNP, NLRP3 rs7525979, alters the efficiency of NLRP3 translation impacting NLRP3 protein stability and hence reducing NLRP3 inflammasome function (von Herrmann et al, 2018, NPJ Parkinsons Dis, 4, 2-10). Similarly, two functional single-nucleotide polymorphisms (SNPs) in the NLRP3 gene (rs2027432 and rs10754558) have been found to be associated with late-onset Alzheimer's disease in a Han Chinese population (Tan et al, 2013, Neuroimmunol, 265, 91-95).

### NLRP3 Disease Association & Therapeutic Potential

These genetic observations have highlighted diseases caused, as with the genetic gain of function mutations, or involving NLRP3 dysfunction in the onset of and ongoing pathological processes. However, NLRP3 has been associated with a diverse range of diseases and conditions (Table 1) and is an important contributor to inflammatory diseases throughout the body (for general reviews, see Mangan et al, 2018, Nat Rev Drug Discov, 17, 588-606).

TABLE 1

| Disease type | Disease | Citations |
|---|---|---|
| Neurodegeneration | Alzheimer's disease | Heneka et al, 2013, Nature, 493, 674-678; |
| | | Dempsey et al, 2017, Brain Behav Immun, 61, 306-316. |
| | Parkinson's disease | Gordon et al, 2018, Sci Transl Med, 10(465), 1-25; |
| | | Haque et al, 2020, Mov Disord, 35(1), 20-33. |
| | Frontal temporal dementia/ | Ising et al, 2019, Nature, 575, 669-673; |
| | progressive supranuclear | Stancu et al, 2019, Acta Neuropathol, 137(4), 599-617. |
| | palsy (PSP)/tauopathies | |
| | Amyotrophic lateral | Deora et al, 2020, Glia, 68(2), 407-421. |
| | sclerosis (ALS)/motor | |
| | neuron disease (MND) | |
| | Traumatic brain injury | Irrera et al, 2017, Front Pharmacol, 8, 459-469; |
| | | Ismael et al, 2018, J Neurotrauma, 35, 1294-1303; |
| | | Kuwar et al, 2019, J Neuroinflam, 16, 81-91. |
| | Multiple sclerosis | Gris et al, 2010, J Immunol, 185, 974-981; |
| | | Coll et al, 2015, Nat Med, 21, 248-255. |
| | Stroke/ischemic insult | Yang et al, 2014, J Cereb Blood Flow |
| | | Metab, 34(4), 660-667; |
| | | Ward et al, 2019, Pharmacol Res, 142, 237-250. |
| Psychiatric | Depression | Kaufmann et al, 2017, Brain Behav Immun, 64, 367-383; |
| | | Su et al, 2017, Behav Brain Res, 322, 1-8. |
| | Stress/anxiety/PTSD | Lei et al, 2017, Brain Res, 1671, 43-54; |
| | | Wang et al, 2018, J Neuroinflammation, 15(1), 21-35; |
| | | Dong et al, 2020, J Neuroinflammation, 17, 205-221. |
| | Schizophrenia/bipolar | Giridharan et al, 2020, Cells, 9(3), 577-591; |
| | disorder | Ventura et al, 2020, Acta Neuropsychiatr, 32(6), |
| | | 321-327; |
| | | Kim et al, 2016, J Psychiatr Res, 72, 43-50. |
| Genetic | Cryopyrin-associated | Coll et al, 2015, Nat Med, 21, 248-255. |
| (GoF mutations) | periodic syndrome (CAPS)/ | |
| | Muckle-Wells syndrome (MWS) | |
| Hearing Loss | Age and genetic related | Shi et al, 2017, Am J Transl Res, 9, 5611-5618; |
| | hearing loss | Nakanishi et al, 2020, Front Neurol, 11, 141-148. |
| Ocular/retinal | Age related macular | Gao et al, 2015, Mediators Inflamm, 2015, 690243; |
| | degeneration | Wooff et al, 2020, Sci Rep, 10(1), 2263-2283; |
| | | Puyang et al, 2016, Sci Reps, 6, 20998-21007; |
| | | Celkova et al, 2015, J Clin Med, 4, 172-192. |
| | Diabetic retinopathy | Zhang et al, 2017, Cell Death Dis, 8, 2941; |
| | | Sui et al, 2020, Cell Death Dis, 1(10), 901-917. |
| Cardiovascular | Atherosclerosis | van der Heijden et al, 2017, Arterioscler Thromb Vasc |
| | | Biol, 37, 1457-1461; |
| | | Duewell et al, 2010, Nature, 464, 1357-1361. |
| | Myocardial infarction/ | Marchetti et al, 2014, J Cardiovasc Pharmacol, 63, |
| | ischemia | 316-322; |
| | | van Hout et al, 2017, Eur Heart J, 38, 828-836; |
| | | Sandanger et al, 2013, Cardiovascular Research, 99(1), |
| | | 164-174. |
| Inflammatory/ | Rheumatoid arthritis/ | Guo, 2018, Clin Exp Immunol, 194(2), 231-243; |
| autoimmune | Lupus | Vande Walle et al, 2014, Nature, 512, 69-73; |
| | | Fu et al, 2017, Arthritis Rheumatol, 69(8), 1636-1646. |
| | Gout | Martinon et al, 2006, Nature, 440, 237-241. |
| | Asthma/respiratory | Ritter et al, 2014, Clin Exp Immunol, 178, 212-223; |
| | inflammation | Kim et al, 2017, Am J Respir Crit Care Med, 196(3), |
| | | 283-297. |
| | Psoriasis/skin disease | Primiano et al, 2016, J Immunol, 197(6), 2421-2433; |
| | | Wang et al, 2020, J Dermatol Sci, 98(3), 146-151; |
| | | Zhu et al, 2020, J Invest Dermatol, |
| | | S0022-202X(20)32276-32284. |
| | Inflammatory bowel | Perera et al, 2018, Sci Reps, 8, 8618-8633; |
| | disease/colitis | Zhen & Zhang, 2019, Front Immunol, 10, 276-286. |
| Metabolic | NASH/NAFLD/fibrosis | Mridha et al, 2017, J Hepatol, 66, 1037-1046; |
| | | Wree et al, 2014, J Mol Med, 92, 1069-1082. |
| | Diabetes | Hu et al, 2015, Proc Natl Acad Sci USA, 112, |
| | | 11318-11323. |
| | Metabolic Syndrome | Cañadas-Lozano et al, 2020, Geroscience, 42, 715-725; |
| | | Marín-Aguilar et al, 2020, J Gerontol A Biol Sci Med |
| | | Sci, 75(8), 1457-1464. |

Diseases of the brain, where neuroinflammation has been demonstrated to be a key driver of ongoing disease pathology, have seen considerable research focus. Many of these have identified microglial NLRP3 as being a key contributor to aberrant inflammatory processes and ongoing disease pathology (Table 1).

Genetic ablation of NLRP3 or pharmacological blockade of the inflammasome has been demonstrated to produce significant improvements in ongoing disease pathology in a range of preclinical models of neurodegenerative disease including Parkinson's (Gordon et al, 2018, Sci Transl Med, 10(465), 1-25; Haque et al, 2020, Mov Disord, 35(1), 20-33), Alzheimer's (Heneka et al, 2013, Nature, 493, 674-678; Dempsey et al, 2017, Brain Behav Immun, 61, 306-316), tauopathies such as Frontal Temporal Dementia (Ising et al, 2019, Nature, 575, 669-673), amyotrophic lateral sclerosis (ALS)/motor neuron disease (MND) (Debye et al, 2018, Brain Pathol, 28(1), 14-27; Gugliandolo et al, 2018, Inflammation, 41, 93-103; Deora et al, 2020, Glia, 68(2), 407-421), traumatic brain insults (Irrera et al, 2020, Int J Mol Sci, 21(17), 6204-6223; Wallisch et al, 2017, Neurocrit Care, 27(1), 44-50; O'Brien et al, 2020, J Neuroinflammation, 17(1), 104-116), multiple sclerosis (MS) (Barclay & Shinohara, 2017, Brain Pathol, 27, 213-219; Olcum et al, 2020, Adv Protein Chem Struct Biol, 119, 247-308) and stroke/ischaemic insults (Luo et al, 2019, Curr Neuropharmacol, 17(7), 582-589; Ward et al, 2019, Pharmacol Res, 142, 237-250) (for general reviews on neurodegeneration, see Heneka et al, 2018, Nat Revs Neurosci, 19, 610-621; Guan & Han, 2020, Front Integr Neurosci, 14, 37-46).

Interestingly, NLRP3 has also been shown to have an additional involvement in the inflammation associated with psychiatric diseases such as depression (Kaufmann et al, 2017, Brain Behav Immun, 64, 367-383; Su et al, 2017, Behav Brain Res, 322, 1-8), anxiety/stress disorders (Lei et al, 2017, Brain Res, 1671, 43-54; Wang et al, 2018, J Neuroinflammation, 15(1), 21-35), and schizophrenia and bipolar disorder (Giridharan et al, 2020, Cells, 9(3), 577-591; Ventura et al, 2020, Acta Neuropsychiatr, 32(6), 321-327; Kim et al, 2016, J Psychiatr Res, 72, 43-50).

Taken together these data suggest that modulating NLRP3 inflammasome-induced neuroinflammation would be of broad therapeutic benefit across a wide range of brain disorders.

Non brain disorders: NLRP3 is associated with a diverse range of diseases and conditions (Table 1) and is an important contributor to inflammatory diseases of the peripheral tissues and organs. These include retinal diseases such as age related macular degeneration and diabetic retinopathy (Gao et al, 2015, Mediators Inflamm, 2015, 690243; Lim et al, 2020, Int J Mol Sci, 21(3), 899-913), hearing loss (Nakanishi et al, 2020, Front Neurol, 11, 141-148; Shi et al, 2017, Am J Transl Res, 9, 5611-5618), cardiovascular diseases such as atherosclerosis (Grebe et al, 2018, Circ Res, 122, 1722-1740; Zhou et al, 2018, J Immunol Res, 2018, 5702103), inflammatory and autoimmune diseases such as psoriasis and asthma (Li et al, 2020, Biomed Pharmaco, 130, 110542-110554; Theofani et al, 2019, J Clin Med, 8, 1615-1643; Wang et al, 2020, J Dermatol Sci, 98(3), 146-151) and metabolic disorders and associated complications (Wan et al, 2016, Can J Gastroenterol Hepatol, 2016, 6489012-6489019; Ding et al, 2019, Biomolecules, 9(12), 850-865).

### KCNK13 (THIK-1)

The central role of K⁺ flux in the activation of the conical NLRP3 activation has been well documented (see paragraph on NLRP3 above) and several channels have been suggested to be the mediators of this K⁺ current in microglia. One such channel is KCNK13 (K_{2P}13.1) or potassium two pore domain channel subfamily K member 13 gene which encodes for a two-pore forming domain potassium channel known as tandem pore domain halothane-inhibited K⁺ channel 1 or THIK-1. KCNK13 together with KCNK12 are members of the leak or background K⁺ channels (K₂P) first cloned by Rajan et al (2001, J Biol Chem, 276, 7302-7311). KCNK12 encodes a closely related channel THIK-2 which is silent as a homodimer but can heterodimerise with THIK-1 to form an active channel, albeit with reduced function vs THIK-1 homodimer (Blin et al, 2014, J Biol Chem, 289, 28202-28212). Electrophysiological studies show that THIK-1 displays an outward rectify current with a very small single-channel conductance (˜5 pS at +100 mV) and short open time duration (<0.5 ms) (Kang et al, 2014, Pflugers Arch, 466(7), 1289-1300). THIK-1 K⁺ channel conductance has been shown to play roles in modulating the biology of microglia and has a central role in mediating the proinflammatory response of microglia via the NLRP3 inflammasome (Madry et al, 2018, Neuron, 97, 299-312). Furthermore, blockade of THIK-1 conductance inhibits lipopolysaccharide (LPS)-induced production of proinflammatory IL-1β (Madry et al, 2018, Neuron, 97, 299-312). Our own data further confirm these findings demonstrating that inhibition of THIK-1 attenuates LPS- and K⁺-induced activation of caspase-1 and subsequent IL-1β production and release from isolated microglia (see example 3 below) and IL-1β release from LPS-treated rodent hippocampus. It can thus be concluded that selective inhibitors of THIK-1 reduce NLRP3 inflammasome mediated inflammation and thus have therapeutic utility in many of the NLRP3 related indications highlighted above and in Table 1.

There is a need for treatment of the above diseases and conditions and others described herein with compounds that are KCNK13 antagonists. The present invention provides antagonists of KCNK13.

### SUMMARY OF THE INVENTION

A first aspect of the present invention provides a compound of formula (I):

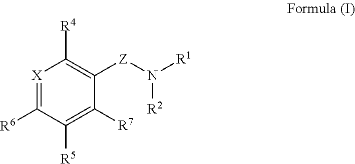

or a pharmaceutically acceptable salt, N-oxide, solvate or prodrug thereof, wherein:
X is N or CH;
Z is —C(O)— or —SO₂—;
R¹ is -L-R³;
R² is hydrogen or C₁-C₃ alkyl;
L is a bond, —CH₂—, —CHMe-, or —C(C₂H₄)—;
R³ is a 5- to 11-membered cyclic group, wherein the cyclic group is optionally substituted with one or more substituents independently selected from halo, cyano, C₁-C₃ alkyl, C₁-C₃ haloalkyl, —O—(C₁-C₃ alkyl), —O—(C₁-C₃ haloalkyl), oxo (═O), or —C(O)O(C₁-C₃ alkyl);
or R¹ and R² and the nitrogen atom to which they are attached together form a 5- to 1n-membered heterocyclic group, wherein the heterocyclic group is optionally substituted with one or more substituents independently selected from halo, cyano, C₁-C₃ alkyl, C₁-C₃ haloalkyl, —O—(C₁-C₃ alkyl), —O—(C₁-C₃ haloalkyl), or halophenyl;
R⁴ is cyano, —O—(C₁-C₃ alkyl), —O—(C₁-C₃ haloalkyl), or —N—(C₁-C₃ alkyl)₂;
or R⁴ and X together with the carbon atom to which they are attached form a 5-membered heteroaryl group;
R⁵ is hydrogen, halo, C₁-C₃ alkyl, C₁-C₃ haloalkyl, or C₃-C₅ cycloalkyl;
R⁶ is hydrogen, methyl or halo; and
R⁷ is hydrogen, methyl or halo.

In one embodiment, Z is —C(O)—, such that the compound is of formula (Ia):

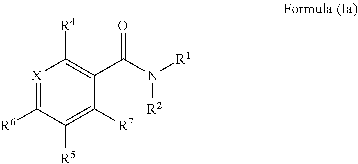

In another embodiment, Z is —SO₂—, such that the compound is of formula (Ib):

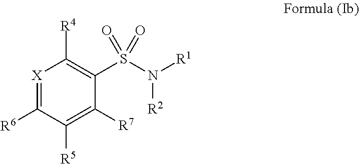

In one embodiment, X is N such that the compound comprises a pyridinyl group. In another embodiment, X is CH such that the compound comprises a phenyl group.

In one embodiment,
R¹ is -L-R³;
R² is hydrogen or C₁-C₃ alkyl;
L is a bond, —CH₂—, —CHMe-, or —C(C₂H₄)—; and
R³ is a 5- to 11-membered cyclic group, wherein the cyclic group is optionally substituted with one or more (such as one, two, three, four or five) substituents independently selected from halo, cyano, C₁-C₃ alkyl, C₁-C₃ haloalkyl, —O—(C₁-C₃ alkyl), —O—(C₁-C₃ haloalkyl), oxo (═O), or —C(O)O(C₁-C₃ alkyl).

Preferably,
R¹ is -L-R³;
R² is hydrogen, methyl, or ethyl;
L is a bond, —CH₂—, —CHMe-, or —C(C₂H₄)—; and
R³ is a 6- to 10-membered cyclic group selected from phenyl or phenyl fused to a 5- or 6-membered cyclic group optionally comprising a nitrogen, oxygen or sulfur ring atom, wherein the 6- to 10-membered cyclic group is optionally substituted with one, two, three or four substituents independently selected from fluoro, chloro, bromo, cyano, C₁-C₂ alkyl, C₁-C₂ haloalkyl, —O—(C₁-C₂ alkyl), —O—(C₁-C₂ haloalkyl), oxo (═O), or —C(O)O(C₁-C₂ alkyl).

Preferably,
R¹ is -L-R³;
R² is methyl or ethyl;
L is a bond, —CH₂—, —CHMe-, or —C(C₂H₄)—; and
R³ is a 6- to 10-membered cyclic group selected from phenyl, indanyl, dihydrobenzo[b]thiophenyl, chromanyl, and 2,3-dihydrobenzofuranyl, wherein the cyclic group is optionally substituted with one, two or three substituents independently selected from fluoro, chloro, cyano, methyl, fluoromethyl, methoxy, fluoromethoxy, oxo (═O), or —CO₂Me.

In another embodiment, R¹ and R² and the nitrogen atom to which they are attached together form a 5- to 11-membered heterocyclic group, wherein the heterocyclic group is optionally substituted with one or more (such as one, two, three, four or five) substituents independently selected from halo, cyano, C₁-C₃ alkyl, C₁-C₃ haloalkyl, —O—(C₁-C₃ alkyl), —O—(C₁-C₃ haloalkyl), or halophenyl. Preferably R¹ and R² and the nitrogen atom to which they are attached together form a 5- to 11-membered non-aromatic heterocyclic group comprising one or two nitrogen and/or oxygen ring atoms, wherein the heterocyclic group is optionally substituted with one, two or three substituents independently selected from fluoro, chloro, bromo, cyano, C₁-C₃ alkyl, C₁-C₃ haloalkyl, —O—(C₁-C₃ alkyl), —O—(C₁-C₃ haloalkyl), or halophenyl. Preferably R¹ and R² and the nitrogen atom to which they are attached together form a heterocyclic group selected from piperidin-1-yl, tetrahydroisoquinolin-2-yl, or 2,3,4,5-tetrahydrobenzo[f][1,4]oxazepin-4-yl, wherein the heterocyclic group is optionally substituted with one or two substituents independently selected from fluoro or fluorophenyl.

In one embodiment, R⁴ is cyano, —O—(C₁-C₃ alkyl), —O—(C₁-C₃ haloalkyl), or —N—(C₁-C₃ alkyl)₂. Preferably R⁴ is cyano, methoxy, ethoxy, halomethoxy, haloethoxy, or —N—(C₁-C₂ alkyl)₂. Preferably R⁴ is cyano, methoxy, ethoxy, fluoromethoxy, or fluoroethoxy.

In another embodiment, R⁴ and X together with the carbon atom to which they are attached form a 5-membered heteroaryl group (such as pyrrolyl, pyrazolyl, imidazolyl, oxazolyl, isoxazolyl, thiazolyl, isothiazolyl, triazolyl, oxadiazolyl, thiadiazolyl, tetrazolyl, oxatriazolyl, or thiatriazolyl).

R⁵ is hydrogen, halo, C₁-C₃ alkyl, C₁-C₃ haloalkyl, or C₃-C₅ cycloalkyl. Preferably R⁵ is hydrogen, fluoro, chloro, bromo, C₁-C₂ alkyl, C₁-C₂ haloalkyl, or C₃-C₄ cycloalkyl. Preferably R⁵ is hydrogen, fluoro, chloro, methyl, fluoromethyl, cyclopropyl, or cyclobutyl.

R⁶ is hydrogen, methyl or halo. Preferably R⁶ is hydrogen, methyl, fluoro, or chloro.

Preferably R⁶ is hydrogen, methyl, or fluoro.

R⁷ is hydrogen, methyl or halo. Preferably R⁷ is hydrogen, methyl, fluoro, or chloro.

Preferably R⁷ is hydrogen or methyl.

In a first specific embodiment of the first aspect, the present invention provides a compound of formula (I), or a pharmaceutically acceptable salt, N-oxide, solvate or prodrug thereof, wherein:
X is N;
Z is —C(O)—;
R¹ is -L-R³;
R² is C₁-C₃ alkyl;
L is a bond, —CH₂—, —CHMe-, or —C(C₂H₄)—;
R³ is a 6- to 11-membered cyclic group, wherein the cyclic group is substituted with one or more substituents independently selected from halo, cyano, C₁-C₃ alkyl, C₁-C₃ haloalkyl, —O—(C₁-C₃ alkyl), —O—(C₁-C₃ haloalkyl), oxo (═O), or —C(O)O(C₁-C₃ alkyl);
R⁴ is cyano, —O—(C₁-C₃ alkyl), —O—(C₁-C₃ haloalkyl), or —N—(C₁-C₃ alkyl)₂;
R⁵ is hydrogen, halo, C₁-C₃ alkyl, C₁-C₃ haloalkyl, or C₃-C₅ cycloalkyl;
R⁶ is hydrogen, methyl or halo;
R⁷ is hydrogen, methyl or halo;
provided that at least one of R⁵, R⁶ and R⁷ is not hydrogen; and

provided that the compound is not:
i) 5-chloro-N-(4-fluorophenyl)-2-methoxy-N-methylnicotinamide;
ii) 5-chloro-2-methoxy-N-methyl-N-(1-methylpiperidin-4-yl)nicotinamide;
iii) 5-chloro-N-ethyl-2-methoxy-N-(m-tolyl)nicotinamide;
iv) 5-chloro-N-ethyl-2-methoxy-N-(o-tolyl)nicotinamide;
v) 5-bromo-N-(4-fluorophenyl)-2-methoxy-N-methylnicotinamide;
vi) 5-bromo-2-methoxy-N-methyl-N-(1-methylpiperidin-4-yl)nicotinamide;
vii) 5-bromo-N-ethyl-2-methoxy-N-(m-tolyl)nicotinamide;
viii) 5-bromo-N-ethyl-2-methoxy-N-(o-tolyl)nicotinamide;
ix) 5-bromo-N-(2,4-difluorophenyl)-2-methoxy-N-methylnicotinamide;
x) 5-bromo-N-(2,4-difluorophenyl)-2-(dimethylamino)-N-methylnicotinamide;
xi) 5-bromo-2-(dimethylamino)-N-(3-isopropylphenyl)-N-methylnicotinamide;
xii) 5-bromo-2-(dimethylamino)-N-((2-ethylbenzofuran-3-yl)methyl)-N-methylnicotinamide;
xiii) N-(4-fluorophenyl)-2-methoxy-N,4,5,6-tetramethylnicotinamide;
xiv) 2-methoxy-N,4,5,6-tetramethyl-N-(1-methylpiperidin-4-yl)nicotinamide;
xv) N-ethyl-2-methoxy-4,5,6-trimethyl-N-(m-tolyl)nicotinamide;
xvi) N-ethyl-2-methoxy-4,5,6-trimethyl-N-(o-tolyl)nicotinamide;
xvii) 2-methoxy-N,6-dimethyl-N-(1-methyl-4,5,6,7-tetrahydro-1H-indazol-4-yl)nicotinamide;
xviii) N-(5-chloro-2-cyanophenyl)-2-methoxy-N,6-dimethylnicotinamide;
xix) 5-chloro-N-(4-fluorophenyl)-2-methoxy-N,4,6-trimethylnicotinamide;
xx) N-(4-cyanobenzyl)-2-methoxy-6-methyl-N-propylnicotinamide;
xxi) 5-chloro-2-methoxy-N,4,6-trimethyl-N-(1-methylpiperidin-4-yl)nicotinamide;
xxii) N-(2-(dimethylamino)pyridin-3-yl)-N,6-dimethyl-2-propoxynicotinamide;
xxiii) 2-methoxy-N,6-dimethyl-N-(2-methyl-4,5,6,7-tetrahydrobenzo[d]thiazol-7-yl)nicotinamide;
xxiv) 5-chloro-N-ethyl-2-methoxy-4,6-dimethyl-N-(m-tolyl)nicotinamide;
xxv) 5-chloro-N-ethyl-2-methoxy-4,6-dimethyl-N-(o-tolyl)nicotinamide;
xxvi) 2-ethoxy-N-((5-methoxy-6-methyl-4-oxo-1,4-dihydropyridin-3-yl)methyl)-N,4-dimethylnicotinamide;
xxvii) 2-ethoxy-N-((5-methoxy-6-methyl-4-oxo-1,4-dihydropyridin-3-yl)methyl)-N,6-dimethylnicotinamide;
xxviii) N-(2-ethyl-4,5,6,7-tetrahydrobenzo[d]thiazol-7-yl)-2-methoxy-N,6-dimethylnicotinamide;
xxix) 2-ethoxy-N,6-dimethyl-N-(2-methyl-4,5,6,7-tetrahydrobenzo[d]thiazol-7-yl)nicotinamide;
xxx) 2-ethoxy-N-(2-ethyl-4,5,6,7-tetrahydrobenzo[d]thiazol-7-yl)-N,6-dimethylnicotinamide;
xxxi) 5-bromo-N-(4-fluorophenyl)-2-methoxy-N,4,6-trimethylnicotinamide;
xxxii) 5-bromo-2-methoxy-N,4,6-trimethyl-N-(1-methylpiperidin-4-yl)nicotinamide;
xxxiii) 5-bromo-N-ethyl-2-methoxy-4,6-dimethyl-N-(m-tolyl)nicotinamide;
xxxiv) 5-bromo-N-ethyl-2-methoxy-4,6-dimethyl-N-(o-tolyl)nicotinamide; or
xxxv) N-(6-bromochroman-4-yl)-2-methoxy-N,6-dimethylnicotinamide;
   or a stereoisomer of any of the above compounds.

Preferably,
R² is methyl or ethyl;
L is a bond, —CH₂—, —CHMe-, or —C(C₂H₄)—; and
R³ is a 6- to 10-membered cyclic group selected from phenyl or phenyl fused to a 5- or 6-membered cyclic group optionally comprising a nitrogen, oxygen or sulfur ring atom, wherein the 6- to 10-membered cyclic group is substituted with one, two, three or four substituents independently selected from fluoro, chloro, bromo, cyano, C₁-C₂ alkyl, C₁-C₂ haloalkyl, —O—(C₁-C₂ alkyl), —O—(C₁-C₂ haloalkyl), oxo (═O), or —C(O)O(C₁-C₂ alkyl).

Preferably,
R² is methyl or ethyl;
L is a bond, —CH₂—, —CHMe-, or —C(C₂H₄)—; and
R³ is a 6- to 10-membered cyclic group selected from phenyl, indanyl, dihydrobenzo[b]thiophenyl, chromanyl, and 2,3-dihydrobenzofuranyl, wherein the cyclic group is substituted with one, two or three substituents independently selected from fluoro, chloro, cyano, methyl, fluoromethyl, methoxy, fluoromethoxy, oxo (═O), or —CO₂Me.

R⁴ is cyano, —O—(C₁-C₃ alkyl), —O—(C₁-C₃ haloalkyl), or —N—(C₁-C₃ alkyl)₂. Preferably R⁴ is cyano, methoxy, ethoxy, halomethoxy, haloethoxy, or —N—(C₁-C₂ alkyl)₂. Preferably R⁴ is cyano, methoxy, ethoxy, fluoromethoxy, or fluoroethoxy.

R⁵ is hydrogen, halo, C₁-C₃ alkyl, C₁-C₃ haloalkyl, or C₃-C₅ cycloalkyl. Preferably R⁵ is hydrogen, fluoro, chloro, bromo, C₁-C₂ alkyl, C₁-C₂ haloalkyl, or C₃-C₄ cycloalkyl. Preferably R⁵ is hydrogen, fluoro, chloro, methyl, fluoromethyl, cyclopropyl, or cyclobutyl.

R⁶ is hydrogen, methyl or halo. Preferably R⁶ is hydrogen, methyl, fluoro, or chloro.

Preferably R⁶ is hydrogen, methyl, or fluoro.

R⁷ is hydrogen, methyl or halo. Preferably R⁷ is hydrogen, methyl, fluoro, or chloro.

Preferably R⁷ is hydrogen or methyl.

In a preferred embodiment of the first specific embodiment of the first aspect of the present invention, R⁵ is halo, C₁-C₃ alkyl, C₁-C₃ haloalkyl, or C₃-C₅ cycloalkyl, provided that the compound is not:
i) 5-chloro-N-(4-fluorophenyl)-2-methoxy-N-methylnicotinamide;
ii) 5-chloro-2-methoxy-N-methyl-N-(1-methylpiperidin-4-yl)nicotinamide;
iii) 5-chloro-N-ethyl-2-methoxy-N-(m-tolyl)nicotinamide;
iv) 5-chloro-N-ethyl-2-methoxy-N-(o-tolyl)nicotinamide;
v) 5-bromo-N-(4-fluorophenyl)-2-methoxy-N-methylnicotinamide;
vi) 5-bromo-2-methoxy-N-methyl-N-(1-methylpiperidin-4-yl)nicotinamide;
vii) 5-bromo-N-ethyl-2-methoxy-N-(m-tolyl)nicotinamide;
viii) 5-bromo-N-ethyl-2-methoxy-N-(o-tolyl)nicotinamide;
ix) 5-bromo-N-(2,4-difluorophenyl)-2-methoxy-N-methylnicotinamide;
x) 5-bromo-N-(2,4-difluorophenyl)-2-(dimethylamino)-N-methylnicotinamide;
xi) 5-bromo-2-(dimethylamino)-N-(3-isopropylphenyl)-N-methylnicotinamide;
xii) 5-bromo-2-(dimethylamino)-N-((2-ethylbenzofuran-3-yl)methyl)-N-methylnicotinamide;
xiii) N-(4-fluorophenyl)-2-methoxy-N,4,5,6-tetramethylnicotinamide;
xiv) 2-methoxy-N,4,5,6-tetramethyl-N-(1-methylpiperidin-4-yl)nicotinamide;
xv) N-ethyl-2-methoxy-4,5,6-trimethyl-N-(m-tolyl)nicotinamide;
xvi) N-ethyl-2-methoxy-4,5,6-trimethyl-N-(o-tolyl)nicotinamide;
xvii) 5-chloro-N-(4-fluorophenyl)-2-methoxy-N,4,6-trimethylnicotinamide;
xviii) 5-chloro-2-methoxy-N,4,6-trimethyl-N-(1-methylpiperidin-4-yl)nicotinamide;
xix) 5-chloro-N-ethyl-2-methoxy-4,6-dimethyl-N-(m-tolyl)nicotinamide;
xx) 5-chloro-N-ethyl-2-methoxy-4,6-dimethyl-N-(o-tolyl)nicotinamide;
xxi) 5-bromo-N-(4-fluorophenyl)-2-methoxy-N,4,6-trimethylnicotinamide;
xxii) 5-bromo-2-methoxy-N,4,6-trimethyl-N-(1-methylpiperidin-4-yl)nicotinamide;
xxiii) 5-bromo-N-ethyl-2-methoxy-4,6-dimethyl-N-(m-tolyl)nicotinamide; or
xxiv) 5-bromo-N-ethyl-2-methoxy-4,6-dimethyl-N-(o-tolyl)nicotinamide;
   or a stereoisomer of any of the above compounds.

In this preferred embodiment of the first specific embodiment of the first aspect of the present invention, preferably R⁵ is fluoro, chloro, bromo, C₁-C₂ alkyl, C₁-C₂ haloalkyl, or C₃-C₄ cycloalkyl. Preferably R⁵ is fluoro, chloro, methyl, fluoromethyl, cyclopropyl, or cyclobutyl.

In a second specific embodiment of the first aspect, the present invention provides a compound of formula (I), or a pharmaceutically acceptable salt, N-oxide, solvate or prodrug thereof, wherein:
X is N;
Z is —C(O)—;
R¹ is -L-R³;
R² is C₁-C₃ alkyl;
L is a bond, —CH₂—, —CHMe-, or —C(C₂H₄)—;
R³ is a 6-membered aryl or heteroaryl group, wherein the 6-membered aryl or heteroaryl group is substituted with one or more substituents independently selected from halo, cyano, C₁-C₃ alkyl, C₁-C₃ haloalkyl, —O—(C₁-C₃ alkyl), —O—(C₁-C₃ haloalkyl), or —C(O)O(C₁-C₃ alkyl);
R⁴ is cyano, —O—(C₁-C₃ alkyl), —O—(C₁-C₃ haloalkyl), or —N—(C₁-C₃ alkyl)₂;
R⁵ is hydrogen, halo, C₁-C₃ alkyl, C₁-C₃ haloalkyl, or C₃-C₅ cycloalkyl;
R⁶ is hydrogen, methyl or halo;
R⁷ is hydrogen, methyl or halo;
provided that at least one of R⁵, R⁶ and R⁷ is not hydrogen; and

provided that the compound is not:
i) 5-chloro-N-(4-fluorophenyl)-2-methoxy-N-methylnicotinamide;
ii) 5-chloro-N-ethyl-2-methoxy-N-(m-tolyl)nicotinamide;
iii) 5-chloro-N-ethyl-2-methoxy-N-(o-tolyl)nicotinamide;
iv) 5-bromo-N-(4-fluorophenyl)-2-methoxy-N-methylnicotinamide;
v) 5-bromo-N-ethyl-2-methoxy-N-(m-tolyl)nicotinamide;
vi) 5-bromo-N-ethyl-2-methoxy-N-(o-tolyl)nicotinamide;
vii) 5-bromo-N-(2,4-difluorophenyl)-2-methoxy-N-methylnicotinamide;
viii) 5-bromo-N-(2,4-difluorophenyl)-2-(dimethylamino)-N-methylnicotinamide;
ix) 5-bromo-2-(dimethylamino)-N-(3-isopropylphenyl)-N-methylnicotinamide;
x) N-(4-fluorophenyl)-2-methoxy-N,4,5,6-tetramethylnicotinamide;
xi) N-ethyl-2-methoxy-4,5,6-trimethyl-N-(m-tolyl)nicotinamide;
xii) N-ethyl-2-methoxy-4,5,6-trimethyl-N-(o-tolyl)nicotinamide;
xiii) N-(5-chloro-2-cyanophenyl)-2-methoxy-N,6-dimethylnicotinamide;
xiv) 5-chloro-N-(4-fluorophenyl)-2-methoxy-N,4,6-trimethylnicotinamide;
xv) N-(4-cyanobenzyl)-2-methoxy-6-methyl-N-propylnicotinamide;
xvi) N-(2-(dimethylamino)pyridin-3-yl)-N,6-dimethyl-2-propoxynicotinamide;
xvii) 5-chloro-N-ethyl-2-methoxy-4,6-dimethyl-N-(m-tolyl)nicotinamide;
xviii) 5-chloro-N-ethyl-2-methoxy-4,6-dimethyl-N-(o-tolyl)nicotinamide;
xix) 5-bromo-N-(4-fluorophenyl)-2-methoxy-N,4,6-trimethylnicotinamide;
xx) 5-bromo-N-ethyl-2-methoxy-4,6-dimethyl-N-(m-tolyl)nicotinamide; or
xxi) 5-bromo-N-ethyl-2-methoxy-4,6-dimethyl-N-(o-tolyl)nicotinamide;
   or a stereoisomer of any of the above compounds.

Preferably,
R² is methyl or ethyl;
L is a bond, —CH₂—, —CHMe-, or —C(C₂H₄)—; and
R³ is phenyl substituted with one, two, three or four substituents independently selected from fluoro, chloro, bromo, cyano, C₁-C₂ alkyl, C₁-C₂ haloalkyl, —O—(C₁-C₂ alkyl), —O—(C₁-C₂ haloalkyl), or —C(O)O(C₁-C₂ alkyl).

Preferably,
R² is methyl or ethyl;
L is a bond, —CH₂—, —CHMe-, or —C(C₂H₄)—; and
R³ is phenyl substituted with one, two or three substituents independently selected from fluoro, chloro, cyano, methyl, fluoromethyl, methoxy, fluoromethoxy, or —CO₂Me.

R⁴ is cyano, —O—(C₁-C₃ alkyl), —O—(C₁-C₃ haloalkyl), or —N—(C₁-C₃ alkyl)₂. Preferably R⁴ is cyano, methoxy, ethoxy, halomethoxy, haloethoxy, or —N—(C₁-C₂ alkyl)₂. Preferably R⁴ is cyano, methoxy, ethoxy, fluoromethoxy, or fluoroethoxy.

R⁵ is hydrogen, halo, C₁-C₃ alkyl, C₁-C₃ haloalkyl, or C₃-C₅ cycloalkyl. Preferably R⁵ is hydrogen, fluoro, chloro, bromo, C₁-C₂ alkyl, C₁-C₂ haloalkyl, or C₃-C₄ cycloalkyl.

Preferably R⁵ is hydrogen, fluoro, chloro, methyl, fluoromethyl, cyclopropyl, or cyclobutyl.

R⁶ is hydrogen, methyl or halo. Preferably R⁶ is hydrogen, methyl, fluoro, or chloro. Preferably R⁶ is hydrogen, methyl, or fluoro.

R⁷ is hydrogen, methyl or halo. Preferably R⁷ is hydrogen, methyl, fluoro, or chloro. Preferably R⁷ is hydrogen or methyl.

In a preferred embodiment of the second specific embodiment of the first aspect of the present invention, R⁵ is halo, C₁-C₃ alkyl, C₁-C₃ haloalkyl, or C₃-C₅ cycloalkyl, provided that the compound is not:
i) 5-chloro-N-(4-fluorophenyl)-2-methoxy-N-methylnicotinamide;
ii) 5-chloro-N-ethyl-2-methoxy-N-(m-tolyl)nicotinamide;
iii) 5-chloro-N-ethyl-2-methoxy-N-(o-tolyl)nicotinamide;
iv) 5-bromo-N-(4-fluorophenyl)-2-methoxy-N-methylnicotinamide;
v) 5-bromo-N-ethyl-2-methoxy-N-(m-tolyl)nicotinamide;
vi) 5-bromo-N-ethyl-2-methoxy-N-(o-tolyl)nicotinamide;
vii) 5-bromo-N-(2,4-difluorophenyl)-2-methoxy-N-methylnicotinamide;
viii) 5-bromo-N-(2,4-difluorophenyl)-2-(dimethylamino)-N-methylnicotinamide;
ix) 5-bromo-2-(dimethylamino)-N-(3-isopropylphenyl)-N-methylnicotinamide;
x) N-(4-fluorophenyl)-2-methoxy-N,4,5,6-tetramethylnicotinamide;
xi) N-ethyl-2-methoxy-4,5,6-trimethyl-N-(m-tolyl)nicotinamide;
xii) N-ethyl-2-methoxy-4,5,6-trimethyl-N-(o-tolyl)nicotinamide;
xiii) N-(5-chloro-2-cyanophenyl)-2-methoxy-N,6-dimethylnicotinamide;
xiv) 5-chloro-N-(4-fluorophenyl)-2-methoxy-N,4,6-trimethylnicotinamide;
xv) 5-chloro-N-ethyl-2-methoxy-4,6-dimethyl-N-(m-tolyl)nicotinamide;
xvi) 5-chloro-N-ethyl-2-methoxy-4,6-dimethyl-N-(o-tolyl)nicotinamide;
xvii) 5-bromo-N-(4-fluorophenyl)-2-methoxy-N,4,6-trimethylnicotinamide;
xviii) 5-bromo-N-ethyl-2-methoxy-4,6-dimethyl-N-(m-tolyl)nicotinamide; or
xix) 5-bromo-N-ethyl-2-methoxy-4,6-dimethyl-N-(o-tolyl)nicotinamide;
   or a stereoisomer of any of the above compounds.

In this preferred embodiment of the second specific embodiment of the first aspect of the present invention, preferably R⁵ is fluoro, chloro, bromo, C₁-C₂ alkyl, C₁-C₂ haloalkyl, or C₃-C₄ cycloalkyl. Preferably R⁵ is fluoro, chloro, methyl, fluoromethyl, cyclopropyl, or cyclobutyl.

In a third specific embodiment of the first aspect, the present invention provides a compound of formula (I), or a pharmaceutically acceptable salt, N-oxide, solvate or prodrug thereof, wherein:
X is N;
Z is —C(O)—;
R¹ is -L-R³;
R² is C₁-C₃ alkyl;
L is —CH₂—, —CHMe-, or —C(C₂H₄)—;
R³ is a 6-membered aryl or heteroaryl group, wherein the 6-membered aryl or heteroaryl group is substituted with one or more substituents independently selected from halo, cyano, C₁-C₃ alkyl, C₁-C₃ haloalkyl, —O—(C₁-C₃ alkyl), —O—(C₁-C₃ haloalkyl), or —C(O)O(C₁-C₃ alkyl);
R⁴ is cyano, —O—(C₁-C₃ alkyl), —O—(C₁-C₃ haloalkyl), or —N—(C₁-C₃ alkyl)₂;
R⁵ is hydrogen, halo, C₁-C₃ alkyl, C₁-C₃ haloalkyl, or C₃-C₅ cycloalkyl;
R⁶ is hydrogen, methyl or halo;
R⁷ is hydrogen, methyl or halo;
provided that at least one of R⁵, R⁶ and R⁷ is not hydrogen; and
provided that the compound is not N-(4-cyanobenzyl)-2-methoxy-6-methyl-N-propylnicotinamide or a stereoisomer thereof.

Preferably,
R² is methyl or ethyl;
L is —CH₂—, —CHMe-, or —C(C₂H₄)—; and
R³ is phenyl substituted with one, two, three or four substituents independently selected from fluoro, chloro, bromo, cyano, C₁-C₂ alkyl, C₁-C₂ haloalkyl, —O—(C₁-C₂ alkyl), —O—(C₁-C₂ haloalkyl), or —C(O)O(C₁-C₂ alkyl).

Preferably,
R² is methyl or ethyl;
L is —CH₂—, —CHMe-, or —C(C₂H₄)—; and
R³ is phenyl substituted with one, two or three substituents independently selected from fluoro, chloro, cyano, methyl, fluoromethyl, methoxy, fluoromethoxy, or —CO₂Me.

R⁴ is cyano, —O—(C₁-C₃ alkyl), —O—(C₁-C₃ haloalkyl), or —N—(C₁-C₃ alkyl)₂. Preferably R⁴ is cyano, methoxy, ethoxy, halomethoxy, haloethoxy, or —N—(C₁-C₂ alkyl)₂. Preferably R⁴ is cyano, methoxy, ethoxy, fluoromethoxy, or fluoroethoxy.

R⁵ is hydrogen, halo, C₁-C₃ alkyl, C₁-C₃ haloalkyl, or C₃-C₅ cycloalkyl. Preferably R⁵ is hydrogen, fluoro, chloro, bromo, C₁-C₂ alkyl, C₁-C₂ haloalkyl, or C₃-C₄ cycloalkyl. Preferably R⁵ is hydrogen, fluoro, chloro, methyl, fluoromethyl, cyclopropyl, or cyclobutyl.

R⁶ is hydrogen, methyl or halo. Preferably R⁶ is hydrogen, methyl, fluoro, or chloro. Preferably R⁶ is hydrogen, methyl, or fluoro.

R⁷ is hydrogen, methyl or halo. Preferably R⁷ is hydrogen, methyl, fluoro, or chloro. Preferably R⁷ is hydrogen or methyl.

In a preferred embodiment of the third specific embodiment of the first aspect of the present invention, R⁵ is halo, C₁-C₃ alkyl, C₁-C₃ haloalkyl, or C₃-C₅ cycloalkyl. Preferably R⁵ is fluoro, chloro, bromo, C₁-C₂ alkyl, C₁-C₂ haloalkyl, or C₃-C₄ cycloalkyl. Preferably R⁵ is fluoro, chloro, methyl, fluoromethyl, cyclopropyl, or cyclobutyl.

A second aspect of the present invention provides a compound selected from:
N-benzyl-5-chloro-2-methoxy-N-methylnicotinamide;
5-chloro-N-(6-cyano-2,3-dihydro-1H-inden-1-yl)-2-methoxy-N-methylnicotinamide;
5-chloro-N-(7-cyano-2,3-dihydro-1H-inden-1-yl)-2-methoxy-N-methylnicotinamide;
(5-fluoro-2-methoxypyridin-3-yl)(2-(3-fluorophenyl)piperidin-1-yl)methanone;
5-fluoro-N-(1-(2-fluorophenyl)ethyl)-2-methoxy-N-methylnicotinamide;
5-fluoro-N-(1-(3-fluorophenyl)ethyl)-2-methoxy-N-methylnicotinamide;
5-fluoro-N-(6-fluoro-2,3-dihydro-1H-inden-1-yl)-2-methoxy-N-methylnicotinamide;
N-(2,4-difluorobenzyl)-2-methoxy-N,6-dimethylnicotinamide;
5-chloro-N-(2,3-dihydro-1H-inden-1-yl)-2-ethoxy-N-methylnicotinamide;
N-(chroman-4-yl)-5-fluoro-2-methoxy-N-methylnicotinamide;
N-(2, 3-dihydro-1H-inden-1-yl)-5-fluoro-2-methoxy-N-methylnicotinamide;
5-chloro-N-(chroman-4-yl)-2-methoxy-N-methylnicotinamide;
5-chloro-N-(6-fluoro-2,3-dihydro-1H-inden-1-yl)-2-methoxy-N-methylnicotinamide;
5-chloro-N-(2,3-dihydro-1H-inden-1-yl)-2-methoxy-N-methylnicotinamide;
5-chloro-N-(3-fluorobenzyl)-2-methoxy-N-methylnicotinamide;
5-chloro-N-(2,3-dihydrobenzofuran-3-yl)-2-methoxy-N-methylnicotinamide;
5-chloro-N-(8-fluorochroman-4-yl)-2-methoxy-N-methylnicotinamide;
5-fluoro-N-(8-fluorochroman-4-yl)-2-methoxy-N-methylnicotinamide;
N-(3-fluorobenzyl)-2-methoxy-N-methylnicotinamide;
N-(2-fluorobenzyl)-2-methoxy-N-methylnicotinamide;
5-fluoro-N-(1-(3-fluorophenyl)cyclopropyl)-2-methoxy-N-methylnicotinamide;
5-fluoro-N-(5-fluoro-2,3-dihydrobenzofuran-3-yl)-2-methoxy-N-methylnicotinamide;
N-(2-cyanobenzyl)-5-fluoro-2-methoxy-N-methylnicotinamide;
5-fluoro-N-(2-fluorobenzyl)-2-methoxy-N-methylnicotinamide;
N-(2,3-difluorobenzyl)-5-fluoro-2-methoxy-N-methylnicotinamide;
N-(2,4-difluorobenzyl)-5-fluoro-2-methoxy-N-methylnicotinamide;
5-chloro-N-(2,3-difluorobenzyl)-2-methoxy-N-methylnicotinamide;
5-chloro-N-(3,5-difluorobenzyl)-2-methoxy-N-methylnicotinamide;
5-fluoro-N-(6-fluoro-2,3-dihydrobenzofuran-3-yl)-2-methoxy-N-methylnicotinamide;
5-chloro-N-(7-fluorochroman-4-yl)-2-methoxy-N-methylnicotinamide;
5-chloro-N-(3-chloro-5-fluorobenzyl)-2-methoxy-N-methylnicotinamide;
5-chloro-N-(6-fluoro-2,3-dihydrobenzofuran-3-yl)-2-methoxy-N-methylnicotinamide;
N-benzyl-5-chloro-N-ethyl-2-methoxynicotinamide;
5-chloro-N-ethyl-N-(2-fluorobenzyl)-2-methoxynicotinamide;
5-chloro-N-ethyl-N-(3-fluorobenzyl)-2-methoxynicotinamide;
5-fluoro-N-(3-fluorobenzyl)-2-methoxy-N-methylnicotinamide;
2-((2-methoxy-5-methylpyridin-3-yl)sulfonyl)-1,2,3,4-tetrahydroisoquinoline;
2-((5-chloro-2-methoxypyridin-3-yl)sulfonyl)-1,2,3,4-tetrahydroisoquinoline;
7-fluoro-4-((2-methoxy-5-methylpyridin-3-yl)sulfonyl)-2,3,4,5-tetrahydrobenzo[f][1,4]oxazepine;
4-((5-chloro-2-methoxypyridin-3-yl)sulfonyl)-7-fluoro-2,3,4,5-tetrahydrobenzo[f][1,4]oxazepine;
7-fluoro-4-((2-methoxypyridin-3-yl)sulfonyl)-2,3,4,5-tetrahydrobenzo[f][1,4]oxazepine;
methyl 4-fluoro-2-((5-fluoro-2-methoxy-N-methylnicotinamido)methyl)benzoate;
5-chloro-N-(4-fluoro-2-(trifluoromethyl)benzyl)-2-methoxy-N-methylnicotinamide;
5-chloro-N-(2-cyano-4-fluorobenzyl)-2-methoxy-N-methylnicotinamide;
5-chloro-N-(1-(2-cyano-5-fluorophenyl)ethyl)-2-methoxy-N-methylnicotinamide;
N-(2-cyano-4,6-difluorobenzyl)-5-fluoro-2-methoxy-N-methylnicotinamide;
N-(1-(2-cyano-5-fluorophenyl)ethyl)-5-fluoro-2-methoxy-N-methylnicotinamide;
N-(1-(2-cyano-4-fluorophenyl)ethyl)-5-fluoro-2-methoxy-N-methylnicotinamide;
5-chloro-N-(1-(2-cyano-4-fluorophenyl)ethyl)-2-methoxy-N-methylnicotinamide;
N-(1-(2,4-difluorophenyl)ethyl)-5-fluoro-2-methoxy-N-methylnicotinamide;
2-(difluoromethoxy)-5-fluoro-N-(1-(2-fluorophenyl)ethyl)-N-methylnicotinamide;
N-(2,4-difluorobenzyl)-2-methoxy-N,5-dimethylnicotinamide;
N-(2,4-difluorobenzyl)-2-methoxy-N,4-dimethylnicotinamide;
2-(difluoromethoxy)-5-fluoro-N-(6-fluoro-2,3-dihydrobenzofuran-3-yl)-N-methylnicotinamide;
2-(difluoromethoxy)-5-fluoro-N-(7-fluoro-2,3-dihydrobenzofuran-3-yl)-N-methylnicotinamide;
5-chloro-2-(difluoromethoxy)-N-(3-fluoro-5-methylbenzyl)-N-methylnicotinamide;
5-chloro-2-(difluoromethoxy)-N-(6-fluoro-2,3-dihydrobenzofuran-3-yl)-N-methylnicotinamide;
5-chloro-N-(3,5-difluorobenzyl)-2-(difluoromethoxy)-N-methylnicotinamide;
5-chloro-N-(3-chloro-5-fluorobenzyl)-2-(difluoromethoxy)-N-methylnicotinamide;
5-chloro-2-(difluoromethoxy)-N-methyl-N-(3-methylbenzyl)nicotinamide;
5-chloro-2-(difluoromethoxy)-N-(3-methoxybenzyl)-N-methylnicotinamide;
5-chloro-2-(difluoromethoxy)-N-methyl-N-(3-(trifluoromethyl)benzyl) nicotinamide;
2-(difluoromethoxy)-5-fluoro-N-(1-(3-fluorophenyl)ethyl)-N-methylnicotinamide;
5-chloro-N-(3-chlorobenzyl)-2-(difluoromethoxy)-N-methylnicotinamide;
2-(difluoromethoxy)-N-(2,3-dihydrobenzofuran-3-yl)-5-fluoro-N-methylnicotinamide;
5-chloro-2-(difluoromethoxy)-N-(1-(3-fluorophenyl)ethyl)-N-methylnicotinamide;
2-(difluoromethoxy)-5-fluoro-N-(1-(3-fluorophenyl)ethyl)-N-methylnicotinamide;
5-chloro-N-(3-cyanobenzyl)-2-(difluoromethoxy)-N-methylnicotinamide;
5-chloro-2-(difluoromethoxy)-N-(2,3-dihydrobenzofuran-3-yl)-N-methylnicotinamide;
5-chloro-2-(difluoromethoxy)-N-(7-fluoro-2,3-dihydrobenzofuran-3-yl)-N-methylnicotinamide;
N-(2,3-difluorobenzyl)-2-(difluoromethoxy)-5-fluoro-N-methylnicotinamide;
N-(2,4-difluorobenzyl)-2-(difluoromethoxy)-5-fluoro-N-methylnicotinamide;
2-(difluoromethoxy)-5-fluoro-N-(3-fluorobenzyl)-N-methylnicotinamide;
2-(difluoromethoxy)-N-(3-fluorobenzyl)-N-methyl-5-(trifluoromethyl) nicotinamide;
5-cyclobutyl-2-(difluoromethoxy)-N-(3-fluorobenzyl)-N-methylnicotinamide;
5-chloro-2-(difluoromethoxy)-N-(3-(difluoromethoxy)benzyl)-N-methylnicotinamide;
5-chloro-2-(difluoromethoxy)-N-methyl-N-(3-(trifluoromethoxy)benzyl) nicotinamide;
5-chloro-N-(2,4-difluorobenzyl)-2-(difluoromethoxy)-N-methylnicotinamide;
5-chloro-2-(difluoromethoxy)-N-(3-fluorobenzyl)-N-methylnicotinamide;
5-chloro-N-(2, 3-difluorobenzyl)-2-(difluoromethoxy)-N-methylnicotinamide;
N-(3,5-difluorobenzyl)-2-(difluoromethoxy)-5-fluoro-N-methylpyridine-3-sulfonamide;
(R)-2-(difluoromethoxy)-5-fluoro-N-(1-(3-fluorophenyl)ethyl)-N-methylpyridine-3-sulfonamide;
(S)-2-(difluoromethoxy)-5-fluoro-N-(1-(3-fluorophenyl)ethyl)-N-methylpyridine-3-sulfonamide;
N-(3-cyanobenzyl)-2-(difluoromethoxy)-5-fluoro-N-methylpyridine-3-sulfonamide;
N-(2,3-difluorobenzyl)-2-(difluoromethoxy)-5-fluoro-N-methylpyridine-3-sulfonamide;
N-(3-chlorobenzyl)-2-(difluoromethoxy)-5-fluoro-N-methylpyridine-3-sulfonamide;
2-(difluoromethoxy)-N-ethyl-5-fluoro-N-(3-fluorobenzyl)pyridine-3-sulfonamide;
2-((2-(difluoromethoxy)-5-fluoropyridin-3-yl)sulfonyl)-1,2,3,4-tetrahydroisoquinoline;
2-((2-(difluoromethoxy)-5-fluoropyridin-3-yl)sulfonyl)-7-fluoro-1,2,3,4-tetrahydroisoquinoline;
2-(difluoromethoxy)-5-fluoro-N-(3-fluorobenzyl)-N-methylpyridine-3-sulfonamide;
5-chloro-2-(difluoromethoxy)-N-(3-fluorobenzyl)-N-methylpyridine-3-sulfonamide;
4-((2-(difluoromethoxy)-5-fluoropyridin-3-yl)sulfonyl)-7-fluoro-2,3,4,5-tetrahydrobenzo[f][1,4]oxazepine;
4-((5-chloro-2-(difluoromethoxy)pyridin-3-yl)sulfonyl)-7-fluoro-2,3,4,5-tetrahydrobenzo[f][1,4]oxazepine;
2-((5-chloro-2-(difluoromethoxy)pyridin-3-yl)sulfonyl)-6,8-difluoro-1,2,3,4-tetrahydroisoquinoline;
5-chloro-N-(2,6-difluorobenzyl)-2-methoxy-N-methylnicotinamide;
5-chloro-N-(2,6-dichlorobenzyl)-2-methoxy-N-methylnicotinamide;
5-chloro-2-(difluoromethoxy)-N-(1,1-dioxido-2,3-dihydrobenzo[b]thiophen-3-yl)-N-methylnicotinamide;
5-fluoro-2-((7-fluoro-2,3-dihydrobenzo[f][1,4]oxazepin-4(5H)-yl)sulfonyl)benzonitrile;
   or an enantiomer of any of the foregoing;
   so or a pharmaceutically acceptable salt, solvate or prodrug of any of the foregoing.

Preferably the compound of the first or second aspect has a chemical purity of 95% or more, preferably 96% or more, preferably 97% or more, preferably 98% or more, preferably 99% or more, preferably 99.5% or more, preferably 99.8% or more, preferably 99.9% or more, as measured by HPLC or UPLC.

Preferably the compound of the first or second aspect has a stereochemical purity of 95% or more, preferably 96% or more, preferably 97% or more, preferably 98% or more, preferably 99% or more, preferably 99.5% or more, preferably 99.8% or more, preferably 99.9% or more, as measured by XRPD or SFC.

A third aspect of the present invention provides a process for the preparation of a compound of formula (I) or a pharmaceutically acceptable salt, N-oxide, solvate or prodrug thereof, according to the first aspect of the present invention, wherein the process comprises:
(a) reacting a compound of formula (II), or a salt thereof, with an amine of formula (III), or a salt thereof:

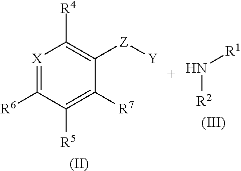

wherein X, Z, R¹, R², R⁴, R⁵, R⁶ and R⁷ are as defined in the first aspect of the present invention; Y is —OH, —OR⁸, or —Cl; and R⁸ is C₁-C₃ alkyl; or
(b) reacting a compound of formula (IV), or a salt thereof, with a compound of formula (V), or a salt thereof:

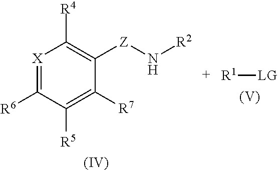

wherein X, Z, R¹, R², R⁴, R⁵, R⁶ and R⁷ are as defined in the first aspect of the present invention, and LG is a leaving group such as halo (such as fluoro, chloro, bromo, or iodo), a sulfate group (such as methyl sulfate), or a sulfonate group (such as mesylate, triflate, or tosylate);
and optionally thereafter carrying out one or more of the following procedures:
   converting a compound of formula (I) into another compound of formula (I);
   removing any protecting groups;
   forming a pharmaceutically acceptable salt or N-oxide.

In step (a), when Y is —OH, the compound of formula (II) is a carboxylic acid or a sulfonic acid. When Y is —OR⁸, the compound of formula (II) is an ester or a sulfonyl ester. When Y is —Cl, the compound of formula (II) is an acid chloride or a sulfonyl chloride.

Step (a) may be carried out by combining a compound of formula (II), or a salt thereof, with an amine of formula (III), or a salt thereof, in the presence of a base such as DIPEA or triethylamine. The reaction may be carried out in the presence of a coupling agent such as HATU or T₃P. Typically, the reaction is carried out in a solvent such as THF or DCM, preferably at a temperature of about 0° C. to about room temperature. Typically the reaction takes from 0.5 to 12 hours.

Step (b) may conveniently be carried out in the presence of a base such as NaH and in a solvent such as DMF. Typically, the reaction takes about 2 to 6 hours and is preferably carried out at a temperature of about 0° C. to about room temperature.

It will be appreciated by those skilled in the art that in the processes of the present invention certain functional groups such as phenol, hydroxy or amino groups in the reagents may need to be protected by protecting groups. Thus, the preparation of the compounds, salts, N-oxides, solvates and prodrugs of the present invention may involve, at an appropriate stage, the introduction and/or removal of one or more protecting groups.

The protection and deprotection of functional groups are described, for example, in ‘Protective Groups in Organic Chemistry’, edited by J. W. F. McOmie, Plenum Press (1973); ‘Greene's Protective Groups in Organic Synthesis’, 4th edition, T. W. Greene and P. G. M. Wuts, Wiley-Interscience (2007); and ‘Protecting Groups’, 3rd edition, P. J. Kocienski, Thieme (2005).

The compounds of formula (I) may be converted into a pharmaceutically acceptable salt thereof, preferably an acid addition salt such as a formate, hemi-formate, hydrochloride, hydrobromide, benzenesulfonate (besylate), saccharin (e.g. monosaccharin), trifluoroacetate, sulfate, nitrate, phosphate, acetate, fumarate, semi-fumarate, maleate, tartrate, lactate, citrate, pyruvate, succinate, valerate, propanoate, butanoate, malonate, oxalate, 1-hydroxy-2-naphthoate (xinafoate), methanesulfonate or p-toluenesulfonate salt. In one embodiment of the invention, the compounds of formula (I) are in the form of a hydrochloride, formate, hemi-formate or fumarate salt.

Compounds of formula (I) and their salts and N-oxides may be in the form of hydrates or solvates which form another embodiment of the present invention. Such solvates may be formed with common organic solvents including, but not limited to alcoholic solvents e.g. methanol, ethanol or isopropanol.

In one embodiment of the present invention, therapeutically inactive prodrugs are provided. Prodrugs are compounds which, when administered to a subject such as a human, are converted in whole or in part to a compound of formula (I). Generally, the prodrugs are pharmacologically inert chemical derivatives that can be converted in vivo to the active drug molecules to exert a therapeutic effect. Any of the compounds of formula (I) can be administered as a prodrug to increase the activity, bioavailability, or stability of the compound of formula (I) or to otherwise alter the properties of the compound of formula (I). Typical examples of prodrugs include compounds that have biologically labile protecting groups on a functional moiety of the active compound. Prodrugs include, but are not limited to compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts, N-oxides and solvates of such prodrugs as described above.

Where the compounds, salts, N-oxides, solvates and prodrugs of the present invention are capable of existing in stereoisomeric forms, it will be understood that the invention encompasses the use of all geometric and optical isomers (including atropisomers) and mixtures thereof. The use of tautomers and mixtures thereof also forms an embodiment of the present invention. The compounds, salts, N-oxides, solvates and prodrugs of the present invention may contain at least one chiral centre. The compounds, salts, N-oxides, solvates and prodrugs may therefore exist in at least two isomeric forms. The present invention encompasses racemic mixtures of the compounds, salts, N-oxides, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, more typically less than 1%, and most typically less than 0.5% by weight. Enantiomerically pure isomers are particularly desired.

The compounds, salts, N-oxides, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I. Therefore, the term “hydrogen”, for example, encompasses ¹H, ²H (D) and ³H (T). Similarly, carbon atoms are to be understood to include ¹¹C, ¹²C, ¹³C and ¹⁴C, nitrogen atoms are to be understood to include ¹³N, ¹⁴N and ¹⁵N, oxygen atoms are to be understood to include ¹⁵O, ¹⁶O, ¹⁷O and ¹⁸O, fluorine atoms are to be understood to include ¹⁸F and ¹⁹F, and iodine atoms are to be understood to include ¹²³I, ¹²⁴I, ¹²⁵I, ¹²⁷I and ¹³¹I.

In one embodiment, the compounds, salts, N-oxides, solvates and prodrugs of the present invention may be isotopically labelled. As used herein, an “isotopically labelled” compound is one in which the abundance of a particular nuclide at a particular atomic position within the molecule is increased above the level at which it occurs in nature. Any of the compounds, salts, N-oxides, solvates and prodrugs of the present invention can be isotopically labelled, for example, any of examples 1 to 120.

In one embodiment, the compounds, salts, N-oxides, solvates and prodrugs of the present invention may bear one or more radiolabels. Such radiolabels may be introduced by using radiolabel-containing reagents in the synthesis of the compounds, salts, N-oxides, solvates or prodrugs, or may be introduced by coupling the compounds, salts, N-oxides, solvates or prodrugs to chelating moieties capable of binding to a radioactive metal atom. Such radiolabelled versions of compounds, salts, N-oxides, solvates and prodrugs may be used, for example, in diagnostic imaging studies.

In one embodiment, the compounds, salts, N-oxides, solvates and prodrugs of the present invention may be tritiated, i.e. they contain one or more 3H (T) atoms. Any of the compounds, salts, N-oxides, solvates and prodrugs of the present invention can be tritiated, for example, any of examples 1 to 120.

The compounds, salts, N-oxides, solvates and prodrugs of the present invention may be amorphous or in a polymorphic form or a mixture of any of these, each of which is an embodiment of the present invention. The compounds, salts, N-oxides, solvates and prodrugs of the present invention have activity as pharmaceuticals and may be used in treating or preventing a disease, disorder or condition associated with KCNK13 activity.

Therefore, a fourth aspect of the present invention provides a compound of formula (I) or a pharmaceutically acceptable salt, N-oxide, solvate or prodrug thereof, according to the first aspect of the present invention, for use in therapy, in particular for use in treating or preventing a neurodegenerative disease, a psychiatric disease, a genetic disease, hearing loss, an ocular or retinal disease, a cardiovascular disease, an inflammatory disease, an autoimmune disease, or a metabolic disease.

The fourth aspect of the present invention also provides a compound of formula (I) or a pharmaceutically acceptable salt, N-oxide, solvate or prodrug thereof, according to the first aspect of the present invention, for use in treating or preventing Alzheimer's disease, Parkinson's disease, frontal temporal dementia, progressive supranuclear palsy (PSP) and related tauopathies, amyotrophic lateral sclerosis (ALS)/motor neuron disease (MND), traumatic brain injury, multiple sclerosis, stroke, ischemic insult, depression, stress, anxiety related disorder (including social and generalised anxiety), post-traumatic stress disorder (PTSD), schizophrenia, bipolar disorder, cryopyrin-associated periodic syndrome (CAPS) (including Muckle-Wells syndrome (MWS), familial cold autoinflammatory syndrome (FCAS), chronic infantile neurological cutaneous and articular (CINCA) syndrome, and neonatal onset multisystem inflammatory disease (NOMID)), age related hearing loss, genetic related hearing loss (including NLRP3 mutation related hearing loss), autoimmune related hearing loss, macular degeneration, age related macular degeneration, diabetic retinopathy, atherosclerosis, myocardial infarction, ischemia, rheumatoid arthritis, gout, Lupus, asthma, respiratory inflammation, inflammatory or autoimmune skin disease, psoriasis, inflammatory bowel disease, colitis, metabolic syndrome, non-alcoholic fatty liver disease (NAFLD), non-alcoholic steatohepatitis (NASH), fibrosis, or diabetes.

A fifth aspect of the present invention provides a use of a compound of formula (I) or a pharmaceutically acceptable salt, N-oxide, solvate or prodrug thereof, according to the first aspect of the present invention, for the manufacture of a medicament for treating or preventing a neurodegenerative disease, a psychiatric disease, a genetic disease, hearing loss, an ocular or retinal disease, a cardiovascular disease, an inflammatory disease, an autoimmune disease, or a metabolic disease.

The fifth aspect of the present invention also provides a use of a compound of formula (I) or a pharmaceutically acceptable salt, N-oxide, solvate or prodrug thereof, according to the first aspect of the present invention, for the manufacture of a medicament for treating or preventing Alzheimer's disease, Parkinson's disease, frontal temporal dementia, progressive supranuclear palsy (PSP) and related tauopathies, amyotrophic lateral sclerosis (ALS)/motor neuron disease (MND), traumatic brain injury, multiple sclerosis, stroke, ischemic insult, depression, stress, anxiety related disorder (including social and generalised anxiety), post-traumatic stress disorder (PTSD), schizophrenia, bipolar disorder, cryopyrin-associated periodic syndrome (CAPS) (including Muckle-Wells syndrome (MWS), familial cold autoinflammatory syndrome (FCAS), chronic infantile neurological cutaneous and articular (CINCA) syndrome, and neonatal onset multisystem inflammatory disease (NOMID)), age related hearing loss, genetic related hearing loss (including NLRP3 mutation related hearing loss), autoimmune related hearing loss, macular degeneration, age related macular degeneration, diabetic retinopathy, atherosclerosis, myocardial infarction, ischemia, rheumatoid arthritis, gout, Lupus, asthma, respiratory inflammation, inflammatory or autoimmune skin disease, psoriasis, inflammatory bowel disease, colitis, metabolic syndrome, non-alcoholic fatty liver disease (NAFLD), non-alcoholic steatohepatitis (NASH), fibrosis, or diabetes.

A sixth aspect of the present invention provides a method of treating or preventing a neurodegenerative disease, a psychiatric disease, a genetic disease, hearing loss, an ocular or retinal disease, a cardiovascular disease, an inflammatory disease, an autoimmune disease, or a metabolic disease; the method comprising administering a therapeutically or prophylactically effective amount of a compound of formula (I) or a pharmaceutically acceptable salt, N-oxide, solvate or prodrug thereof, according to the first aspect of the present invention, to a patient in need thereof.

The sixth aspect of the present invention also provides a method of treating or preventing Alzheimer's disease, Parkinson's disease, frontal temporal dementia, progressive supranuclear palsy (PSP) and related tauopathies, amyotrophic lateral sclerosis (ALS)/motor neuron disease (MND), traumatic brain injury, multiple sclerosis, stroke, ischemic insult, depression, stress, anxiety related disorder (including social and generalised anxiety), post-traumatic stress disorder (PTSD), schizophrenia, bipolar disorder, cryopyrin-associated periodic syndrome (CAPS) (including Muckle-Wells syndrome (MWS), familial cold autoinflammatory syndrome (FCAS), chronic infantile neurological cutaneous and articular (CINCA) syndrome, and neonatal onset multisystem inflammatory disease (NOMID)), age related hearing loss, genetic related hearing loss (including NLRP3 mutation related hearing loss), autoimmune related hearing loss, macular degeneration, age related macular degeneration, diabetic retinopathy, atherosclerosis, myocardial infarction, ischemia, rheumatoid arthritis, gout, Lupus, asthma, respiratory inflammation, inflammatory or autoimmune skin disease, psoriasis, inflammatory bowel disease, colitis, metabolic syndrome, non-alcoholic fatty liver disease (NAFLD), non-alcoholic steatohepatitis (NASH), fibrosis, or diabetes; the method comprising administering a therapeutically or prophylactically effective amount of a compound of formula (I) or a pharmaceutically acceptable salt, N-oxide, solvate or prodrug thereof, according to the first aspect of the present invention, to a patient in need thereof.

Unless stated otherwise, in any of the fourth, fifth or sixth aspects of the invention, the subject or patient may be any human or other animal. Typically, the subject or patient is a mammal, more typically a human or a domesticated mammal such as a cow, pig, lamb, sheep, goat, horse, cat, dog, rabbit, mouse etc. Most typically, the subject is a human.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disorder or condition in question. Persons at risk of developing a particular disorder or condition generally include those having a family history of the disorder or condition, or those who have been identified by genetic testing or screening to be particularly susceptible to developing the disorder or condition or those in the prodromal phase of a disorder.

The terms “treat”, “treatment” and “treating” include improvement of the conditions described herein. The terms “treat”, “treatment” and “treating” include all processes providing slowing, interrupting, arresting, controlling, or stopping of the state or progression of the conditions described herein, but does not necessarily indicate a total elimination of all symptoms or a cure of the condition. The terms “treat”, “treatment” and “treating” are intended to include therapeutic as well as prophylactic treatment of such conditions.

For the above-mentioned therapeutic uses the dosage administered will, of course, vary with the compound employed, the mode of administration, the treatment desired and the disorder indicated. For example, the daily dosage of a compound of the invention (that is, a compound of formula (I) or a pharmaceutically acceptable salt, N-oxide, solvate or prodrug thereof), if inhaled, may be in the range from 0.05 micrograms per kilogram body weight (μg/kg) to 1 milligram per kilogram body weight (mg/kg). Alternatively, if the compound is administered orally or parenterally, then the daily dosage of the compound of the invention may be in the range from 0.01 micrograms per kilogram body weight (μg/kg) to 500 milligrams per kilogram body weight (mg/kg). The desired dosage may be presented at an appropriate interval such as once every other day, once a day, twice a day, three times a day or four times a day.

The compounds of formula (I) and pharmaceutically acceptable salts, N-oxides, solvates and prodrugs thereof may be used on their own, but will generally be administered in the form of a pharmaceutical composition in which the active ingredient is in association with a pharmaceutically acceptable adjuvant, diluent or carrier.

Therefore, a seventh aspect of the present invention provides a pharmaceutical composition comprising a compound of formula (I) or a pharmaceutically acceptable salt, N-oxide, solvate or prodrug thereof, according to the first aspect of the present invention, in association with a pharmaceutically acceptable adjuvant, diluent or carrier, and optionally one or more other therapeutic agents.

The invention still further provides a process for the preparation of a pharmaceutical composition of the invention which comprises mixing a compound of formula (I) or a pharmaceutically acceptable salt, N-oxide, solvate or prodrug thereof, according to the first aspect of the present invention, with a pharmaceutically acceptable adjuvant, diluent or carrier.

Conventional procedures for the selection and preparation of suitable pharmaceutical formulations are described in, for example, “Pharmaceutics—The Science of Dosage Form Design”, M. E. Aulton, Churchill Livingstone, 1988.

Pharmaceutically acceptable adjuvants, diluents or carriers that may be used in the pharmaceutical compositions of the invention are those conventionally employed in the field of pharmaceutical formulation, and include, but are not limited to sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, dipotassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the present invention may be administered orally, parenterally, by inhalation spray, rectally, nasally, buccally, vaginally, ocularly, topically or via an implanted reservoir. Oral administration is preferred. The pharmaceutical compositions of the invention may contain any conventional non-toxic pharmaceutically acceptable adjuvants, diluents or carriers. The term parenteral as used herein includes subcutaneous, intracutaneous, intradermal, intravenous, intramuscular, intra-articular, intrasynovial, intrasternal, intrathecal, intralesional, intracranial, intratracheal, intraperitoneal, intraarticular, and epidural injection or infusion techniques. The term topical as used herein includes transdermal, mucosal, sublingual and topical ocular administration.

The pharmaceutical compositions may be in the form of a sterile injectable preparation, for example, as a sterile injectable aqueous or oleaginous suspension. The suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable diluents and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

The pharmaceutical compositions of this invention may be orally administered in any orally acceptable dosage form including, but not limited to capsules, tablets, caplets, troches, lozenges, powders, granules, and aqueous suspensions, solutions and dispersions. These dosage forms are prepared according to techniques well-known in the art of pharmaceutical formulation. In the case of tablets for oral use, carriers which are commonly used include lactose, sodium and calcium carbonate, sodium and calcium phosphate, and corn starch. Lubricating agents, such as magnesium stearate, stearic acid or talc, are also typically added. If desired, the tablets may be coated with a material, such as glyceryl monostearate or glyceryl distearate, to delay absorption in the gastrointestinal tract. Tablets may also be effervescent and/or dissolving tablets. For oral administration in capsule form, useful diluents include lactose and dried corn starch. When aqueous suspensions are administered orally, the active ingredient may be combined with emulsifying and suspending agents. If desired, certain sweetening and/or flavouring and/or colouring agents and/or preservatives may be added to any oral dosage form.

The pharmaceutical compositions of the invention may also be administered in the form of suppositories for rectal administration. These compositions can be prepared by mixing the active ingredient with a suitable non-irritating excipient which is solid at room temperature but liquid at the rectal temperature and therefore will melt in the rectum to release the active ingredient. Such materials include, but are not limited to cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilising or dispersing agents known in the art.

For ocular administration, the compounds, salts, N-oxides, solvates or prodrugs of the invention will generally be provided in a form suitable for topical administration, e.g. as eye drops. Suitable forms may include ophthalmic solutions, gel-forming solutions, sterile powders for reconstitution, ophthalmic suspensions, ophthalmic ointments, ophthalmic emulsions, ophthalmic gels, and ocular inserts. Alternatively, the compounds, salts, N-oxides, solvates or prodrugs of the invention may be provided in a form suitable for other types of ocular administration, for example as intraocular preparations (including as irrigating solutions, as intraocular, intravitreal or juxtascleral injection formulations, or as intravitreal implants), as packs or corneal shields, as intracameral, subconjunctival or retrobulbar injection formulations, or as iontophoresis formulations.

For transdermal and other topical administration, the compounds, salts, N-oxides, solvates or prodrugs of the invention will generally be provided in the form of ointments, cataplasms (poultices), pastes, powders, dressings, creams, plasters or patches.

Depending on the mode of administration, the pharmaceutical composition will preferably comprise from 0.05 to 99% by weight, more preferably from 0.05 to 80% by weight, still more preferably from 0.1 to 70% by weight, and even more preferably from 0.1 to 50% by weight of active ingredient, all percentages by weight being based on total composition.

The compounds of the invention may also be administered in conjunction with other compounds used for the treatment of the above conditions.

The invention therefore further relates to combination therapies wherein a compound of the invention or a pharmaceutical composition or formulation comprising a compound of the invention is administered with another therapeutic agent or agents for the treatment of one or more of the conditions previously indicated. The compound of the invention or the pharmaceutical composition or formulation comprising the compound of the invention may be administered simultaneously with, separately from or sequentially to the one or more other therapeutic agents. The compound of the invention and the one or more other therapeutic agents may be comprised in the same pharmaceutical composition or formulation, or in separate pharmaceutical compositions or formulations, i.e. in the form of a kit. The one or more other therapeutic agents may, for example, be an antibody designed to clear forms of tau, alpha synuclein, or fragments of amyloid.

Typically, the mode of administration selected is that most appropriate to the disorder, disease or condition to be treated or prevented. Where one or more further active agents are administered, the mode of administration may be the same as or different to the mode of administration of the compound or pharmaceutical composition of the invention.

Such combination products employ the compounds of this invention within the dosage range described herein and the other pharmaceutically active agent(s) within approved dosage ranges.

### Definitions

An “alkyl” group may be linear (i.e. straight-chained) or branched. Examples of alkyl groups include methyl, ethyl, n-propyl, iso-propyl, n-butyl, iso-butyl, sec-butyl, tert-butyl, n-pentyl, 2-methyl-1-butyl, 3-methyl-1-butyl, 3-methyl-2-butyl, and 2,2-dimethyl-1-propyl groups. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” group is an unsaturated alkyl group having one or more carbon-carbon double bonds. Examples of alkenyl groups include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” group is an unsaturated alkyl group having one or more carbon-carbon triple bonds. Examples of alkynyl groups include ethynyl, propargyl, but-1-ynyl and but-2-ynyl groups. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” group refers to a hydrocarbyl ring, wherein the hydrocarbyl ring may be saturated or unsaturated (including aromatic) and may include one or more (such as one, two, three or four) heteroatoms, e.g. N, O or S, in its carbon skeleton. Examples of cyclic groups include cycloalkyl, cycloalkenyl, aryl, heterocyclic and heteroaryl groups as discussed below. A cyclic group may be monocyclic, bicyclic (e.g. bridged, fused or spiro), or polycyclic. Typically, a cyclic group is a 5- to 11-membered group, which means that it contains from 5 to 11 ring atoms.

A “cycloalkyl” group is a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl group may be monocyclic, bicyclic (e.g. bridged, fused or spiro), or polycyclic.

A “cycloalkenyl” group is a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl group may be monocyclic, bicyclic (e.g. bridged, fused or spiro), or polycyclic.

An “aryl” group is an aromatic hydrocarbyl ring. The term “aryl” includes monocyclic aromatic hydrocarbons (such as phenyl) and polycyclic fused-ring aromatic hydrocarbons (such as naphthyl, anthracenyl and phenanthrenyl). Unless stated otherwise, the term “aryl” does not include “heteroaryl”.

A “heterocyclic” group is a non-aromatic cyclic group which includes one or more carbon atoms and one or more (such as one, two, three or four) heteroatoms, e.g. N, O or S, in the ring structure. A heterocyclic group may be monocyclic, bicyclic (e.g. bridged, fused or spiro), or polycyclic. Typically, a heterocyclic group is a 4- to 14-membered heterocyclic group, which means it contains from 4 to 14 ring atoms. More typically, a heterocyclic group is a 5- to 11-membered heterocyclic group, which means it contains from 5 to 11 ring atoms. Heterocyclic groups include unsaturated heterocyclic groups (such as azetinyl, tetrahydropyridinyl, and 2-oxo-1H-pyridinyl) and saturated heterocyclic groups. Examples of saturated monocyclic heterocyclic groups are azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl and thiomorpholinyl groups. Examples of saturated bicyclic heterocyclic groups are quinuclidinyl, 8-azabicyclo[3.2.1]octanyl, 2-azaspiro[3.3]heptanyl, 6-azaspiro[2.5]octanyl and hexahydro-1H-pyrrolizinyl groups.

A “heteroaryl” group is an aromatic cyclic group which includes one or more carbon atoms and one or more (such as one, two, three or four) heteroatoms, e.g. N, O or S, in the ring structure. Typically, a heteroaryl group is a 5- to 14-membered heteroaryl group, which means it contains from 5 to 14 ring atoms. More typically, a heteroaryl group is a 5- to 10-membered heteroaryl group, which means it contains from 5 to 10 ring atoms. The term “heteroaryl” includes monocyclic aromatic heterocycles (such as pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyridazinyl, pyrimidinyl, pyrazinyl, triazinyl and tetrazinyl) and polycyclic fused-ring aromatic heterocycles (such as indolyl, benzofuranyl, benzothiophenyl, benzoxazolyl, benzisoxazolyl, benzothiazolyl, benzoisothiazolyl, benzimidazolyl, 1H-imidazo[4,5-b]pyridinyl, 1H-imidazo[4,5-c]pyridinyl, quinolinyl, isoquinolinyl, quinoxalinyl, quinazolinyl, phthalazinyl and cinnolinyl). Examples of heteroaryl groups include the following:

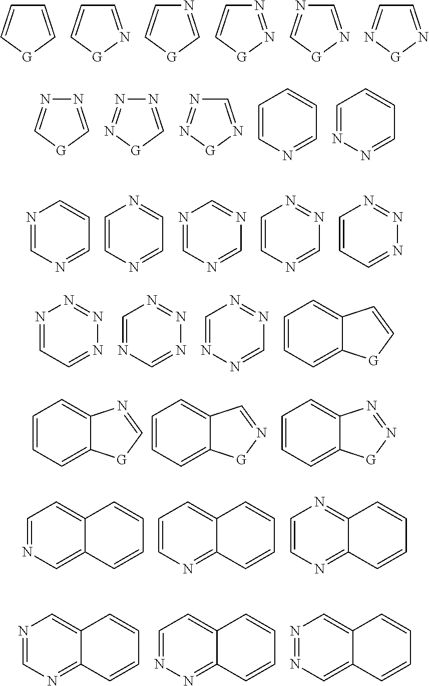

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

The term “halo” includes fluoro, chloro, bromo and iodo.

Unless stated otherwise, where a group is prefixed by the term “halo”, such as a “haloalkyl” or “halomethyl” group, it is to be understood that the group in question is substituted with one or more (such as one, two, three, four or five) halo groups independently selected from fluoro, chloro, bromo and iodo. Typically, the maximum number of halo substituents is limited only by the number of hydrogen atoms available for substitution on the corresponding group without the halo prefix. For example, a “halomethyl” group may contain one, two or three halo substituents. A “haloethyl” or “halophenyl” group may contain one, two, three, four or five halo substituents. Similarly, unless stated otherwise, where a group is prefixed by a specific halo group, it is to be understood that the group in question is substituted with one or more (such as one, two, three, four or five) of the specific halo groups. For example, the term “fluoromethyl” refers to a methyl group substituted with one, two or three fluoro groups, and the term “fluoroethyl” refers to an ethyl group substituted with one, two, three, four or five fluoro groups.

A “hydroxyalkyl” group is an alkyl group substituted with one or more (such as one, two or three) hydroxyl (—OH) groups. Typically a hydroxyalkyl group has one or two hydroxyl substituents, more typically a hydroxyalkyl group has one hydroxyl substituent.

Unless stated otherwise, any reference to an element is to be considered a reference to all isotopes of that element. Thus, for example, unless stated otherwise, any reference to hydrogen is considered to encompass all isotopes of hydrogen including ¹H, ²H (D) and ³H (T). Therefore, for the avoidance of doubt, it is noted that, for example, the terms “alkyl” and “methyl” include, for example, trideuteriomethyl.

Unless stated otherwise, any reference to a compound or group is to be considered a reference to all tautomers of that compound or group.

When any chemical group or moiety is described as substituted, it will be appreciated that the number and nature of substituents will be selected so as to avoid sterically undesirable combinations.

### EXAMPLES

The present invention will now be further explained by reference to the following illustrative examples, in which the starting materials and reagents used are available from commercial suppliers or prepared via literature procedures or procedures similar to the ones described in this application.

‘Room temperature’, as used in the present specification, means a temperature in the range from about 18° C. to about 25° C.

For the purposes of the present invention, for all of the experimental details described below, where there are reaction conditions described, such as reagents, solvents and temperatures, above and/or below an arrow in a graphical representation, it is to be understood that these reaction conditions, in particular solvents and temperatures, are not essential to the reaction being carried out and may be varied.

### Abbreviations

DIPEA N,N-diisopropylethylamine
DMF dimethylformamide
dppp 3-diphenylphosphanylpropyl(diphenyl)phosphane
EtOAc ethyl acetate
EtOH ethanol
h hour
HATU 1-[bis(dimethylamino)methylene]-1H-1,2,3-triazolo[4,5-b]pyridinium 3-oxide hexafluorophosphate
HPLC high-performance liquid chromatography
IPA isopropanol
MeCN acetonitrile
MeOH methanol
min minutes
MW microwave
NCS N-chlorosuccinimide
py pyridine
Pd(amphos)Cl₂ bis(di-tert-butyl(4-dimethylaminophenyl)phosphine) dichloropalladium (II)
Pd₂(dba)₃ tris(dibenzylideneacetone)dipalladium (0)
Pd(dppf)Cl₂ [1,1′-bis(diphenylphosphino)ferrocene]dichloropalladium (II)
RT room temperature
SFC supercritical fluid chromatography
T₃P 2,4,6-tripropyl-1,3,5,2,4,6-trioxatriphosphorinane-2,4,6-trioxide
TEA triethylamine
TPP triphenylphosphine
THF tetrahydrofuran
Xantphos 4,5-bis(diphenylphosphino)-9,9-dimethylxanthene

### General Procedures

Nuclear magnetic resonance (NMR) spectra were recorded at 400 MHz and at 300.3K, 298.2K, 294.1K or 293K unless otherwise stated; the chemical shifts (8) are reported in parts per million. Spectra were recorded using a Bruker™ 400 AVANCE instrument fitted with a 5 mm BBFO probe with instrument controlled by Bruker TopSpin 2.1 software, or a Bruker 400 AVANCE-III HD instrument fitted with a 5 mm BBO smart probe or a 5 mm BBFO probe with instrument controlled by Bruker TopSpin 3.5 software, or a Bruker 400 AVANCE-III instrument fitted with a 5 mm BBFO probe with instrument controlled by Bruker Topspin 3.0 software, or a Bruker 300 MHz AVANCE II instrument fitted with a 5 mm DUL probe with instrument controlled by Bruker TopSpin 1.3 software, or 5 mm BBFO probe controlled by Bruker Topspin 3.2 software, or a Bruker 400 AVANCE instrument fitted with a 5 mm iprobe or smart probe with instrument controlled by Bruker TopSpin 4.0.9 or Bruker TopSpin 4.1.1 software.

Reactions were monitored using one or more of the following:
Waters ACQUITY UPLC H-Class with single quadrupole LCMS; Chromatographic Conditions: Mobile Phase A: 0.1% FA in H2O; Mobile Phase B: 0.1% FA in ACN; or Mobile Phase A: 0.1% TFA in H2O; Mobile Phase B: 0.1% TFA in ACN; or Mobile Phase A: 10 mM ammonium acetate in H2O; Mobile Phase B: CAN. Column: Acquity UPLC BEH C18 (50 mm×2.1 mm) 1.7 μm; Column Temp: 40° C.; Sample Temp: RT; Detection (nm): 220; Flow Rate: 0.6 mL/min. Analysis Time: 4.6 min.
Agilent 1290 infinity II UPLC coupled with 6130 quadrupole LCMS; Chromatographic Conditions: Mobile Phase A: 0.1% Formic acid (FA) in H2O; Mobile Phase B: 0.1% FA in ACN; Column: Acquity UPLC BEH C18 (50 mm×2.1 mm) 1.7 μm; Column Temp: 40° C.; Sample Temp: RT; Detection (nm): 220; Flow Rate: 0.6 mL/min; Analysis Time: 4.6 min.
Agilent 1290 infinity II UPLC coupled with 6130 quadrupole LCMS; Chromatographic Conditions: Mobile Phase A: 0.037% TFA in water; Mobile Phase B: 0.018% TFA in acetonitrile; Column: Xtimate® C18 2.1*30 mm, 3 μm; Column Temp.: 50° C.; Sample Temp.: RT; Detection (nm): 220 nm and 254 nm; Flow Rate: 1.0 mL/min; Analysis Time: 4.0 min. Measured Mass Range: 100 to 1500.
Dionex Ultima 3000 UHPLC Coupled with Thermo Scientific LCQ Fleet Ion Trap; Chromatographic Conditions: Mobile Phase A: 10 mM Ammonium Formate in Water: ACN (95:5); Mobile Phase B: 10 mM Ammonium Formate in Water: ACN (5:95); Column: XBridge BEH C18 (50 mm×3.0 mm) 2.5 μm; Column Temp: 40° C.; Sample Temp: RT; Detection (nm): 220; Flow Rate: 0.6 mL/min; Analysis Time: 4.6 min.

MS conditions: Measured Mass Range: 100 to 1500. Ion mode: +ve & −ve. Desolvation Temp.: 350° C. Source Gas Flow: Desolvation [L/Hr]: 800; Cone [L/Hr]: 50. Source Voltage: Capillary (Kv): 3.50; Cone (V): 35.

Purity was assessed using one or more of the following:
Ultra Performance Liquid Chromatography (UPLC) with UV (photodiode array) detection over a wide range of wavelengths, normally 220-450 nm, using a Waters™ Acquity UPLC system equipped with Acquity UPLC BEH, HSS or HSS T3, Xbridge C18 columns (2.1 mm id×50 mm long) operated at 35° C. Mobile phases typically consisted of acetonitrile mixed with water containing either 0.1% formic acid, 0.1% TFA or 10 mM ammonium acetate.
UPLC with UV (photodiode array) detection over a wide range of wavelengths, normally 220-450 nm, using Shimadzu™ Nexera X2 UPLC controlled by Lab Solution software equipped with Acquity UPLC BEH, HSS or HSS T3, Xbridge C18 columns (2.1 mm id×50 mm long) operated at 35° C. Mobile phases typically consisted of acetonitrile mixed with water containing either 0.1% formic acid, 0.1% TFA or 10 mM ammonium acetate.
UPLC with UV (photodiode array) detection over a wide range of wavelengths, normally 220-254 nm, using Shimadzu Nexera X2 UPLC controlled by Lab Solution software equipped with Acquity UPLC BEH, HSS or HSS T3 C18 columns (2.1 mm id×50 mm long) operated at 50° C. Mobile phases typically consisted of acetonitrile mixed with water containing 0.037% TFA. Mass spectra were recorded with a Shimadzu single quadrupole mass spectrometer using DUIS ionisation.

Compounds were purified using Biotage or ISCO® instrument using normal phase chromatography on silica or by preparative high performance liquid chromatography (HPLC).

Compounds were purified using Grace purifier, Buchi Reveleris X2 flash purification system or Biotage using normal phase chromatography on silica, using Reveleris SRC flash cartridges, Interchim™ PuriFlash cartridges or Kinesis™ Telos silica cartridges, or on basic silica using Biotage KP-NH cartridges, or by reverse phase chromatographic methods using Reveleris RP flash cartridges or by Biotage Isolute SCX-2 or Phenomenex™ Strata ABW catch-release cartridges, or by preparative high performance liquid chromatography (HPLC).

Preparative HPLC was performed using Gilson GX-281 system using Phenomenex C18 75*30 mm*3 μm; Xtimate C18 100*30 mm*10 μm; Xtimate C18 150*40 mm*10 μm; Xtimate C18 150*40 mm*10 μm; Phenomenex C18 75*30 mm*3 μm or Gemini NX C18 10*150 mm*5 μm columns at room temperature. Mobile phases typically consisted of acetonitrile mixed with water containing either 0.225% formic acid or 0.05% ammonia+10 nM NH₄HCO₃, unless otherwise stated.

Preparative HPLC was performed using Agilent Technologies™ 1100 Series system or a Waters autopurification LC/MS system typically using Waters (19 mm id×250 mm long) C18 columns such as XBridge™ or SunFire™ 5 m materials at RT. Mobile phases typically consisted of acetonitrile mixed with water containing either 0.1% formic acid or 10 mM ammonium acetate, unless otherwise stated.

Super Critical Fluid Chromatography (SFC) chiral separations were performed on a Waters UPCC/Investigator/UPCC with QDA/UPCC with ELSD investigator system, using a flow rate of 60 g to 120 g/min, temperature of RT to 40° C. and a pressure of 100 bar. Mobile phases typically consisted of supercritical CO2 and a polar solvent such as acetonitrile, methanol, ethanol or isopropanol with or without modifier like diethylamine. Column type and eluent are detailed for individual examples. Columns: Chiralcel OJ-H (250*21 mm, 5 μm), Chiralpak-IG (250*30 mm, 5 μm), Chiralpak-IE (250*30 mm, 5 μm), Chiralpak-AD-H (250*30 mm, 5 μm), Chiralcel OX-H (250*21 mm, 5 μm), YMC SC (250*30 mm, 5 μm), Chiralpak AS-H (250*30 mm, 5 μm), R,R Whelk-01 (250*30 mm, 5 μm), Chromega chiral CCO (250*30 mm, 5 μm), Chromega Chiral CCA (250*30 mm, 5 μm), Chiralpak IA (250*30 mm, 5 μm), Lux Cellulose-02 (250*30 mm, 5 μm), Chiralpak OD-H (250*30 mm, 5 μm); Detection: 200 nm to 400 nm; Sample Diluent: Acetonitrile, Methanol; Injection: 0.1 ml to 5 ml; Isocratic Ratio: 5% to 50% of mobile phase.

### 1. Synthesis of Intermediates

### Intermediate 1: N-(Chroman-4-yl)-5-fluoro-2-methoxynicotinamide

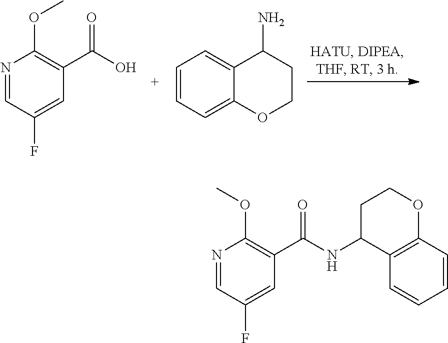 [SMILES: COc1ncc(F)cc1C(=O)O ; NC1CCOc2ccccc21 ; COc1ncc(F)cc1C(=O)NC1CCOc2ccccc21]

A solution of 5-fluoro-2-methoxynicotinic acid (0.15 g, 0.8 mmol) in THF (15 mL) was treated with DIPEA (0.309 g, 2.4 mmol), HATU (0.456 g, 1.2 mmol) and chroman-4-amine (0.116 g, 0.96 mmol) and stirred at RT for 12 h. Water was added to the reaction mixture which was then extracted with EtOAc (3×20 mL). The organic phases were dried over Na₂SO₄ and evaporated under reduced pressure to obtain crude product. Purification by reverse phase column chromatography (0-80% MeOH in 0.1% aq. ammonium bicarbonate) gave N-(chroman-4-yl)-5-fluoro-2-methoxynicotinamide (0.10 g, 38% yield) as an off-white solid. MS m/z: 303.21 (M+H).

### Intermediate 2: 5-Chloro-N-(2,3-dihydro-1H-inden-1-yl)-2-methoxynicotinamide

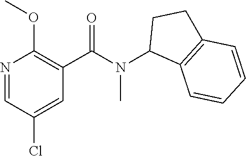 [SMILES: COc1ncc(Cl)cc1C(=O)N(C)C1CCc2ccccc21]

By following the procedure employed in Intermediate 1 using 5-chloro-2-methoxynicotinic acid (0.1 g, 0.5 mmol) and 2,3-dihydro-1H-inden-1-amine (0.08 g, 0.6 mmol), in DMF (10 mL), crude product was obtained. Purification by flash column chromatography (Davisil silica, 10-30% EtOAc/Pet ether) afforded 5-chloro-N-(2,3-dihydro-1H-inden-1-yl)-2-methoxynicotinamide (0.07 g, 43%) as a thick gum. ¹H NMR (400 MHz, CDCl₃): δ 8.54 (d, J=2.0 Hz, 1H), 8.21 (d, J=1.6 Hz, 1H), 8.15-8.05 (br s, 1H), 7.35-7.20 (m, 4H), 5.70 (q, J=8.0 Hz, 1H), 4.02 (s, 3H), 3.1-2.90 (m, 2H), 2.78-2.68 (m, 1H), 1.95-1.85 (m, 1H). MS m/z: 303.0 (M+H).

### Intermediate 3: N-(2, 3-dihydro-1H-inden-1-yl)-5-fluoro-2-methoxynicotinamide

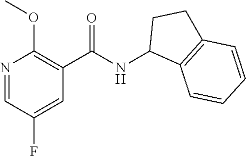 [SMILES: COc1ncc(F)cc1C(=O)NC1CCc2ccccc21]

By following the procedure employed in Intermediate 1 using 5-fluoro-2-methoxynicotinic acid (0.3 g, 1.754 mmol) and 2,3-dihydro-1H-inden-1-amine (0.35 g, 2.6315 mmol) in DMF (10 mL), crude product was obtained. Purification by flash column chromatography (Davisil silica, 10-30% EtOAc/Pet ether) afforded N-(2,3-dihydro-1H-inden-1-yl)-5-fluoro-2-methoxynicotinamide (0.3 g, 60%) as a thick gum. ¹H NMR (400 MHz, CDCl₃): δ 8.34 (d, J=2.8 Hz, 1H), 8.30-8.10 (m, 2H), 7.40-7.10 (m, 5H), 5.70 (q, J=8.0 Hz, 1H), 4.00 (s, 3H), 3.15-2.85 (m, 2H), 2.80-2.70 (m, 1H), 1.90-1.75 (m, 1H). MS m/z: 287.20 (M+H)

### Intermediate 4: 5-Chloro-N-(chroman-4-yl)-2-methoxynicotinamide

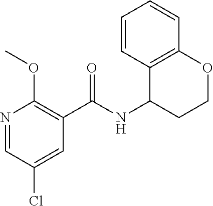 [SMILES: COc1ncc(Cl)cc1C(=O)NC1CCOc2ccccc21]

By following the procedure employed in Intermediate 1 using 5-chloro-2-methoxynicotinic acid (0.3 g, 1.604 mmol) and chroman-4-amine (0.36 g, 2.4064 mmol) in DMF (10 mL), crude product was obtained. Purification by flash column chromatography (Davisil silica, 10-30% EtOAc/Pet ether) afforded 5-chloro-N-(chroman-4-yl)-2-methoxynicotinamide (0.3 g, 59%) as gummy material. ¹H NMR (400 MHz, DMSO-d₆): δ 8.67 (s, 1H), 8.34 (d, J=2.8 Hz, 1H), 8.03 (d, J=2.4 Hz, 1H), 7.18 (d, J=1.2 Hz, 1H), 7.16-7.14 (m, 1H), 6.92-6.90 (m, 1H), 6.89-6.77 (m, 1H), 5.30-5.15 (m, 1H), 4.25-4.20 (m, 2H), 3.91 (s, 3H), 2.10-1.90 (m, 2H). MS m/z: 319.25 (M+H).

### Intermediate 5: 5-Chloro-N-(2,3-dihydro-1H-inden-1-yl)-2-ethoxynicotinamide

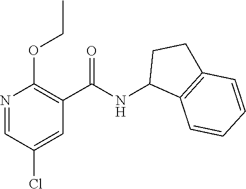 [SMILES: CCOc1ncc(Cl)cc1C(=O)NC1CCc2ccccc21]

By following the procedure employed in Intermediate 1 using 5-chloro-2-ethoxynicotinic acid (0.9 g, 4.5 mmol) and 2,3-dihydro-1H-inden-1-amine (0.6 g, 4.5 mmol) in THF (15 mL), crude product was obtained. Purification by flash column chromatography (Davisil silica, 10-30% EtOAc/Pet ether) afforded 5-chloro-N-(2,3-dihydro-1H-inden-1-yl)-2-ethoxynicotinamide (0.5 g, 36%) as a thick gum. MS m/z: 317.14 (M+H)

### Intermediate 6: 5-Chloro-N-(6-fluoro-2,3-dihydro-1H-inden-1-yl)-2-methoxynicotinamide

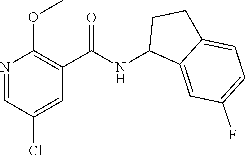 [SMILES: COc1ncc(Cl)cc1C(=O)NC1CCc2ccc(F)cc21]

By following the procedure employed in Intermediate 1 using 5-chloro-2-methoxynicotinic acid (0.5 g, 2.7 mmol) and 6-fluoro-2,3-dihydro-1H-inden-1-amine (0.4 g, 2.7 mmol) in THF (10 mL), crude product was obtained. Purification by flash column chromatography (Davisil silica, 10-40% EtOAc/Pet ether) afforded 5-chloro-N-(6-fluoro-2,3-dihydro-1H-inden-1-yl)-2-methoxynicotinamide (0.6 g, 70%) as a brown liquid. ¹H NMR (400 MHz, CDCl₃): δ 8.53 (s, 1H), 8.22 (s, 1H), 8.10 (br s, 1H), 7.20-7.10 (m, 1H), 7.10-6.90 (m, 1H), 5.50-5.45 (m, 1H), 4.05 (s, 3H), 3.10-3.00 (m, 2H), 2.00-1.85 (m, 1H), 1.50-1.40 (m, 1H). MS m/z: 321.26 (M+H), 71.85%.

### Intermediate 7: 8-Fluorochroman-4-amine

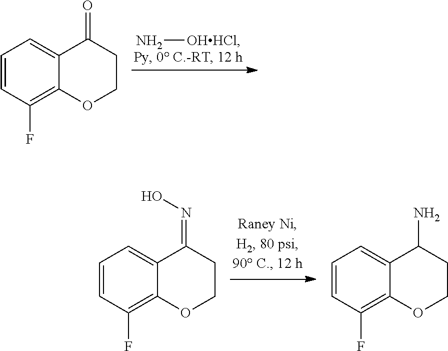

Step 1: A stirred solution of 8-fluorochroman-4-one (3.0 g, 18.07 mmol) in pyridine (3 mL) was treated with hydroxylamine hydrochloride (1.9 g, 27.11 mmol) at 0° C. and stirred at RT for 16 h. The reaction was quenched with ice cold water and extracted with EtOAc (2×25 mL). The organic phase was washed with 1N HCl solution, brine solution, before being dried over Na₂SO₄ and evaporated under reduced pressure to obtain (Z)-8-fluorochroman-4-one oxime (2.25 g, 69%) as a thick liquid. ¹H NMR (400 MHz, CDCl₃): δ 8.05-7.80 (br s, 1H), 7.59 (d, J=8.4 Hz, 1H), 7.09-7.06 (m, 1H), 6.90-6.84 (m, 1H), 4.33 (t, J=6.0 Hz, 2H), 3.02 (t, J=6.4 Hz, 2H). MS m/z: 182.18 (M+H)

Step 2: To a stirred solution of (Z)-8-fluorochroman-4-one oxime (2.0 g, 11.0 mmol) in ethanol (30 mL) was added Raney nickel (1.88 g, 22 mmol), under 80 psi Hydrogen pressure and the reaction was stirred at 90° C. for 12 h. After this time, the reaction mixture was filtered through a celite bed, washed with ethanol and the combined filtrate was evaporated under reduced pressure to give the crude product. Purification by flash column chromatography using (Davisil silica, 0-10% MeOH/DCM) afforded 8-fluorochroman-4-amine (1.1 g, 60%) as a pale-yellow semi solid. MS m/z: 168.19 (M+H)

### Intermediate 8: 5-Chloro-N-(8-fluorochroman-4-yl)-2-methoxynicotinamide

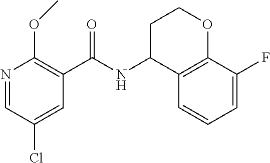 [SMILES: COc1ncc(Cl)cc1C(=O)NC1CCOc2c(F)cccc21]

By following the procedure employed in Intermediate 1 using 5-chloro-2-methoxynicotinic acid (0.35 g, 1.9 mmol) and 8-fluorochroman-4-amine (0.28 g, 2.07 mmol) in DMF (10 mL), crude product was obtained. Purification by flash column chromatography (Davisil silica, 10-40% EtOAc/Pet ether) afforded 5-chloro-N-(8-fluorochroman-4-yl)-2-methoxynicotinamide (0.35 g, 56%) as a pale yellow thick liquid. ¹H NMR (400 MHz, CDCl₃): δ 8.51 (d, J=2.4 Hz, 1H), 8.22 (d, J=2.8 Hz, 1H), 8.15-8.05 (m, 1H), 7.10-6.80 (m, 2H), 6.90-6.80 (m, 1H), 5.45-5.35 (m, 1H), 4.45-4.35 (m, 1H), 4.35-4.25 (m, 1H), 4.00 (s, 3H), 2.42-2.32 (m, 1H), 2.20-2.10 (m, 1H). MS m/z: 337.26 (M+H).

### Intermediate 9: 5-Fluoro-N-(8-fluorochroman-4-yl)-2-methoxynicotinamide

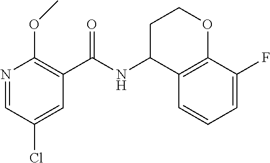 [SMILES: COc1ncc(Cl)cc1C(=O)NC1CCOc2c(F)cccc21]

By following the procedure employed in Intermediate 2 using 5-fluoro-2-methoxynicotinic acid (0.4 g, 2.3 mmol) and 8-fluorochroman-4-amine (Intermediate 7) (0.34 g, 2.53 mmol) in DMF (10 mL), crude product was obtained.

Purification by flash column chromatography (Davisil silica, 10-40% EtOAc/Pet ether) afforded 5-fluoro-N-(8-fluorochroman-4-yl)-2-methoxynicotinamide (0.6 g, 80%) as an off-white solid. ¹H NMR (400 MHz, CDCl₃): δ 8.51 (d, J=2.4 Hz, 1H), 8.22 (d, J=2.8 Hz, 1H), 8.15-8.05 (m, 1H), 7.10-6.95 (m, 2H), 6.90-6.80 (m, 1H), 5.45-5.35 (m, 1H), 4.45-4.35 (m, 1H), 4.35-4.25 (m, 1H), 4.00 (s, 3H), 2.42-2.32 (m, 1H), 2.20-2.10 (m, 1H). MS m/z: 321.30 (M+H).

### Intermediate 10: N-Benzyl-5-chloro-2-methoxynicotinamide

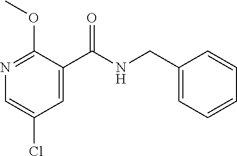 [SMILES: COc1ncc(Cl)cc1C(=O)NCc1ccccc1]

By following the procedure employed in Intermediate 1 using 5-chloro-2-methoxynicotinic acid (0.25 g, 1.34 mmol) and phenylmethanamine (0.16 g, 1.47 mmol) in THF (10 mL), crude product was obtained. Purification by flash column chromatography using (Davisil silica, 10-20% EtOAc/Pet ether) afforded N-benzyl-5-chloro-2-methoxynicotinamide (0.33 g, 89%) as an off-white solid. ¹H NMR (400 MHz, CDCl₃): δ 8.53 (d, J=2.8 Hz, 1H), 8.25-8.15 (m, 2H), 7.40-7.25 (m, 4H), 4.68 (d, J=6.0 Hz, 2H), 4.05 (s, 3H). MS m/z: 277.20 (M+H)

### Intermediate 11: 5-Chloro-N-(2-fluorobenzyl)-2-methoxynicotinamide

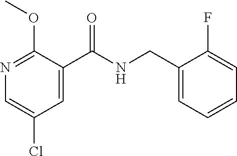 [SMILES: COc1ncc(Cl)cc1C(=O)NCc1ccccc1F]

By following the procedure employed in Intermediate 1 using 5-chloro-2-methoxynicotinic acid (0.2 g, 1.07 mmol) and (2-fluorophenyl)methanamine (0.15 g, 1.18 mmol) in THF (10 mL), crude product was obtained. Purification by flash column chromatography (Davisil silica, 10-20% EtOAc/Pet ether) afforded 5-chloro-N-(2-fluorobenzyl)-2-methoxynicotinamide (0.27 g, 86%) as an off-white solid. ¹H NMR (400 MHz, CDCl₃): δ 8.49 (d, J=2.8 Hz, 1H), 8.31 (br s, 1H), 8.20 (d, J=2.8 Hz, 1H), 7.45-7.35 (m, 1H), 7.30-7.22 (m, 1H), 7.15-7.05 (m, 2H), 4.71 (d, J=6.0 Hz, 2H), 4.07 (s, 3H). MS m/z: 295.22 (M+H).

### Intermediate 12: 5-Chloro-N-(3-fluorobenzyl)-2-methoxynicotinamide

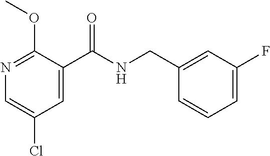 [SMILES: COc1ncc(Cl)cc1C(=O)NCc1cccc(F)c1]

By following the procedure employed in Intermediate 1 using 5-chloro-2-methoxynicotinic acid (0.2 g, 1.07 mmol) and (3-fluorophenyl)methanamine (0.15 g, 1.18 mmol) in THF (10 mL), crude product was obtained. Purification by flash column chromatography using (Davisil silica, 10-20% EtOAc/Pet ether) afforded 5-chloro-N-(3-fluorobenzyl)-2-methoxynicotinamide (0.15 g, 48%) as an off-white solid. MS m/z: 295.08 (M+H).

### Intermediate 13: 3-(Methylamino)-2,3-dihydro-1H-indene-5-carbonitrile

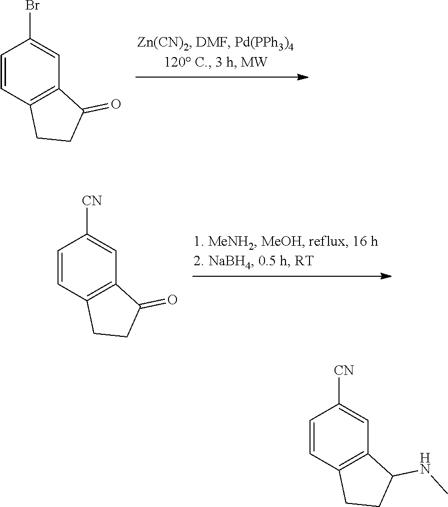

Step 1: To a stirred solution of 6-bromo-2,3-dihydro-1H-inden-1-one (0.5 g, 2.3 mmol) in DMF (10 ml) in a microwave vial was added Zn(CN)₂ (0.55 g 4.7 mmol). The vial was then purged with nitrogen for to min, after which time Pd(PPh₃)₄ (0.139, 0.11 mmol) was added and the resulting mixture irradiated at 120° C. in a microwave for 3 h. After this time, the reaction mixture was diluted with water and extracted with EtOAc (3×20 mL). The combined organic phases were washed with ice-cold water, dried over Na₂SO₄ and evaporated under reduced pressure to obtain the crude material. Purification by flash column chromatography using (Davisil silica, 0-20% EtOAc/Pet ether) afforded 3-oxo-2,3-dihydro-1H-indene-5-carbonitrile (0.30 g, 80%) as a brown liquid. MS m/z: 158.15 (M+H).

Step 2: A stirred solution of 3-oxo-2, 3-dihydro-1H-indene-5-carbonitrile (0.3 g, 1.9 mmol) in MeOH (8 mL) was treated with MeNH₂ (1 M in MeOH, 0.05 mL, 1.9 mmol) and the resulting mixture was refluxed for 16 h. After this time, the reaction mixture was cooled to 0° C., at which point NaBH₄ (0.14 g, 3.8 mmol) was added then and stirred for 0.5 h at RT. The reaction was then quenched with sat. NH₄Cl solution and extracted with 10% MeOH/DCM (3×15 mL). The organic phases were dried over sodium sulphate and evaporated under reduced pressure to give the crude product. This was diluted with water and the pH of the solution was adjusted to 3 by using 5N HCl. The resultant mixture was then washed with ethyl acetate (2×25 mL) to remove impurities. The aqueous layer was then adjusted to neutral pH by using sat. NaHCO₃ solution. The aqueous layer was extracted with ethyl acetate (2×25 mL) and the combined organic layers were dried over sodium sulphate and concentrated under reduced pressure to afford 3-(methyl amino)-2,3-dihydro-1H-indene-5-carbonitrile (0.15 g, 46%) as a thick gum. MS m/z: 173.12 (M+H).

### Intermediate 14: 3-(Methylamino)-2,3-dihydro-1H-indene-4-carbonitrile

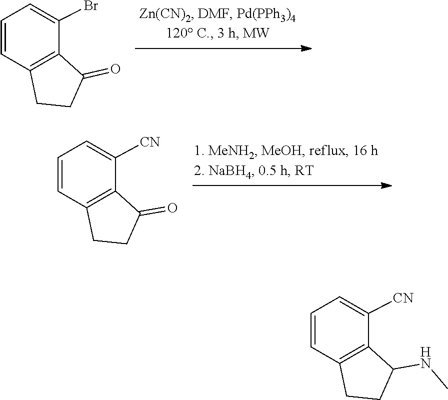

Step 1: By following the procedure employed in Intermediate 13 Step 1 using 7-bromo-2,3-dihydro-1H-inden-1-one (0.5 g, 2.3 mmol) crude product was obtained. Purification by flash column chromatography using (Davisil silica, 0-20% EtOAc/Pet ether) afforded 3-oxo-2,3-dihydro-1H-indene-4-carbonitrile (0.3 g, 80%) a pale yellow liquid. ¹H NMR (400 MHz, CDCl₃): δ 7.80-7.65 (m, 3H), 3.20 (t, J=6.0 Hz, 2H), 2.82-2.78 (m, 2H). MS m/z: 158.15 (M+H).

Step 2: By following the procedure employed in Intermediate 13 Step 2 using 3-oxo-2,3-dihydro-1H-indene-4-carbonitrile (0.3 g, 1.9 mmol), 3-(methylamino)-2,3-dihydro-1H-indene-4-carbonitrile (0.15 g, 46%) was obtained as a clear liquid. MS m/z: 173.22 (M+H).

### Intermediate 15: 5-Chloro-N-(2,3-dihydrobenzofuran-3-yl)-2-methoxynicotinamide

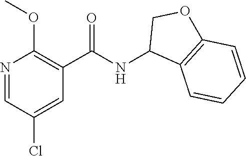 [SMILES: COc1ncc(Cl)cc1C(=O)NC1COc2ccccc21]

By following the procedure employed in Intermediate 1 using 5-chloro-2-methoxynicotinic acid (0.3 g, 1.6 mmol) and 2,3-dihydrobenzofuran-3-amine (0.24 g, 1.76 mmol) in DMF (10 mL), crude product was obtained. Purification by flash column chromatography using (Davisil silica, 20-30% EtOAc/Pet ether) afforded 5-chloro-N-(2,3-dihydrobenzofuran-3-yl)-2-methoxynicotinamide (0.25 g, 51%) as an off-white solid. ¹H NMR (400 MHz, CDCl₃): δ 8.49 (d, J=2.8 Hz, 1H), 8.22 (d, J=2.8 Hz, 1H) 8.20-8.10 (m, 1H), 7.40-7.35 (m, 1H), 7.30-7.22 (m, 1H), 7.00-6.92 (m, 1H), 6.92-6.85 (m, 1H), 5.85-5.75 (m, 1H), 4.85-4.70 (m, 1H), 4.45-4.40 (m, 1H), 3.98 (s, 3H). MS m/z: 305.17 (M+H).

### Intermediate 16: 5-Fluoro-N-(5-fluoro-2,3-dihydrobenzofuran-3-yl)-2-methoxynicotinamide

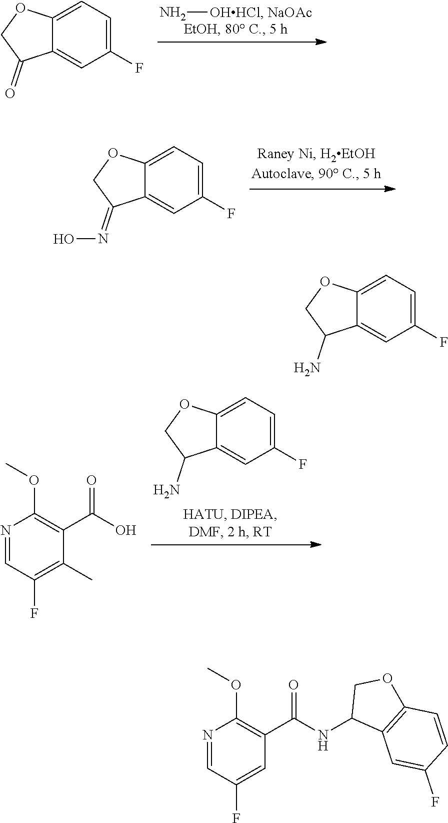

Step 1: To a stirred solution of 5-fluorobenzofuran-3(2H)-one (0.8 g, 5.2631 mmol) in EtOH (60 mL) was added NaOAc (0.89 g, 10.5263 mmol), NH₂—OH·HCl (0.75 g, 10.5263 mmol) and stirred at 80° C. for 5 h. After this time, the solvent was evaporated under reduced pressure to give a residue which was dissolved in ethyl acetate (3×20 mL) before being washed with water (20 mL), dried over Na₂SO₄ and evaporated under reduced pressure to obtain crude (Z)-5-fluorobenzofuran-3(2H)-one oxime (0.8 g, 90%) as pale yellow solid. MS m/z: 168.05 (M+H).

Step 2: To a stirred solution of (Z)-5-fluorobenzofuran-3(2H)-one oxime (0.9 g, 5.3892 mmol) in ethanol (30 mL) was added Raney nickel (3 g) and under 120 psi hydrogen pressure the reaction was stirred at 90° C. for 5 h. After this time, the reaction mixture was filtered through a celite bed, washed with ethanol and the filtrate was evaporated under reduced pressure to give the crude product. Purification by flash column chromatography (Davisil silica, 0-20% MeOH/DCM) afforded 5-fluoro-2,3-dihydrobenzofuran-3-amine (0.4 g, 48%) as a pale yellow semi solid. MS m/z: 154.05 (M+H).

Step 3: By following the procedure employed in Intermediate 1 using 5-fluoro-2-methoxynicotinic acid (0.12 g, 0.78 mmol) and 5-fluoro-2,3-dihydrobenzofuran-3-amine (0.15 g, 1.02 mmol) in DMF (10 mL). Purification gave 5-fluoro-N-(5-fluoro-2,3-dihydrobenzofuran-3-yl)-2-methoxynicotinamide (0.2 g, 83%) as a thick liquid. MS m/z: 307.15 (M+H).

### Intermediate 17: 5-fluoro-2-methoxy-N-methylnicotinamide

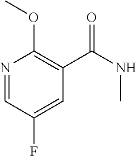

By following the procedure employed in Intermediate 1 using 5-fluoro-2-methoxynicotinic acid (0.25 g, 1.46 mmol) and methylamine in THF solution (5 mL) the crude product was obtained. Purification by column chromatography (Davisil silica, 20-30% EtOAc/Pet ether), gave 5-fluoro-2-methoxy-N-methylnicotinamide (0.21 g, 78%) as a pale-yellow solid. ¹H NMR (400 MHz, CDCl₃): δ 8.30-8.27 (m, 1H), 8.09 (d, J=3.2 Hz, 1H) 8.00-7.90 (br s, 1H), 4.09 (s, 3H), 3.03 (d, J=4.8 Hz, 3H). MS m/z: 185.05 (M+H).

### Intermediate 18: 5-Chloro-N-(2,3-difluorobenzyl)-2-methoxynicotinamide

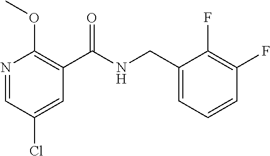 [SMILES: COc1ncc(Cl)cc1C(=O)NCc1cccc(F)c1F]

By following the procedure employed in Intermediate 1 using 5-chloro-2-methoxynicotinic acid (0.12 g, 0.78 mmol) and (2,3-difluorophenyl)methanamine (0.15 g, 1.02 mmol) in DMF (10 mL), the crude product was obtained. Purification by column chromatography (Davisil silica, 20-30% EtOAc/Pet ether), gave 5-chloro-N-(2,3-difluorobenzyl)-2-methoxynicotinamide (0.18 g, 90%) as a pale-yellow gum. MS m/z: 313.07 (M+H).

Intermediate 19: N-(3-Fluorobenzyl)-2-methoxynicotinamide

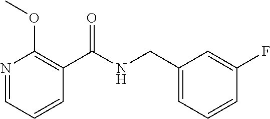 [SMILES: COc1ncccc1C(=O)NCc1cccc(F)c1]

By following the procedure employed in Intermediate 1 using 2-methoxynicotinic acid (0.3 g, 1.96 mmol) and (3-fluorophenyl)methanamine (0.37 g, 2.94 mmol) in DMF (10 mL), the crude product was obtained. Purification by flash column chromatography (Davisil silica, 20-30% EtOAc/Pet ether) afforded N-(3-fluorobenzyl)-2-methoxynicotinamide (0.4 g, 78%) as an off-white solid. ¹H NMR (400 MHz, DMSO-d₆): δ 8.90-8.80 (m, 1H), 8.35-8.30 (m, 1H), 8.15-8.10 (m, 1H), 7.40-7.35 (m, 1H), 7.20-7.00 (m, 4H), 4.52 (d, J=6.0 Hz, 2H), 4.00 (s, 3H). MS m/z: 261.23 (M+H).

### Intermediate 20: N-(2-Fluorobenzyl)-2-methoxynicotinamide

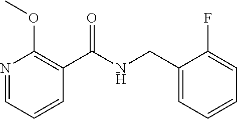 [SMILES: COc1ncccc1C(=O)NCc1ccccc1F]

By following the procedure employed in Intermediate 1 using 2-methoxynicotinic acid (0.3 g, 1.96 mmol) and (2-fluorophenyl)methanamine (0.37 g, 2.94 mmol) in DMF (10 mL), the crude product was obtained. Purification by flash column chromatography using (Davisil silica, 20-30% EtOAc/Pet ether) afforded N-(2-fluorobenzyl)-2-methoxynicotinamide (0.4 g, 78%) as an off-white solid. ¹H NMR (400 MHz, DMSO-d₆): δ 8.79 (d, J=5.6 Hz, 1H), 8.35-8.30 (m, 1H), 8.15-8.10 (m, 1H), 7.40-7.35 (m, 1H), 7.31-7.29 (m, 1H), 7.20-7.12 (m, 3H), 4.55 (d, J=6.0 Hz, 2H), 3.99 (s, 3H). MS m/z: 261.11 (M+H).

### Intermediate 21: 5-Fluoro-N-(1-(3-fluorophenyl)cyclopropyl)-2-methoxynicotinamide

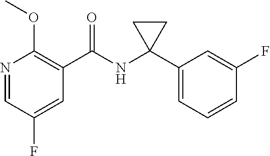 [SMILES: COc1ncc(F)cc1C(=O)NC1(c2cccc(F)c2)CC1]

By following the procedure employed in Intermediate 1 using 5-fluoro-2-methoxynicotinic acid (0.2 g, 1.16 mmol) and 1-(3-fluorophenyl)cyclopropan-1-amine (0.94 g, 1.29 mmol) in THF (10 mL), crude product was obtained. The crude was purified by flash column chromatography (Davisil silica, 10-30% EtOAc/Pet ether) to afford 5-fluoro-N-(1-(3-fluorophenyl)cyclopropyl)-2-methoxynicotinamide (0.22 g, 62%) as an off-white solid. ¹H NMR (400 MHz, DMSO-d₆): δ 8.53 (s, 1H), 8.25 (dd, J=3.20, 8.40 Hz, 1H), 8.12 (d, J=3.20 Hz, 1H), 7.22-7.28 (m, 1H), 7.03 (dd, J=0.80, 1.80 Hz, 1H), 7.01 (dd, J=0.80, 1.60 Hz, 1H), 6.99-6.86 (m, 1H), 4.12 (s, 3H), 1.38 (s, 4H). MS m/z: 305.22 (M+H).

### Intermediate 22: N-(2-Cyanobenzyl)-5-fluoro-2-methoxynicotinamide

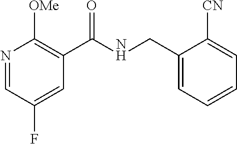 [SMILES: COc1ncc(F)cc1C(=O)NCc1ccccc1C#N]

By following the procedure employed in Intermediate 1 using 5-fluoro-2-methoxynicotinic acid (0.1 g, 0.6 mmol) and 2-(aminomethyl)benzonitrile (0.12 g, 0.72 mmol) in DMF (10 mL), the crude product was obtained. Purification by flash column chromatography (Davisil silica, 20-30% EtOAc/Pet ether) afforded N-(2-cyanobenzyl)-5-fluoro-2-methoxynicotinamide (0.12 g, 72%) as an off-white solid. MS m/z: 286.19 (M+H).

### Intermediate 23: 5-Fluoro-N-(2-fluorobenzyl)-2-methoxynicotinamide

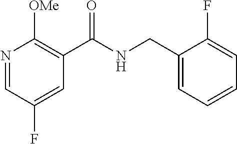 [SMILES: COc1ncc(F)cc1C(=O)NCc1ccccc1F]

By following the procedure employed in Intermediate 1 using 5-fluoro-2-methoxynicotinic acid (0.2 g, 1.2 mmol) and (2-fluorophenyl)methanamine (0.15 g, 1.2 mmol) in DMF (10 mL), the crude product was obtained. Purification by flash column chromatography (Davisil silica, 10-20% EtOAc/Pet ether) afforded 5-fluoro-N-(2-fluorobenzyl)-2-methoxynicotinamide (0.25 g, 77%) as an off-white solid. ¹H NMR (400 MHz, CDCl₃): δ 8.45-8.35 (br s, 1H), 8.30-8.27 (dd, J=3.2 Hz, 8.4 Hz, 1H), 8.10 (d, J=3.2 Hz, 1H), 7.45-7.35 (m, 1H), 7.15-7.05 (m, 2H), 4.71 (d, J=6.0 Hz, 2H), 4.07 (s, 3H). MS m/z: 279.22 (M+H).

### Intermediate 24: N-(2,3-Difluorobenzyl)-5-fluoro-2-methoxynicotinamide

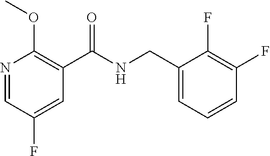 [SMILES: COc1ncc(F)cc1C(=O)NCc1cccc(F)c1F]

By following the procedure employed in Intermediate 1 using 5-fluoro-2-methoxynicotinic acid (0.15 g, 0.87 mmol) and (2,3-difluorophenyl)methanamine (0.165 g, 1.14 mmol) in DMF (10 mL), the crude product was obtained. Purification by flash column chromatography (Davisil silica and 20-30% EtOAc/Pet ether), gave N-(2,3-difluorobenzyl)-5-fluoro-2-methoxynicotinamide (0.2 g, 77%) as a pale brown sticky solid. MS m/z: 297.23 (M+H).

### Intermediate 25: N-(2,4-Difluorobenzyl)-5-fluoro-2-methoxynicotinamide

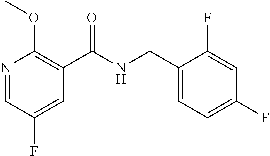 [SMILES: COc1ncc(F)cc1C(=O)NCc1ccc(F)cc1F]

By following the procedure employed in Intermediate 1 using 5-fluoro-2-methoxynicotinic acid (0.15 g, 0.87 mmol) and (2,4-difluorophenyl)methanamine (0.165 g, 1.14 mmol) in DMF (10 mL), the crude product was obtained. Purification by flash column chromatography (Davisil silica, 20-30% EtOAc/Pet ether) afforded N-(2,4-difluorobenzyl)-5-fluoro-2-methoxynicotinamide (0.20 g, 77%) as a pale brown sticky solid. MS m/z: 297.23 (M+H).

### Intermediate 26: 5-Chloro-N-(3,5-difluorobenzyl)-2-methoxynicotinamide

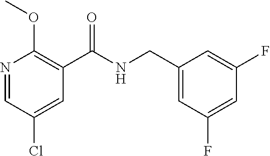 [SMILES: COc1ncc(Cl)cc1C(=O)NCc1cc(F)cc(F)c1]

By following the procedure employed in Intermediate 1 using 5-chloro-2-methoxynicotinic acid (0.2 g, 1.06 mmol) and (3,5-difluorophenyl)methanamine (0.2 g, 1.38 mmol) in DMF (10 mL), the crude product was obtained. Purification by flash column chromatography (Davisil silica, 20-30% EtOAc/Pet. ether) afforded 5-chloro-N-(3,5-difluorobenzyl)-2-methoxynicotinamide (0.15 g, 45%) as a pale brown sticky solid. MS m/z: 297.28 (M+H).

### Intermediate 27: N-(2-Bromo-4,6-difluorobenzyl)-5-fluoro-2-methoxy-N-methylnicotinamide

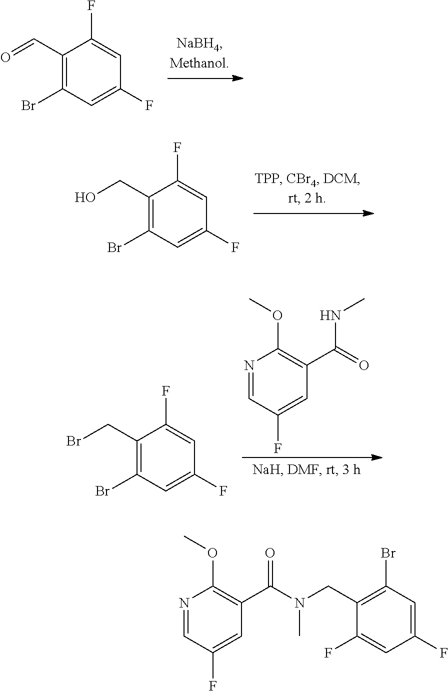 [SMILES: O=Cc1c(F)cc(F)cc1Br ; OCc1c(F)cc(F)cc1Br ; Fc1cc(F)c(CBr)c(Br)c1 ; CNC(=O)c1cc(F)cnc1OC ; COc1ncc(F)cc1C(=O)N(C)Cc1c(F)cc(F)cc1Br]

Step 1: To a stirred solution of 2-bromo-4,6-difluorobenzaldehyde (0.25 g, 1.136 mmol) in methanol (15 mL) was added sodium borohydride (0.042 g, 1.136 mmol) at 0° C. and stirred at RT for 3 h. After this time, the reaction mixture was concentrated under vacuum to give a residue which was dissolved in water and extracted with EtOAc (2×30 mL). The combined organic phases were dried over Na₂SO₄ and evaporated under reduced pressure to give (2-bromo-4,6-difluorophenyl)methanol (0.23 mg, 91%) as a pale yellow sticky solid. ¹H NMR (400 MHz, DMSO-d₆): δ 7.50-7.40 (m, 1H), 7.40-7.25 (m, 1H), 5.25 (br s, 1H), 4.54 (s, 2H). MS m/z: 222.98 (M+H).

Step 2: To a stirred solution of (2-bromo-4,6-difluorophenyl)methanol (0.2 g, 0.885 mmol) in DCM (15 mL) was added triphenylphosphine (0.524 g, 1.70 mmol). The reaction was stirred for 10 min at RT at which point CBr₄ (0.592 g, 1.70 mmol) was added and the reaction mixture was stirred at RT for 3 h. After this time, the reaction was quenched with ice cold water at 0° C. and extracted with EtOAc (2×30 mL). The combined organic phases were dried over Na₂SO₄ and evaporated under reduced pressure to give 1-bromo-2-(bromomethyl)-3,5-difluorobenzene (0.2 g, 78%) as a pale yellow oil. ¹H NMR (400 MHz, DMSO-d₆): δ 7.20-7.17 (m, 1H), 6.88-6.80 (m, 1H), 4.60 (s, 2H). MS m/z: 284.91 (M+H).

Step 3: To a stirred solution of 5-fluoro-2-methoxy-N-methylnicotinamide (Intermediate 17) (0.2 g, 1.15 mmol) in THF (5 mL) was added NaH (0.23 g, 5.77 mmol) at 0° C. and stirred for 10 min at which point 1-bromo-2-(bromomethyl)-3,5-difluorobenzene (0.33 g, 1.15 mmol) was added at 0° C. After stirring for 2 h, the reaction was quenched with water at 0° C. and extracted with EtOAc (3×20 mL). The combined organic layers were washed with ice-cold water, dried over Na₂SO₄ and evaporated under reduced pressure to obtain the crude product. Purification by flash column chromatography using (Davisil silica, 50-70% EtOAc/Pet ether) afforded N-(2-bromo-4,6-difluorobenzyl)-5-fluoro-2-methoxy-N-methylnicotinamide (0.17 g, 40%) as a yellow sticky solid. MS m/z: 389.13 (M+H).

### Intermediate 28: 5-Chloro-2-methoxy-N-methylnicotinamide

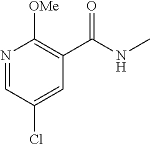

By following the procedure employed in Intermediate 1 using 5-chloro-2-methoxynicotinic acid (0.25 g, 1.33 mmol) and methylamine·HCl (0.18 g, 2.66 mmol) in THF (10 mL), the crude product was obtained. Purification by flash column chromatography using (Davisil silica, 30-40% EtOAc/Pet ether) afforded 5-chloro-2-methoxy-N-methylnicotinamide (0.23 g, 86%) as an off-white solid. ¹H NMR (400 MHz, CDCl₃): δ 8.49 (d, J=2.4 Hz, 1H), 8.19 (d, J=2.8 Hz, 1H), 7.90-7.80 (br s, 1H), 4.09 (s, 3H), 3.02 (d, J=4.8 Hz, 2H). MS m/z: 201.13 (M+H).

### Intermediate 29: 5-Chloro-N-(3-chloro-5-fluorobenzyl)-2-methoxynicotinamide

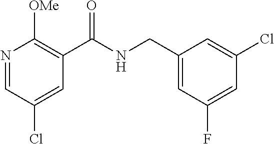 [SMILES: COc1ncc(Cl)cc1C(=O)NCc1cc(F)cc(Cl)c1]

By following the procedure employed in Intermediate 1 using 5-chloro-2-methoxynicotinic acid (0.25 g, 1.33 mmol) and (3-chloro-5-fluorophenyl)methanamine (0.25 g, 1.59 mmol) in THF (10 mL), the crude product was obtained. The crude was purified by flash column chromatography using (Davisil silica, 20-40% EtOAc/Pet ether) to afford 5-chloro-N-(3-chloro-5-fluorobenzyl)-2-methoxynicotinamide (0.33 g, 75%) as off-white solid. ¹H NMR (400 MHz, CDCl₃): δ 8.51 (d, J=2.8 Hz, 1H), 8.30-8.20 (m, 2H), 7.13 (s, 1H), 7.00-6.94 (m, 2H), 4.64 (d, J=6.0 Hz, 2H), 4.10 (s, 3H). MS m/z: 329.13 (M+H).

### Intermediate 30: 1-(3-Fluorophenyl)-N-methylethan-1-amine

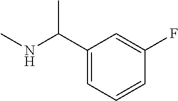

To a stirred solution of 1-(3-fluorophenyl)ethan-1-one (1.0 g, 7.2 mmol), methyl amine (7 mL) in EtOH (5 mL) was added acetic acid (0.2 mL) and the reaction mixture was stirred for 16 h at 80° C. After this time, the reaction mixture was cooled to 0° C., at which point NaBH₄ (0.326 g, 8.64 mmol) was added and stirred for 3 h at 80° C. At this point, the reaction mixture was concentrated under reduced pressure to give a residue which was dissolved in 4N HCl and washed with DCM (3×20 mL). The aqueous layer was basified with 4N NaOH and extracted with DCM (3×20 mL). The combined organic phase was dried over Na₂SO₄ and concentrated under reduced pressure to afford 1-(3-fluorophenyl)-N-methylethan-1-amine (0.5 g, 45% of yield) as a colourless liquid. 1H NMR (400 MHz, CDCl₃): δ 7.30-7.20 (m, 1H), 7.10-7.00 (m, 2H), 6.95-6.90 (m, 1H), 3.64 (q, J=6.8 Hz, 1H), 2.31 (s, 3H), 1.34 (d, J=6.4 Hz, 3H). MS m/z: 154.13 (M+H).

### Intermediate 31: 5-Fluoro-N-(6-fluoro-2,3-dihydrobenzofuran-3-yl)-2-methoxynicotinamide

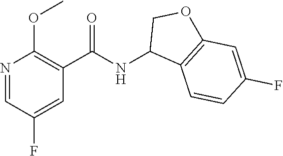 [SMILES: COc1ncc(F)cc1C(=O)NC1COc2cc(F)ccc21]

By following the procedure employed in Intermediate 1 using 5-fluoro-2-methoxynicotinic acid (0.25 g, 1.33 mmol) and 6-fluoro-2,3-dihydrobenzofuran-3-amine hydrochloride (Intermediate 48) (0.25 g, 1.59 mmol) in THF (10 mL), the crude product was obtained. Purification by flash column chromatography (Davisil silica, 20-40% EtOAc/Pet ether) afforded 5-fluoro-N-(6-fluoro-2,3-dihydrobenzofuran-3-yl)-2-methoxynicotinamide (0.33 g, 74%) as an off-white solid. MS m/z: 307.12 (M+H).

### Intermediate 32: 5-Chloro-N-(6-fluoro-2,3-dihydrobenzofuran-3-yl)-2-methoxynicotinamide

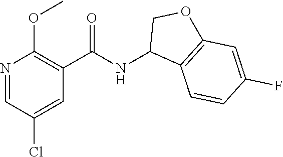 [SMILES: COc1ncc(Cl)cc1C(=O)NC1COc2cc(F)ccc21]

By following the procedure employed in Intermediate 1 using 5-chloro-2-methoxynicotinic acid (0.5 g, 2.67 mmol) and 6-fluoro-2,3-dihydrobenzofuran-3-amine hydrochloride (Intermediate 48) (0.45 g, 2.94 mmol) in THF (10 mL), the crude product was obtained. Purification by flash column chromatography (Davisil silica, 20-40% EtOAc/Pet ether) afforded 5-chloro-N-(6-fluoro-2,3-dihydrobenzofuran-3-yl)-2-methoxynicotinamide (0.67 g, 78%) as an off-white solid. ¹H NMR (400 MHz, CDCl₃): δ 8.48 (d, J=2.8 Hz, 1H), 8.22 (d, J=2.8 Hz, 1H), 8.15-8.00 (br s, 1H), 7.32-7.30 (m, 1H), 6.70-6.55 (m, 2H), 5.80-5.70 (m, 1H), 4.90-4.80 (m, 1H), 4.50-4.40 (m, 1H), 4.00 (s, 3H). MS m/z: 323.10 (M+H).

### Intermediate 33: 5-Fluoro-N-methyl-2,3-dihydro-1H-inden-1-amine

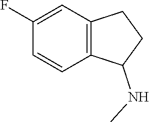

To a stirred solution 5-fluoro-2,3-dihydro-1H-inden-1-one (1 g, 6.6577 mmol) in EtOH (10 mL) in a glass tube, was added methylamine in EtOH (3.1 ml, 99.9 mmol) and the reaction mixture was stirred at RT for 16 h. After this time, the mixture was cooled to 0° C. and NaBH₄ (0.4 g, 10.5263 mmol) was added and resulting mixture was stirred at RT for 1 h. After this time, the mixture was evaporated under reduced pressure to give a residue which was dissolved in water and extracted with ethyl acetate (3×30 mL). The combined organic phases were dried over Na₂SO₄ and concentrated under reduced pressure to afford 5-fluoro-N-methyl-2,3-dihydro-1H-inden-1-amine (1.0 g, 90%) as a pale-yellow solid. MS m/z: 166.14 (M+H), 61.78%.

### Intermediate 34: N-(1-(2-Bromo-5-fluorophenyl)ethyl)-5-fluoro-2-methoxy-N-methylnicotinamide

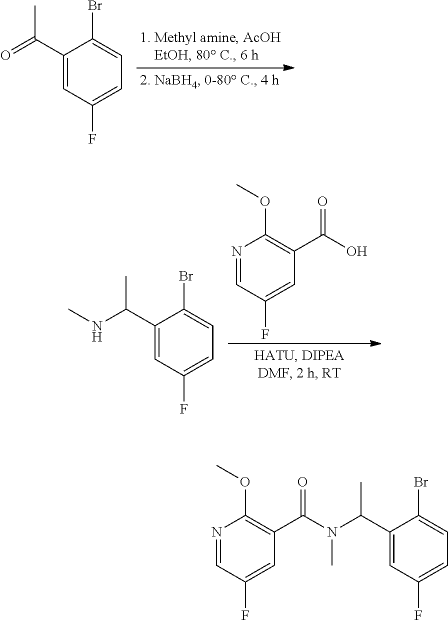 [SMILES: CC(=O)c1cc(F)ccc1Br ; CNC(C)c1cc(F)ccc1Br ; COc1ncc(F)cc1C(=O)O ; COc1ncc(F)cc1C(=O)N(C)C(C)c1cc(F)ccc1Br]

Step 1: A stirred solution of 1-(2-bromo-5-fluorophenyl)ethan-1-one (1.0 g, 4.6 mmol) and methyl amine (7 mL) in EtOH (5 mL) was treated with acetic acid (0.2 mL) and stirred for 6 h at 80° C. After this time, the reaction mixture was cooled to 0° C., NaBH₄ (0.34 g, 9.2 mmol) was added and the reaction mixture was stirred for 4 h at 80° C. After this time, the solvent was evaporated from the reaction mixture to give a residue which was dissolved in 4N HCl and washed with DCM (1×20 mL). The aqueous layer was basified by 4N NaOH and extracted with DCM (3×20 mL). The combined organic phases were dried over Na₂SO₄ and concentrated under reduced pressure to give 1-(2-bromo-5-fluorophenyl)-N-methylethan-1-amine (0.5 g, 47%) as a colourless liquid. 1H NMR (400 MHz, CDCl₃): δ 8.41 (s, 1H), 7.20-7.15 (m, 1H), 6.90-6.80 (m, 1H), 6.80-6.75 (m, 1H), 3.89 (s, 3H), 2.47 (s, 3H). MS m/z: 232.11 (M+H).

Step 2: By following the procedure employed in Intermediate 1 using 5-fluoro-2-methoxynicotinic acid (0.25 g, 1.5 mmol) and 1-(2-bromo-5-fluorophenyl)-N-methylethan-1-amine (0.38 g, 0.99 mmol) in DMF (10 mL), the crude product was obtained. Purification by flash column chromatography (Davisil silica, 10-20% EtOAc/Pet ether) afforded N-(1-(2-bromo-5-fluorophenyl)ethyl)-5-fluoro-2-methoxy-N-methylnicotinamide (0.25 g, 60%) as a colourless liquid. ¹H NMR (400 MHz, CDCl₃): δ 8.03 (s, 1H), 7.60-7.55 (m, 1H), 7.40-7.32 (m, 1H), 7.20-7.15 (m, 1H), 6.95-6.85 (m, 1H), 6.00-5.90 (m, 1H), 3.94 (s, 3H), 2.55 (br s, 3H), 1.60 (d, J=2.8 Hz, 3H). MS m/z: 385.14 (M+H).

### Intermediate 35: N-(1-(2-Bromo-5-fluorophenyl)ethyl)-5-chloro-2-methoxy-N-methylnicotinamide

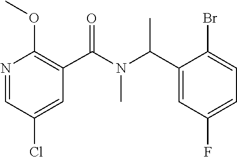 [SMILES: COc1ncc(Cl)cc1C(=O)N(C)C(C)c1cc(F)ccc1Br]

By following the procedure employed in Intermediate 1 using 5-chloro-2-methoxynicotinic acid (0.3 g, 1.6 mmol) and 1-(2-bromo-5-fluorophenyl)-N-methylethan-1-amine (Intermediate 34, Step 1) (0.24 g, 1.76 mmol) in DMF (10 mL), the crude product was obtained. Purification by flash column chromatography (Davisil silica, 20-30% EtOAc/Pet ether) afforded N-(1-(2-bromo-5-fluorophenyl)ethyl)-5-chloro-2-methoxy-N-methylnicotinamide (0.4 g, 62%) as a pale yellow gum. ¹H NMR (400 MHz, CDCl₃): δ 8.13 (d, J=2.8 Hz, 1H), 7.65-7.55 (m, 2H), 7.20-7.15 (dd, J=3.2 Hz, 9.6 Hz, 1H), 6.95-6.85 (m, 1H), 6.00-5.90 (m, 1H), 3.95 (s, 3H), 3.20-2.50 (m, 3H), 1.60 (d, J=6.8 Hz, 3H). MS m/z: 401.11 (M+H).

### Intermediate 36: N-(1-(2-Bromo-4-fluorophenyl)ethyl)-5-fluoro-2-methoxy-N-methylnicotinamide

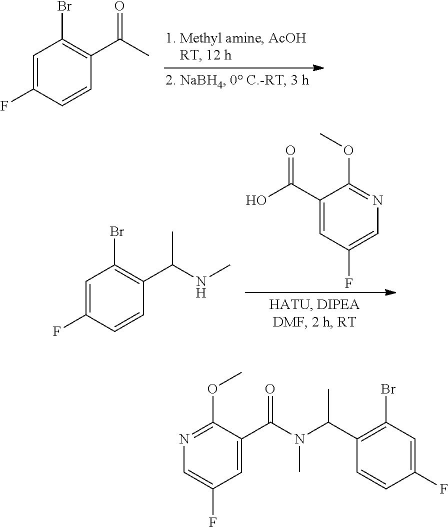 [SMILES: CC(=O)c1ccc(F)cc1Br ; CNC(C)c1ccc(F)cc1Br ; COc1ncc(F)cc1C(=O)O ; COc1ncc(F)cc1C(=O)N(C)C(C)c1ccc(F)cc1Br]

Step 1: By following the procedure employed in Intermediate 34 (Step 1) using 1-(2-bromo-4-fluorophenyl)ethan-1-one (1 g, 4.6 mmol), gave 1-(2-bromo-4-fluorophenyl)-N-methylethan-1-amine (0.6 g, 56%) as a colourless liquid. MS m/z: 232.11 (M+H), 84.56%.

Step 2: By following the procedure employed in Intermediate 1 using 5-fluoro-2-methoxynicotinic acid (0.3 g, 1.8 mmol) and 1-(2-bromo-4-fluorophenyl)-N-methylethan-1-amine (0.5 g, 2.16 mmol) in DMF (10 mL), the crude product was obtained. Purification by flash column chromatography (Davisil silica, 10-20% EtOAc/Pet ether) afforded N-(1-(2-bromo-4-fluorophenyl)ethyl)-5-fluoro-2-methoxy-N-methylnicotinamide (0.35 g, 52%) as a colourless thick gum. ¹H NMR (400 MHz, CDCl₃): δ 8.02 (d, J=3.2 Hz, 1H), 7.45-7.35 (m, 3H), 7.12-7.05 (m, 1H), 6.05-5.95 (m, 1H), 3.93 (s, 3H), 2.43 (s, 3H), 1.61 (d, J=7.2 Hz, 3H). MS m/z: 385.14 (M+H).

### Intermediate 37: N-(1-(2-Bromo-4-fluorophenyl)ethyl)-5-chloro-2-methoxy-N-methylnicotinamide

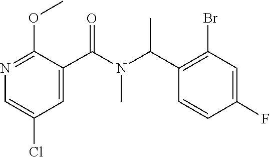 [SMILES: COc1ncc(Cl)cc1C(=O)N(C)C(C)c1ccc(F)cc1Br]

By following the procedure employed in Intermediate 1 using 5-chloro-2-methoxynicotinic acid (0.15 g, 0.87 mmol) and 1-(2-bromo-4-fluorophenyl)-N-methylethan-1-amine (Intermediate 36, Step 1) (0.16 g, 1.14 mmol) in THF (10 mL), the crude product was obtained. Purification by flash column chromatography (Davisil silica, 30-50% EtOAc/Pet ether) afforded N-(1-(2-bromo-4-fluorophenyl)ethyl)-5-chloro-2-methoxy-N-methylnicotinamide (0.2 g, 62%) as a colourless liquid. MS m/z: 401.14 (M+H).

### Intermediate 38: 1-(2,4-difluorophenyl)-N-methylethan-1-amine

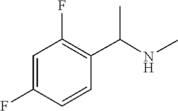

By following the procedure employed in Intermediate 34 (Step 1) using 1-(2,4-difluorophenyl)ethan-1-one (2 g, 9.3 mmol) gave 1-(2,4-difluorophenyl)-N-methylethan-1-amine (1.5 g, 68%) as a colourless liquid. MS m/z: 172.16 (M+H).

### Intermediate 39: 1-(2-Fluorophenyl)-N-methylethan-1-amine

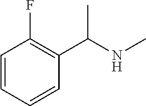

By following the procedure employed in Intermediate 34 (Step 1) using 1-(2-fluorophenyl)ethan-1-one (0.8 g, 5.8 mmol) afforded 1-(2-fluorophenyl)-N-methylethan-1-amine (0.6 g, 68%) as a colourless liquid. MS m/z: 154.10 (M+H).

### Intermediate 40: 5-Fluoro-N-(1-(2-fluorophenyl)ethyl)-2-hydroxy-N-methylnicotinamide

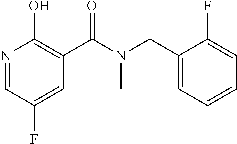 [SMILES: CN(Cc1ccccc1F)C(=O)c1cc(F)cnc1O]

To a stirred solution of 5-fluoro-N-(1-(2-fluorophenyl)ethyl)-2-methoxy-N-methylnicotinamide (Example 4 and 5) (0-1 g, 0.3 mmol) in DCM (3 ml) was added 1.0 M BBr₃ at 0° C. and the reaction mixture was stirred at RT for 4 h. After this time, the reaction was quenched with ice cold water and extracted with DCM (2×30 mL).

The organic phase was washed with sodium bicarbonate solution, dried over Na₂SO₄ and evaporated under reduced pressure to give 5-fluoro-N-(1-(2-fluorophenyl)ethyl)-2-hydroxy-N-methylnicotinamide (0.07 g, 74%) as a white solid. ¹H NMR (400 MHz, DMSO-d₆): δ 11.80 (br s, 1H), 7.75 (br s, 1H), 7.60-7.45 (m, 2H), 7.40-7.32 (m, 1H), 7.25-7.12 (m, 2H), 5.95 (q, J=6.8 Hz, 0.52H), 5.01 (q, J=7.2 Hz, 0.43H), 2.68-2.60 (m, 3H), 1.62-1.49 (m, 3H). MS m/z: 293.15 (M+H).

### Intermediate 41: 5-Chloro-N-(2,4-difluorobenzyl)-2-methoxy-N-methylnicotinamide

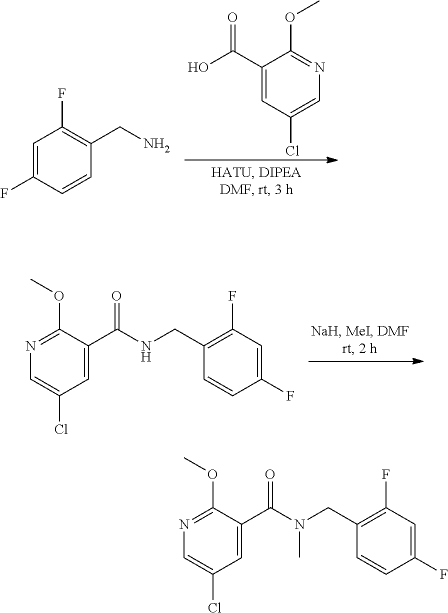 [SMILES: NCc1ccc(F)cc1F ; COc1ncc(Cl)cc1C(=O)O ; COc1ncc(Cl)cc1C(=O)NCc1ccc(F)cc1F ; COc1ncc(Cl)cc1C(=O)N(C)Cc1ccc(F)cc1F]

Step 1: By following the procedure employed in Intermediate 1 using 5-chloro-2-methoxynicotinic acid (0.5 g, 2.66 mmol) and (2,4-difluorophenyl)methanamine (0.457 g, 3.198 mmol) in DMF (10 mL), the crude product was obtained. Purification by flash column chromatography (Davisil silica, 20-30% EtOAc/Pet ether) afforded 5-chloro-N-(2,4-difluorobenzyl)-2-methoxynicotinamide (0.75 g, 90%) as an off-white solid. 1H NMR (400 MHz, CDCl₃): δ 8.48 (d, J=2.8 Hz, 1H), 8.28 (s, 1H), 8.20 (d, J=2.8 Hz, 1H), 7.42-7.38 (m, 1H), 6.90-6.80 (m, 2H), 4.65 (d, J=6.0 Hz, 2H), 4.08 (s, 3H). MS m/z: 313.10 (M+H).

Step 2: To a stirred solution of 5-chloro-N-(2,4-difluorobenzyl)-2-methoxynicotinamide (0.75 g, 2.4 mmol) in DMF (5 mL) at 0° C. was added NaH (0.04 g, 1.6 mmol). After 10 mins, methyl iodide (0.41 g, 2.88 mmol) was added at 0° C. and the resulting mixture was stirred at RT for 2 h. After this time, the reaction was quenched with water at 0° C. and extracted with EtOAc (3×20 mL). The combined organic phases were washed with ice-cold water, dried over Na₂SO₄ and evaporated under reduced pressure to obtain the crude product. Purification by flash column chromatography (Davisil silica, 10-30% EtOAc/Pet ether) afforded 5-chloro-N-(2,4-difluorobenzyl)-2-methoxy-N-methylnicotinamide (0.64 g, 82%) as an off-white solid. MS m/z: 327.13 (M+H).

### Intermediate 42: 4-Chloro-N-(2,4-difluorobenzyl)-2-methoxy-N-methylnicotinamide

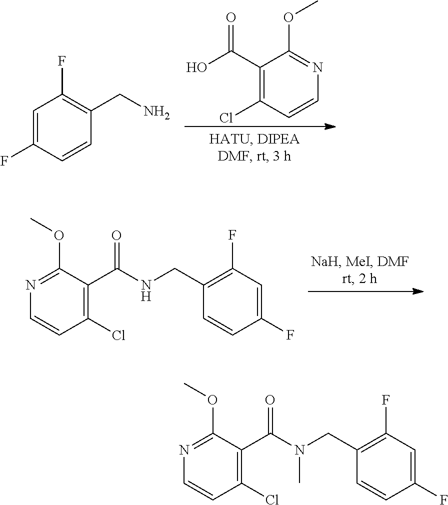 [SMILES: NCc1ccc(F)cc1F ; COc1nccc(Cl)c1C(=O)O ; COc1nccc(Cl)c1C(=O)NCc1ccc(F)cc1F ; COc1nccc(Cl)c1C(=O)N(C)Cc1ccc(F)cc1F]

Step 1: By following the procedure employed in Intermediate 1 using 4-chloro-2-methoxynicotinic acid (0.25 g, 1.3 mmol) and (2,4-difluorophenyl)methanamine (0.204 g, 1.43 mmol) in DMF (10 mL), gave 4-chloro-N-(2,4-difluorobenzyl)-2-methoxynicotinamide (0.25 g, 60%) as pale-yellow thick liquid. ¹H NMR (400 MHz, CDCl₃: δ 8.07 (d, J=5.6 Hz, 1H), 7.52-7.45 (m, 1H), 6.94 (d, J=5.6 Hz, 1H), 6.90-6.75 (m, 2H), 6.20-6.10 (m, 1H), 4.65 (d, J=6.4 Hz, 2H), 3.96 (s, 3H). MS m/z: 313.10 (M+H), 96.05%.

Step 2: By following the procedure employed in Intermediate 41 (Step 2) using 4-chloro-N-(2,4-difluorobenzyl)-2-methoxynicotinamide (0.25 g, 0.8 mmol), gave 4-chloro-N-(2,4-difluorobenzyl)-2-methoxy-N-methylnicotinamide (0.2 g, 77%) as a colourless gum. ¹H NMR (400 MHz, CDCl₃): δ 8.08 (d, J=4.4 Hz, 1H), 7.58-7.48 (m, 1H), 6.94 (d, J=5.6 Hz, 1H), 6.92-6.75 (m, 2H), 4.90-4.75 (m, 2H), 3.97 (s, 3H), 2.79 (s, 3H). MS m/z: 327.13 (M+H), 96.43%.

### Intermediate 43: 1-(2,4-Difluorophenyl)-N-methylmethanamine

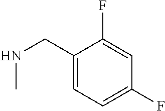

By following the procedure employed in Intermediate 34 (step 1) using 2,4-difluorobenzaldehyde (0.5 g, 4.03 mmol), afforded 1-(2,4-difluorophenyl)-N-methylmethanamine (0.4 g, 72%) as a colourless liquid. MS m/z: 140.01 (M+H), 70.32%.

### Intermediate 44: 7-Fluoro-2,3,4,5-tetrahydrobenzo[f][1,4]oxazepine

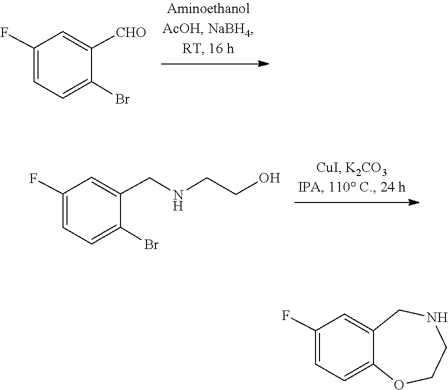 [SMILES: O=Cc1cc(F)ccc1Br ; OCCNCc1cc(F)ccc1Br ; Fc1ccc2c(c1)CNCCO2]

Step 1: To a stirred solution of 2-bromo-5-fluorobenzaldehyde (4.0 g, 19.70 mmol) and aminoethanol (1.2 g, 23.64 mmol) in EtOH (5 mL) was added acetic acid (0.2 mL) and the resulting mixture was stirred for 16 h at RT. The reaction mixture was cooled to 0° C., NaBH₄ (0.34 g, 9.2 mmol) was added and the reaction mixture stirred for a further 1 h at r. The solvent was evaporated from the reaction mixture under reduced pressure to give a residue which was dissolved in water and extracted with DCM (3×20 mL). The combined organic phases were dried over Na₂SO₄ and concentrated under reduced pressure to give 2-((2-bromo-5-fluorobenzyl)amino)ethan-1-ol (4.3 g, 88%) as a pale yellow solid, which was used in the next step without further purification. MS m/z: 247.83 (M+H).

Step 2: To a stirred solution of 2-((2-bromo-5-fluorobenzyl)amino)ethan-1-ol (4.3 g, 17.4 mmol) in IPA (40 mL) was added CuI (4.2 g., 17.4 mmol) and K₂CO₃ (9.1 g, 52.2 mmol) and the reaction mixture stirred for 24 h at 110° C. After this time, the reaction mixture was filtered through celite, washed with IPA and the filtrate was concentrated under reduced pressure. Purification by reverse phase column chromatography (60-70% MeOH/0.1% ABC (ammonium bicarbonate) solution as mobile phase) gave 7-fluoro-2,3,4,5-tetrahydrobenzo[f][1,4]oxazepine (1.2 g, 41%) as a colourless liquid. 1H NMR (400 MHz, CDCl₃): δ 7.02-6.95 (m, 1H), 6.90-6.80 (m, 2H), 4.03 (t, J=4.4 Hz, 2H), 3.95 (s, 2H), 3.25 (d, J=4.4 Hz, 2H). MS m/z: 168.14 (M+H).

### Intermediate 45: 2-(Difluoromethoxy)-5-fluoro-pyridine-3-carbonyl chloride

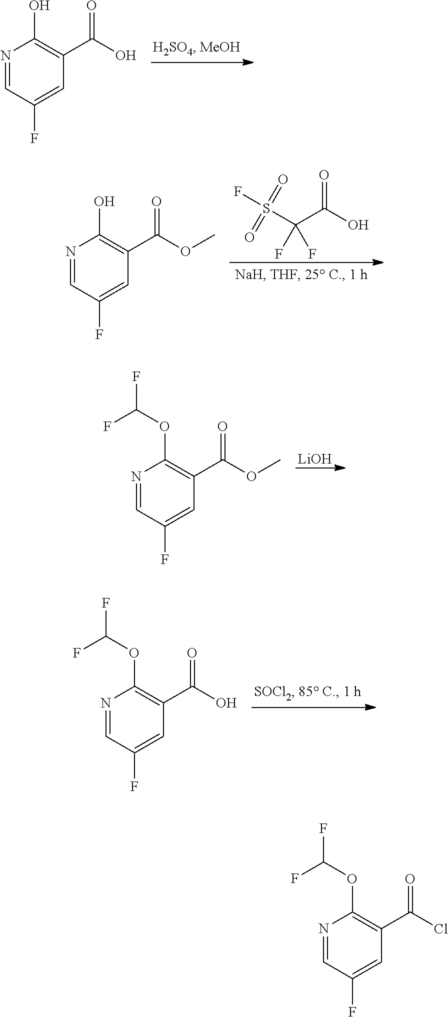

Step 1: A solution of 5-fluoro-2-hydroxy-pyridine-3-carboxylic acid (1 g, 6.37 mmol) and con. H₂SO₄ (5.2 mL) in MeOH (5 mL) was stirred at 90° C. for 1 hour under microwave. Then the mixture was cooled down to RT and evaporated to remove most of the MeOH under reduced pressure. The obtained mixture was treated with sat. NaHCO₃ (aq.) until the pH was around 7-8 and extracted with CH₂Cl₂ (30 mL×3). The combined organic layers were dried over anhydrous Na₂SO₄, and evaporated to dryness to give methyl 5-fluoro-2-hydroxy-pyridine-3-carboxylate (700 mg, 4.09 mmol, 64.26% yield) as a yellow solid.

Step 2: NaH (490.75 mg, 12.27 mmol, 60% purity) was added into methyl 5-fluoro-2-hydroxy-pyridine-3-carboxylate (1.4 g, 8.18 mmol) in THF (1 mL) in portions at 0° C. under N₂, and then 2,2-difluoro-2-fluorosulfonyl-acetic acid (1.46 g, 8.18 mmol) in THF (1 mL) was added in one portion at 0° C. under N₂. The mixture was stirred at 25° C. for 1 hour. The mixture was poured into sat. NH₄Cl (aq.) (30 mL) and extracted with EtOAc (3×30 mL). The combined organic layers were concentrated to afford the crude product which was purified by flash silica gel chromatography (ISCO®; 4 g SepaFlash® Silica Flash Column, Eluent of 0˜25% Ethyl acetate/Petroleum ether gradient @30 mL/min) to give methyl 2-(difluoromethoxy)-5-fluoro-pyridine-3-carboxylate (740 mg, 3.10 mmol, 37.9% yield) as a yellow liquid. ¹H NMR (400 MHz, DMSO-d₆): δ 8.58-8.20 (m, 2H), 7.70 (t, J=72.0 Hz, 1H), 3.87 (s, 3H).

Step 3: A mixture of methyl 2-(difluoromethoxy)-5-fluoro-pyridine-3-carboxylate (690 mg, 3.12 mmol) and 1 M LiOH (aq.) (15.60 mL) in THF (15 mL) was stirred at 25° C. for 1 hour. The reaction mixture was poured into H₂O (20 mL), and adjusted to pH=5 with 1 M HCl (aq.), then extracted with ethyl acetate (50 mL×3). The combined organic phases were washed with brine (20 mL), dried over anhydrous Na₂SO₄, concentrated in vacuum to give 2-(difluoromethoxy)-5-fluoro-pyridine-3-carboxylic acid (600 mg, 2.53 mmol, 81.1% yield, 87.4% purity) as yellow powder which was used for next step directly.

Step 4: A mixture of 2-(difluoromethoxy)-5-fluoro-pyridine-3-carboxylic acid (300 mg, 1.45 mmol) in SOCl₂ (3 mL) was stirred at 85° C. for 1 hour. The mixture was concentrated to afford the crude product 2-(difluoromethoxy)-5-fluoro-pyridine-3-carbonyl chloride (300 mg, crude) as a yellow solid, which was used directly without further purification

### Intermediate 46: 7-fluoro-2,3-dihydrobenzofuran-3-amine hydrochloride

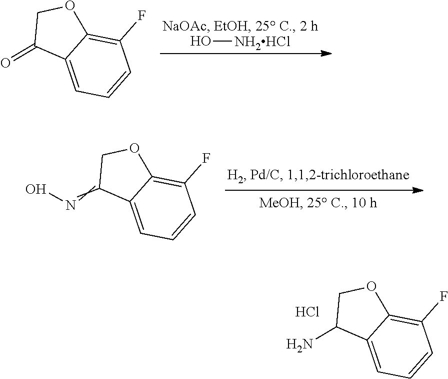

Step 1: A mixture of 7-fluorobenzofuran-3(2H)-one (800 mg, 4.45 mmol, 84.7% purity) (Prepared as described in EP3312184), hydroxylamine hydrochloride (366 mg, 5.26 mmol) and sodium acetate (863 mg, 10.52 mmol) in ethanol (5 mL) was stirred at 25° C. for 2 hours. The mixture was filtered and the cake was washed with ethanol (20 mL) to give the crude product 7-fluorobenzofuran-3(2H)-one oxime (1.5 g, crude) as white solid. MS ES⁺: 168.2

Step 2: To a solution of 7-fluorobenzofuran-3(2H)-one oxime (500 mg, crude) in methanol (5 mL) was added palladium on activated charcoal (500 mg, 10% purity) and 1, 1, 2-trichloroethane (599 mg, 4.49 mmol) under Ar. The suspension was degassed under vacuum and purged with H₂ several times. The mixture was stirred under H₂ (30 psi) at 25° C. for 10 hours. The mixture was filtered and then concentrated to give 7-fluoro-2,3-dihydrobenzofuran-3-amine hydrochloride (460 mg, crude) as a brown solid. ¹H NMR (400 MHz, DMSO-d₆): 8.94 (s, 3H), 7.51 (d, J=7.5 Hz, 1H), 7.39-7.18 (m, 2H), 7.05-6.92 (m, 1H), 5.11 (d, J=4.6 Hz, 1H), 4.88-4.74 (m, 1H), 4.65 (dd, J=2.9, 10.7 Hz, 1H).

### Intermediate 47: 5-chloro-2-(difluoromethoxy)pyridine-3-carbonyl chloride

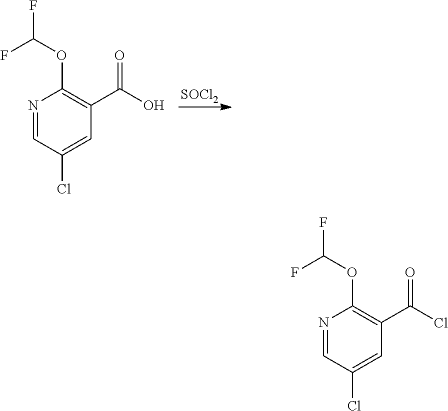

A mixture of 5-chloro-2-(difluoromethoxy)pyridine-3-carboxylic acid (Intermediate 54) (155 mg, 0.693 mmol) in SOCl₂ (2 mL) was stirred at 90° C. for 0.5 hour. The mixture was concentrated to dryness to give 5-chloro-2-(difluoromethoxy)pyridine-3-carbonyl chloride (166 mg, crude) as yellow gum which was used for the next step directly.

### Intermediate 48: 6-fluoro-2,3-dihydrobenzofuran-3-amine hydrochloride

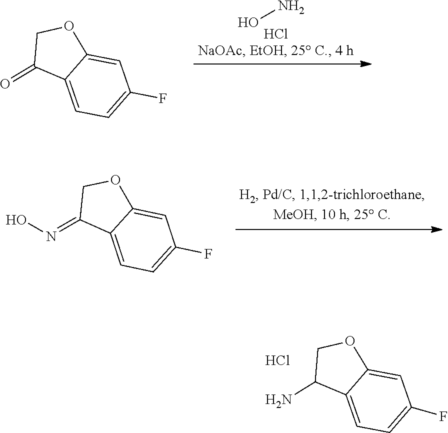

Step 1: To a mixture of 6-fluorobenzofuran-3-one (900 mg, 5.92 mmol) and hydroxylamine hydrochloride (822 mg, 7.76 mmol) in ethanol (5 mL) was added sodium acetate (970 mg, 11.83 mmol) in one portion at 25° C. The mixture was stirred at 25° C. for 4 hours with white precipitation formed. The precipitation was collected to afford 6-fluorobenzofuran-3-one oxime (900 mg, crude) as a white powder. MS ES⁺: 168.2

Step 2: To a mixture of 6-fluorobenzofuran-3-one oxime (900 mg, crude) in methanol (5 mL) was added 1, 1, 2-trichloroethane (1.44 g, 10.77 mmol) and palladium on activated charcoal (1 g, 10% purity). The suspension was degassed under vacuum and purged with H₂ several times. The mixture was stirred for 10 hours at 25° C. under H₂ (30 psi). The resulting mixture was filtered and evaporated to dryness to afford the crude product 6-fluoro-2,3-dihydrobenzofuran-3-amine hydrochloride (1.4 g, crude) as a yellow powder. ¹H NMR (400 MHz, DMSO-d₆): 7.79-7.57 (m, 1H), 6.94-6.65 (m, 2H), 5.06-4.90 (m, 1H), 4.76 (dd, J=8.3, 10.8 Hz, 1H), 4.65-4.48 (m, 1H).

### Intermediate 49: 7-fluoro-N-methyl-2,3-dihydrobenzofuran-3-amine trifluoroacetate

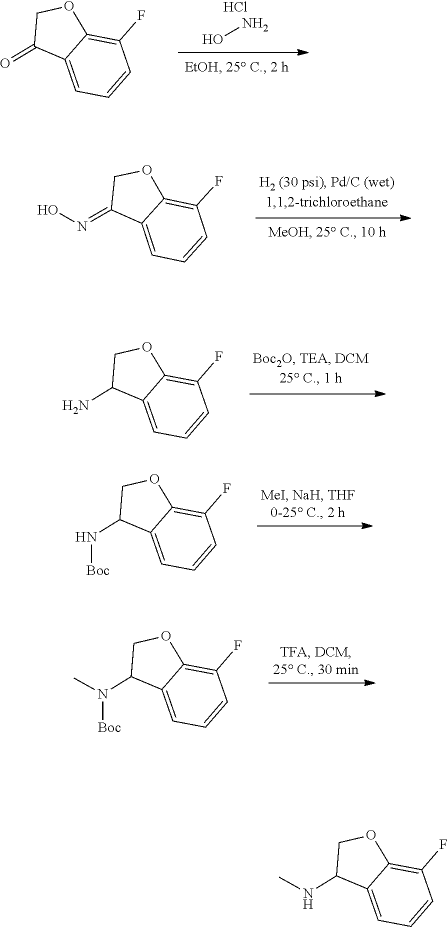

Step 1: To a mixture of 7-fluorobenzofuran-3(2H)-one (800 mg, 4.45 mmol, 84.7% purity) and hydroxylamine hydrochloride (366 mg, 5.26 mmol) in ethanol (5 mL) was added sodium acetate (863 mg, 10.52 mmol). The mixture was stirred at 25° C. for 2 hours with white precipitation formed. The precipitation was collected to give 7-fluorobenzofuran-3(2H)-one oxime (1.5 g, crude) as a white solid. MS ES⁺: 168.2

Step 2: To a solution of 7-fluorobenzofuran-3(2H)-one oxime (500 mg, 2.99 mmol) and 1, 1, 2-trichloroethane (599 mg, 4.49 mmol) in methanol (2 mL) was added palladium on activated charcoal (500 mg, 10% purity) under N₂. The suspension was degassed under vacuum and purged with H₂ several times. The mixture was stirred under H₂ (30 psi) at 25° C. for 10 hours. The mixture was filtered and concentrated in vacuum to afford the crude product 7-fluoro-2,3-dihydrobenzofuran-3-amine hydrochloride (438 mg, 2.31 mmol, 77.2% yield) as a white solid. ¹H NMR (400 MHz, DMSO-d₆): 8.80 (s, 2H), 7.47 (d, J=7.5 Hz, 1H), 7.32-7.25 (m, 1H), 7.00 (dt, J=4.4, 7.9 Hz, 1H), 5.12 (s, 1H), 4.81 (dd, J=8.3, 10.9 Hz, 1H), 4.63 (dd, J=3.6, 10.9 Hz, 1H).

Step 3: To a mixture of 7-fluoro-2,3-dihydrobenzofuran-3-amine hydrochloride (300 mg, 1.58 mmol) and tert-butoxycarbonyl tert-butyl carbonate (345 mg, 1.58 mmol) in dichloromethane (2 mL) was added triethylamine (480 mg, 4.75 mmol) in one portion at 25° C. The mixture was stirred at 25° C. for 1 hour. The mixture was poured into water (10 mL) and extracted with dichloromethane (10 mL×3), the combined organic layers were concentrated to dryness which was purified by flash silica gel chromatography (ISCO®; 4 g SepaFlash® Silica Flash Column, Eluent of 0˜15% Ethyl acetate/Petroleum ether gradient 30 mL/min) to afford tert-butyl N-(7-fluoro-2,3-dihydrobenzofuran-3-yl) carbamate (241 mg, 0.952 mmol, 60.14% yield) as a white solid. ¹H NMR (400 MHz, DMSO-d₆): 7.59 (d, J=7.4 Hz, 1H), 7.18-7.05 (m, 2H), 6.88 (dt, J=4.4, 7.8 Hz, 1H), 5.45-5.26 (m, 1H), 4.74 (t, J=9.3 Hz, 1H), 4.31 (dd, J=5.5, 9.4 Hz, 1H), 1.40 (s, 9H)

Step 4: NaH (47.4 mg, 1.18 mmol, 60% purity) was added into the mixture of tert-butyl N-(7-fluoro-2,3-dihydrobenzofuran-3-yl)carbamate (100 mg, 0.395 mmol) in THF (2 mL) at 0° C. under N₂ and then Mel (168 mg, 1.18 mmol) was added. The mixture was stirred at 25° C. for 2 hours. The mixture was poured into saturated NH₄Cl (aq.) (10 mL) and extracted with ethyl acetate (10 mL×3), the combined organic layers were concentrated to dryness and the residue was purified by flash silica gel chromatography (ISCO®; 4 g SepaFlash® Silica Flash Column, Eluent of 0˜10% Ethyl acetate/Petroleum ether gradient 30 mL/min) to afford tert-butyl N-(7-fluoro-2,3-dihydrobenzofuran-3-yl)-N-methyl-carbamate (100 mg, 0.374 mmol, 94.75% yield) as colourless oil. ¹H NMR (400 MHz, DMSO-d₆): 7.18 (dd, J=8.4, 10.8 Hz, 1H), 7.06 (s, 1H), 6.92 (dt, J=4.4, 7.8 Hz, 1H), 6.06-5.65 (m, 1H), 6.06-5.65 (m, 1H), 4.55 (s, 1H), 3.32 (s, 3H), 1.41 (s, 9H).

Step 5: To a mixture of tert-butyl N-(7-fluoro-2,3-dihydrobenzofuran-3-yl)-N-methyl-carbamate (100 mg, 0.374 mmol) in dichloromethane (0.5 mL) was added 2,2,2-trifluoroacetic acid (2.31 g, 20.26 mmol) in one portion at 25° C. The mixture was stirred at 25° C. for 30 minutes. The mixture was concentrated to afford the crude product 7-fluoro-N-methyl-2,3-dihydrobenzofuran-3-amine trifluoroacetate (80 mg, crude) as brown oil, which was used without further purification.

### Intermediate 50: 5-fluoro-2-hydroxynicotinoyl chloride

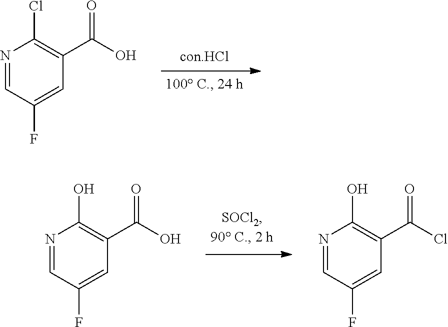 [SMILES: O=C(O)c1cc(F)cnc1Cl ; O=C(O)c1cc(F)cnc1O ; O=C(Cl)c1cc(F)cnc1O]

Step 1: 2-chloro-5-fluoronicotinic acid (4 g, 22.79 mmol) was added to con. HCl (20 mL) in one portion at 25° C. The mixture was stirred at 100° C. for 24 hours. The mixture was concentrated to afford the crude product 5-fluoro-2-hydroxynicotinic acid (3.5 g, crude) as an off-white solid.

Step 2: A mixture of 5-fluoro-2-hydroxynicotinic acid (500 mg, crude) in SOCl₂ (1.5 mL) was stirred at 90° C. for 2 hours. The mixture was concentrated to afford the crude 5-fluoro-2-hydroxynicotinoyl chloride (550 mg, crude) as a white solid, which was used in the next steps without further purification.

### Intermediate 51: 2-hydroxy-5-(trifluoromethyl)pyridine-3-carbonyl chloride

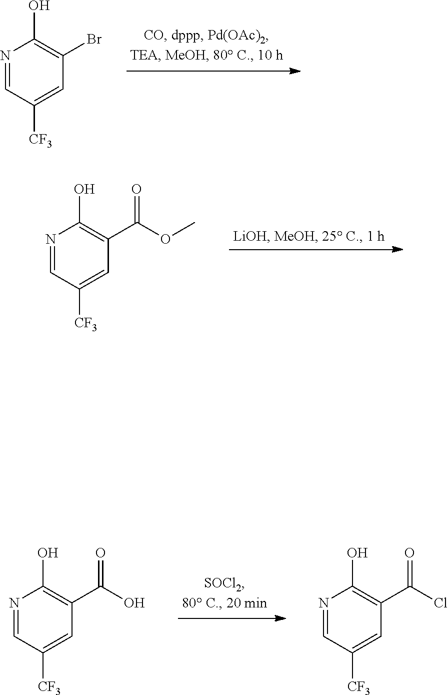

Step 1: To a mixture of 3-bromo-5-(trifluoromethyl)pyridin-2-ol (1 g, 4.13 mmol), triethylamine (1.25 g, 12.40 mmol) and Pd(OAc)₂ (463.9 mg, 2.07 mmol) in methanol (10 mL) was added 3-diphenylphosphanylpropyl(diphenyl)phosphane (1.19 g, 2.89 mmol) in one portion at 25° C. under N₂. The mixture was stirred under CO (50 psi) at 80° C. for 10 hours. The mixture was filtered and concentrated to afford the crude product methyl 2-hydroxy-5-(trifluoromethyl)pyridine-3-carboxylate (800 mg, crude) as a yellow solid. MS ES⁺: 221.9

Step 2: A mixture of methyl 2-hydroxy-5-(trifluoromethyl)pyridine-3-carboxylate (200 mg, crude) in methanol (0.1 mL) and 1M LiOH (aq.) (1.81 mL) was stirred at 25° C. for 1 hour. The mixture was adjusted to pH=5 with HCl (1M in water), then extracted with ethyl acetate (20 mL×3) and concentrated to afford the crude product 2-hydroxy-5-(trifluoromethyl)pyridine-3-carboxylic acid (150 mg, crude) as a brown solid. MS ES⁺: 208.0

Step 3: A mixture of 2-hydroxy-5-(trifluoromethyl)pyridine-3-carboxylic acid (150 mg, crude) in SOCl₂ (2 mL) was stirred at 80° C. for 20 minutes. The mixture was concentrated to afford the crude product 2-hydroxy-5-(trifluoromethyl)pyridine-3-carbonyl chloride (150 mg, crude) as brown oil, which was used without further purification.

### Intermediate 52: 5-cyclobutyl-2-(difluoromethoxy)pyridine-3-carbonyl chloride

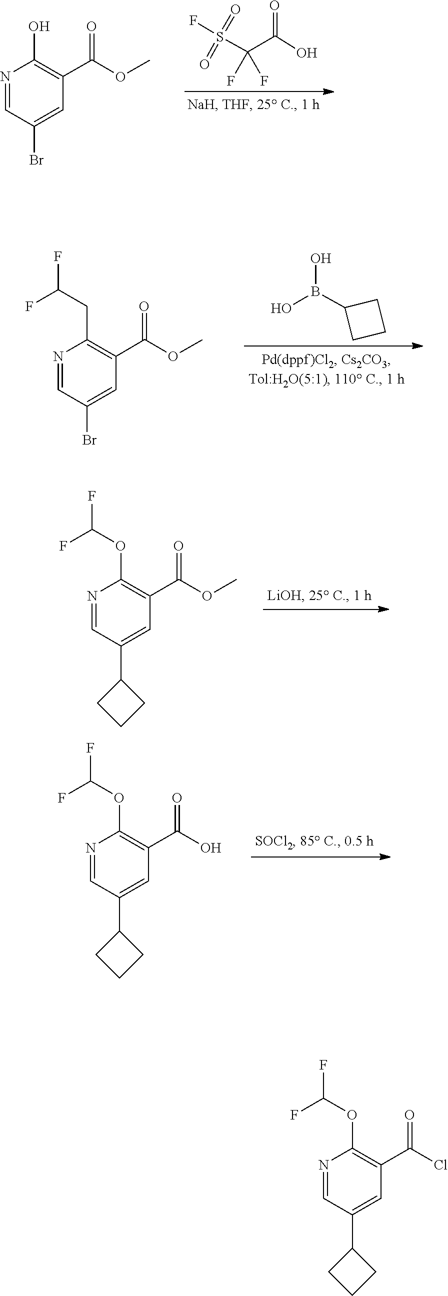

Step 1: NaH (517 mg, 12.93 mmol, 60% purity) was added into methyl 5-bromo-2-hydroxynicotinate (2.00 g, 8.62 mmol) in THF (10 mL) in one portion at 0° C. under N₂, and then 2,2-difluoro-2-(fluorosulfonyl)acetic acid (1.84 g, 10.34 mmol) was added in one portion at 0° C. under N₂. The mixture was stirred at 25° C. for 1 hour. The mixture was poured into saturated NH₄Cl (aq.) (50 mL) and extracted with ethyl acetate (3×150 mL), the combined organic layers were concentrated to dryness to give a residue which was purified by flash silica gel chromatography (ISCO®; 20 g SepaFlash® Silica Flash Column, Eluent of 0-15% Ethyl acetate/Petroleum ether gradient @45 mL/min) to afford methyl 5-bromo-2-(difluoromethoxy)nicotinate (1.28 g, 4.54 mmol, 52.65% yield) as a white solid. ¹H NMR (400 MHz, DMSO-d₆): 8.65 (d, J=2.5 Hz, 1H), 8.49 (d, J=2.5 Hz, 1H), 8.06-7.43 (m, 1H), 3.87 (s, 3H).

Step 2: To a solution of methyl 5-bromo-2-(difluoromethoxy)pyridine-3-carboxylate (500 mg, 1.77 mmol) and cyclobutylboronic acid (266 mg, 2.66 mmol) in toluene (2.5 mL) and H₂O (0.5 mL) was added a solution of Pd(dppf)Cl₂ (259 mg, 0.355 mmol) and Cs₂CO₃ (1.73 g, 5.32 mmol) under N₂ at 25° C. The reaction mixture was stirred at 110° C. for 1 hour. The mixture was poured into brine (20 mL) and extracted with ethyl acetate (3×25 mL), the combined organic layers were concentrated to dryness to give a residue, which was purified by flash silica gel chromatography (ISCO®; 4 g SepaFlash® Silica Flash Column, Eluent of 0˜30% Ethyl acetate/Petroleum ether gradient @30 mL/min) to afford methyl 5-cyclobutyl-2-(difluoromethoxy)pyridine-3-carboxylate (100 mg, 0.365 mmol, 20.39% yield, 93% purity) as a white solid. MS ES⁺: 258.0

Step 3: A mixture of methyl 5-cyclobutyl-2-(difluoromethoxy)pyridine-3-carboxylate (50 mg, 0.194 mmol) in 1M LiOH (aq.) (0.972 mL) and THF (0.3 mL) was stirred at 25° C. for 1 hour. The mixture was adjusted to pH=5 with 1 M HCl (aq.), and extracted with ethyl acetate (3×10 mL), the combined organic layers were concentrated to afford 5-cyclobutyl-2-(difluoromethoxy)pyridine-3-carboxylic acid (36 mg, crude) as a white oil. MS ES⁺: 244.0

Step 4: 5-cyclobutyl-2-(difluoromethoxy)pyridine-3-carboxylic acid (36 mg, crude) was dissolved in SOCl₂ (0.5 mL). The reaction mixture was stirred at 85° C. for 0.5 hour. The mixture was concentrated to dryness to afford 5-cyclobutyl-2-(difluoromethoxy)pyridine-3-carbonyl chloride (30 mg, crude) as a yellow solid, which was used without further purification

### Intermediate 53: [3-(difluoromethoxy)phenyl]methanamine hydrochloride

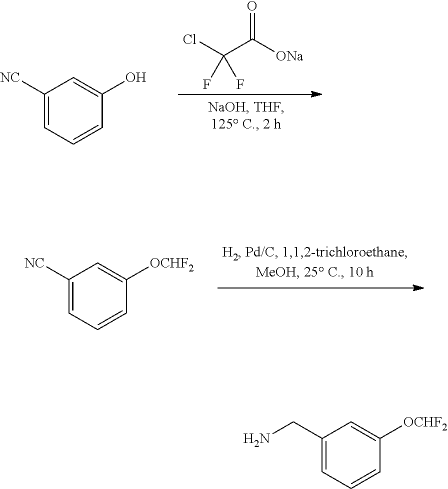

Step 1: To a stirred solution of 3-hydroxybenzonitrile (100 mg, 0.839 mmol) in THF (1 mL) were added NaOH (40.3 mg, 1.01 mmol) and 2-chloro-2,2-difluoro-acetate (153 mg, 1.01 mmol). The reaction mixture was stirred at 125° C. for 2 hours. The reaction mixture was quenched with water (10 mL) and extracted with ethyl acetate (3×10 mL). The combined organic layers were dried over anhydrous Na₂SO₄ and concentrated to dryness under reduced pressure, to give a residue which was purified by flash silica gel chromatography (ISCO®; 4 g SepaFlash® Silica Flash Column, Eluent of 0˜20% Ethyl acetate/Petroleum ether gradient @25 L/min) to give 3-(difluoromethoxy)benzonitrile (70 mg, 0.414 mmol, 49.3% yield) as colourless oil. ¹H NMR (400 MHz, DMSO-d₆): 7.76-7.71 (m, 2H), 7.68-7.62 (m, 1H), 7.58-7.52 (m, 1H), 7.37-7.14 (m, 1H).

Step 2: To a solution of 3-(difluoromethoxy)benzonitrile (70 mg, 0.414 mmol) in methanol (2 mL) was added palladium on activated charcoal (100 mg, 10% purity) and 1,1,2-trichloroethane (82.8 mg, 0.621 mmol) under Ar. The suspension was degassed under vacuum and purged with H₂ several times. The mixture was stirred under H₂ (30 psi) at 25° C. for 10 hours. The mixture was filtered and concentrated to dryness to give [3-(difluoromethoxy)phenyl]methanamine hydrochloride (58 mg, crude) as a white solid, which was used without further purification.

### Intermediate 54: 5-chloro-2-(difluoromethoxy)nicotinic acid

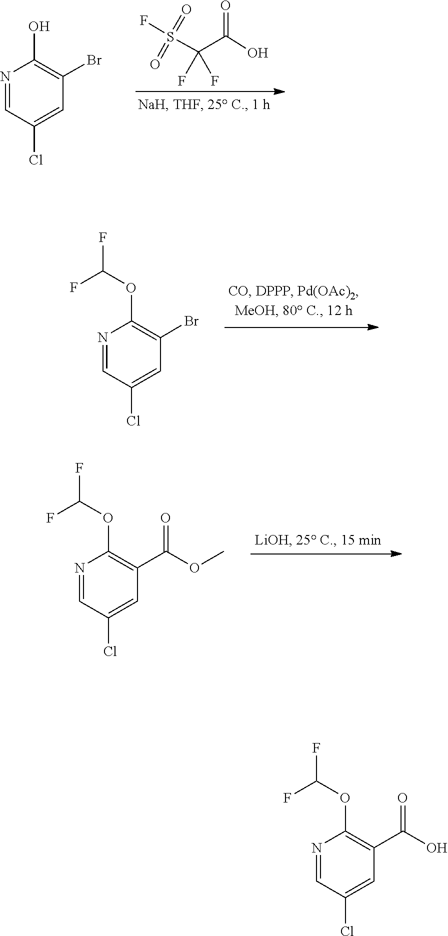

Step 1: NaH (374 mg, 9.36 mmol, 60% purity) was added to a mixture of 3-bromo-5-chloropyridin-2-ol (1.3 g, 6.24 mmol) in THF (10 mL) for 30 minutes, then 2, 2-difluoro-2-fluorosulfonyl-acetic acid (1.11 g, 6.24 mmol) was added in portions. The reaction mixture was stirred at 25° C. for 1 hour. The mixture was quenched by saturated NH₄Cl (aq.) (50 mL). The mixture was extracted with ethyl acetate (3×50 mL). The combined organic phases were washed with brine (100 mL), dried over anhydrous Na₂SO₄, concentrated in vacuum to give a residue, which was purified by silica gel column chromatography (Petroleum ether/Ethyl acetate=3:1) to afford the product 3-bromo-5-chloro-2-(difluoromethoxy)pyridine (950 mg, 3.68 mmol, 58.94% yield) as a yellow solid. ¹H NMR (400 MHz, DMSO-d₆): 8.45 (d, J=2.25 Hz, 1H); 8.32 (d, J=2.38 Hz, 1H); 7.66 (t, J=71.73 Hz, 1H)

Step 2: To a solution of 3-bromo-5-chloro-2-(difluoromethoxy) pyridine (550 mg, 2.13 mmol), 3-diphenylphosphanylpropyl(diphenyl)phosphane (439 mg, 1.06 mmol), Pd(OAc)₂ (334 mg, 1.49 mmol) in methanol (5 mL) was added triethylamine (646 mg, 6.38 mmol). The suspension was degassed under vacuum and purged with CO several times. The mixture was stirred under CO (50 psi) at 80° C. for 12 hours. The mixture was cooled to room temperature and filtered with celite, the filter cake was washed with methanol (50 mL), the filtrate was concentrated to afford the crude product, which was purified by silica gel column chromatography (Petroleum ether/Ethyl acetate=20:1) to afford the compound methyl 5-chloro-2-(difluoromethoxy)nicotinate (250 mg, 1.05 mmol, 49.45% yield) as a white solid.

Step 3: A mixture of methyl 5-chloro-2-(difluoromethoxy)nicotinate (900 mg, 3.79 mmol), 1M LiOH (aq.) (18.94 mL) in methanol (5 mL) was stirred at 25° C. for 15 minutes. The reaction mixture was poured into H₂O (50 mL), and adjusted to pH=5 with 1 M HCl (aq.), then extracted with ethyl acetate (3×50 mL). The combined organic phases were washed with brine (100 mL), dried over anhydrous Na₂SO₄, concentrated in vacuum to give the crude product 5-chloro-2-(difluoromethoxy)nicotinic acid (650 mg, crude) as a white solid which was used in the next step directly. ¹H NMR (400 MHz, DMSO-d₆): 13.76 (br s, 1H) 8.52 (d, J=2.63 Hz, 1H) 8.34 (d, J=2.63 Hz, 1H) 7.52-7.92 (m, 1H)

### Intermediate 55: 1-(3-fluorophenyl)-N-methylmethanamine

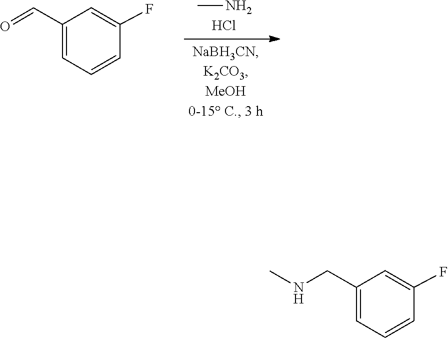

To a solution of methanamine hydrochloride (1.63 g, 24.2 mmol) in methanol (10 mL) was added K₂CO₃ (1.67 g, 12.1 mmol). After stirring the mixture for 0.5 hour, 3-fluorobenzaldehyde (2 g, 16.11 mmol) was added. The mixture was stirred at 15° C. for 2 hours, then sodium cyanotrihydroborate (1.52 g, 24.17 mmol) was added at 0° C. and the mixture was stirred at 0° C. for 1 hour. The reaction mixture was quenched by H₂O (5 mL) and then diluted with H₂O (30 mL) and extracted with ethyl acetate (3×30 mL). The combined organic layers were washed with brine (60 mL), dried over anhydrous Na₂SO₄, filtered and concentrated under reduced pressure to give a residue which was purified by flash silica gel chromatography (ISCO®; 40 g SepaFlash® Silica Flash Column, Eluent of 0˜20% methanol/dichloromethane gradient @100 mL/min) to afford 1-(3-fluorophenyl)-N-methylmethanamine (380 mg, 2.73 mmol, 16.94% yield) as colourless oil, which was used without further purification.

### Intermediate 56: 1-(2,3-difluorophenyl)-N-methylmethanamine

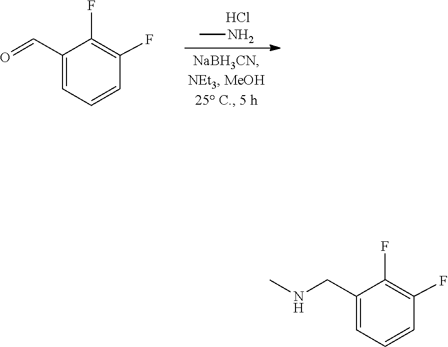

Step 1: To a mixture of 2,3-difluorobenzaldehyde (1 g, 7.04 mmol), methanamine hydrochloride (475 mg, 7.04 mmol) in methanol (20 mL) was added triethylamine (2.14 g, 21.1 mmol) in one portion at 25° C. The mixture was stirred at 25° C. for 2 hours. Then sodium cyanotrihydroborate (1.33 g, 21.1 mmol) was added to the mixture in portions and the mixture was stirred at 25° C. for 3 hours. The mixture was poured into water (100 mL), and extracted with ethyl acetate (3×100 mL). The combined organic layers were washed with brine (100 mL), dried over Na₂SO₄ and filtered. The filtrate was evaporated to dryness to give a residue which was purified by flash silica gel chromatography (SiO₂, dichloromethane/methanol=1/0 to 10/1) to afford 1-(2,3-difluorophenyl)-N-methylmethanamine (350 mg, 1.31 mmol, 18.7% yield, 59% purity) as yellow oil, which was used in the next step without further purification. MS ES⁺: 158.1.

### Intermediate 57: 2-(difluoromethoxy)-5-fluoropyridine-3-sulfonyl chloride

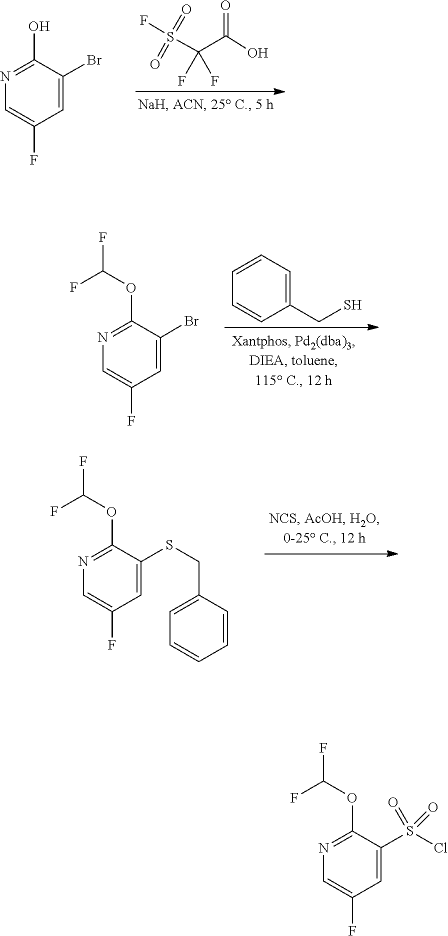

Step 1: To a solution of 3-bromo-5-fluoropyridin-2-ol (4 g, 20.8 mmol) in MeCN (20 mL) was added NaH (2.50 g, 62.50 mmol, 60% purity) in one portion at 0° C. under N₂, then 2,2-difluoro-2-(fluorosulfonyl)acetic acid (5.57 g, 31.25 mmol) was added to the mixture. The mixture was stirred at 25° C. for 5 hours. The mixture was poured into saturated NH₄Cl (aq.) (200 mL) and extracted with ethyl acetate (3×200 mL), the combined organic layers were concentrated to dryness to give a residue which was purified by flash silica gel chromatography (ISCO®; 20 g SepaFlash® Silica Flash Column, Eluent of 0-15% Ethyl acetate/Petroleum ether gradient 35/min) to afford 3-bromo-2-(difluoromethoxy)-5-fluoropyridine (2.5 g, 9.30 mmol, 44.6% yield, 90% purity) as yellow oil. ¹H NMR (400 MHz, DMSO-d₆): 8.43 (dd, J=2.8, 7.6 Hz, 1H), 8.34 (d, J=2.8 Hz, 1H), 7.86-7.47 (m, 1H); MS ES⁺: 243.9.

Step 2: A mixture of 3-bromo-2-(difluoromethoxy)-5-fluoropyridine (1 g, 4.13 mmol), phenylmethanethiol (513 mg, 4.13 mmol), DIPEA (801.1 mg, 6.20 mmol) and Xantphos (478.2 mg, 0.826 mmol) in toluene (5 mL) was degassed and purged with N₂ 3 times. Pd₂(dba)₃ (378 mg, 0.413 mmol) was then added and the mixture was stirred at 115° C. for 12 hours under N₂. The reaction mixture was diluted with water (30 mL) and extracted with ethyl acetate (3×40 mL). The combined organic layers were washed with brine (30 mL), dried over anhydrous Na₂SO₄, filtered and concentrated under reduced pressure to dryness to give a residue which was purified by flash silica gel chromatography (ISCO®; 20 g SepaFlash® Silica Flash Column, Eluent of 0˜15% Ethyl acetate/Petroleum ether gradient @40 mL/min) to afford 3-(benzylthio)-2-(difluoromethoxy)-5-fluoropyridine (1.2 g, 4.00 mmol, 96.7% yield, 95% purity) as yellow liquid. ¹H NMR (400 MHz, DMSO-d₆): 8.02 (d, J=2.63 Hz, 1H) 7.88 (dd, J=8.76, 2.75 Hz, 1H) 7.64 (t, J=72.29 Hz, 1H) 7.43 (d, J=7.25 Hz, 2H) 7.34 (t, J=7.44 Hz, 2H) 7.25-7.30 (m, 1H) 4.35 (s, 2H); MS ES⁺: 285.9.

Step 3: To a solution of 3-(benzylthio)-2-(difluoromethoxy)-5-fluoropyridine (500 mg, 1.75 mmol) in AcOH (2 mL) and H₂O (1 mL) was added NCS (936.1 mg, 7.01 mmol) at 0° C. The mixture was stirred at 25° C. for 12 hours. The reaction mixture was diluted with H₂O (30 mL) and extracted with ethyl acetate (3×30 mL). The combined organic layers were washed with brine (20 mL), dried over anhydrous Na₂SO₄, filtered and concentrated under reduced pressure to dryness to give a residue which was purified by flash silica gel chromatography (ISCO®; 12 g SepaFlash® Silica Flash Column, Eluent of 0˜3% Ethyl acetate/Petroleum ether gradient @30 mL/min) to afford 2-(difluoromethoxy)-5-fluoropyridine-3-sulfonyl chloride (370 mg, 1.41 mmol, 80.70% yield) as a yellow liquid, which was used without further purification.

### Intermediate 58: 5-chloro-2-(difluoromethoxy)pyridine-3-sulfonyl chloride

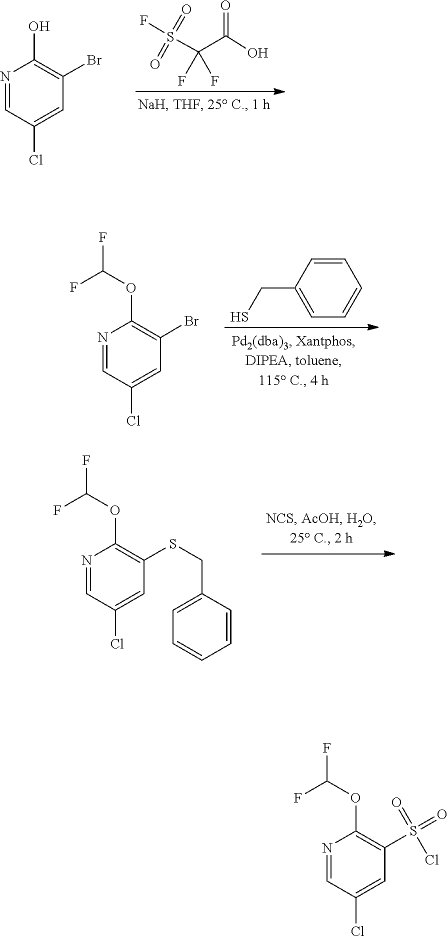 [SMILES: Oc1ncc(Cl)cc1Br ; O=C(O)C(F)(F)S(=O)(=O)F ; FC(F)Oc1ncc(Cl)cc1Br ; SCc1ccccc1 ; FC(F)Oc1ncc(Cl)cc1SCc1ccccc1 ; O=S(=O)(Cl)c1cc(Cl)cnc1OC(F)F]

Step 1: To a mixture of 3-bromo-5-chloro-pyridin-2-ol (3 g, 14.39 mmol) in THF (12 mL) was added NaH (1.15 g, 28.79 mmol, 60% purity) in portions at 0° C. under N₂. Then 2,2-difluoro-2-fluorosulfonyl-acetic acid (2.56 g, 14.39 mmol) was added to the mixture and the mixture was stirred at 25° C. for 2 hours. The mixture was quenched by saturated NH₄Cl (aq.) (60 mL) and extracted with ethyl acetate (3×60 mL). The combined organic layers were evaporated to dryness to give a residue which was purified by flash silica gel chromatography (ISCO®; 20 g SepaFlash® Silica Flash Column, Eluent of 0-0% Ethyl acetate/Petroleum ether gradient @65 mL/min) to give the product 3-bromo-5-chloro-2-(difluoromethoxy)pyridine (7-53 g, 28.07 mmol, 75.6% yield, 96.4% purity) as a yellow solid. MS ES⁺: 259.8.

Step 2: To a stirred mixture of phenylmethanethiol (96.1 mg, 0.774 mmol) and 3-bromo-5-chloro-2-(difluoromethoxy)pyridine (200 mg, 0.774 mmol) in toluene (2 mL) were added DIPEA (150 mg, 1.16 mmol), Xantphos (89.6 mg, 0.155 mmol) and Pd₂(dba)₃ (70.9 mg, 0.077 mmol) at 25° C. under N₂ atmosphere. The resulting mixture was stirred for 4 hours at 115° C. under N₂ atmosphere. The mixture was poured into ice water (30 mL) and adjusted to pH=9 with saturated NaHCO₃ (aq.), then extracted with ethyl acetate (3×15 mL). The combined organic layers were dried with anhydrous Na₂SO₄ and concentrated to dryness to give a residue which was purified by flash silica gel chromatography (ISCO®; 4 g SepaFlash® Silica Flash Column, Eluent of 0˜100% Ethyl acetate/Petroleum ether gradient @30 mL/min) to afford 3-benzylsulfanyl-5-chloro-2-(difluoromethoxy)pyridine (100 mg, 0.325 mmol, 42.03% yield, 98.1% purity) as a yellow liquid. ¹H NMR (400 MHz, DMSO-d₆): 8.08 (d, J=2.3 Hz, 1H), 7.99-7.78 (m, 1H), 7.69-7.46 (m, 1H), 7.45-7.38 (m, 2H), 7.35-7.25 (m, 3H), 4.51-4.15 (m, 2H); MS ES⁺: 302.0.

Step 3: To a solution of 3-benzylsulfanyl-5-chloro-2-(difluoromethoxy)pyridine (50 mg, 0.166 mmol) in H₂O (0.5 mL) and AcOH (1 mL) was added NCS (88.5 mg, 0.663 mmol) at 0° C. The reaction mixture was gradually warmed to 25° C. and then stirred for 5 hours. The mixture was poured into water (10 mL) and extracted with ethyl acetate (10 mL×3). The combined organic layers were concentrated to afford 5-chloro-2-(difluoromethoxy)pyridine-3-sulfonyl chloride (45 mg, crude) as a white solid, which was used without further purification. MS ES⁺: 279.0.

### Intermediate 59: 6,8-difluoro-1,2,3,4-tetrahydroisoquinoline

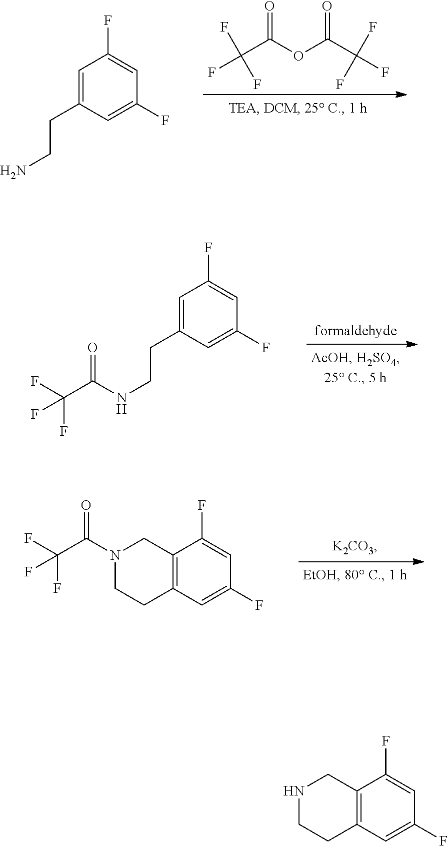 [SMILES: NCCc1cc(F)cc(F)c1 ; O=C(OC(=O)C(F)(F)F)C(F)(F)F ; O=C(NCCc1cc(F)cc(F)c1)C(F)(F)F ; O=C(N1CCc2cc(F)cc(F)c2C1)C(F)(F)F ; Fc1cc(F)c2c(c1)CCNC2]

Step 1: To a solution of 2-(3,5-difluorophenyl)ethanamine (500 mg, 3.18 mmol) in dichloromethane (10 mL) was added 2,2,2-trifluoroacetic anhydride (1.34 g, 6.36 mmol). The mixture was cooled to −15° C. before triethylamine (483 mg, 4.77 mmol) was dropwise added into the mixture. The mixture was allowed to warm to 25° C. and stirred at 25° C. for 30 minutes. The mixture was concentrated and extracted with dichloromethane (3×10 mL). The combined organic layers washed with brine (10 mL×2), dried with anhydrous Na₂SO₄, filtered and concentrated in vacuum to afford the crude product which was purified by flash silica gel chromatography (ISCO®; 12 g SepaFlash® Silica Flash Column, Eluent of 0˜10% Ethyl acetate/Petroleum ether gradient @30 mL/min) to afford N-[2-(3,5-difluorophenyl)ethyl]-2,2,2-trifluoro-acetamide (565 mg, 2.23 mmol, 70.15% yield) as a white solid. ¹H NMR (400 MHz, DMSO-d₆): 9.48 (br s, 1H), 7.07 (tt, J=2.2, 9.5 Hz, 1H), 6.99-6.96 (m, 1H), 6.96-6.92 (m, 1H), 3.50-3.39 (m, 2H), 2.88-2.78 (m, 2H).

Step 2: To a solution of N-[2-(3,5-difluorophenyl)ethyl]-2,2,2-trifluoro-acetamide (200 mg, 0.790 mmol) in AcOH (2.4 mL) at 0° C. was added con. H₂SO₄ (1.6 mL, 98% purity) slowly. Then formaldehyde (624 mg, 20.78 mmol) was added into the mixture. The mixture was allowed to warm to 25° C. and stirred for 5 hours. The mixture was poured into ice water, and extracted with ethyl acetate (3×20 mL). The combined organic layers were washed with 2 M K₂CO₃ (aq.) (20 mL), dried over anhydrous Na₂SO₄, filtered and concentrated to dryness to give a residue which was purified by flash silica gel chromatography (ISCO®; 4 g SepaFlash® Silica Flash Column, Eluent of 0˜30% Ethyl acetate/Petroleum ether gradient @30 mL/min) to afford 1-(6,8-difluoro-3,4-dihydro-1H-isoquinolin-2-yl)-2,2,2-trifluoro-ethanone (58.3 mg, 0.220 mmol, 27.83% yield) as a yellow oil. ¹H NMR (400 MHz, DMSO-d₆): 7.22-7.13 (m, 1H), 7.06-6.98 (m, 1H), 4.74-4.67 (m, 2H), 3.81 (br t, J=5.9 Hz, 2H), 2.99-2.90 (m, 2H).

Step 3: To a solution of 1-(6,8-difluoro-3,4-dihydro-1H-isoquinolin-2-yl)-2,2,2-trifluoro-ethanone (58 mg, 0.220 mmol) in EtOH (1.4 mL) was added a solution of K₂CO₃ (175.3 mg, 1.27 mmol) in H₂O (0.5 mL), and the resulting mixture was stirred at 80° C. for 1 hour. The mixture was concentrated to dryness to give a residue which was extracted with dichloromethane (3×25 mL). The combined organic layers were washed with brine (30 mL), dried over anhydrous Na₂SO₄, filtered and concentrated to dryness to afford 6,8-difluoro-1,2,3,4-tetrahydroisoquinoline (35 mg, crude) as a yellow oil, which was used without further purification. MS ES⁺: 170.1

### Intermediate 60: 1,1-dioxo-2,3-dihydrobenzothiophen-3-amine hydrochloride

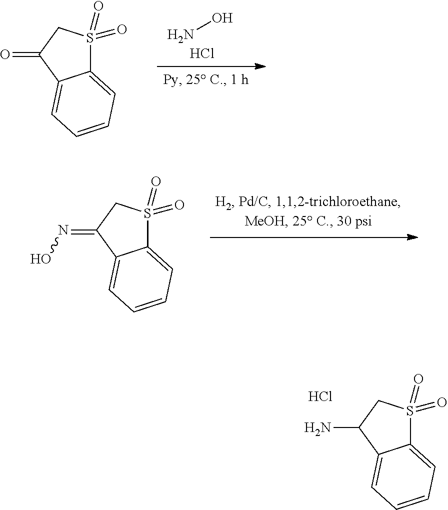

Step 1: A mixture of 1,1-dioxobenzothiophen-3-one (1 g, 5.49 mmol) and hydroxylamine hydrochloride (763 mg, 10.98 mmol) in pyridine (10 mL) was stirred at 25° C. for 1 hour. The mixture was concentrated to dryness which was purified by flash silica gel chromatography (ISCO®; 12 g SepaFlash® Silica Flash Column, Eluent of 0-50% Ethyl acetate/Petroleum ether gradient @30 mL/min) to afford 1,1-dioxobenzothiophen-3-one oxime (1.01 g, 5.12 mmol, 93.3% yield) as a light-yellow solid. ¹H NMR (400 MHz, DMSO-d₆): 12.3 (s, 1H) 7.9-8.1 (m, 2H) 7.7-7.8 (m, 2H) 4.5 (s, 2H)

Step 2: To a solution of 1,1-dioxobenzothiophen-3-one oxime (1.01 g, 5.12 mmol) in MeOH (1 mL) was added 1,1,2-trichloroethane (1.37 g, 10.24 mmol) and palladium on activated charcoal (1 g, 10% purity). The suspension was degassed under vacuum and purged with H₂ several times. The mixture was stirred for 10 hours at 25° C. under H₂ (30 psi). The mixture was filtered and concentrated to afford 1,1-dioxo-2,3-dihydrobenzothiophen-3-amine hydrochloride (900 mg, 4.66 mmol, 91.1% yield, 94.9% purity) as a yellow powder which was used in the next step directly. ¹H NMR (400 MHz, DMSO-d₆): 9.38 (br s, 2H), 8.12 (d, J=7.8 Hz, 1H), 7.96-7.81 (m, 2H), 7.77-7.64 (m, 1H), 5.27 (br t, J=6.5 Hz, 1H), 3.72 (dd, J=5.9, 13.9 Hz, 1H), 3.16 (s, 1H); MS ES⁺: 184.1

### Intermediate 61: 5-chloro-N-(7-fluorochroman-4-yl)-2-methoxynicotinamide

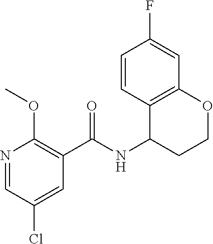 [SMILES: COc1ncc(Cl)cc1C(=O)NC1CCOc2cc(F)ccc21]

By following the procedure employed for Intermediate 1 using 5-chloro-2-methoxynicotinic acid (0.1 g, 0.53 mmol) and 7-Fluorochroman-4-amine (0.1 g, 0.6 mmol) in DMF (10 mL), crude product was obtained. Purification by flash column chromatography (Davisil silica, 20-30% EtOAc/Pet ether) gave 5-chloro-N-(7-fluorochroman-4-yl)-2-methoxynicotinamide (0.07 g, 39%) as a thick gum. MS m/z: 337.26 (M+H), 89.43%.

### 2. Synthesis of Examples

### Example 1: N-benzyl-5-chloro-2-methoxy-N-methylnicotinamide

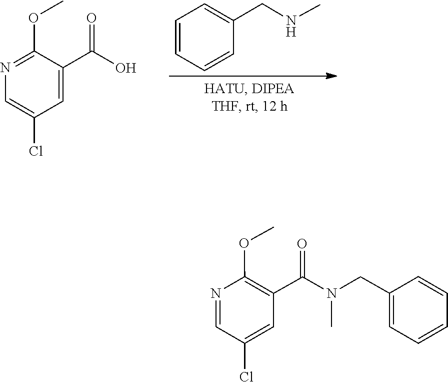

To a solution of 5-chloro-2-methoxynicotinic acid (0.15 g, 0.8 mmol) in THF was added DIPEA (0.309 g, 2.4 mmol), HATU (0.456 g, 1.2 mmol) and N-methyl-1-phenylmethanamine (0.116 g, 0.96 mmol). The reaction mixture was stirred at RT for 12 h. Water was added to the reaction mixture which was then extracted with EtOAc (3 15×20 mL). The combined organic phases were dried over Na₂SO₄ and evaporated under reduced pressure. Purification by reverse phase column chromatography (using C18, 40 g snap cartridge, 0-80% MeOH in 0.1% ammonium bicarbonate in water) gave N-benzyl-5-chloro-2-methoxy-N-methylnicotinamide (0.020 g, 8.6% yield) as an off-white solid.

¹H NMR (400 MHz, DMSO-d₆ at 90° C.): δ 8.24 (s, 1H), 7.79 (s, 1H), 7.40-7.10 (m, 5H), 4.67-4.20 (m, 2H), 4.0-3.80 (m, 3H), 2.95-2.70 (m, 3H); MS m/z: 291.18 (M+H).

### Example 2: 5-chloro-N-(6-cyano-2,3-dihydro-1H-inden-1-yl)-2-methoxy-N-methylnicotinamide

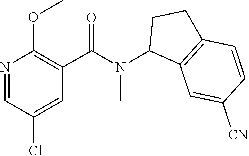 [SMILES: COc1ncc(Cl)cc1C(=O)N(C)C1CCc2ccc(C#N)cc21]

By following the procedure employed in Example 1 using 5-chloro-2-methoxynicotinic acid (0.13 g, 0.72 mmol) and 3-(methylamino)-2,3-dihydro-1H-indene-5-carbonitrile (Intermediate 13) gave 5-chloro-N-(6-cyano-2,3-dihydro-1H-inden-1-yl)-2-methoxy-N-methylnicotinamide (0.03 g, 15%) as an off-white solid.

¹H NMR (400 MHz, DMSO-d₆ at 90° C.): δ 8.24 (br s, 1H), 8.05-7.90 (m, 1H), 7.70-7.60 (m, 2H), 7.50-7.40 (m, 1H), 6.17 (br s, 0.45H), 5.09 (br s, 0.53H), 3.94 (s, 3H), 3.15-2.80 (m, 2H), 2.70-2.50 (m, 3H), 2.30-2.05 (m, 2H); MS m/z: 342.19 (M+H).

### Example 3: 5-chloro-N-(7-cyano-2,3-dihydro-1H-inden-1-yl)-2-methoxy-N-methylnicotinamide

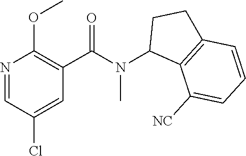 [SMILES: COc1ncc(Cl)cc1C(=O)N(C)C1CCc2cccc(C#N)c21]

By following the procedure employed in Example 1 using 5-chloro-2-methoxynicotinic acid (0.16 g, 0.87 mmol) and 3-(methylamino)-2,3-dihydro-1H-indene-4-carbonitrile (Intermediate 14) gave 5-chloro-N-(7-cyano-2,3-dihydro-1H-inden-1-yl)-2-methoxy-N-methylnicotinamide (0.02 g, 6.9%) as an off-white solid.

¹H NMR (400 MHz, DMSO-d₆ at 90° C.): δ 8.26 (d, J=2.4 Hz, 1H), 7.80-7.70 (m, 1H), 7.67-7.55 (m, 2H), 7.44 (t, J=7.6 Hz, 1H), 6.25 (s, 0.4H), 5.30 (s, 0.47H), 3.94 (s, 3H), 3.20-2.75 (m, 2H), 2.70-2.50 (m, 3H), 2.50-2.05 (m, 2H); MS m/z: 342.22 (M+H).

### Example 4: (5-fluoro-2-methoxypyridin-3-yl)(2-(3-fluorophenyl)piperidin-1-yl)methanone

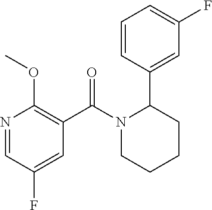 [SMILES: COc1ncc(F)cc1C(=O)N1CCCCC1c1cccc(F)c1]

By following the procedure employed in Example 1 using 5-fluoro-2-methoxynicotinic acid (0.20 g, 1.17 mmol) and 2-(3-fluorophenyl)piperidine (0.23 g, 1.29 mmol), gave (5-fluoro-2-methoxypyridin-3-yl)(2-(3-fluorophenyl)piperidin-1-yl)methanone (0.25 g, 65%) as a thick liquid.

¹H NMR (400 MHz, DMSO-d₆ at 90° C.): δ 8.17 (s, 1H), 7.74 (s, 1H), 7.43 (q, J=7.4 Hz, 1H), 7.25-7.10 (m, 1H), 7.06 (t, J=8.0 Hz, 1H), 5.82 (br s, 1H), 3.92 (s, 3H), 3.25 (br s, 1H), 2.40-2.30 (m, 1H), 1.95-1.80 (m, 1H), 1.65-1.30 (m, 5H); MS m/z: 333.27 (M+H).

### Example 5 (enantiomer 1) and Example 6 (enantiomer 2): 5-fluoro-N-(1-(2-fluorophenyl)ethyl)-2-methoxy-N-methylnicotinamide

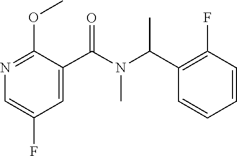 [SMILES: COc1ncc(F)cc1C(=O)N(C)C(C)c1ccccc1F]

By following the procedure employed in Example 1 using 5-fluoro-2-methoxynicotinic acid (0.3 g, 1.8 mmol) and 1-(2-fluorophenyl)-N-methylethan-1-amine (Intermediate 39) (0.30 g, 1.98 mmol), gave racemic 5-fluoro-N-(1-(2-fluorophenyl)ethyl)-2-methoxy-N-methylnicotinamide (0.3 g, 50%) as a colourless liquid. Enantiomers were separated by preparative SFC to afford Example 5 (0.25 g, 46%) & Example 6 (0.25 g, 46%) as colourless gums.

Preparative SFC condition: Column/dimensions: Chiralpak IC (30×250) mm, 5 μm; % CO₂: 75%; % Co solvent: 25% (MeOH); Total Flow: 120.0 g/min; Back Pressure: 100 bar; Temperature: 30° C.; UV: 220 nm; Solubility: Methanol.

Example 5: ¹H NMR (400 MHz, DMSO-d₆): δ 8.28-8.20 (m, 1H), 8.00-7.55 (m, 1H), 7.53-7.45 (m, 1H), 7.43-7.30 (m, 1H), 7.27-7.10 (m, 2H), 5.97 (q, J=6.8 Hz, 0.50H), 5.00-4.85 (br s, 0.47H), 3.95-3.60 (m, 3H), 2.90-2.50 (m, 3H), 1.60-1.50 (m, 3H); MS m/z: 307.28 (M+H). SFC Chiral Purity: 99.91%, Rt 1.15 mins.

Example 6: ¹H NMR (400 MHz, DMSO-d₆): δ 8.28-8.20 (m, 1H), 8.00-7.55 (m, 1H), 7.53-7.45 (m, 1H), 7.43-7.30 (m, 1H), 7.30-7.10 (m, 2H), 5.97 (q, J=7.2 Hz, 0.49H), 5.00-4.85 (br s, 0.47H), 3.95-3.60 (m, 3H), 2.90-2.50 (m, 3H), 1.60-1.50 (m, 3H); MS m/z: 307.28 (M+H). SFC Chiral Purity: 99.25%, Rt 1.53 mins.

### Example 7 (enantiomer 1) and Example 8 (enantiomer 2): 5-fluoro-N-(1-(3-fluorophenyl)ethyl)-2-methoxy-N-methylnicotinamide

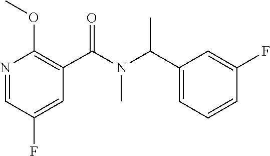 [SMILES: COc1ncc(F)cc1C(=O)N(C)C(C)c1cccc(F)c1]

By following the procedure employed in Example 1 using 5-fluoro-2-methoxynicotinic acid (0.15 g, 0.9 mmol) and 1-(3-fluorophenyl)-N-methylethan-1-amine (Intermediate 30) (0.15 g, 0.99 mmol), gave racemic 5-fluoro-N-(1-(3-fluorophenyl)ethyl)-2-methoxy-N-methylnicotinamide as an off-white semi solid. Enantiomers were separated by preparative SFC to afford Example 7 (0.10 g, 39%) & Example 8 (0.10 g, 39%) as pale red sticky solids.

Preparative SFC condition: Column/dimensions: Chiralpak AS-H (30×250 mm), 5 μm; ^{%}CO₂: 85%; Co-solvent: 15% (IPA); Total Flow: 95.0 g/min; Back Pressure: 100 bar; Temperature: 35° C.; UV: 220 nm. Solubility: Methanol.

Example 7: ¹H NMR (400 MHz, DMSO-d₆): δ 8.26-8.22 (m, 1H), 7.97-7.83 (m, 1H), 7.50-7.38 (m, 1H), 7.25-7.07 (m, 3H), 5.88 (q, J=6.8 Hz, 0.52H), 4.68 (q, J=6.8 Hz, 0.44H), 3.95-3.80 (m, 3H), 2.75-2.48 (m, 3H), 1.60-1.48 (m, 3H); MS m/z: 307.11 (M+H). SFC Chiral Purity: 99.59%, Rt 2.68 mins.

Example 8: ¹H NMR (400 MHz, DMSO-d₆): δ 8.26-8.22 (m, 1H), 7.97-7.83 (m, 1H), 7.50-7.38 (m, 1H), 7.25-7.07 (m, 3H), 5.88 (q, J=6.8 Hz, 0.53H), 4.68 (q, J=6.8 Hz, 0.44H), 3.95-3.80 (m, 3H), 2.75-2.48 (m, 3H), 1.60-1.48 (m, 3H); MS m/z: 307.08 (M+H). SFC Chiral Purity: 99.19%, Rt 4.64 mins.

### Example 9 (enantiomer 1) and Example 10 (enantiomer 2): 5-fluoro-N-(6-fluoro-2,3-dihydro-1H-inden-1-yl)-2-methoxy-N-methylnicotinamide

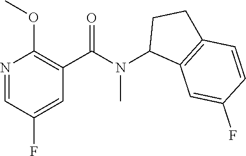 [SMILES: COc1ncc(F)cc1C(=O)N(C)C1CCc2ccc(F)cc21]

By following the procedure employed in Example 1 using 5-fluoro-2-methoxynicotinic acid (0.45 g, 2.63 mmol) and 5-fluoro-N-methyl-2,3-dihydro-1H-inden-1-amine (Intermediate 33) (0.48 g, 2.89 mmol), gave racemic 5-fluoro-N-(6-fluoro-2,3-dihydro-1H-inden-1-yl)-2-methoxy-N-methylnicotinamide as a pale yellow gum. Enantiomers were separated by preparative SFC to afford Example 9 & Example 10 (0.6 g, 70%) as white solids.

Preparative SFC condition: Column/dimensions: Chiralpak IE (30×250) mm, 5 μm; % CO₂: 80%; % Co-solvent: 20% (0.2% IPA in IPA); Total Flow: 100.0 g/min; Back Pressure: 100 bar; Temperature: 30° C.; UV: 220 nm. Solubility: IPA.

Example 9: ¹H NMR (401 MHz, DMSO-d₆): δ 8.28-8.22 (m, 1H), 8.13-7.80 (m, 1H), 7.35-7.00 (m, 3H), 6.16 (t, J=7.8 Hz, 0.33H), 4.99 (t, J=7.5 Hz, 0.65H), 3.95-3.85 (m, 3H), 3.10-2.60 (m, 4H), 2.45-2.00 (m, 3H); MS m/z: 319.19 (M+H). SFC Chiral Purity: 99.95%, Rt 2.71 mins.

Example 10: ¹H NMR (401 MHz, DMSO-d₆): δ 8.27-8.21 (m, 1H), 8.05-7.80 (m, 1H), 7.35-7.00 (m, 3H), 6.16 (t, J=7.8 Hz, 0.33H), 4.99 (t, J=7.5 Hz, 0.64H), 3.95-3.85 (m, 3H), 3.10-2.60 (m, 4H), 2.45-2.00 (m, 3H); MS m/z: 319.19 (M+H). SFC Chiral Purity: 99.80%, Rt 3.50 mins

### Example 11: N-(2,4-difluorobenzyl)-2-methoxy-N,6-dimethylnicotinamide

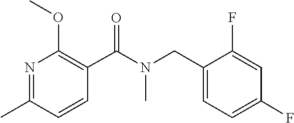 [SMILES: COc1nc(C)ccc1C(=O)N(C)Cc1ccc(F)cc1F]

By following the procedure employed in Example 1 using 2-methoxy-6-methylnicotinic acid (0.3 g, 1.8 mmol) and 1-(2,4-difluorophenyl)-N-methylmethanamine (Intermediate 43) (0.30 g, 1.98 mmol), gave N-(2,4-difluorobenzyl)-2-methoxy-N,6-dimethylnicotinamide (0.25 g, 46%) as a colourless liquid.

¹H NMR (400 MHz, DMSO-d₆): δ 7.63-7.50 (m, 1H), 7.48-7.05 (m, 3H), 6.96-6.86 (m, 1H), 4.75-4.33 (m, 2H), 3.90-3.81 (m, 3H), 2.89-2.73 (m, 3H), 2.45-2.40 (m, 3H); MS m/z: 307.18 (M+H).

### Example 12 (enantiomer 1) and Example 13 (enantiomer 2): 5-chloro-N-(2,3-dihydro-1H-inden-1-yl)-2-ethoxy-N-methylnicotinamide

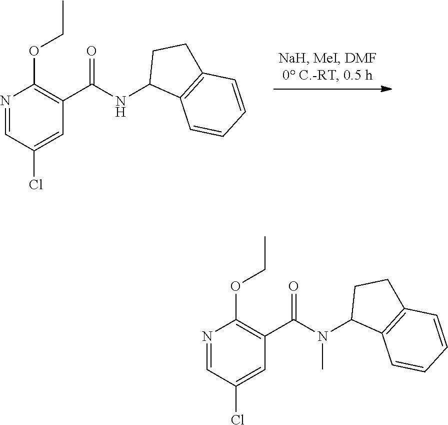 [SMILES: CCOc1ncc(Cl)cc1C(=O)NC1CCc2ccccc21 ; CCOc1ncc(Cl)cc1C(=O)N(C)C1CCc2ccccc21]

To a stirred solution of 5-chloro-N-(2,3-dihydro-1H-inden-1-yl)-2-ethoxynicotinamide (Intermediate 5) (0.18 g, 0.6 mmol) in DMF (5 mL) at 0° C., was added NaH (0.29 g, 1.2 mmol). After 10 mins, methyl iodide (0.128 g, 0.9 mmol) was added at 0° C. and the resulting mixture stirred at RT for 2 h. The reaction mixture was cooled to 0° C., quenched with water and extracted with EtOAc (3×20 mL). The combined organic phases were washed with ice-cold water, dried over Na₂SO₄ and evaporated under reduced pressure. Purification by preparative HPLC afforded 5-chloro-N-(2,3-dihydro-1H-inden-1-yl)-2-ethoxy-N-methylnicotinamide (0.1 g, 53%) as an off-white gum. Enantiomers were separated by preparative SFC to afford Example 12 (0.04 g, 21%) & Example 13 (0.04 g, 21%) as off-white solids.

Preparative SFC condition: Column/dimensions: Chiralpak AS-H (30*250) mm, 5 μm; % CO₂: 80%; % Co-solvent: 20% (0.2% 7M Methanolic Ammonia in Methanol). Total Flow: 85.0 g/min; Back Pressure: 100 bar; Temperature: 35° C.; UV: 220 nm. Solubility: Methanol.

Example 12: ¹H NMR (400 MHz, DMSO-d₆): δ 8.21 (s, 1H), 7.85-7.80 (m, 1H), 7.30-7.20 (br s, 4H), 6.19 (s, 0.42H), 5.02 (s, 0.49H), 4.40 (q, J=7.2 Hz, 2H), 3.00-2.70 (m, 2H), 2.65-2.50 (m, 3H), 2.50-1.90 (m, 2H), 1.33 (br s, 3H). MS m/z: 331.10 (M+H). SFC Chiral Purity: 99.78%, Rt 5.12 mins

Example 13: ¹H NMR (400 MHz, DMSO-d₆): δ 8.21 (s, 1H), 7.84-7.80 (m, 1H), 7.28-7.20 (br s, 4H), 6.19 (s, 0.4H), 5.02 (s, 0.49H), 4.40 (q, J=7.2 Hz, 2H), 3.00-2.70 (m, 2H), 2.65-2.40 (m, 3H), 2.40-1.90 (m, 2H), 1.33 (br s, 3H); MS m/z: 331.10 (M+H). SFC Chiral Purity: 99.61%, Rt 6.77 mins

Following the general procedure as described in the preparation of Example 12 and Example 13, the following Examples were prepared:

### Example 14 (enantiomer 1) and Example 15 (enantiomer 2): N-(chroman-4-yl)-5-fluoro-2-methoxy-N-methylnicotinamide

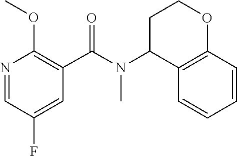 [SMILES: COc1ncc(F)cc1C(=O)N(C)C1CCOc2ccccc21]

Following the general procedure as described in the preparation of Example 12 and Example 13, using N-(chroman-4-yl)-5-fluoro-2-methoxynicotinamide (Intermediate 1) (0.33 g, 1.0 mmol), gave racemic N-(chroman-4-yl)-5-fluoro-2-methoxy-N-methylnicotinamide (0.15 g, 43%) as an off-white solid. Enantiomers were separated by preparative SFC to obtain Example 14 and Example 15 (0.04 g, 11%) as off-white solids.

Preparative SFC condition: Column/dimensions: Chiralcel-OX-H (30×250) mm, 5 μm; % CO₂: 70%; % Co solvent: 30% (0.2% Isopropylamine in Isopropanol); Total Flow: 120.0 g/min; Back Pressure: 100 bar; Temperature: 30° C.; UV: 220 nm; Solubility: Methanol.

Example 14: ¹H NMR (400 MHz, DMSO-d₆ at 90° C.): δ 8.20-8.17 (m, 1H), 7.85-7.70 (m, 1H), 7.20-7.10 (m, 2H), 7.00-6.85 (m, 1H), 6.83-6.70 (m, 1H), 5.95-5.85 (m, 0.5H), 4.70-4.60 (m, 0.48H), 4.40-4.00 (m, 2H), 4.00-3.80 (m, 3H), 2.67 (s, 3H), 2.25-1.95 (m, 2H); MS m/z: 317.01 (M+H). SFC Chiral Purity: 99.30%, Rt 2.50 mins.

Example 15: ¹H NMR (400 MHz, DMSO-d₆ at 90° C.): δ 8.20-8.15 (m, 1H), 7.83-7.73 (m, 1H), 7.20-7.10 (m, 2H), 6.95-6.90 (m, 1H), 6.85-6.70 (m, 1H), 5.95-5.85 (m, 0.46H), 4.80-4.70 (m, 0.45H), 4.40-4.00 (m, 2H), 4.00-3.85 (m, 3H), 2.67 (s, 3H), 2.25-1.95 (m, 2H); MS m/z: 317.03 (M+H). SFC Chiral Purity: 99.95% Rt 4.62 mins.

### Example 16 (enantiomer 1) and Example 17 (enantiomer 2): N-(2, 3-dihydro-1H-inden-1-yl)-5-fluoro-2-methoxy-N-methylnicotinamide

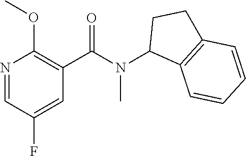 [SMILES: COc1ncc(F)cc1C(=O)N(C)C1CCc2ccccc21]

Following the general procedure as described in the preparation of Example 12 and Example 12, using N-(2,3-dihydro-1H-inden-1-yl)-5-fluoro-2-methoxynicotinamide (Intermediate 3) (0.31 g, 1.089 mmol), gave racemic N-(2,3-dihydro-1H-inden-1-yl)-5-fluoro-2-methoxy-N-methylnicotinamide as an off-white gum. The enantiomers were separated by preparative SFC to afford Example 16 & Example 17 (0.048 g, 15%) as off white gums.

Preparative SFC condition: Column/dimensions: Chiralpak AD-H (30*250) mm, 5 μm; % CO₂: 85%; % Co solvent: 15% (0.2% Isopropyl Amine in IPA). Total Flow: 85.0 g/min; Back Pressure: 100 bar. Temperature: 35° C.; UV: 220 nm; Solubility: IPA.

Example 16: ¹H NMR (400 MHz, DMSO-d₆ at 90° C.): δ 8.17 (s, 1H), 7.75 (br s, 1H), 7.23 (s, 4H), 6.18 (s, 0.33H), 5.01 (s, 0.51H), 3.92 (s, 3H), 3.00-2.70 (m, 2H), 2.70-2.40 (m, 3H), 2.40-1.95 (m, 2H); MS m/z: 301.21 (M+H). SFC Chiral Purity: 99.79%, Rt 3.36 mins

Example 17: ¹H NMR (400 MHz, DMSO-d₆ at 90° C.): δ 8.17 (s, 1H), 7.75 (br s, 1H), 7.23 (s, 4H), 6.18 (s, 0.32H), 5.01 (s, 0.54H), 3.92 (s, 3H), 3.00-2.70 (m, 2H), 2.70-2.40 (m, 3H), 2.40-1.95 (m, 2H); MS m/z: 301.21 (M+H). SFC Chiral Purity: 99.30%, Rt 4.39 mins.

### Example 18 (enantiomer 1) and Example 19 (enantiomer 2): 5-chloro-N-(chroman-4-yl)-2-methoxy-N-methylnicotinamide

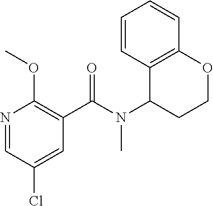 [SMILES: COc1ncc(Cl)cc1C(=O)N(C)C1CCOc2ccccc21]

Following the general procedure as described in the preparation of Example 12 and Example 13, using 5-chloro-N-(chroman-4-yl)-2-methoxynicotinamide (Intermediate 4) (0.24 g, 0.7547 mmol) gave racemic s-chloro-N-(chroman-4-yl)-2-methoxy-N-methylnicotinamide as an off-white gummy solid. Enantiomers were separated by preparative SFC to afford Example 18 & Example 19 (0.04 g, 16%) as off-white solids.

Preparative SFC condition: Column/dimensions: Chiralpak AS-H (30*250) mm, 5 μm. % CO₂: 80%; % Co-solvent: 20% (0.2% 7M Methanolic Ammonia in Methanol). Total Flow: 85.0 g/min. Back Pressure: 100 bar. Temperature: 35° C. UV: 220 nm; Solubility: Methanol.

Example 18: ¹H NMR (400 MHz, DMSO-d₆ at 90° C.): δ 8.26-8.22 (m, 1H), 7.88 (br s, 1H), 7.20-7.10 (m, 2H), 6.93 (t, J=7.6 Hz, 1H), 6.82-6.72 (m, 1H), 5.89 (s, 0.48H), 4.76 (s, 0.43H), 4.30-4.10 (m, 2H), 4.00-3.90 (m, 3H), 2.70-2.50 (m, 3H), 2.25-1.90 (m, 2H); MS m/z: 333.21 (M+H). SFC Chiral Purity: 99.77%, Rt 2.82 mins.

Example 19: ¹H NMR (400 MHz, DMSO-d₆ at 90° C.): δ 8.30-8.20 (m, 1H), 7.89 (br s, 1H), 7.20-7.10 (m, 2H), 6.93 (t, J=7.6 Hz, 1H), 6.82-6.72 (m, 1H), 5.89 (s, 0.47H), 4.76 (s, 0.43H), 4.30-4.10 (m, 2H), 4.00-3.90 (m, 3H), 2.70-2.50 (m, 3H), 2.25-1.90 (m, 2H); MS m/z: 333.21 (M+H). SFC Chiral Purity: 99.80%, Rt 4.56 mins.

### Example 20 (enantiomer 1) and Example 21 (enantiomer 2): 5-chloro-N-(6-fluoro-2,3-dihydro-1H-inden-1-yl)-2-methoxy-N-methylnicotinamide

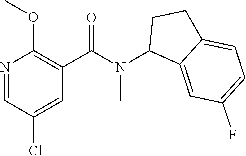 [SMILES: COc1ncc(Cl)cc1C(=O)N(C)C1CCc2ccc(F)cc21]

Following the general procedure as described in the preparation of Example 12 and Example 13, using 5-chloro-N-(6-fluoro-2,3-dihydro-1H-inden-1-yl)-2-methoxynicotinamide (Intermediate 6) (0.5 g, 2.6 mmol), gave racemic 5-chloro-N-(6-fluoro-2,3-dihydro-1H-inden-1-yl)-2-methoxynicotinamide as a colourless gum. The enantiomers were separated by preparative SFC to afford Example 20 & Example 21 (0.04 g, 7.7%) as off-white solids.

Preparative SFC condition: Column/dimensions: Chiralpak AS-H (30*250) mm, 5 μm. % CO₂: 80%; % Co solvent: 20% (0.2% Isopropylamine in IPA); Total Flow: 90.0 g/min; Back Pressure: 100 bar; Temperature: 35° C.; UV: 220 nm. Solubility: IPA.

Example 20: ¹H NMR (400 MHz, DMSO-d₆): δ 8.24 (s, 1H), 7.89 (s, 1H), 7.26 (s, 1H), 7.05-7.00 (br s, 2H), 6.15 (s, 0.36H), 5.02 (s, 0.49H), 3.94 (s, 3H), 3.00-2.70 (m, 2H), 2.65-2.50 (m, 3H), 2.50-1.90 (m, 2H); MS m/z: 335.29 (M+H). SFC Chiral Purity: 99.80%, Rt 4.82 mins.

Example 21: ¹H NMR (400 MHz, DMSO-d₆): δ 8.24 (s, 1H), 7.89 (s, 1H), 7.24 (s, 1H), 7.05-7.00 (br s, 2H), 6.15 (s, 0.42H), 5.02 (s, 0.56H), 3.93 (s, 3H), 3.00-2.70 (m, 2H), 2.65-2.50 (m, 3H), 2.50-1.90 (m, 2H); MS m/z: 335.29 (M+H). SFC Chiral Purity: 99.77%, Rt 7.27 mins.

### Example 22 (enantiomer 1) and Example 23 (enantiomer 2): 5-chloro-N-(2,3-dihydro-1H-inden-1-yl)-2-methoxy-N-methylnicotinamide

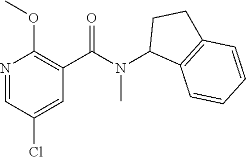 [SMILES: COc1ncc(Cl)cc1C(=O)N(C)C1CCc2ccccc21]

Following the general procedure as described in the preparation of Example 12 and Example 13, using 5-chloro-N-(2,3-dihydro-1H-inden-1-yl)-2-methoxynicotinamide (Intermediate 2) (0.7 g, 2.3 mmol), gave racemic 5-chloro-N-(2,3-dihydro-1H-inden-1-yl)-2-methoxy-N-methylnicotinamide as an off-white gum. The enantiomers were separated by preparative SFC to afford Example 22 & Example 23 (0.4 g, 41%) as off-white solids.

SFC preparative condition: (Column/dimensions: Chiralpak AD-H (30*250) mm, 5 μm; % CO₂: 85%; % Co solvent: 15% (0.2% Isopropylamine in IPA). Total Flow: 90.0 g/min. Back Pressure: 100 bar. Temperature: 35° C.; UV: 220 nm.

Example 22: ¹H NMR (400 MHz, DMSO-d₆ at 90° C.): δ 8.24 (s, 1H), 7.85 (s, 1H), 7.23 (s, 4H), 6.17 (s, 0.3H), 5.01 (s, 0.5H), 3.94 (s, 3H), 3.00-2.60 (m, 2H), 2.64-2.45 (m, 3H), 2.40-2.00 (m, 2H); MS m/z: 317.18 (M+H). SFC Chiral Purity: 99.94%, Rt 5.13 mins.

Example 23: ¹H NMR (400 MHz, DMSO-d₆ at 90° C.): δ 8.24 (s, 1H), 7.85 (br s, 1H), 7.23 (s, 4H), 6.18 (s, 0.35H), 5.01 (s, 0.52H), 3.94 (s, 3H), 3.0-2.70 (m, 2H), 2.65-2.40 (m, 3H), 2.40-2.00 (m, 2H); MS m/z: 317.18 (M+H). Chiral Purity: 99.87%, Rt. 7.13 mins.

### Example 24: 5-chloro-N-(3-fluorobenzyl)-2-methoxy-N-methylnicotinamide

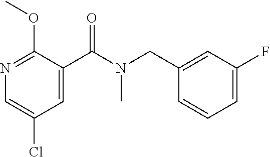 [SMILES: COc1ncc(Cl)cc1C(=O)N(C)Cc1cccc(F)c1]

Following the general procedure as described in the preparation of Example 12 and Example 13, using 5-chloro-N-(3-fluorobenzyl)-2-methoxynicotinamide (Intermediate 12) (0.18 g, 0.6 mmol), gave 5-chloro-N-(3-fluorobenzyl)-2-methoxy-N-methylnicotinamide (0.17 g, 90%) as a pale yellow solid.

¹H NMR (400 MHz, DMSO-d₆ at 90° C.): δ 8.26-8.20 (br s, 1H), 7.85-7.75 (m, 1H), 7.45-7.35 (br s, 1H), 7.25-6.95 (m, 3H), 4.80-4.20 (m, 2H), 3.95-3.80 (m, 3H), 2.95-2.70 (m, 3H); MS m/z: 309.16 (M+H).

### Example 25 (racemate), Example 26 (enantiomer 1) and Example 27 (enantiomer 2): 5-chloro-N-(2,3-dihydrobenzofuran-3-yl)-2-methoxy-N-methylnicotinamide

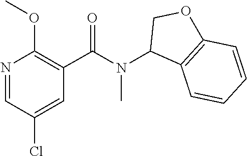 [SMILES: COc1ncc(Cl)cc1C(=O)N(C)C1COc2ccccc21]

Following the general procedure as described in the preparation of Example 12 and Example 13, using 5-chloro-N-(2,3-dihydrobenzofuran-3-yl)-2-methoxynicotinamide (Intermediate 15) (0.3 g, 1.0 mmol), gave Example 25 5-chloro-N-(2,3-dihydrobenzofuran-3-yl)-2-methoxy-N-methylnicotinamide (0.40 g, 78%) as an off-white solid. The enantiomers were separated by preparative SFC to afford Example 26 & Example 27 (0.16 g, 30%) as an off-white solid.

SFC preparative condition: Column/dimensions Chiralcel OX-H (30*250) mm, 5 μm; % CO₂: 80%; % Co-solvent: 20% (0.2% 7M Methanolic Ammonia in Methanol); Total Flow: 90.0 g/min; Back Pressure: 100 bar; Temperature: 35° C.; UV: 220 nm. Solubility: Methanol.

Example 25: ¹H NMR (400 MHz, DMSO-d₆ at 90° C.): δ 8.26 (s, 1H), 7.92-7.80 (m, 1H), 7.35-7.20 (m, 2H), 6.95 (t, J=7.1 Hz, 1H), 6.87-6.80 (m, 1H), 6.34 (br s, 0.35H), 5.31 (br s, 0.44H), 4.70-4.40 (m, 2H), 3.95 (s, 3H), 2.65-2.40 (m, 3H); MS m/z: 319.20 (M+H).

Example 26: ¹H NMR (400 MHz, DMSO-d₆ at 90° C.): δ 8.26 (d, J=2.0 Hz, 1H), 7.92-7.80 (m, 1H), 7.33-7.20 (m, 2H), 6.95 (t, J=7.6 Hz, 1H), 6.87-6.80 (m, 1H), 6.34 (br s, 0.38H), 5.31 (br s, 0.47H), 4.75-4.40 (m, 2H), 3.95 (s, 3H), 2.65-2.40 (m, 3H); MS m/z: 319.20 (M+H). SFC Chiral purity: 99.99%, Rt 2.52 mins.

Example 27: ¹H NMR (400 MHz, DMSO-d₆ at 90° C.): δ 8.25 (s, 1H), 7.92-7.80 (m, 1H), 7.34-7.20 (m, 2H), 7.00-6.90 (m, 1H), 6.87-6.80 (m, 1H), 6.34 (s, 0.34H), 5.31 (s, 0.44H), 4.70-4.40 (m, 2H), 3.94 (s, 3H), 2.70-2.40 (m, 3H); MS m/z: 319.20 (M+H).

SFC Chiral purity: 99.55%. Rt 3.40 mins.

### Example 28 (enantiomer 1) and Example 29 (enantiomer 2): 5-chloro-N-(8-fluorochroman-4-yl)-2-methoxy-N-methylnicotinamide

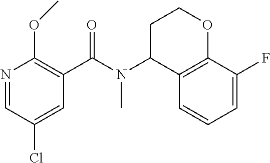 [SMILES: COc1ncc(Cl)cc1C(=O)N(C)C1CCOc2c(F)cccc21]

Following the general procedure as described in the preparation of Example 12 and Example 13, using 5-chloro-N-(8-fluorochroman-4-yl)-2-methoxynicotinamide (Intermediate 8) (0.35 g, 1.0 mmol), gave racemic 5-chloro-N-(8-fluorochroman-4-yl)-2-methoxy-N-methylnicotinamide as a colourless gum. The enantiomers were separated by preparative SFC to afford Example 28 & Example 29 (0.04 g, 11%) as off-white solids.

SFC preparative condition: Column/dimensions: (Chiralpak AS-H (30*250) mm, 5 μm; % CO₂: 90%; % Co-solvent: 15% (0.2% 7M Methanolic Ammonia In MeOH); Total Flow: 90.0 g/min; Back Pressure: 100 bar; Temperature: 35° C.; UV: 260 nm. Solubility: MeOH.

Example 28: ¹H NMR (400 MHz, DMSO-d₆ at 90° C.): δ 8.24 (s, 1H), 7.89 (s, 1H), 7.10-6.80 (m, 3H), 5.91 (s, 0.51H), 4.80 (s, 0.43H), 4.50-4.10 (m, 2H), 3.97-3.90 (m, 3H), 2.70-2.50 (m, 3H), 2.40-2.20 (m, 1H), 2.10-2.00 (m, 1H); MS m/z: 351.21 (M+H). SFC Chiral Purity: 99.98%, Rt 3.49 mins.

Example 29: ¹H NMR (400 MHz, DMSO-d₆ at 90° C.): δ 8.24 (s, 1H), 7.89 (s, 1H), 7.08-6.88 (m, 3H), 5.93 (s, 0.52H), 4.80 (s, 0.41H), 4.40-4.11 (m, 2H), 3.97-3.90 (m, 3H), 2.70-2.50 (m, 3H), 2.40-2.20 (m, 1H), 2.10-2.00 (m, 1H); MS m/z: 351.21 (M+H). SFC Chiral Purity: 99.88%, Rt 5.59 mins.

### Example 30 (enantiomer 1) and Example 31 (enantiomer 2): 5-fluoro-N-(8-fluorochroman-4-yl)-2-methoxy-N-methylnicotinamide

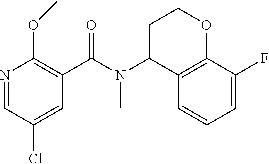 [SMILES: COc1ncc(Cl)cc1C(=O)N(C)C1CCOc2c(F)cccc21]

Following the general procedure as described in the preparation of Example 12 and Example 13, using 5-fluoro-N-(8-fluorochroman-4-yl)-2-methoxynicotinamide (Intermediate 9) (0.6 g, 1.9 mmol), gave racemic 5-fluoro-N-(8-fluorochroman-4-yl)-2-methoxy-N-methylnicotinamide as a colourless gum. The enantiomers were separated by preparative SFC to afford Example 30 & Example 31 (0.04 g, 6.4%) as off-white solids.

Preparative SFC condition: Column/dimensions: Chiralpak AS-H (30*250) mm, 5 μm; % CO₂: 90%; % Co-solvent: 15% (0.2% 7M Methanolic Ammonia In MeOH); Total Flow: 90.0 g/min; Back Pressure: 100 bar; Temperature: 35° C.; UV: 260 nm. Solubility: MeOH.

Example 30: ¹H NMR (400 MHz, DMSO-d₆ at 90° C.): δ 8.22-8.17 (m, 1H), 7.82-7.72 (br s, 1H), 7.10-6.85 (m, 3H), 5.95-5.88 (m, 0.50H), 4.85-4.75 (m, 0.42H), 4.50-4.10 (m, 2H), 3.97-3.90 (m, 3H), 2.70-2.50 (m, 3H), 2.35-2.18 (m, 1H), 2.15-2.00 (m, 1H); MS m/z: 335.24 (M+H). SFC Chiral Purity: 99.88%, Rt 2.71 mins.

Example 31: ¹H NMR (400 MHz, DMSO-d₆ at 90° C.): δ 8.22-8.17 (m, 1H), 7.82-7.72 (m, 1H), 7.10-6.85 (m, 3H), 5.95-5.88 (m, 0.49H), 4.85-4.75 (m, 0.43H), 4.50-4.10 (m, 2H), 3.97-3.90 (m, 3H), 2.70-2.50 (m, 3H), 2.35-2.18 (m, 1H), 2.15-2.00 (m, 1H); MS m/z: 335.24 (M+H). SFC Chiral Purity: 98.96%, Rt 3.67 mins.

### Example 32: N-(3-fluorobenzyl)-2-methoxy-N-methylnicotinamide

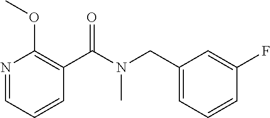 [SMILES: COc1ncccc1C(=O)N(C)Cc1cccc(F)c1]

Following the general procedure as described in the preparation of Example 12 and Example 13, using N-(3-fluorobenzyl)-2-methoxynicotinamide (Intermediate 19) (0.4 g, 1.5 mmol), gave N-(3-fluorobenzyl)-2-methoxy-N-methylnicotinamide (0.15 g, 60%) as an off-white solid.

¹H NMR (400 MHz, DMSO-d₆ at 90° C.): δ 8.22 (br s, 1H), 7.67 (br s, 1H), 7.45-6.85 (m, 5H), 4.70-4.30 (m, 2H), 3.95-3.80 (m, 3H), 2.95-2.70 (m, 3H); MS m/z: 275.17 (M+H).

### Example 33: N-(2-fluorobenzyl)-2-methoxy-N-methylnicotinamide

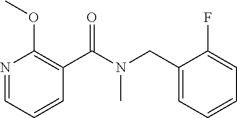 [SMILES: COc1ncccc1C(=O)N(C)Cc1ccccc1F]

Following the general procedure as described in the preparation of Example 12 and Example 13, using N-(2-fluorobenzyl)-2-methoxynicotinamide (Intermediate 20) (0.4 g, 1.5 mmol), gave N-(2-fluorobenzyl)-2-methoxy-N-methylnicotinamide (0.15 g, 60%) as a pale yellow gum.

¹H NMR (400 MHz, DMSO-d₆ at 90° C.): δ 8.23 (s, 1H), 7.65 (br s, 1H), 7.48-7.00 (m, 5H), 4.80-4.35 (m, 2H), 3.95-3.80 (m, 3H), 3.05-2.70 (m, 3H). HPLC Purity: 99.65%; MS m/z: 275.22 (M+H), 98.98%.

### Example 34: 5-fluoro-N-(1-(3-fluorophenyl)cyclopropyl)-2-methoxy-N-methylnicotinamide

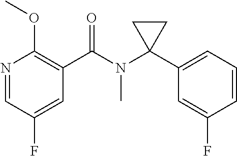 [SMILES: COc1ncc(F)cc1C(=O)N(C)C1(c2cccc(F)c2)CC1]

Following the general procedure as described in the preparation of Example 12 and Example 13, using s-fluoro-N-(i-(3-fluorophenyl)cyclopropyl)-2-methoxynicotinamide (Intermediate 21) (0.2 g, 0.66 mmol), gave 5-fluoro-N-(1-(3-fluorophenyl)cyclopropyl)-2-methoxy-N-methylnicotinamide (0.18 g, 86%) as a white solid.

¹H NMR (400 MHz, DMSO-d₆ at 90° C.): δ 8.20-8.05 (m, 1H), 7.75-7.65 (br s, 1H), 7.40-6.50 (m, 4H), 4.00-3.65 (m, 3H), 3.16 (br s, 1H), 2.81 (s, 2H), 1.50-1.11 (m, 4H); MS m/z: 319.24 (M+H).

### Example 35: 5-fluoro-N-(5-fluoro-2,3-dihydrobenzofuran-3-yl)-2-methoxy-N-methylnicotinamide

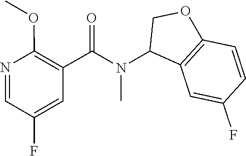 [SMILES: COc1ncc(F)cc1C(=O)N(C)C1COc2ccc(F)cc21]

Following the general procedure as described in the preparation of Example 12 and Example 13, using 5-fluoro-N-(5-fluoro-2,3-dihydrobenzofuran-3-yl)-2-methoxynicotinamide (Intermediate 16) (0.2 g, 0.69 mmol), gave 5-fluoro-N-(5-fluoro-2,3-dihydrobenzofuran-3-yl)-2-methoxy-N-methylnicotinamide (0.06 g, 78%) as an off-white solid.

¹H NMR (400 MHz, DMSO-d₆): δ 8.30-8.25 (m, 1H), 8.05-7.70 (m, 1H), 7.33-6.85 (m, 3H), 6.40-6.35 (m, 0.36H), 5.40-5.32 (m, 0.62H), 4.80-4.45 (m, 2H), 4.05-3.85 (m, 3H), 2.65-2.40 (m, 3H); MS m/z: 321.21 (M+H).

### Example 36: N-(2-cyanobenzyl)-5-fluoro-2-methoxy-N-methylnicotinamide

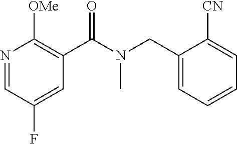 [SMILES: COc1ncc(F)cc1C(=O)N(C)Cc1ccccc1C#N]

Following the general procedure as described in the preparation of Example 12 and Example 13, using N-(2-cyanobenzyl)-5-fluoro-2-methoxynicotinamide (0.12 g, 0.4 mmol) (Intermediate 22), gave N-(2-cyanobenzyl)-5-fluoro-2-methoxy-N-methylnicotinamide (0.1 g, 83%) as an off-white solid

¹H NMR (400 MHz, DMSO-d₆ at 90° C.): δ 8.29-8.19 (m, 1H), 7.92-7.70 (m, 3H), 7.58-7.45 (m, 2H), 4.95-4.55 (m, 2H), 3.95-3.75 (m, 3H), 3.00-2.80 (m, 3H); MS m/z: 300.25 (M+H).

### Example 37: 5-fluoro-N-(2-fluorobenzyl)-2-methoxy-N-methylnicotinamide

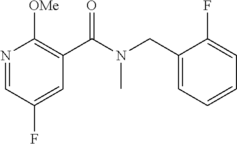 [SMILES: COc1ncc(F)cc1C(=O)N(C)Cc1ccccc1F]

Following the general procedure as described in the preparation of Example 12 and Example 13, using 5-fluoro-N-(2-fluorobenzyl)-2-methoxynicotinamide (Intermediate 23) (0.25 g, 0.9 mmol), gave 5-fluoro-N-(2-fluorobenzyl)-2-methoxy-N-methylnicotinamide (0.17 g, 68%) as an off-white solid.

¹H NMR (400 MHz, DMSO-d₆): δ 8.30-8.20 (m, 1H), 7.85-7.70 (m, 1H), 7.45-7.10 (m, 4H), 4.80-4.39 (m, 2H), 3.95-3.80 (m, 3H), 2.95-2.75 (m, 3H); MS m/z: 293.20 (M+H).

### Example 38: N-(2,3-difluorobenzyl)-5-fluoro-2-methoxy-N-methylnicotinamide

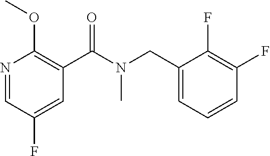 [SMILES: COc1ncc(F)cc1C(=O)N(C)Cc1cccc(F)c1F]

Following the general procedure as described in the preparation of Example 12 and Example 13, using N-(2,3-difluorobenzyl)-5-fluoro-2-methoxynicotinamide (Intermediate 24) (0.2 g, 0.67 mmol), gave N-(2,3-difluorobenzyl)-5-fluoro-2-methoxy-N-methylnicotinamide (0.1 g, 60%) as an off-white solid.

¹H NMR (400 MHz, DMSO-d₆): δ 8.28-8.20 (m, 1H), 7.85-7.65 (m, 1H), 7.43-7.05 (m, 3H), 4.85-4.45 (m, 2H), 3.93-3.80 (m, 3H), 2.95-2.78 (m, 3H); MS m/z: 311.19 (M+H).

### Example 39: N-(2,4-difluorobenzyl)-5-fluoro-2-methoxy-N-methylnicotinamide

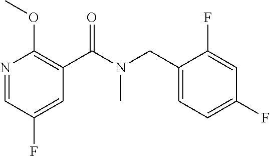 [SMILES: COc1ncc(F)cc1C(=O)N(C)Cc1ccc(F)cc1F]

Following the general procedure as described in the preparation of Example 12 and Example 13, using N-(2,4-difluorobenzyl)-5-fluoro-2-methoxynicotinamide (Intermediate 25) (0.2 g, 0.67 mmol), gave N-(2,4-difluorobenzyl)-5-fluoro-2-methoxy-N-methylnicotinamide (0.2 g, 90%) as an off-white solid.

¹H NMR (400 MHz, DMSO): δ 8.30-8.22 (m, 1H), 7.85-7.73 (m, 1H), 7.50-7.05 (m, 3H), 4.80-4.35 (m, 2H), 3.92-3.80 (m, 3H), 2.92-2.75 (m, 3H); MS m/z: 311.19 (M+H).

### Example 40: 5-chloro-N-(2,3-difluorobenzyl)-2-methoxy-N-methylnicotinamide

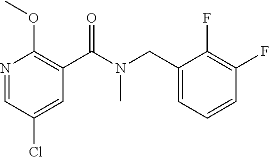 [SMILES: COc1ncc(Cl)cc1C(=O)N(C)Cc1cccc(F)c1F]

Following the general procedure as described in the preparation of Example 12 and Example 13, using 5-chloro-N-(2,3-difluorobenzyl)-2-methoxynicotinamide (Intermediate 18) (0.2 g, 0.69 mmol), gave 5-chloro-N-(2,3-difluorobenzyl)-2-methoxy-N-methylnicotinamide (0.06 g, 78%) as an off-white solid.

¹HNMR (400 MHz, DMSO-d₆): δ 8.35-8.27 (m, 1H), 7.93-7.81 (m, 1H), 7.45-7.05 (m, 3H), 4.85-4.45 (m, 2H), 3.93-3.81 (m, 3H), 2.95-2.78 (m, 3H); MS m/z: 327.16 (M+H).

### Example 41: 5-chloro-N-(3,5-difluorobenzyl)-2-methoxy-N-methylnicotinamide

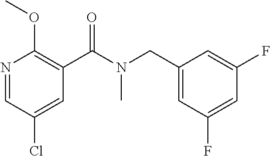 [SMILES: COc1ncc(Cl)cc1C(=O)N(C)Cc1cc(F)cc(F)c1]

Following the general procedure as described in the preparation of Example 12 and Example 13, using 5-chloro-N-(3,5-difluorobenzyl)-2-methoxynicotinamide (Intermediate 26) (0.15 g, 0.48 mmol), gave 5-chloro-N-(3,5-difluorobenzyl)-2-methoxy-N-methylnicotinamide (0.12 g, 90%) as an off-white solid.

¹H NMR (400 MHz, DMSO-d₆) δ 8.35-8.25 (m, 1H), 8.05-7.94 (m, 1H), 7.20-6.90 (m, 3H), 5.20-4.20 (m, 2H), 3.95-3.80 (m, 3H), 2.92-2.75 (m, 3H); MS m/z: 327.24 (M+H).

### Example 42: 5-fluoro-N-(6-fluoro-2,3-dihydrobenzofuran-3-yl)-2-methoxy-N-methylnicotinamide

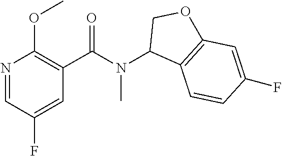 [SMILES: COc1ncc(F)cc1C(=O)N(C)C1COc2cc(F)ccc21]

Following the general procedure as described in the preparation of Example 12 and Example 13, using 5-fluoro-N-(6-fluoro-2,3-dihydrobenzofuran-3-yl)-2-methoxynicotinamide (Intermediate 31) (0.33 g, 1.0 mmol), gave 5-fluoro-N-(6-fluoro-2,3-dihydrobenzofuran-3-yl)-2-methoxy-N-methylnicotinamide (0.28 g, 81%) as a white solid.

¹H NMR (400 MHz, DMSO-d₆): δ 8.30-8.25 (m, 1H), 8.05-7.75 (m, 1H), 7.40-7.02 (m, 1H), 6.85-6.70 (m, 2H), 6.36-6.32 (m, 0.34H), 5.35-5.25 (m, 0.60H), 4.85-4.50 (m, 2H), 4.05-3.83 (m, 3H), 2.65-2.45 (m, 3H). HPLC Purity: 97.49%; MS m/z: 321.28 (M+H), 97.10%.

### Example 43: 5-chloro-N-(7-fluorochroman-4-yl)-2-methoxy-N-methylnicotinamide

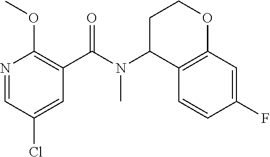 [SMILES: COc1ncc(Cl)cc1C(=O)N(C)C1CCOc2cc(F)ccc21]

Following the general procedure as described in the preparation of Example 12 and Example 13, using 5-chloro-N-(7-fluorochroman-4-yl)-2-methoxynicotinamide (Intermediate 61) (0.33 g, 1.0 mmol), gave 5-chloro-N-(7-fluorochroman-4-yl)-2-methoxy-N-methylnicotinamide (0.28 g, 81%) as an off-white solid.

¹H NMR (400 MHz, DMSO-d₆): δ 8.35-8.27 (m, 1H), 8.15-7.90 (m, 1H), 7.30-7.15 (m, 1H), 6.90-6.70 (m, 1H), 6.70-6.60 (m, 1H), 5.90-5.85 (m, 0.46H), 4.82-4.70 (m, 0.54H), 4.40-4.00 (m, 2H), 4.00-3.85 (m, 3H), 2.65-2.50 (m, 3H), 2.30-1.90 (m, 2H); MS m/z: 351.16 (M+H).

### Example 44: 5-chloro-N-(3-chloro-5-fluorobenzyl)-2-methoxy-N-methylnicotinamide

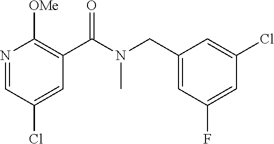 [SMILES: COc1ncc(Cl)cc1C(=O)N(C)Cc1cc(F)cc(Cl)c1]

Following the general procedure as described in the preparation of Example 12 and Example 13, using 5-chloro-N-(3-chloro-5-fluorobenzyl)-2-methoxynicotinamide (Intermediate 29) (0.33 g, 1.0 mmol), gave 5-chloro-N-(3-chloro-5-fluorobenzyl)-2-methoxy-N-methylnicotinamide (0.28 g, 81%) as an off-white solid.

¹H NMR (400 MHz, DMSO-d₆): δ 8.35-8.25 (m, 1H), 8.00-7.92 (m, 1H), 7.40-7.02 (m, 3H), 5.20-4.35 (m, 2H), 3.97-3.83 (m, 3H), 2.92-2.75 (m, 3H); MS m/z: 343.21 (M+H).

### Example 45 (enantiomer 1) and Example 46 (enantiomer 2): 5-chloro-N-(6-fluoro-2,3-dihydrobenzofuran-3-yl)-2-methoxy-N-methylnicotinamide

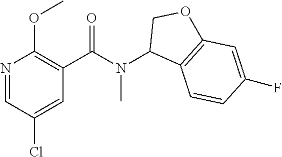 [SMILES: COc1ncc(Cl)cc1C(=O)N(C)C1COc2cc(F)ccc21]

Following the general procedure as described in the preparation of Example 12 and Example 13, using 5-chloro-N-(6-fluoro-2,3-dihydrobenzofuran-3-yl)-2-methoxynicotinamide (Intermediate 32) (0.67 g, 2.08 mmol), gave racemic 5-chloro-N-(6-fluoro-2,3-dihydrobenzofuran-3-yl)-2-methoxy-N-methylnicotinamide as a gummy material. Enantiomers were separated by preparative SFC to afford Example & Example 46 (0.28 g, 75%) as off-white solids.

Preparative SFC condition: Column/dimensions: Chiralpak IE (30×250) mm, 5 μm; % CO₂: 80%; % Co-solvent: 20% (0.2% IPA in IPA); Total Flow: 100.0 g/min; Back Pressure: 100 bar; Temperature: 30° C.; UV: 220 nm; Solubility: IPA.

Example 45: ¹H NMR (400 MHz, DMSO-d₆): δ 8.35-8.28 (m, 1H), 8.10-7.85 (m, 1H), 7.40-7.18 (m, 1H), 6.85-6.70 (m, 2H), 6.33 (q, J=4.3 Hz, 0.35H), 5.30 (t, J=6.2 Hz, 0.59H), 4.85-4.50 (m, 2H), 4.05-3.85 (m, 3H), 2.62-2.40 (m, 3H); MS m/z: 337.13 (M+H). SFC Chiral Purity: 99.97%, Rt 1.72 mins.

Example 46: ¹H NMR (400 MHz, DMSO-d₆): δ 8.35-8.30 (m, 1H), 8.10-7.85 (m, 1H), 7.40-7.18 (m, 1H), 6.85-6.70 (m, 2H), 6.33 (q, J=4.3 Hz, 0.35H), 5.30 (t, J=6.2 Hz, 0.59H), 4.85-4.50 (m, 2H), 4.05-3.85 (m, 3H), 2.60-2.40 (m, 3H); MS m/z: 337.17 (M+H). SFC Chiral Purity: 99.85%, Rt 3.07 mins.

### Example 47: N-benzyl-5-chloro-N-ethyl-2-methoxynicotinamide

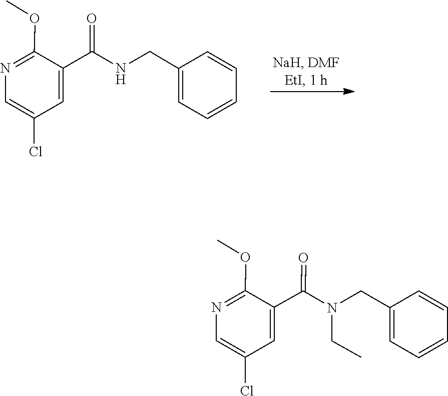 [SMILES: COc1ncc(Cl)cc1C(=O)NCc1ccccc1 ; CCN(Cc1ccccc1)C(=O)c1cc(Cl)cnc1OC]

To a stirred solution of N-benzyl-5-chloro-2-methoxynicotinamide (Intermediate to) (0.3 g, 1.09 mmol) in DMF (5 mL) at 0° C., was added NaH (0.04 g, 1.6 mmol) and the reaction mixture stirred for 10 min before ethyl iodide (0.34 g, 2.17 mmol) was added at 0° C. The reaction mixture was stirred at RT for 1 h. The reaction mixture was cooled to 0° C., quenched with water and extracted with EtOAc (3×20 mL). The combined organic phases were washed with ice-cold water, dried over Na₂SO₄ and evaporated under reduced pressure. Purification by flash column chromatography (Davisil silica, 10-30% EtOAc/Pet ether) gave N-benzyl-5-chloro-N-ethyl-2-methoxynicotinamide (0.25 g, 77%) as an off-white solid.

¹H NMR (400 MHz, DMSO-d₆): δ 8.25-8.17 (m, 1H), 7.82-7.70 (m, 1H), 7.40-7.15 (m, 5H), 4.75-4.30 (m, 2H), 3.95-3.85 (m, 3H), 3.50-3.00 (m, 2H), 1.15-0.90 (m, 3H); MS m/z: 305.17 (M+H).

### Example 48: 5-chloro-N-ethyl-N-(2-fluorobenzyl)-2-methoxynicotinamide

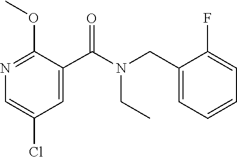 [SMILES: CCN(Cc1ccccc1F)C(=O)c1cc(Cl)cnc1OC]

By following the procedure employed in Example 47 using 5-chloro-N-(2-fluorobenzyl)-2-methoxynicotinamide (Intermediate 11) (0.15 g, 0.51 mmol), gave 5-chloro-N-ethyl-N-(2-fluorobenzyl)-2-methoxynicotinamide (0.02 g, 12%) as an off-white solid.

¹H NMR (400 MHz, DMSO-d₆, at 90° C.) δ: 8.24 (br s, 1H), 7.85-7.60 (m, 1H), 7.60-7.05 (m, 4H), 4.70-4.30 (m, 2H), 4.00-3.80 (m, 3H), 3.50-3.05 (m, 2H), 1.15-0.90 (m, 3H).

HPLC Purity: 99-45%; MS m/z: 323.19 (M+H), 99.75%.

### Example 49: 5-chloro-N-ethyl-N-(3-fluorobenzyl)-2-methoxynicotinamide

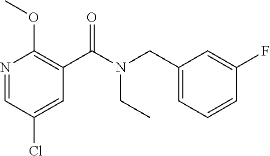 [SMILES: CCN(Cc1cccc(F)c1)C(=O)c1cc(Cl)cnc1OC]

By following the procedure employed in Example 47 using 5-chloro-N-(3-fluorobenzyl)-2-methoxynicotinamide (Intermediate 12) (0.2 g, 0.68 mmol), gave 5-chloro-N-ethyl-N-(3-fluorobenzyl)-2-methoxynicotinamide (0.02 g, 9%) as a pale-yellow gum.

¹H NMR (400 MHz, DMSO-d₆, at 90° C.): δ 8.30-8.18 (m, 1H), 7.88-7.65 (m, 1H), 7.45-7.30 (m, 1H), 7.25-7.10 (m, 1H), 7.06 (t, J=8.8 Hz, 2H), 4.75-4.30 (m, 2H), 4.00-3.80 (m, 3H), 3.45-3.00 (m, 2H), 1.15-0.90 (m, 3H); MS m/z: 323.27 (M+H).

### Example 50: 5-fluoro-N-(3-fluorobenzyl)-2-methoxy-N-methylnicotinamide

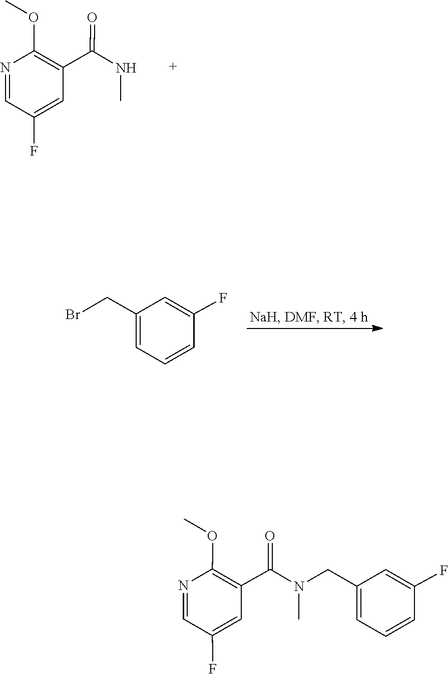 [SMILES: CNC(=O)c1cc(F)cnc1OC ; Fc1cccc(CBr)c1 ; COc1ncc(F)cc1C(=O)N(C)Cc1cccc(F)c1]

To a stirred solution of 5-fluoro-2-methoxy-N-methylnicotinamide (Intermediate 17) (0.1 g, 0.5 mmol) in DMF (5 mL) at 0° C., was added NaH (0.03 g, 1.3 mmol) and the reaction mixture stirred for 10 min before 1-(bromomethyl)-3-fluorobenzene (0.1 g, 0.6 mmol) at 0° C. was added. After stirring at RT for 4 h, the reaction was quenched with water at 0° C. and extracted with EtOAc (3×20 mL). The combined organic phases were washed with ice-cold water, dried over Na₂SO₄ and evaporated under reduced pressure. Purification by flash column chromatography (Davisil silica, 10-30% EtOAc/Pet ether) afforded 5-fluoro-N-(3-fluorobenzyl)-2-methoxy-N-methylnicotinamide (0.15 g, 90%) as an off-white solid.

¹H NMR (DMSO-d₆, 400 MHz, at 90° C.): δ 8.19 (br s, 1H), 7.70 (br s, 1H), 7.39 (br s, 1H), 7.20-6.95 (m, 3H), 4.72-4.30 (d, 2H), 3.95-3.80 (m, 3H), 2.95-2.70 (m, 3H); MS m/z: 293.20 (M+H).

### Example 51: 2-((2-methoxy-5-methylpyridin-3-yl)sulfonyl)-1,2,3,4-tetrahydroisoquinoline

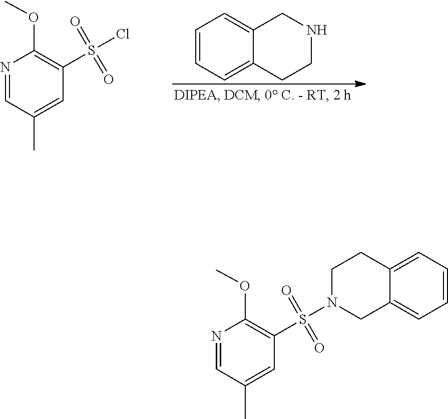 [SMILES: COc1ncc(C)cc1S(=O)(=O)Cl ; c1ccc2c(c1)CCNC2 ; COc1ncc(C)cc1S(=O)(=O)N1CCc2ccccc2C1]

A stirred solution of 1,2,3,4-tetrahydroisoquinoline (0.05 g, 0.4 mmol) in DCM (5 mL) at 0° C. was treated with DIPEA (0.18 mL, 1.0 mmol) and 2-methoxy-5-methylpyridine-3-sulfonyl chloride (0.1 g, 0.5 mmol) and then stirred at RT for 2 h. The reaction mixture was diluted with DCM, washed with 1N HCl (10 mL) followed by brine solution (10 mL). The organic phase was separated, then dried over Na₂SO₄, and concentrated under reduced pressure. Purification by flash column chromatography (Davisil silica, 0-30% EtOAc/Pet ether) afforded 2-((2-methoxy-5-methylpyridin-3-yl)sulfonyl)-1,2,3,4-tetrahydroisoquinoline (90 mg, 75%) as a white solid.

¹H NMR (DMSO-d₆, 400 MHz): δ 8.22-8.20 (m, 1H), 8.05-8.03 (m, 1H), 7.18-7.08 (m, 4H), 4.44 (s, 2H), 3.74 (s, 3H), 3.54 (t, J=6.0 Hz, 2H), 2.70 (t, J=6.0 Hz, 2H), 2.29 (s, 3H); MS m/z: 319.22 (M+H).

Following the general procedure as described in the preparation of Example 51, the following Examples were prepared:

### Example 52: 2-((5-chloro-2-methoxypyridin-3-yl)sulfonyl)-1,2,3,4-tetrahydroisoquinoline

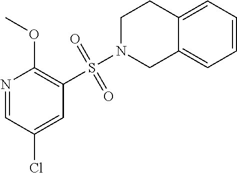 [SMILES: COc1ncc(Cl)cc1S(=O)(=O)N1CCc2ccccc2C1]

By following the procedure employed in Example 51 using 1,2,3,4-tetrahydroisoquinoline (0.05 g, 0.4 mmol) and 5-chloro-2-methoxypyridine-3-sulfonyl chloride (0.1 g, 0.5 mmol), gave 2-((5-chloro-2-methoxypyridin-3-yl)sulfonyl)-1,2,3,4-tetrahydroisoquinoline (55 mg, 66%) as white solid.

¹H NMR (DMSO-d₆, 400 MHz): δ 8.48 (d, J=2.4 Hz, 1H), 8.20 (d, J=2.4 Hz, 1H), 7.18-7.10 (m, 4H), 4.48 (s, 2H), 3.79 (s, 3H), 3.59 (t, J=6.0 Hz, 2H), 2.72 (t, J=5.6 Hz, 2H); MS m/z: 339.17 (M+H).

### Example 53: 7-fluoro-4-((2-methoxy-5-methylpyridin-3-yl)sulfonyl)-2,3,4,5-tetrahydrobenzo[f][1,4]oxazepine

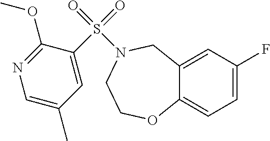 [SMILES: COc1ncc(C)cc1S(=O)(=O)N1CCOc2ccc(F)cc2C1]

By following the procedure employed in Example 51 using 7-fluoro-2,3,4,5-tetrahydrobenzo[f][1,4]oxazepine (Intermediate 44) (0.05 g, 0.29 mmol) and 2-methoxy-5-methylpyridine-3-sulfonyl chloride (0.06 g, 0.29 mmol), gave 7-fluoro-4-((2-methoxy-5-methylpyridin-3-yl)sulfonyl)-2,3,4,5-tetrahydrobenzo[f][1,4]oxazepine (20 mg, 20%) as an off-white solid.

¹H NMR (400 MHz, DMSO-d₆): δ 8.20-8.18 (m, 1H), 7.98-7.96 (m, 1H), 7.09-7.05 (dd, J=2.8 Hz, 8.8 Hz, 1H), 7.04-6.96 (m, 1H), 6.95-6.91 (m, 1H), 4.47 (s, 2H), 4.07 (t, J=4.6 Hz, 2H), 3.90 (s, 3H), 3.70 (t, J=4.6 Hz, 2H), 2.26 (s, 3H); MS m/z: 353.30 (M+H).

### Example 54: 4-((5-chloro-2-methoxypyridin-3-yl)sulfonyl)-7-fluoro-2,3,4,5-tetrahydrobenzo[f][1,4]oxazepine

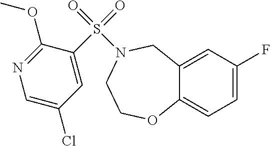 [SMILES: COc1ncc(Cl)cc1S(=O)(=O)N1CCOc2ccc(F)cc2C1]

By following the procedure employed in Example 51 using 7-fluoro-2,3,4,5-tetrahydrobenzo[f][1,4]oxazepine (Intermediate 44) (0.05 g, 0.29 mmol) and 5-chloro-2-methoxypyridine-3-sulfonyl chloride (0.06 g, 0.29 mmol), gave 4-((5-chloro-2-methoxypyridin-3-yl)sulfonyl)-7-fluoro-2,3,4,5-tetrahydrobenzo[f][1,4]oxazepine (28 mg, 20%) as an off-white solid.

¹H NMR (400 MHz, DMSO-d₆): δ 8.45 (d, J=2.8 Hz, 1H), 8.12 (d, J=2.4 Hz, 1H), 7.10 (dd, J=3.2 Hz, 8.8 Hz, 1H), 7.03-6.96 (m, 1H), 6.95-6.86 (m, 1H), 4.51 (s, 2H), 4.09 (t, J=4.8 Hz, 2H), 3.94 (s, 3H), 3.76 (t, J=4.4 Hz, 2H); MS m/z: 373.25 (M+H).

### Example 55: 7-fluoro-4-((2-methoxypyridin-3-yl)sulfonyl)-2,3,4,5-tetrahydrobenzo[f][1,4]oxazepine

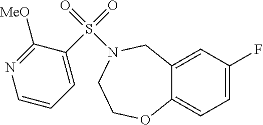 [SMILES: COc1ncccc1S(=O)(=O)N1CCOc2ccc(F)cc2C1]

By following the procedure employed in Example 51 using 7-fluoro-2,3,4,5-tetrahydrobenzo[f][1,4]oxazepine (Intermediate 44) (0.06 g, 0.6 mmol) and 2-methoxypyridine-3-sulfonyl chloride (0.0621 g, 0.3 mmol), gave 7-fluoro-4-((2-methoxypyridin-3-yl)sulfonyl)-2,3,4,5-tetrahydrobenzo[f][1,4]oxazepine as off-white solid (40 mg, 42%)

¹H NMR (400 MHz, DMSO-d₆): δ 8.39 (dd, J=1.60 Hz, 4.80 Hz, 1H), 8.16 (dd, J=2.00 Hz, 7.60 Hz, 1H), 7.17-7.14 (m, 1H), 7.07-6.96 (m, 3H), 4.47 (s, 2H), 4.08 (t, J=4.80 Hz, 2H), 3.94 (s, 3H), 3.70 (t, J=4.40 Hz, 2H). HPLC Purity: 99.32%; MS m/z: 339.17 (M+H), 99.61%.

### Example 56: methyl 4-fluoro-2-((5-fluoro-2-methoxy-N-methylnicotinamido)methyl)benzoate

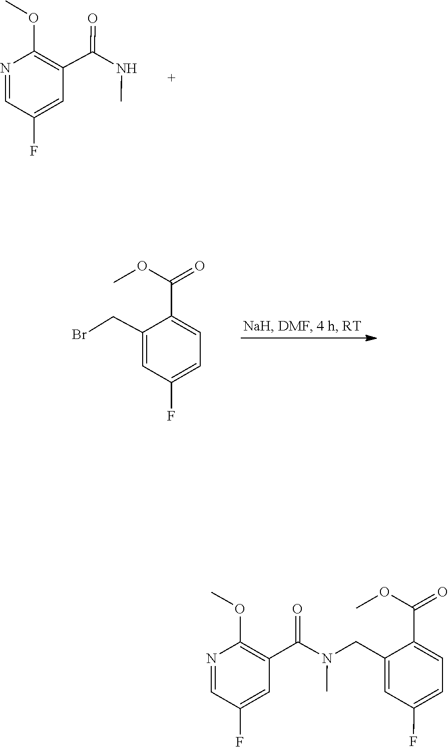 [SMILES: CNC(=O)c1cc(F)cnc1OC ; COC(=O)c1ccc(F)cc1CBr ; COC(=O)c1ccc(F)cc1CN(C)C(=O)c1cc(F)cnc1OC]

To a stirred solution of 5-fluoro-2-methoxy-N-methylnicotinamide (Intermediate 17) (0.1 g, 0.5 mmol) in dry DMF (5 mL) at 5° C., was added NaH (0.04 g, 1.5 mmol) and the reaction mixture stirred for 15 minutes before methyl 2-(bromomethyl)-4-fluorobenzoate (0.09 g, 0.6 mmol) was added. After stirring for 4 h at RT, the reaction was quenched by adding ice, the pH was adjusted with 1N HCl and then evaporated to dryness. The residue was diluted with EtOAc (15 mL) and washed successively with brine (3×20 mL) and water (20 mL). The organic layer was then dried over Na₂SO₄ and concentrated in vacuo. Purification by flash chromatography (Davisil silica, 0-40% EtOAc in pet ether) gave methyl 4-fluoro-2-((5-fluoro-2-methoxy-N-methylnicotinamido)methyl)benzoate as a white solid (130 mg, 69%).

¹H NMR (DMSO-d₆, 400 MHz): δ 8.30-8.18 (m, 1H), 8.08-8.03 (m, 1H), 7.93-7.75 (m, 1H), 7.35-7.15 (m, 2H), 5.50-4.50 (m, 2H), 4.00-3.75 (m, 6H), 3.00-2.80 (m, 3H); MS m/z: 351.25 (M+H).

Following the general procedure as described in the preparation of Example 56, the following Example was prepared:

### Example 57: 5-chloro-N-(4-fluoro-2-(trifluoromethyl)benzyl)-2-methoxy-N-methylnicotinamide

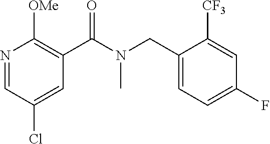 [SMILES: COc1ncc(Cl)cc1C(=O)N(C)Cc1ccc(F)cc1C(F)(F)F]

By following the procedure employed in Example 56 using 5-chloro-2-methoxy-N-methylnicotinamide (Intermediate 28) and (bromomethyl)-4-fluoro-2-(trifluoromethyl)benzene (0.29 g, 1.15 mmol), gave 5-chloro-N-(4-fluoro-2-(trifluoromethyl)benzyl)-2-methoxy-N-methylnicotinamide (0.03 g, 8%) as a white solid.

¹H NMR (400 MHz, DMSO-d₆): δ 8.37-8.23 (m, 1H), 7.98-7.80 (m, 1H), 7.72-7.55 (m, 3H), 5.00-4.50 (m, 2H), 4.00-3.75 (m, 3H), 2.99-2.80 (m, 3H); MS m/z: 377.21 (M+H).

### Example 58: 5-chloro-N-(2-cyano-4-fluorobenzyl)-2-methoxy-N-methylnicotinamide

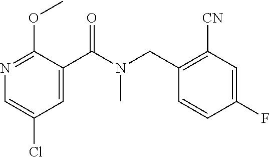 [SMILES: COc1ncc(Cl)cc1C(=O)N(C)Cc1ccc(F)cc1C#N]

By following the procedure employed for Example 56, using 5-chloro-2-methoxy-N-methylnicotinamide (Intermediate 28) (0.12 g, 0.6 mmol) and 2-(bromomethyl)-5-fluorobenzonitrile (0.15 g, 0.72 mmol) gave 5-chloro-N-(2-cyano-4-fluorobenzyl)-2-methoxy-N-methylnicotinamide (0.1 g, 60%) as an off-white solid.

¹H NMR (400 MHz, DMSO-d₆): δ 8.35-8.25 (m, 1H), 7.97-7.88 (m, 1H), 7.85-7.45 (m, 3H), 4.90-4.55 (m, 2H), 3.97-3.78 (m, 3H), 2.97-2.80 (m, 3H); MS m/z: 334.24 (M+H).

### Example 59 (enantiomer 1) and Example 60 (enantiomer 2): 5-chloro-N-(1-(2-cyano-5-fluorophenyl)ethyl)-2-methoxy-N-methylnicotinamide

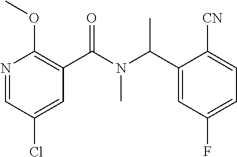 [SMILES: COc1ncc(Cl)cc1C(=O)N(C)C(C)c1cc(F)ccc1C#N]

To a stirred solution of N-(1-(2-bromo-5-fluorophenyl)ethyl)-5-chloro-2-methoxy-N-methylnicotinamide (Intermediate 35) (0.2 g, 0.75 mmol) in NMP (10 ml) was added copper(I) cyanide (0.067 g, 0.75 mmol) in a microwave vial. The reaction was purged with a nitrogen atmosphere for 10 min, then Pd(PPh₃)₄ (0.028 g, 0.025 mmol) was added and the reaction irradiated at 170° C. for 1 h. Water was added and the mixture was extracted with EtOAc (3×20 mL). The organic phase was washed with ice-cold water (3×20 mL). dried over Na2SO4 and evaporated under reduced pressure to obtain the crude product. Purification by reverse phase column chromatography, (60-70% MeOH/0.1% aq. Ammonium bicarbonate solution) gave racemic 5-chloro-N-(1-(2-cyano-5-fluorophenyl)ethyl)-2-methoxy-N-methylnicotinamide (0.10 g, 55%) as colourless liquid. Enantiomers were separated by preparative SFC; fractions were concentrated under reduced pressure to afford Example 59 & Example 60 (0.09 g, 50%) as off-white solids.

SFC preparative condition: Column/dimensions: (Chiralpak OX-H (30×250 mm), 5 μm; % CO₂: 83%; % Co solvent: 17% (IPA); Total Flow: 100.0 g/min; Back Pressure: 100 bar; Temperature: 35° C.; UV: 220 nm. Solubility: IPA

Example 59: ¹H NMR (400 MHz, DMSO-d₆): δ 8.32-8.25 (m, 1H), 8.03-7.95 (m, 1H), 7.95-7.70 (m, 1H), 7.55-7.35 (m, 2H), 5.80-4.98 (m, 1H), 3.90 (s, 3H), 3.30-2.50 (m, 3H), 1.58 (d, J=7.2 Hz, 3H); MS m/z: 348.25 (M+H). SFC Chiral Purity: 99.73%, Rt 4.75 mins.

Example 60: ¹H NMR (400 MHz, DMSO-d₆): δ 8.32-8.25 (m, 1H), 8.03-7.95 (m, 1H), 7.95-7.70 (m, 1H), 7.55-7.35 (m, 2H), 5.90-4.98 (m, 1H), 3.90 (s, 3H), 3.30-2.50 (m, 3H), 1.58 (d, J=7.2 Hz, 3H); MS m/z: 348.25 (M+H). SFC Chiral Purity: 94.85%, Rt 5.34 mins.

### Example 61: N-(2-cyano-4,6-difluorobenzyl)-5-fluoro-2-methoxy-N-methylnicotinamide

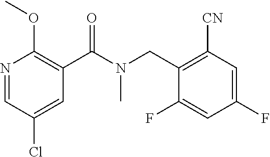 [SMILES: COc1ncc(Cl)cc1C(=O)N(C)Cc1c(F)cc(F)cc1C#N]

By following the procedure employed in preparation of Examples 59 & 60 using N-(2-bromo-4,6-difluorobenzyl)-5-fluoro-2-methoxy-N-methylnicotinamide (Intermediate 27) (0.4 g, 1.0 mmol), gave N-(2-cyano-4,6-difluorobenzyl)-5-fluoro-2-methoxy-N-methylnicotinamide (0.058 g, 44%) as a yellow sticky solid.

¹H NMR (400 MHz, DMSO-d₆): δ 8.26-8.21 (m, 1H), 7.85-7.66 (m, 3H), 4.80-4.55 (m, 2H), 3.85-3.75 (m, 3H), 2.97-2.80 (m, 3H); MS m/z: 336.16 (M+H).

### Example 62 (enantiomer 1) and Example 63 (enantiomer 2): N-(1-(2-cyano-5-fluorophenyl)ethyl)-5-fluoro-2-methoxy-N-methylnicotinamide

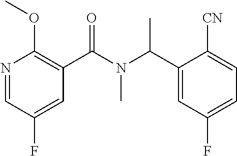 [SMILES: COc1ncc(F)cc1C(=O)N(C)C(C)c1cc(F)ccc1C#N]

By following the procedure employed in preparation of Examples 59 & 60 using N-(1-(2-bromo-5-fluorophenyl)ethyl)-5-fluoro-2-methoxy-N-methylnicotinamide (0.2 g, 0.75 mmol) (Intermediate 34), gave racemic N-(1-(2-cyano-5-fluorophenyl)ethyl)-5-fluoro-2-methoxy-N-methylnicotinamide (0.10 g, 55%) as a colourless liquid. Enantiomers were separated by preparative SFC to afford Example 62 & Example 63 (0.09 g, 50%) as off-white solids.

Preparative SFC condition: Column/dimensions: (Chiralpak OX-H (30×250 mm), 5 μm; % CO₂: 83%; % Co-solvent: 17% (IPA); Total Flow: 100.0 g/min; Back Pressure: 100 bar; Temperature: 35° C.; UV: 220 nm. Solubility: IPA.

Example 62: ¹H NMR (400 MHz, DMSO): δ 8.26 (d, J=3.0 Hz, 1H), 7.84 (m, J=33.7 Hz, 2H), 7.44 (m, J=8.0 Hz, 2H), 5.38 (t, J=154.6 Hz, 1H), 3.88 (s, 2.6H), 2.67 (t, J=1.8 Hz, 3.4H), 1.58 (d, J=7.1 Hz, 3H); MS m/z: 332.35 (M+H). SFC Chiral Purity: 99.65%, Rt 4.59 mins.

Example 63: ¹H NMR (400 MHz, DMSO): δ 8.26 (d, J=3.0 Hz, 1H), 7.84 (m, J=33.8 Hz, 2H), 7.44 (m, J=8.0 Hz, 2H), 5.38 (d, J=300.1 Hz, 1H), 3.88 (s, 2.6H), 2.67 (t, J=1.8 Hz, 3.4H), 1.58 (d, J=7.1 Hz, 3H); MS m/z: 332.35 (M+H). SFC Chiral Purity: 99.32%, Rt 7.18 mins.

### Example 64 (enantiomer 1) and Example 65 (enantiomer 2): N-(1-(2-cyano-4-fluorophenyl)ethyl)-5-fluoro-2-methoxy-N-methylnicotinamide

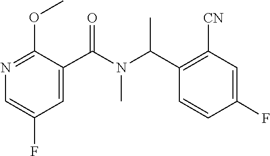 [SMILES: COc1ncc(F)cc1C(=O)N(C)C(C)c1ccc(F)cc1C#N]

By following the procedure employed in preparation of Examples 59 & 60 using N-(1-(2-bromo-4-fluorophenyl)ethyl)-5-fluoro-2-methoxy-N-methylnicotinamide (Intermediate 36) (0.3 g, 0.8 mmol), gave racemic N-(1-(2-cyano-4-fluorophenyl)ethyl)-5-fluoro-2-methoxy-N-methylnicotinamide as a semi solid. Enantiomers were separated by preparative SFC to afford Example 64 & Example 65 (0.14 g, 54%) as off-white semi solids.

SFC preparative condition: Column/dimensions: (Chiralpak OX-H (30×250 mm), 5 μm; % CO₂: 83%; % Co solvent: 17% (IPA); Total Flow: 100.0 g/min; Back Pressure: 100 bar; Temperature: 35° C.; UV: 220 nm; Solubility: IPA

Example 64: ¹H NMR (400 MHz, DMSO-d₆): δ 8.28-8.18 (m, 1H), 7.93-7.87 (m, 1H), 7.85-7.57 (m, 3H), 5.90-4.95 (m, 1H), 3.88 (s, 3H), 3.20-2.50 (m, 3H), 1.58 (d, J=7.2 Hz, 3H); MS m/z: 332.17 (M+H). SFC Chiral Purity: 99.98%, Rt 1.45 mins

Example 65: ¹H NMR (400 MHz, DMSO-d₆): δ 8.27-8.19 (m, 1H), 7.93-7.87 (m, 1H), 7.85-7.57 (m, 3H), 5.85-4.95 (m, 1H), 3.88 (s, 3H), 3.20-2.50 (m, 3H), 1.58 (d, J=7.2 Hz, 3H); MS m/z: 332.17 (M+H). SFC Chiral Purity: 99.28%, Rt 1.76 mins.

### Example 66 (enantiomer 1) and Example 67 (enantiomer 2): 5-chloro-N-(1-(2-cyano-4-fluorophenyl)ethyl)-2-methoxy-N-methylnicotinamide

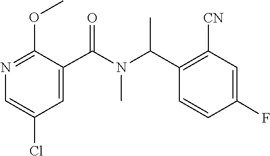 [SMILES: COc1ncc(Cl)cc1C(=O)N(C)C(C)c1ccc(F)cc1C#N]

By following the procedure employed in preparation of Examples 59 & 60 using N-(1-(2-bromo-4-fluorophenyl)ethyl)-5-chloro-2-methoxy-N-methylnicotinamide (Intermediate 37) (0.3 g, 0.8 mmol), gave racemic 5-chloro-N-(1-(2-cyano-4-fluorophenyl)ethyl)-2-methoxy-N-methylnicotinamide as a sticky solid. Enantiomers were separated by preparative SFC to afford Example 66 & Example 67 (0.1 g, 60%) as off white semisolids.

Preparative SFC condition: Column/dimensions: Chiralpak IC (30×250) mm, 5 μm; % CO₂: 75%; % Co solvent: 25% (MeOH); Total Flow: 120.0 g/min; Back Pressure: 100 bar; Temperature: 30° C.; UV: 220 nm; Solubility: Methanol.

Example 66: ¹H NMR (400 MHz, DMSO-d₆): δ 8.35-8.25 (m, 1H), 7.92-7.85 (m, 1H), 7.85-7.60 (m, 3H), 5.85-4.95 (m, 1H), 3.89 (s, 3H), 3.25-2.50 (m, 3H), 1.57 (d, J=7.2 Hz, 3H); MS m/z: 348.17 (M+H). SFC Chiral Purity: 99.95%, Rt 2.17 mins.

Example 67: ¹H NMR (400 MHz, DMSO-d₆): δ 8.35-8.25 (m, 1H), 7.92-7.85 (m, 1H), 7.85-7.60 (m, 3H), 5.85-4.95 (m, 1H), 3.89 (s, 3H), 3.25-2.50 (m, 3H), 1.57 (d, J=7.2 Hz, 3H); MS m/z: 348.17 (M+H). SFC Chiral Purity: 98.37%, Rt 2.53 mins.

### Example 68 (enantiomer 1) and Example 69 (enantiomer 2): N-(1-(2,4-difluorophenyl)ethyl)-5-fluoro-2-methoxy-N-methylnicotinamide

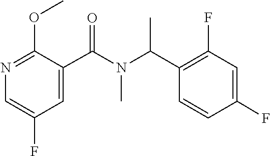 [SMILES: COc1ncc(F)cc1C(=O)N(C)C(C)c1ccc(F)cc1F]

By following the procedure employed for Example 1 using 5-fluoro-2-methoxynicotinic acid (0.15 g, 2.9 mmol) and 1-(2,4-difluorophenyl)-N-methylethan-1-amine (Intermediate 38) (0.165 g, 1.14 mmol) gave racemic N-(1-(2,4-difluorophenyl)ethyl)-5-fluoro-2-methoxy-N-methylnicotinamide as an off-white solid.

Enantiomers were separated by preparative SFC to afford Example 68 & Example 69 (0.1 g, 60%) as yellow gums.

Preparative SFC condition: Column/dimensions: Chiralpak IC (30×250) mm, 5 μm; % CO₂: 75%; % Co solvent: 25% (MeOH); Total Flow: 120.0 g/min; Back Pressure: 100 bar; Temperature: 30° C.; UV: 220 nm; Solubility: Methanol.

Example 68: ¹H NMR (400 MHz, DMSO-d₆): δ 8.30-8.20 (m, 1H), 8.00-7.50 (m, 2H), 7.35-7.08 (m, 2H), 5.93 (q, J=7.0 Hz, 0.51H), 4.95-4.80 (m, 0.45H), 3.95-3.60 (m, 3H), 2.85-2.50 (m, 3H), 1.60-1.48 (m, 3H); MS m/z: 325.11 (M+H). SFC Chiral Purity: 99.94%, Rt 0.99 mins.

Example 69: ¹H NMR (400 MHz, DMSO-d₆): δ 8.30-8.20 (m, 1H), 8.00-7.50 (m, 2H), 7.35-7.08 (m, 2H), 5.93 (q, J=7.0 Hz, 0.5H), 4.95-4.80 (m, 0.44H), 3.95-3.65 (m, 3H), 2.85-2.50 (m, 3H), 1.60-1.50 (m, 3H); MS m/z: 325.10 (M+H). SFC Chiral Purity: 99.32%, Rt 1.42 mins.

### Example 70 (enantiomer 1) and Example 71 (enantiomer 2): 2-(difluoromethoxy)-5-fluoro-N-(1-(2-fluorophenyl)ethyl)-N-methylnicotinamide

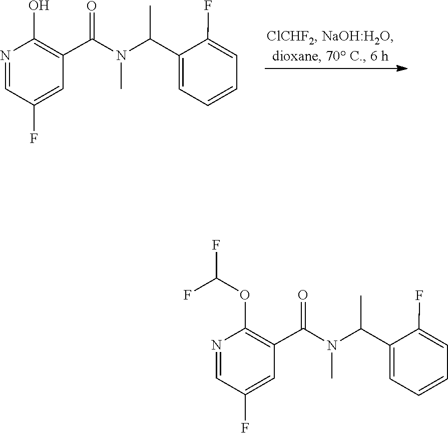 [SMILES: CC(c1ccccc1F)N(C)C(=O)c1cc(F)cnc1O ; CC(c1ccccc1F)N(C)C(=O)c1cc(F)cnc1OC(F)F]

A stirred solution of 5-fluoro-N-(1-(2-fluorophenyl)ethyl)-2-hydroxy-N-methylnicotinamide (Intermediate 40) (0.9 g, 0.31 mmol), NaOH (0.62 g, 15.5 mmol), H₂O (1 mL) in 1,4 dioxane (10 ml) in a glass tube, was purged with chlorodifluoromethane at 0° C. for 10 mins. The tube was sealed and the mixture was stirred at 70° C. for 6 h. The reaction mixture was concentrated under reduced pressure and the resulting residue was purified by reverse phase column chromatography, (60-70% MeOH/0.1% aq. Ammonium bicarbonate solution) to afford racemic 2-(difluoromethoxy)-5-fluoro-N-(1-(2-fluorophenyl)ethyl)-N-methylnicotinamide as a gummy material. Enantiomers were separated by preparative SFC to afford Example 70 & Example 71 (0.3 g, 28%) as colourless gums.

Preparative SFC condition: Column/dimensions: Chiralcel-OX-H (30×250) mm, 5 μm; % CO₂: 70%; % Co solvent: 30% (0.2% Isopropylamine in Isopropanol); Total Flow: 120.0 g/min; Back Pressure: 100 bar; Temperature: 30° C.; UV: 220 nm; Solubility: Methanol.

Example 70: ¹H NMR (400 MHz, DMSO-d₆): δ 8.45-8.35 (m, 1H), 8.25-7.30 (m, 4H), 7.30-7.15 (m, 2H), 6.00 (q, J=7.0 Hz, 0.57H), 5.05-4.90 (br s, 0.37H), 2.85-2.50 (m, 3H), 1.65-1.50 (m, 3H); MS m/z: 343.11 (M+H). SFC Chiral Purity: 99.90%, Rt 1.07 mins.

Example 71: ¹H NMR (400 MHz, DMSO-d₆) δ 8.39 (t, J=8.3 Hz, 1H), 7.67 (m, J=22.1 Hz, 4H), 7.22 (q, J=8.0 Hz, 2H), 6.00 (q, J=7.0 Hz, 0.59H), 4.96 (d, J=6.2 Hz, 0.38H), 2.76 (d, J=22.1 Hz, 3H), 1.57 (q, J=7.2 Hz, 3H); MS m/z: 343.09 (M+H). SFC Chiral Purity: 97.50%, Rt 1.40 mins.

### Example 72: N-(2,4-difluorobenzyl)-2-methoxy-N,5-dimethylnicotinamide

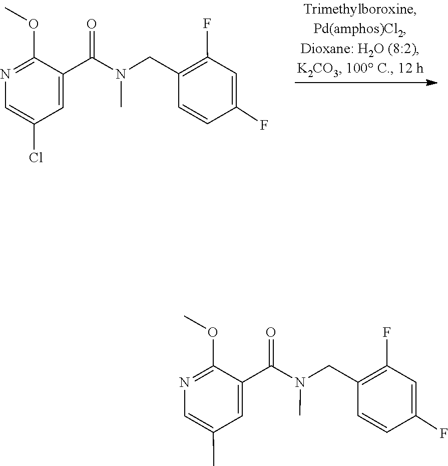 [SMILES: COc1ncc(Cl)cc1C(=O)N(C)Cc1ccc(F)cc1F ; COc1ncc(C)cc1C(=O)N(C)Cc1ccc(F)cc1F]

To a stirred solution of 4-chloro-N-(2,4-difluorobenzyl)-2-methoxy-N-methylnicotinamide (Intermediate 41) (0.350 g, 1.07 mmol) in dioxane:water (8:2, 10 mL) in a glass tube was added trimethylboroxine (0.671 g, 5.35 mmol) and potassium carbonate (0.442 g, 3.21 mmol). The tube was then purged with nitrogen for 10 min before Pd(amphos)Cl₂ (0.075 g, 0.107 mmol) under nitrogen atmosphere was added. The tube was sealed and stirred at 100° C. for 12 h. After this time, the reaction mixture was cooled to RT, filtered through celite and washed with DCM (3×10 mL). The filtrate and washings were combined and concentrated under reduced pressure. Purification by reverse phase column chromatography, (60-70% MeOH/0.1% aq. Ammonium bicarbonate solution) gave N-(2,4-difluorobenzyl)-2-methoxy-N,5-dimethylnicotinamide (0.035 g, 62%) as an off-white solid.

¹H NMR (400 MHz, DMSO-d₆): δ 8.08-8.03 (m, 1H), 7.60-7.40 (m, 2H), 7.33-7.05 (m, 2H), 4.75-4.30 (m, 2H), 3.90-3.75 (m, 3H), 2.90-2.75 (m, 3H), 2.25-2.15 (m, 3H); MS m/z: 307.36 (M+H).

### Example 73: N-(2,4-difluorobenzyl)-2-methoxy-N,4-dimethylnicotinamide

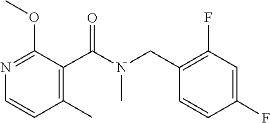 [SMILES: COc1nccc(C)c1C(=O)N(C)Cc1ccc(F)cc1F]

By following the procedure employed in Example 72 using 4-chloro-N-(2,4-difluorobenzyl)-2-methoxy-N-methylnicotinamide (Intermediate 42) (0.06 g, 0.2 mmol), gave N-(2,4-difluorobenzyl)-2-methoxy-N,4-dimethylnicotinamide (0.035 g, 62%) as a colourless gum.

¹H NMR (400 MHz, DMSO-d₆ at 90° C.): δ 8.28-8.05 (m, 1H), 7.73-7.43 (m, 1H), 7.32-6.85 (m, 3H), 4.75-4.20 (m, 2H), 3.93-3.75 (m, 3H), 3.00-2.65 (m, 3H), 2.18-2.07 (m, 3H); MS m/z: 307.14 (M+H).

### Example 74: 2-(difluoromethoxy)-5-fluoro-N-(6-fluoro-2,3-dihydrobenzofuran-3-yl)-N-methylnicotinamide

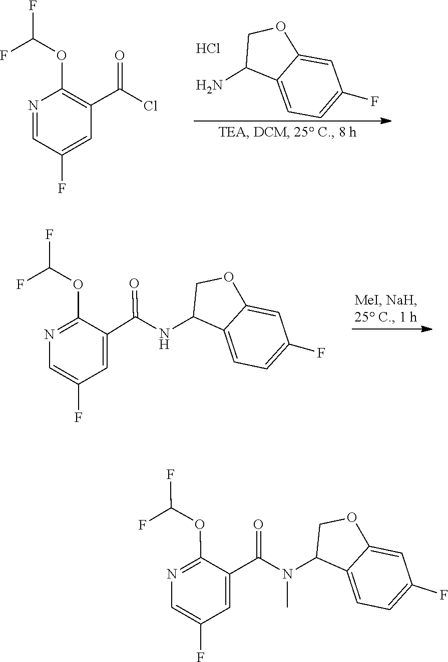 [SMILES: O=C(Cl)c1cc(F)cnc1OC(F)F ; Cl ; NC1COc2cc(F)ccc21 ; O=C(NC1COc2cc(F)ccc21)c1cc(F)cnc1OC(F)F ; CN(C(=O)c1cc(F)cnc1OC(F)F)C1COc2cc(F)ccc21]

Step 1: To a mixture of 2-(difluoromethoxy)-5-fluoro-pyridine-3-carbonyl chloride (Intermediate 45) (300 mg, crude) and 6-fluoro-2,3-dihydrobenzofuran-3-amine hydrochloride (199.2 mg, 1.05 mmol) (Intermediate 48) in dichloromethane (2 mL) was added triethylamine (134.6 mg, 1.33 mmol) in one portion at 25° C. and stirred for 8 hours. The mixture was concentrated to afford the crude, which was purified by flash silica gel chromatography (ISCO®; 4 g SepaFlash® Silica Flash Column, Eluent of 0˜20% Ethyl acetate/Petroleum ether gradient @20 mL/min) to give 2-(difluoromethoxy)-5-fluoro-N-(6-fluoro-2, 3-dihydrobenzofuran-3-yl)pyridine-3-carboxamide (140 mg, 0.188 mmol, 14.15% yield, 46% purity) as a white solid. MS ES⁺: 343.1

Step 2: Mel (33.2 mg, 0.234 mmol) was added to the mixture of 2-(difluoromethoxy)-5-fluoro-N-(6-fluoro-2,3-dihydrobenzofuran-3-yl)pyridine-3-carboxamide (80 mg, 0.234 mmol) and NaH (9.4 mg, 0.234 mmol, 60% purity in oil) in THF (1 mL) at 0° C. under N₂. Then the mixture was stirred for 1 hour under N₂. The mixture was poured into saturated NH₄Cl (aq.) (20 mL) and extracted with ethyl acetate (3×20 mL), the combined organic layers were concentrated to afford the crude which was purified by prep. HPLC (Column: Phenomenex luna 30*30 mm*10 μm+YMC AQ 100*30*10 μm, Mobile Phase A: water (0.225% HCOOH), Mobile Phase B: acetonitrile, Flow rate: 25 mL/min, gradient condition from 35% B to 95%). The pure fractions were collected and the volatiles were removed under vacuum. The residue was partitioned between acetonitrile (2 mL) and water (10 mL). The solution was lyophilized to dryness to give 2-(difluoromethoxy)-5-fluoro-N-(6-fluoro-2,3-dihydrobenzofuran-3-yl)-N-methylnicotinamide (20 mg, 0.056 mmol, 23.97% yield) as a colourless gum.

¹H NMR (400 MHz, DMSO-d₆): 8.43-8.32 (m, 1H), 8.31-8.00 (m, 1H), 7.95-7.47 (m, 1H), 7.45-7.08 (m, 1H), 6.85-6.34 (m, 2H), 5.49-4.48 (m, 3H), 2.65 (s, 2H), 2.46 (s, 1H). MS ES⁺: 357.1

Following the general procedure as described in the preparation of Example 74, the following Examples were prepared:

### Example 75: 2-(difluoromethoxy)-5-fluoro-N-(7-fluoro-2,3-dihydrobenzofuran-3-yl)-N-methylnicotinamide

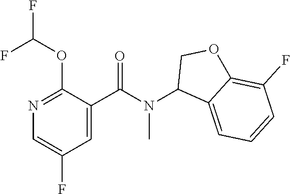 [SMILES: CN(C(=O)c1cc(F)cnc1OC(F)F)C1COc2c(F)cccc21]

Following the procedure employed for Example 74 using 7-fluoro-2,3-dihydrobenzofuran-3-amine hydrochloride (Intermediate 46) (230 mg, crude) and 2-(difluoromethoxy)-5-fluoropyridine-3-carbonyl chloride (Intermediate 45) gave 2-(difluoromethoxy)-5-fluoro-N-(7-fluoro-2,3-dihydrobenzofuran-3-yl)-N-methylnicotinamide (79.2 mg, 0.221 mmol, 34.3% yield) as a white solid.

¹H NMR (400 MHz, DMSO-d₆): 8.46-8.26 (m, 2H), 7.88-7.49 (m, 1H), 7.23-6.97 (m, 3H), 6.48-5.50 (m, 1H), 4.89-4.61 (m, 2H), 3.33-2.64 (m, 3H). MS ES⁺: 357.1

### Example 76: 5-chloro-2-(difluoromethoxy)-N-(3-fluoro-5-methylbenzyl)-N-methylnicotinamide

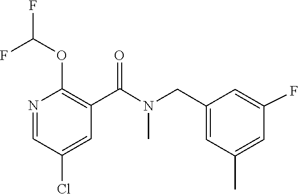 [SMILES: Cc1cc(F)cc(CN(C)C(=O)c2cc(Cl)cnc2OC(F)F)c1]

Following the procedure employed for Example 74 using 5-chloro-2-(difluoromethoxy)pyridine-3-carbonyl chloride (Intermediate 47) (90 mg, crude) and (3-fluoro-5-methylphenyl)methanamine (51.8 mg, 0.372 mmol) gave 5-chloro-2-(difluoromethoxy)-N-(3-fluoro-5-methylbenzyl)-N-methylnicotinamide (39-3 mg, 0.108 mmol, 19.7% yield) as a white powder.

¹H NMR (400 MHz, DMSO-d₆): 8.49-8.38 (m, 1H), 8.27-8.20 (m, 1H), 7.96-7.50 (m, 1H), 7.01-6.93 (m, 2H), 6.88-6.81 (m, 1H), 5.25-4.20 (m, 2H), 2.93-2.75 (m, 3H), 2.35-2.28 (m, 3H). MS ES⁺: 359.3

### Example 77: 5-chloro-2-(difluoromethoxy)-N-(6-fluoro-2,3-dihydrobenzofuran-3-yl)-N-methylnicotinamide

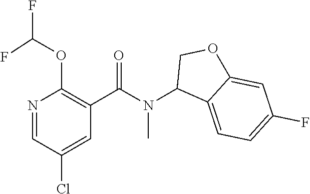 [SMILES: CN(C(=O)c1cc(Cl)cnc1OC(F)F)C1COc2cc(F)ccc21]

Following the procedure employed for Example 74 using 5-chloro-2-(difluoromethoxy)pyridine-3-carbonyl chloride (Intermediate 47) (100 mg, 0.413 mmol) and 6-fluoro-2,3-dihydrobenzofuran-3-amine hydrochloride (Intermediate 48) gave 5-chloro-2-(difluoromethoxy)-N-(6-fluoro-2,3-dihydrobenzofuran-3-yl)-N-methylnicotinamide (10 mg, 0.027 mmol, 9.59% yield) as a yellow gum.

¹H NMR (400 MHz, DMSO-d₆): 8.49-8.40 (m, 1H), 8.39-7.94 (m, 1H), 7.91-7.48 (m, 1H), 7.45-7.16 (m, 1H), 6.81-6.30 (m, 2H), 5.47-4.49 (m, 3H), 2.64 (s, 2H), 2.46 (s, 1H). MS ES⁺: 373.1

### Example 78: 5-chloro-N-(3,5-difluorobenzyl)-2-(difluoromethoxy)-N-methylnicotinamide

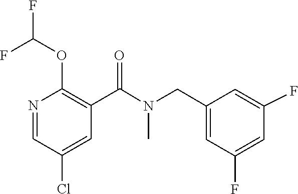 [SMILES: CN(Cc1cc(F)cc(F)c1)C(=O)c1cc(Cl)cnc1OC(F)F]

Following the procedure employed for Example 74 using 5-chloro-2-(difluoromethoxy)pyridine-3-carbonyl chloride (Intermediate 47) (105 mg, 0.434 mmol) and (3,5-difluorophenyl)methanamine (74-5 mg, 0.521 mmol) gave 5-chloro-N-(3,5-difluorobenzyl)-2-(difluoromethoxy)-N-methylnicotinamide (11.81 mg, 0.032 mmol, 22.51% yield) as a white solid.

¹H NMR (400 MHz, DMSO-d₆): 8.49-8.37 (m, 1H), 8.29-8.20 (m, 1H), 7.94-7.49 (m, 1H), 7.21-7.02 (m, 2H), 6.95 (br d, J=6.6 Hz, 1H), 4.96-4.36 (m, 2H), 2.98-2.75 (m, 3H). MS ES⁺: 363.2

### Example 79: 5-chloro-N-(3-chloro-5-fluorobenzyl)-2-(difluoromethoxy)-N-methylnicotinamide

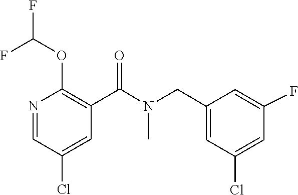 [SMILES: CN(Cc1cc(F)cc(Cl)c1)C(=O)c1cc(Cl)cnc1OC(F)F]

Following the procedure employed for Example 74 using 5-chloro-2-(difluoromethoxy)pyridine-3-carbonyl chloride (Intermediate 47) (105 mg, crude) and (3-chloro-5-fluorophenyl)methanamine (83.1 mg, 0.521 mmol) gave 5-chloro-N-(3-chloro-5-fluorobenzyl)-2-(difluoromethoxy)-N-methylnicotinamide (10.68 mg, 0.0275 mmol, 16.74% yield) as a white solid.

¹H NMR (400 MHz, DMSO-d₆): 8.50-8.41 (m, 1H), 8.30-8.22 (m, 1H), 7.96-7.51 (m, 1H), 7.42-7.34 (m, 1H), 7.30 (s, 1H), 7.22-7.07 (m, 1H), 5.04-4.33 (m, 2H), 2.99-2.80 (m, 3H). MS ES⁺: 381.2

### Example 80: 5-chloro-2-(difluoromethoxy)-N-methyl-N-(3-methylbenzyl)nicotinamide

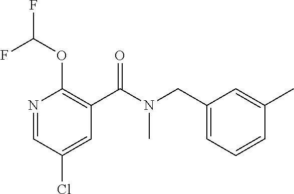 [SMILES: Cc1cccc(CN(C)C(=O)c2cc(Cl)cnc2OC(F)F)c1]

Following the procedure employed in Example 74 Step 1 using 5-chloro-2-(difluoromethoxy)pyridine-3-carbonyl chloride (Intermediate 47) (77 mg, crude) and N-methyl-1-(m-tolyl)methanamine (51.6 mg, 0.382 mmol) gave 5-chloro-2-(difluoromethoxy)-N-methyl-N-(3-methylbenzyl)nicotinamide (49.84 mg, 0.146 mmol, 45.94% yield) as a white powder.

¹H NMR (400 MHz, DMSO-d₆): 8.54-8.45 (m, 1H), 8.31-8.24 (m, 1H), 8.01-7.58 (m, 1H), 7.35-7.24 (m, 1H), 7.22-7.14 (m, 2H), 7.09-7.02 (m, 1H), 4.40 (s, 1H), 3.38 (s, 1H), 2.97-2.79 (m, 3H), 2.39-2.33 (m, 3H). MS ES⁺: 341.3.

### Example 81: 5-chloro-2-(difluoromethoxy)-N-(3-methoxybenzyl)-N-methylnicotinamide

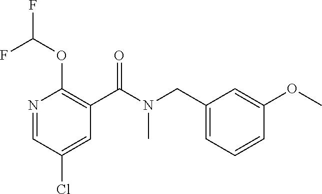 [SMILES: COc1cccc(CN(C)C(=O)c2cc(Cl)cnc2OC(F)F)c1]

Following the procedure employed in Example 74 Step 1 using 1-(3-methoxyphenyl)-N-methylmethanamine (48.7 mg, 0.322 mmol) and 5-chloro-2-(difluoromethoxy)pyridine-3-carbonyl chloride (Intermediate 47) (65 mg, crude), gave 5-chloro-2-(difluoromethoxy)-N-(3-methoxybenzyl)-N-methylnicotinamide (80.83 mg, 0.226 mmol, 84.19% yield) as a white solid.

¹H NMR (400 MHz, DMSO-d₆): 8.46-8.38 (m, 1H), 8.25-8.14 (m, 1H), 7.95-7.48 (m, 1H), 7.26 (q, J=7.8 Hz, 1H), 6.94-6.83 (m, 2H), 6.79-6.70 (m, 1H), 5.02-4.30 (m, 2H), 3.82-3.69 (m, 3H), 2.94-2.71 (m, 3H). MS ES⁺: 357.3

### Example 82: 5-chloro-2-(difluoromethoxy)-N-methyl-N-(3-(trifluoromethyl)benzyl)nicotinamide

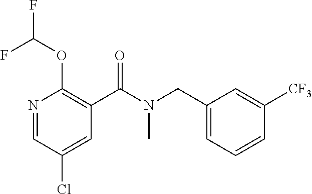 [SMILES: CN(Cc1cccc(C(F)(F)F)c1)C(=O)c1cc(Cl)cnc1OC(F)F]

Following the procedure employed in Example 74 Step 1 using N-methyl-1-(3-(trifluoromethyl)phenyl)methanamine (61 mg, 0.322 mmol) and 5-chloro-2-(difluoromethoxy)pyridine-3-carbonyl chloride (Intermediate 47) (65.0 mg, crude) gave 5-chloro-2-(difluoromethoxy)-N-methyl-N-(3-(trifluoromethyl)benzyl) nicotinamide (84.07 mg, 0.213 mmol, 79.30% yield) as a white solid.

¹H NMR (400 MHz, DMSO-d₆): 8.4-8.5 (m, 1H) 8.2-8.3 (m, 1H) 7.5-7.7 (m, 5H) 4.5 (br s, 2H) 2.7-2.9 (m, 3H). MS ES⁺: 395.2

### Example 83: 2-(difluoromethoxy)-5-fluoro-N-(1-(3-fluorophenyl)ethyl)-N-methylnicotinamide

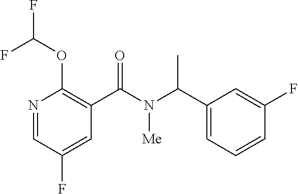 [SMILES: CC(c1cccc(F)c1)N(C)C(=O)c1cc(F)cnc1OC(F)F]

Following the procedure employed in Example 74 Step 1 using 1-(3-fluorophenyl)-N-methyl-ethanamine (Intermediate 30) (77.4 mg, 0.505 mmol) and 2-(difluoromethoxy)-5-fluoro-pyridine-3-carbonyl chloride (Intermediate 45) (95 mg, crude) gave 2-(difluoromethoxy)-5-fluoro-N-(1-(3-fluorophenyl)ethyl)-N-methylnicotinamide (48.49 mg, 0.139 mmol, 32.99% yield) as a colourless gum.

¹H NMR (400 MHz, DMSO-d₆): 8.44-7.49 (m, 3H), 7.46-7.32 (m, 1H), 7.26-7.09 (m, 3H), 5.88 (q, J=6.7 Hz, 0.6H), 4.84-4.69 (m, 0.4H), 2.75-2.65 (m, 1.5H), 2.52 (s, 1.5H), 1.56 (d, J=7.1 Hz, 3H). MS ES⁺: 343.1

### Example 84: 5-chloro-N-(3-chlorobenzyl)-2-(difluoromethoxy)-N-methylnicotinamide

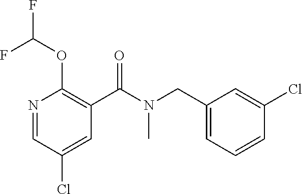 [SMILES: CN(Cc1cccc(Cl)c1)C(=O)c1cc(Cl)cnc1OC(F)F]

Following the procedure employed in Example 74 Step 1 using 5-chloro-2-(difluoromethoxy)pyridine-3-carbonyl chloride (Intermediate 47) (65 mg, 0.269 mmol) and 1-(3-chlorophenyl)-N-methylmethanamine (50.2 mg, 0.322 mmol), gave 5-chloro-N-(3-chlorobenzyl)-2-(difluoromethoxy)-N-methylnicotinamide (63.29 mg, 0.175 mmol, 65.24% yield) as a white solid.

¹H NMR (400 MHz, DMSO-d₆): 8.4-8.5 (m, 1H) 8.1-8.3 (m, 1H) 7.6 (s, 1H) 7.1-7.5 (m, 4H) 4.3-5.0 (m, 2H) 2.7-3.0 (m, 3H). MS ES⁺: 361.2

### Example 85: 2-(difluoromethoxy)-N-(2,3-dihydrobenzofuran-3-yl)-5-fluoro-N-methylnicotinamide

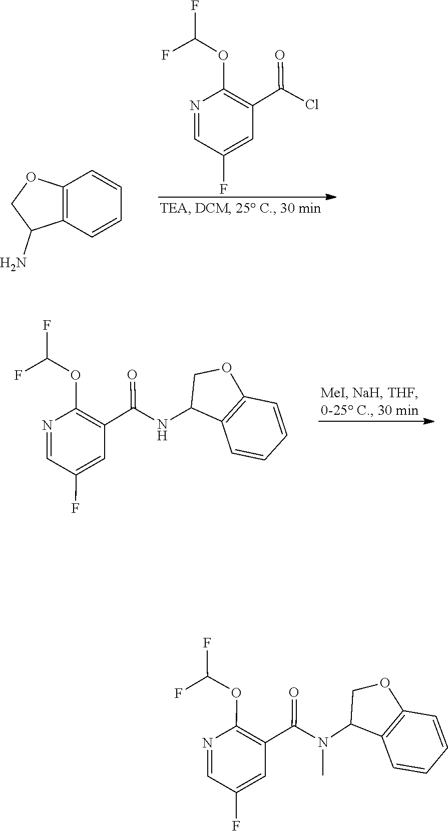 [SMILES: NC1COc2ccccc21 ; O=C(Cl)c1cc(F)cnc1OC(F)F ; O=C(NC1COc2ccccc21)c1cc(F)cnc1OC(F)F ; CN(C(=O)c1cc(F)cnc1OC(F)F)C1COc2ccccc21]

Following the procedure employed for Example 74 using 2,3-dihydrobenzofuran-3-amine (50 mg, 0.201 mmol) (prepared as described in Tetrahedron Letters, 2012, vol. 53, #46, p. 6273-6276) and 2-(difluoromethoxy)-5-fluoro-pyridine-3-carbonyl chloride (Intermediate 45) (45.3 mg, 0.201 mmol), gave 2-(difluoromethoxy)-N-(2,3-dihydrobenzofuran-3-yl)-5-fluoro-N-methylnicotinamide (11.66 mg, 0.0345 mmol, 37.2% yield) as a colourless gum.

¹H NMR (400 MHz, DMSO-d₆): 8.45-7.99 (m, 2H), 7.90-7.47 (m, 1H), 7.43-7.20 (m, 2H), 7.02-6.84 (m, 2H), 6.40 (m, 0.5H), 5.42 (m, 0.5H), 4.79-4.42 (m, 2H), 2.68-2.63 (m, 2H), 2.34-2.32 (m, 1H). MS ES⁺: 339.3

### Example 86 (racemate) and Example 87 (enantiomer 1): 5-chloro-2-(difluoromethoxy)-N-(1-(3-fluorophenyl)ethyl)-N-methylnicotinamide

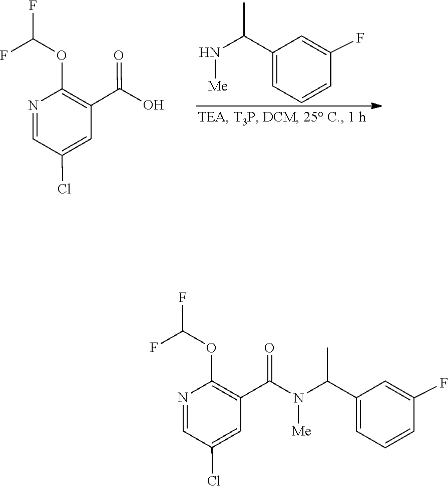 [SMILES: O=C(O)c1cc(Cl)cnc1OC(F)F ; CNC(C)c1cccc(F)c1 ; CC(c1cccc(F)c1)N(C)C(=O)c1cc(Cl)cnc1OC(F)F]

Step 1: A mixture of 5-chloro-2-(difluoromethoxy)nicotinic acid (250 mg, 1.12 mmol), 1-(3-fluorophenyl)-N-methyl-ethanamine (Intermediate 30) (171 mg, 1.12 mmol), triethylamine (340 mg, 3.35 mmol) and T₃P (1.07 g, 1.68 mmol, 50% purity in ethyl acetate) in dichloromethane (3 mL) was stirred at 25° C. for 1 hour. The mixture was poured into H₂O (50 mL) and extracted with dichloromethane (50 mL×3) and the combined organic layers were concentrated to dryness. The crude was purified by prep. HPLC (Column: Xtimate C18 100*30 mm*10 μm, Mobile Phase A: water (0.225% HCOOH), Mobile Phase B: acetonitrile, Flow rate: 25 mL/min, gradient condition from 50% B to 80%). The residue was partitioned between acetonitrile (2 mL) and water (10 mL). The solution was lyophilized to dryness to give 5-chloro-2-(difluoromethoxy)-N-(1-(3-fluorophenyl)ethyl)-N-methylnicotinamide (64.01 mg, 0.178 mmol, 91.0% yield) as a white solid.

¹H NMR (400 MHz, DMSO-d₆): 8.36-8.51 (m, 1H) 8.22-8.35 (m, 1H) 7.51-7.94 (m, 1H) 7.37-7.47 (m, 1H) 7.09-7.26 (m, 3H) 5.83-5.93 (m, 0.64H) 4.78 (br d, J=6.25 Hz, 0.37H) 2.67-2.74 (m, 1H) 2.53 (s, 2H) 1.56 (br d, J=7.00 Hz, 3H); MS ES⁺: 359.3.

Preparative SFC condition: REGIS (s, s) WHELK-01 (250 mm*30 mm*5 μm); A: Supercritical CO₂, B: 0.1% NH₃H₂O EtOH, A:B=83:17 at 80 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The pure fractions were collected and the volatiles were removed under vacuum. The residue was partitioned between acetonitrile (2 mL) and water (10 mL). The mixture was lyophilized to dryness to give 5-chloro-2-(difluoromethoxy)-N-(1-(3-fluorophenyl)ethyl)-N-methylnicotinamide (18.65 mg, 0.052 mmol, 29.6% yield) as a white solid.

¹H NMR (400 MHz, DMSO-d₆): 8.40-8.46 (m, 1H) 8.22-8.34 (m, 1H) 7.53-7.95 (m, 1H) 7.38-7.47 (m, 1H) 7.10-7.26 (m, 3H) 4.72-5.96 (m, 1H) 2.52 (s, 3H) 1.53-1.59 (m, 3H); MS ES⁺: 359.3; SFC Chiral Purity 99.8%, Rt=1.30 min

The other enantiomer was not isolated

### Example 88 (enantiomer 1) and Example 89 (enantiomer 2): 2-(difluoromethoxy)-5-fluoro-N-(1-(3-fluorophenyl)ethyl)-N-methylnicotinamide

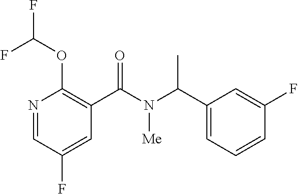 [SMILES: CC(c1cccc(F)c1)N(C)C(=O)c1cc(F)cnc1OC(F)F]

Following the procedure employed in Example 74 Step 1 using 1-(3-fluorophenyl)-N-methyl-ethanamine (Intermediate 30) (77.4 mg, 0.51 mmol) and 2-(difluoromethoxy)-5-fluoro-pyridine-3-carbonyl chloride (Intermediate 45) (95 mg, 0.421 mmol) gave racemic 2-(difluoromethoxy)-5-fluoro-N-(1-(3-fluorophenyl)ethyl)-N-methylnicotinamide (48.49 mg, 0.139 mmol, 33.0% yield) as a colourless gum. SFC Separation Conditions: REGIS (s, s) WHELK-01 (250 mm*30 mm*5 μm); A: Supercritical CO₂, B: 0.1% NH₃H₂O EtOH, A:B=83:17 at 80 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The pure fractions were collected and the volatiles were removed under vacuum. The residue was partitioned between acetonitrile (2 mL) and water (10 mL). The mixture was lyophilized to dryness to give Peak 1 Example 88 (5.25 mg, 0.015 mmol, 10.9% yield) as a colourless gum and Peak 2 Example 89 (6.41 mg, 0.018 mmol, 13.3% yield) as a white solid.

Example 88 (Peak 1): ¹H NMR (400 MHz, DMSO-d₆): 8.35-8.41 (m, 1H); 8.08-8.26 (m, 1H); 7.50-7.94 (m, 1H); 7.36-7.46 (m, 1H); 7.08-7.25 (m, 3H); 4.72-5.97 (m, 1H); 2.52 (s, 3H); 1.56 (d, J=7.13 Hz, 3H); SFC: 98.4% chiral purity, Rt=1.11 min,

Example 89 (Peak 2): ¹H NMR (400 MHz, DMSO-d₆): 8.34-8.42 (m, 1H); 8.08-8.27 (m, 1H); 7.50-7.94 (m, 1H); 7.35-7.48 (m, 1H); 7.10-7.25 (m, 3H); 4.72-5.96 (m, 1H); 2.53 (s, 3H); 1.56 (d, J=7.00 Hz, 3H); MS ES⁺: 343.1; SFC: 100.0% chiral purity, Rt=1.26 min.

### Example 90: 5-chloro-N-(3-cyanobenzyl)-2-(difluoromethoxy)-N-methylnicotinamide

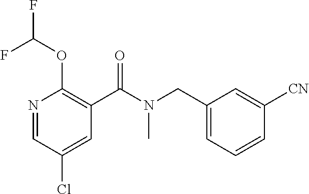 [SMILES: CN(Cc1cccc(C#N)c1)C(=O)c1cc(Cl)cnc1OC(F)F]

Following the procedure employed for Example 74 using 5-chloro-2-(difluoromethoxy)pyridine-3-carbonyl chloride (Intermediate 47) (210 mg, 0.868 mmol) and 3-(aminomethyl)benzonitrile (138 mg, 1.04 mmol), gave 5-chloro-N-(3-cyanobenzyl)-2-(difluoromethoxy)-N-methylnicotinamide (2.78 mg, 0.008 mmol, 5.28% yield) as a white solid.

¹H NMR (400 MHz, DMSO-d₆): 8.37-8.48 (m, 1H) 8.20-8.28 (m, 1H) 7.48-7.93 (m, 5H) 4.34-5.01 (m, 2H) 2.77-2.94 (m, 3H); MS ES⁺: 351.8

### Example 91: 5-chloro-2-(difluoromethoxy)-N-(2,3-dihydrobenzofuran-3-yl)-N-methylnicotinamide

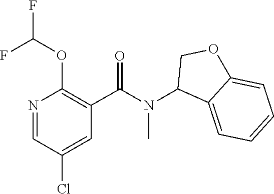 [SMILES: CN(C(=O)c1cc(Cl)cnc1OC(F)F)C1COc2ccccc21]

Following the procedure employed for Example 74 using 2,3-dihydrobenzofuran-3-amine hydrochloride (103 mg, 0.600 mmol) (prepared as described in WO2016/100050) and 5-chloro-2-(difluoromethoxy)pyridine-3-carbonyl chloride (Intermediate 47) (100 mg, 0.413 mmol), gave 5-chloro-2-(difluoromethoxy)-N-(2,3-dihydrobenzofuran-3-yl)-N-methylnicotinamide (23.29 mg, 0.066 mmol, 44.66% yield) as a white powder.

¹H NMR (400 MHz, DMSO-d₆): 8.48-8.17 (m, 2H), 7.90-7.51 (m, 1H), 7.42-7.22 (m, 2H), 6.99-6.81 (m, 2H), 6.39 (m, 0.5H), 5.49-5.40 (m, 0.5H), 4.77-4.46 (m, 2H), 2.68-2.61 (m, 2H), 2.44 (s, 1H); MS ES⁺: 355.3

### Example 92: 5-chloro-2-(difluoromethoxy)-N-(7-fluoro-2,3-dihydrobenzofuran-3-yl)-N-methylnicotinamide

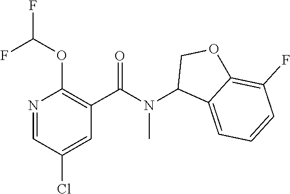 [SMILES: CN(C(=O)c1cc(Cl)cnc1OC(F)F)C1COc2c(F)cccc21]

Following the procedure employed in Example 74 Step 1 using 7-fluoro-N-methyl-2,3-dihydrobenzofuran-3-amine trifluoroacetate (Intermediate 49) (80 mg, crude) and 5-chloro-2-(difluoromethoxy)pyridine-3-carbonyl chloride (Intermediate 47) (50 mg, 0.207 mmol), gave 5-chloro-2-(difluoromethoxy)-N-(7-fluoro-2,3-dihydrobenzofuran-3-yl)-N-methylnicotinamide (17.88 mg, 0.0479 mmol, 16.83% yield) as a white solid.

¹H NMR (400 MHz, DMSO-d₆): 8.59-8.09 (m, 2H), 7.94-7.47 (m, 1H), 7.31-6.89 (m, 3H), 6.45 (m, 0.5H), 5.74-5.37 (m, 0.5H), 4.91-4.54 (m, 2H), 2.66 (s, 2H), 2.48 (s, 1H); MS ES⁺: 373.2

### Example 93: N-(2,3-difluorobenzyl)-2-(difluoromethoxy)-5-fluoro-N-methylnicotinamide

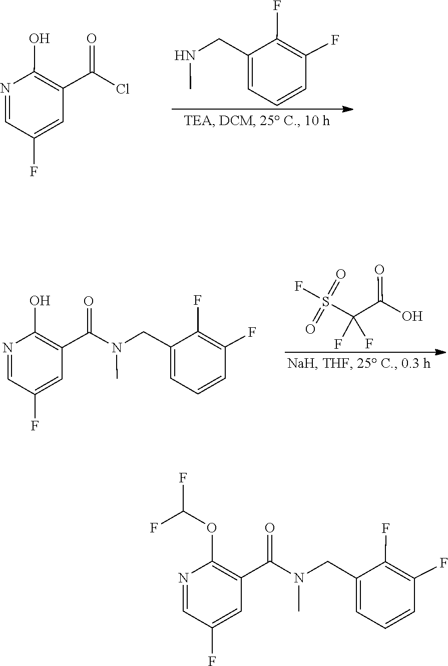 [SMILES: O=C(Cl)c1cc(F)cnc1O ; CNCc1cccc(F)c1F ; CN(Cc1cccc(F)c1F)C(=O)c1cc(F)cnc1O ; O=C(O)C(F)(F)S(=O)(=O)F ; CN(Cc1cccc(F)c1F)C(=O)c1cc(F)cnc1OC(F)F]

Step 1: A mixture of 5-fluoro-2-hydroxynicotinoyl chloride (Intermediate 50) (400 mg, crude) and 1-(2, 3-difluorophenyl)-N-methyl-methanamine (286.5 mg, 1.82 mmol) (Intermediate 56) and triethylamine (576 mg, 5.70 mmol) in dichloromethane (1 mL) was stirred at 25° C. for 10 hours. The mixture was concentrated to afford the crude product which was purified by flash silica gel chromatography (ISCO®; 20 g SepaFlash® Silica Flash Column, Eluent of 0˜100% Ethyl acetate/Petroleum ether gradient @45 mL/min) to give N-(2,3-difluorobenzyl)-5-fluoro-2-hydroxy-N-methylnicotinamide (450 mg, 1.39 mmol, 61.2% yield, 91.8% purity) as a yellow oil. MS ES⁺: 296.9

Step 2: 2,2-Difluoro-2-fluorosulfonyl-acetic acid (112 mg, 0.628 mmol) was added into a mixture of N-(2,3-difluorobenzyl)-5-fluoro-2-hydroxy-N-methylnicotinamide (186 mg, 0.628 mmol) and NaH (50.2 mg, 1.26 mmol, 60% purity) in THF (2 mL). The mixture was stirred at 25° C. for 0.3 hour. The mixture was quenched by saturated NH₄Cl (aq.) (10 mL) and extracted with ethyl acetate (3×10 mL), then evaporated to dryness to obtain crude product which was further purified by prep. HPLC (Column: Phenomenex luna30*30 mm*10 μm+YMC AQ 100*30*10 um, Mobile Phase A: water (0.05% HCl), Mobile Phase B: acetonitrile, Flow rate: 25 mL/min, gradient condition from 30% B to 80%). The pure fractions were collected and the volatiles were removed under vacuum. The residue was partitioned between acetonitrile (2 mL) and water (10 mL). The solution was lyophilized to dryness to give N-(2,3-difluorobenzyl)-2-(difluoromethoxy)-5-fluoro-N-methylnicotinamide (77.87 mg, 0.224 mmol, 35.64% yield) as a white solid.

¹H NMR (400 MHz, DMSO-d₆): 8.56-8.28 (m, 1H), 8.19-7.98 (m, 1H), 7.94-7.45 (m, 1H), 7.44-7.34 (m, 1H), 7.27-7.22 (m, 1H), 7.21-6.97 (m, 1H), 5.04-4.45 (m, 2H), 3.03-2.77 (m, 3H); MS ES⁺: 347.2

### Example 94: N-(2,4-difluorobenzyl)-2-(difluoromethoxy)-5-fluoro-N-methylnicotinamide

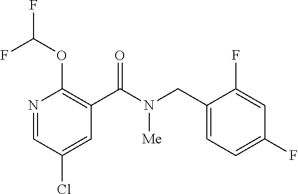 [SMILES: CN(Cc1ccc(F)cc1F)C(=O)c1cc(Cl)cnc1OC(F)F]

Following the procedure employed for Example 93 using 5-fluoro-2-hydroxynicotinoyl chloride (Intermediate 50) (100 mg, crude) and 1-(2,4-difluorophenyl)-N-methyl-methanamine (Intermediate 43) (98.5 mg, 0.627 mmol), gave N-(2,4-difluorobenzyl)-2-(difluoromethoxy)-5-fluoro-N-methylnicotinamide (24.72 mg, 0.0711 mmol, 27.01% yield) as a white powder.

¹H NMR (400 MHz, DMSO-d₆): 8.49-8.29 (m, 1H), 8.13-7.97 (m, 1H), 7.88-7.35 (m, 2H), 7.27 (br dd, J=2.0, 9.9 Hz, 1H), 7.15-7.07 (m, 1H), 4.90-4.31 (m, 2H), 2.93-2.79 (m, 3H); MS ES⁺: 347.2

### Example 95: 2-(difluoromethoxy)-5-fluoro-N-(3-fluorobenzyl)-N-methylnicotinamide

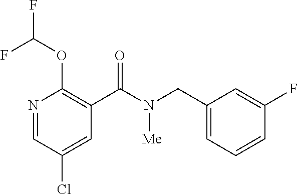 [SMILES: CN(Cc1cccc(F)c1)C(=O)c1cc(Cl)cnc1OC(F)F]

Following the procedure employed for Example 93 using 5-fluoro-2-hydroxy-pyridine-3-carboxylic acid (Intermediate 50) (500 mg, 3.18 mmol) and 1-(3-fluorophenyl)-N-methyl-methanamine (Intermediate 55) (131 mg, 0.94 mmol) gave 2-(difluoromethoxy)-5-fluoro-N-(3-fluorobenzyl)-N-methylnicotinamide (40.03 mg, 0.122 mmol, 19.9% yield) as a white solid.

¹H NMR (400 MHz, DMSO-d₆): 8.46-8.20 (m, 1H), 8.01 (d, J=7.4 Hz, 1H), 7.86-7.31 (m, 2H), 7.30-6.89 (m, 3H), 4.83-4.33 (m, 2H), 2.99-2.72 (m, 3H); MS ES⁺: 329.3

### Example 96: 2-(difluoromethoxy)-N-(3-fluorobenzyl)-N-methyl-5-(trifluoromethyl)nicotinamide

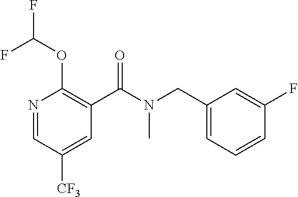 [SMILES: CN(Cc1cccc(F)c1)C(=O)c1cc(C(F)(F)F)cnc1OC(F)F]

Following the procedure employed for Example 93 using 2-hydroxy-5-(trifluoromethyl)pyridine-3-carbonyl chloride (Intermediate 51) (150 mg, crude) and 1-(3-fluorophenyl)-N-methyl-methanamine (Intermediate 55) (92.6 mg, 0.665 mmol), gave 2-(difluoromethoxy)-N-(3-fluorobenzyl)-N-methyl-5-(trifluoromethyl)nicotinamide (8.45 mg, 0.022 mmol, 28.85% yield) as an off-white solid.

¹H NMR (400 MHz, DMSO-d₆): 8.90-8.37 (m, 2H), 8.10-7.58 (m, 1H), 7.50-7.31 (m, 1H), 7.29-6.97 (m, 3H), 5.12-4.27 (m, 2H), 2.98-2.75 (m, 3H); MS ES⁺: 379.3

### Example 97: 5-cyclobutyl-2-(difluoromethoxy)-N-(3-fluorobenzyl)-N-methylnicotinamide

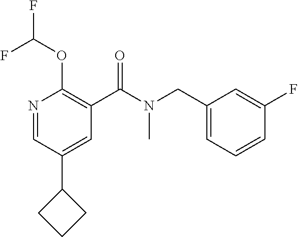 [SMILES: CN(Cc1cccc(F)c1)C(=O)c1cc(C2CCC2)cnc1OC(F)F]

Following the procedure employed for Example 93 using 5-cyclobutyl-2-(difluoromethoxy)pyridine-3-carbonyl chloride (Intermediate 52) (30 mg, crude) and 1-(3-fluorophenyl)-N-methyl-methanamine (Intermediate 55) (31.9 mg, 0.229 mmol), gave 5-cyclobutyl-2-(difluoromethoxy)-N-(3-fluorobenzyl)-N-methylnicotinamide (9.27 mg, 0.025 mmol, 21.88% yield) as a colourless oil.

¹H NMR (400 MHz, MeOD-d₄): 8.20-8.08 (m, 1H), 7.84-7.57 (m, 2H), 7.45-7.31 (m, 1H), 7.24-7.12 (m, 1H), 7.09-6.90 (m, 2H), 4.60 (s, 2H), 3.70-3.57 (m, 1H), 3.10-2.84 (m, 3H), 2.48-2.28 (m, 2H), 2.26-2.02 (m, 3H), 1.98-1.83 (m, 1H); MS ES⁺: 365.3

### Example 98: 5-chloro-2-(difluoromethoxy)-N-(3-(difluoromethoxy)benzyl)-N-methylnicotinamide

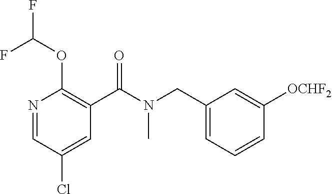 [SMILES: CN(Cc1cccc(OC(F)F)c1)C(=O)c1cc(Cl)cnc1OC(F)F]

Following the procedure employed for Example 74 using [3-(difluoromethoxy)phenyl]methanamine hydrochloride (Intermediate 53) (58 mg, crude) and 5-chloro-2-(difluoromethoxy)pyridine-3-carbonyl chloride (Intermediate 47), gave 5-chloro-2-(difluoromethoxy)-N-(3-(difluoromethoxy)benzyl)-N-methylnicotinamide (15.27 mg, 0.038 mmol, 11.15% yield) as a white powder.

¹H NMR (400 MHz, DMSO-d₆): 8.49-8.40 (m, 1H), 8.26-8.19 (m, 1H), 7.94-7.51 (m, 1H), 7.46-7.39 (m, 1H), 7.27-7.23 (m, 1H), 7.19-6.98 (m, 3H), 5.06-4.30 (m, 2H), 2.95-2.77 (m, 3H); MS ES⁺: 393.0

### Example 99: 5-chloro-2-(difluoromethoxy)-N-methyl-N-(3-(trifluoromethoxy)benzyl)nicotinamide

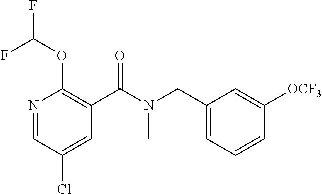 [SMILES: CN(Cc1cccc(OC(F)(F)F)c1)C(=O)c1cc(Cl)cnc1OC(F)F]

Following the procedure employed for Example 74 using 5-chloro-2-(difluoromethoxy)pyridine-3-carbonyl chloride (Intermediate 47) (100 mg, crude) and [3-(trifluoromethoxy)phenyl]methanamine (158 mg, 0.826 mmol), gave 5-chloro-2-(difluoromethoxy)-N-methyl-N-(3-(trifluoromethoxy)benzyl)nicotinamide (19.01 mg, 0.045 mmol, 19.9% yield) as a white solid.

¹H NMR (400 MHz, DMSO-d₆): 8.53-8.39 (m, 1H), 8.28-8.16 (m, 1H), 8.01-7.66 (m, 1H), 7.61-7.46 (m, 1H), 7.44-7.11 (m, 3H), 5.23-4.25 (m, 2H), 2.95-2.78 (m, 3H); MS ES⁺: 410.9

### Example 100: 5-chloro-N-(2,4-difluorobenzyl)-2-(difluoromethoxy)-N-methylnicotinamide

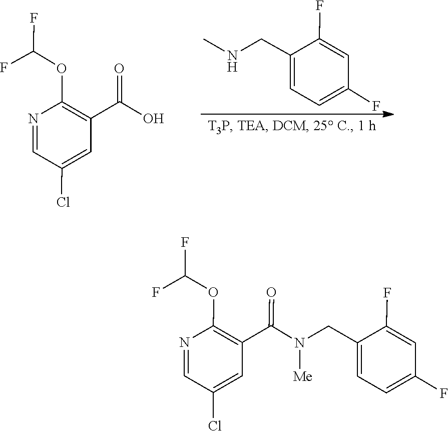 [SMILES: O=C(O)c1cc(Cl)cnc1OC(F)F ; CNCc1ccc(F)cc1F ; CN(Cc1ccc(F)cc1F)C(=O)c1cc(Cl)cnc1OC(F)F]

A mixture of 5-chloro-2-(difluoromethoxy)nicotinic acid (Intermediate 54) (50 mg, crude), 1-(2,4-difluorophenyl)-N-methyl-methanamine (Intermediate 43) (35.2 mg, 0.22 mmol), T₃P (213.5 mg, 0.34 mmol, 50% purity in ethyl acetate) and triethylamine (67.9 mg, 0.671 mmol) in dichloromethane (1 mL) was stirred at 25° C. for 1 hour. The mixture was concentrated to dryness which was purified by prep. HPLC (Column: Xtimate C18 100*30 mm*10 μm, Mobile Phase A: water (0.225% HCOOH), Mobile Phase B: acetonitrile, Flow rate: 10 mL/min, gradient condition from 50% B to 80%). The pure fractions were collected and the volatiles were removed under vacuum. The residue was partitioned between acetonitrile (2 mL) and water (10 mL). The solution was lyophilized to dryness to give the title compound 5-chloro-N-(2,4-difluorobenzyl)-2-(difluoromethoxy)-N-methylnicotinamide (32.32 mg, 0.081 mmol, 39.84% yield) as a white powder. ¹H NMR (400 MHz, DMSO-d₆): 8.40-8.46 (m, 1H) 8.09-8.21 (m, 1H) 7.52-7.91 (m, 1H) 7.18-7.50 (m, 2H) 7.03-7.11 (m, 1H) 4.37-4.90 (m, 2H) 2.77-2.94 (m, 3H); MS ES⁺: 363.2

### Example 101: 5-chloro-2-(difluoromethoxy)-N-(3-fluorobenzyl)-N-methylnicotinamide

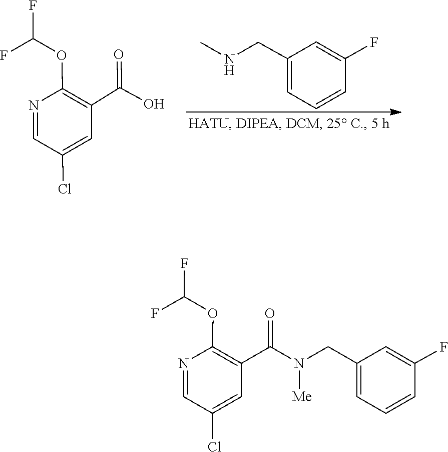

To a solution of 5-chloro-2-(difluoromethoxy)pyridine-3-carboxylic acid (Intermediate 54) (80.3 mg, 0.359 mmol), 1-(3-fluorophenyl)-N-methylmethanamine (Intermediate 55) (50 mg, 0.359 mmol) and DIPEA (139 mg, 1.08 mmol) in dichloromethane (3 mL) was added HATU (205 mg, 0.539 mmol). The mixture was stirred at 25° C. for 5 hours. The reaction mixture was diluted with H₂O (20 mL) and extracted with ethyl acetate (3×20 mL). The combined organic layers were washed with brine (40 mL), dried over anhydrous Na₂SO₄, filtered and concentrated under reduced pressure to dryness which was purified by flash silica gel chromatography (ISCO®; 4 g SepaFlash® Silica Flash Column, Eluent of 0˜18% Ethyl acetate/Petroleum ether gradient @50 mL/min) to afford the title compound (67.7 mg, 0.193 mmol, 53.60% yield) as a white solid.

¹H NMR (400 MHz, DMSO-d₆): 8.48-8.36 (m, 1H), 8.27-8.18 (m, 1H), 7.94-7.49 (m, 1H), 7.45-7.34 (m, 1H), 7.24-7.00 (m, 3H), 5.16-4.35 (m, 2H), 2.95-2.75 (m, 3H); MS ES⁺: 345.2.

### Example 102: 5-chloro-N-(2,3-difluorobenzyl)-2-(difluoromethoxy)-N-methylnicotinamide

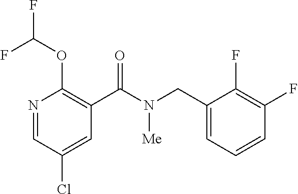 [SMILES: CN(Cc1cccc(F)c1F)C(=O)c1cc(Cl)cnc1OC(F)F]

Following the procedure employed for Example 101 using 5-chloro-2-(difluoromethoxy)pyridine-3-carboxylic acid (Intermediate 54) (50 mg, 0.224 mmol) and 1-(2,3-difluorophenyl)-N-methylmethanamine (Intermediate 56) (35.2 mg, 0.224 mmol) gave 5-chloro-N-(2, 3-difluorobenzyl)-2-(difluoromethoxy)-N-methylnicotinamide (46.96 mg, 0.129 mmol, 57.8% yield) as a white solid.

¹H NMR (400 MHz, DMSO-d₆): 8.52-8.35 (m, 1H), 8.28-8.06 (m, 1H), 7.94-7.45 (m, 1H), 7.44-7.30 (m, 1H), 7.27-7.08 (m, 2H), 5.12-4.43 (m, 2H), 3.02-2.74 (m, 3H); MS ES⁺: 363.2.

### Example 103: N-(3,5-difluorobenzyl)-2-(difluoromethoxy)-5-fluoro-N-methylpyridine-3-sulfonamide

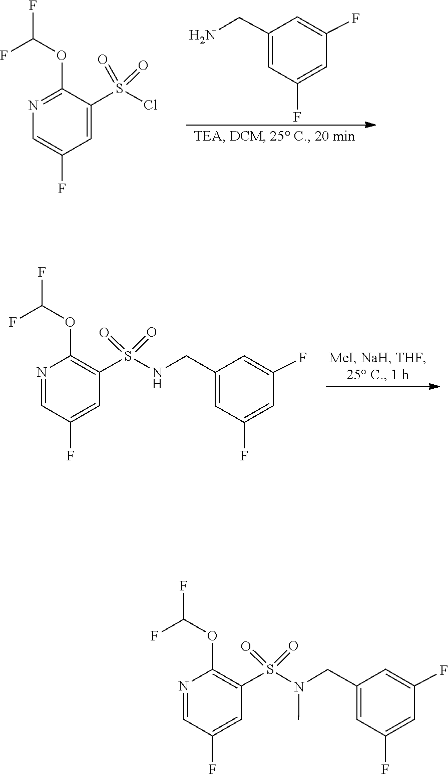 [SMILES: O=S(=O)(Cl)c1cc(F)cnc1OC(F)F ; NCc1cc(F)cc(F)c1 ; O=S(=O)(NCc1cc(F)cc(F)c1)c1cc(F)cnc1OC(F)F ; CN(Cc1cc(F)cc(F)c1)S(=O)(=O)c1cc(F)cnc1OC(F)F]

Step 1: To a mixture of 2-(difluoromethoxy)-5-fluoropyridine-3-sulfonyl chloride (Intermediate 57) (80 mg, 0.306 mmol) and (3,5-difluorophenyl)methanamine (52.5 mg, 0.367 mmol) in dichloromethane (0.1 mL) was added triethylamine (92.8 mg, 0.917 mmol) in one portion at 25° C. The mixture was stirred at 25° C. for 20 minutes. The mixture was poured into water (10 mL) and extracted with dichloromethane (10 mL) and the combined organic layers were concentrated to afford N-(3,5-difluorobenzyl)-2-(difluoromethoxy)-5-fluoropyridine-3-sulfonamide (100 mg, crude) as a yellow oil. MS ES⁺: 369.0

Step 2: To a mixture of N-(3,5-difluorobenzyl)-2-(difluoromethoxy)-5-fluoropyridine-3-sulfonamide (100 mg, crude) and NaH (32.6 mg, 0.815 mmol, 60% purity) in THF (1 mL) was added Mel (116 mg, 0.815 mmol) in one portion at 0° C. under N₂. The mixture was stirred at 25° C. for 1 hour. The mixture was poured into saturated NH₄Cl (aq.) (10 mL) and extracted with ethyl acetate (10 mL×3), the combined organic layers were concentrated to dryness which was purified by prep. HPLC (Column: Xtimate C18 100*30 mm*10 μm, Mobile Phase A: water (HCOOH), Mobile Phase B: acetonitrile, Flow rate: 25 mL/min, gradient condition from 60% B to 90%). The pure fractions were collected and the volatiles were removed under vacuum. The residue was partitioned between acetonitrile (2 mL) and water (10 mL). The solution was lyophilized to dryness to give N-(3,5-difluorobenzyl)-2-(difluoromethoxy)-5-fluoro-N-methylpyridine-3-sulfonamide (27.52 mg, 0.072 mmol, 26.4% yield) as a white solid.

¹H NMR (400 MHz, DMSO-d₆): 8.62 (d, J=3.0 Hz, 1H), 8.32 (dd, J=2.9, 7.3 Hz, 1H), 8.04-7.56 (m, 1H), 7.20 (tt, J=2.3, 9.4 Hz, 1H), 7.07-6.97 (m, 2H), 4.41 (s, 2H), 2.83 (s, 3H); MS ES⁺: 383.2

### Example 104: (R)-2-(difluoromethoxy)-5-fluoro-N-(i-(3-fluorophenyl)ethyl)-N-methylpyridine-3-sulfonamide

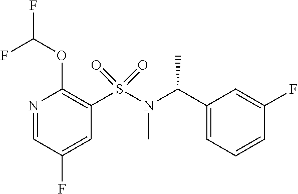 [SMILES: C[C@H](c1cccc(F)c1)N(C)S(=O)(=O)c1cc(F)cnc1OC(F)F]

Following the procedure employed for Example 103 using 2-(difluoromethoxy)-5-fluoro-pyridine-3-sulfonyl chloride (Intermediate 57) (90 mg, 0.344 mmol) and (1R)-1-(3-fluorophenyl)ethanamine (57-5 mg, 0.413 mmol), gave (R)-2-(difluoromethoxy)-5-fluoro-N-(1-(3-fluorophenyl)ethyl)-N-methylpyridine-3-sulfonamide (10.69 mg, 0.0273 mmol, 9.93% yield) as a white solid.

¹H NMR (400 MHz, DMSO-d₆): 8.60 (d, J=2.88 Hz, 1H) 8.30 (dd, J=7.32, 2.94 Hz, 1H) 7.79 (t, J=71.35 Hz, 1H) 7.34-7.42 (m, 1H) 7.06-7.16 (m, 3H) 5.11 (q, J=7.00 Hz, 1H) 2.77 (s, 3H) 1.40 (d, J=7.00 Hz, 3H); MS ES⁺: 379.3; SFC: 99.5% chiral purity, Rt=0.657 min.

### Example 105: (S)-2-(difluoromethoxy)-5-fluoro-N-(1-(3-fluorophenyl)ethyl)-N-methylpyridine-3-sulfonamide

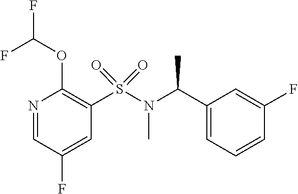 [SMILES: C[C@@H](c1cccc(F)c1)N(C)S(=O)(=O)c1cc(F)cnc1OC(F)F]

Following the procedure employed for Example 103 using 2-(difluoromethoxy)-5-fluoro-pyridine-3-sulfonyl chloride (Intermediate 57) (90 mg, 0.344 mmol) and (1S)-1-(3-fluorophenyl)ethanamine (57-5 mg, 0.413 mmol) gave (S)-2-(difluoromethoxy)-5-fluoro-N-(1-(3-fluorophenyl)ethyl)-N-methylpyridine-3-sulfonamide (10.62 mg, 0.027 mmol, 9.66% yield) as a white solid.

¹H NMR (400 MHz, DMSO-d₆): 8.60 (d, J=3.00 Hz, 1H) 8.31 (dd, J=7.32, 2.94 Hz, 1H) 7.79 (t, J=71.35 Hz, 1H) 7.34-7.42 (m, 1H) 7.06-7.17 (m, 3H) 5.11 (q, J=7.00 Hz, 1H) 2.77 (s, 3H) 1.40 (d, J=7.00 Hz, 3H); MS ES⁺: 379.2; SFC: 99.7% chiral purity, Rt=0.477 min.

### Example 106: N-(3-cyanobenzyl)-2-(difluoromethoxy)-5-fluoro-N-methylpyridine-3-sulfonamide

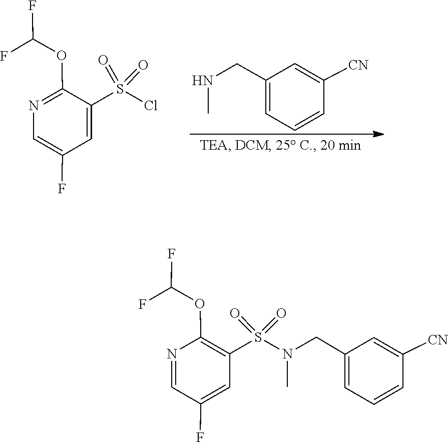 [SMILES: O=S(=O)(Cl)c1cc(F)cnc1OC(F)F ; CNCc1cccc(C#N)c1 ; CN(Cc1cccc(C#N)c1)S(=O)(=O)c1cc(F)cnc1OC(F)F]

To a mixture of 2-(difluoromethoxy)-5-fluoro-pyridine-3-sulfonyl chloride (Intermediate 57) (50 mg, 0.191 mmol) and 3-((methylamino)methyl)benzonitrile (33.5 mg, 0.229 mmol) in dichloromethane (0.1 mL) was added triethylamine (58.0 mg, 0.573 mmol) in one portion at 25° C. The mixture was stirred at 25° C. for 20 minutes. The mixture was poured into water (10 mL) and extracted with dichloromethane (3×10 mL), the combined organic layers were concentrated to afford the crude product which was purified by prep. HPLC (Column: Xtimate C18 100*30 mm*10 μm, Mobile Phase A: water (HCOOH), Mobile Phase B: acetonitrile, Flow rate: 25 mL/min, gradient condition from 50% B to 80%). The pure fractions were collected and the volatiles were removed under vacuum. The residue was partitioned between acetonitrile (2 mL) and water (10 mL). The solution was lyophilized to dryness to give N-(3-cyanobenzyl)-2-(difluoromethoxy)-5-fluoro-N-methylpyridine-3-sulfonamide (3.53 mg, 0.009 mmol, 4.95% yield) as an off-white gum.

¹H NMR (400 MHz, DMSO-d₆): 8.62 (d, J=3.0 Hz, 1H), 8.32 (dd, J=2.9, 7.4 Hz, 1H), 7.98-7.67 (m, 3H), 7.67-7.59 (m, 2H), 4.45 (s, 2H), 2.83 (s, 3H); MS ES⁺: 372.2

### Example 107: N-(2,3-difluorobenzyl)-2-(difluoromethoxy)-5-fluoro-N-methylpyridine-3-sulfonamide

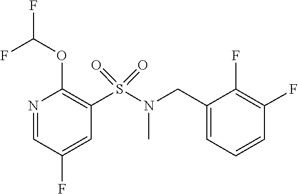 [SMILES: CN(Cc1cccc(F)c1F)S(=O)(=O)c1cc(F)cnc1OC(F)F]

Following the procedure employed for Example 106 using 2-(difluoromethoxy)-5-fluoropyridine-3-sulfonyl chloride (Intermediate 57) (50 mg, 0.191 mmol) and 1-(2,3-difluorophenyl)-N-methyl-methanamine (Intermediate 56) (36.1 mg, 0.229 mmol) gave N-(2,3-difluorobenzyl)-2-(difluoromethoxy)-5-fluoro-N-methylpyridine-3-sulfonamide (4.93 mg, 0.0127 mmol, 6.65% yield) as a colourless oil.

¹H NMR (400 MHz, MeOH-d₄): 8.36 (d, J=3.00 Hz, 1H) 8.19 (dd, J=7.25, 3.00 Hz, 1H) 7.65 (t, J=71.67 Hz, 1H) 7.10-7.29 (m, 3H) 4.58 (s, 2H) 3.31 (s, 3H); MS ES⁺: 383.2

### Example 108: N-(3-chlorobenzyl)-2-(difluoromethoxy)-5-fluoro-N-methylpyridine-3-sulfonamide

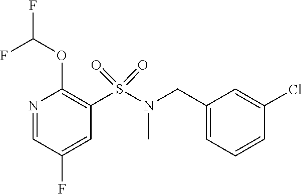 [SMILES: CN(Cc1cccc(Cl)c1)S(=O)(=O)c1cc(F)cnc1OC(F)F]

Following the procedure employed for Example 106 using 2-(difluoromethoxy)-5-fluoropyridine-3-sulfonyl chloride (Intermediate 57) (50 mg, 0.191 mmol) and 1-(3-chlorophenyl)-N-methylmethanamine (35-7 mg, 0.229 mmol) gave N-(3-chlorobenzyl)-2-(difluoromethoxy)-5-fluoro-N-methylpyridine-3-sulfonamide (8.32 mg, 0.022 mmol, 11.3% yield) as a colourless oil.

¹H NMR (400 MHz, MeOD-d₄): 8.36 (d, J=3.00 Hz, 1H) 8.22 (dd, J=7.32, 2.94 Hz, 1H) 7.65 (t, J=71.67 Hz, 1H) 7.24-7.38 (m, 4H) 4.43 (s, 2H) 2.85 (s, 3H); MS ES⁺: 381.2

### Example 109: 2-(difluoromethoxy)-N-ethyl-5-fluoro-N-(3-fluorobenzyl)pyridine-3-sulfonamide

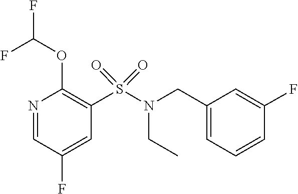 [SMILES: CCN(Cc1cccc(F)c1)S(=O)(=O)c1cc(F)cnc1OC(F)F]

Following the procedure employed for Example 106 using 2-(difluoromethoxy)-5-fluoropyridine-3-sulfonyl chloride (Intermediate 57) (50 mg, 0.191 mmol) and N-[(3-fluorophenyl)methyl]ethanamine (35.1 mg, 0.229 mmol) gave 2-(difluoromethoxy)-N-ethyl-5-fluoro-N-(3-fluorobenzyl)pyridine-3-sulfonamide (3.67 mg, 0.010 mmol, 5.03% yield) as a colourless oil.

¹H NMR (400 MHz, MeOD-d₄): 8.34 (d, J=3.00 Hz, 1H) 8.17 (dd, J=7.25, 3.00 Hz, 1H) 7.66 (t, J=71.73 Hz, 1H) 7.29-7.37 (m, 1H) 7.14 (d, J=8.00 Hz, 1H) 7.08 (dd, J=9.82, 2.06 Hz, 1H) 7.01 (td, J=8.47, 2.44 Hz, 1H) 4.57 (s, 2H) 3.39 (q, J=7.13 Hz, 2H) 0.99 (t, J=7.19 Hz, 3H); MS ES⁺: 379.2

### Example 110: 2-((2-(difluoromethoxy)-5-fluoropyridin-3-yl)sulfonyl)-1,2,3,4-tetrahydroisoquinoline

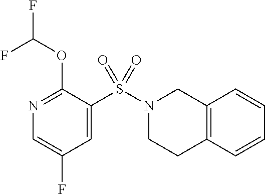 [SMILES: O=S(=O)(c1cc(F)cnc1OC(F)F)N1CCc2ccccc2C1]

Following the procedure employed for Example 106 using 2-(difluoromethoxy)-5-fluoropyridine-3-sulfonyl chloride (Intermediate 57) (50 mg, 0.191 mmol) and 1,2,3,4-tetrahydroisoquinoline (30.6 mg, 0.229 mmol) gave 2-((2-(difluoromethoxy)-5-fluoropyridin-3-yl)sulfonyl)-1,2,3,4-tetrahydroisoquinoline (18.43 mg, 0.051 mmol, 26.69% yield) as a white solid.

¹H NMR (400 MHz, DMSO-d₆): 8.59 (d, J=3.00 Hz, 1H) 8.32 (dd, J=7.32, 2.94 Hz, 1H) 7.69 (t, J=71.35 Hz, 1H) 7.09-7.21 (m, 4H) 4.45 (s, 2H) 3.62 (t, J=6.00 Hz, 2H) 2.85 (t, J=5.88 Hz, 2H); MS ES⁺: 358.9

### Example 111: 2-((2-(difluoromethoxy)-5-fluoropyridin-3-yl)sulfonyl)-7-fluoro-1,2,3,4-tetrahydroisoquinoline

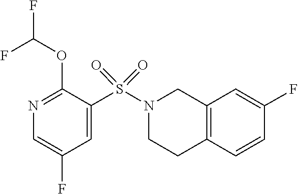 [SMILES: O=S(=O)(c1cc(F)cnc1OC(F)F)N1CCc2ccc(F)cc2C1]

Following the procedure employed for Example 106 using 2-(difluoromethoxy)-5-fluoropyridine-3-sulfonyl chloride (Intermediate 57) (60 mg, 0.229 mmol) and 7-fluoro-1,2,3,4-tetrahydroisoquinoline hydrochloride (51.6 mg, 0.275 mmol) gave 2-((2-(difluoromethoxy)-5-fluoropyridin-3-yl)sulfonyl)-7-fluoro-1,2,3,4-tetrahydroisoquinoline (12.01 mg, 0.0317 mmol, 13.83% yield) as a white solid.

¹H NMR (400 MHz, DMSO-d₆): 8.58 (d, J=3.00 Hz, 1H) 8.31 (dd, J=7.38, 2.88 Hz, 1H) 7.68 (t, J=71.35 Hz, 1H) 7.19 (dd, J=9.44, 5.69 Hz, 1H) 7.02 (td, J=4.94, 2.88 Hz, 2H) 4.47 (s, 2H) 3.61 (t, J=6.00 Hz, 2H) 2.80 (t, J=5.82 Hz, 2H); MS ES⁺: 376.9

### Example 112: 2-(difluoromethoxy)-5-fluoro-N-(3-fluorobenzyl)-N-methylpyridine-3-sulfonamide

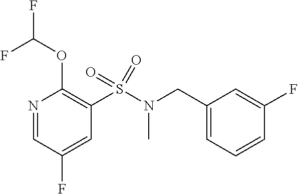 [SMILES: CN(Cc1cccc(F)c1)S(=O)(=O)c1cc(F)cnc1OC(F)F]

Following the procedure employed for Example 106 using 2-(difluoromethoxy)-5-fluoropyridine-3-sulfonyl chloride (Intermediate 57) (50 mg, crude) and 1-(3-fluorophenyl)-N-methyl-methanamine (Intermediate 55) (26.6 mg, 0.191 mmol) gave 2-(difluoromethoxy)-5-fluoro-N-(3-fluorobenzyl)-N-methylpyridine-3-sulfonamide (3.16 mg, 0.008 mmol, 4.39% yield) as a white solid.

¹H NMR (400 MHz, DMSO-d₆): 8.61 (d, J=2.5 Hz, 1H), 8.31 (dd, J=2.5, 7.1 Hz, 1H), 7.78 (t, J=7.14 Hz, 1H), 7.43 (q, J=7.5 Hz, 1H), 7.21-7.07 (m, 3H), 4.40 (s, 2H), 2.80 (s, 3H); MS ES⁺: 365.3

### Example 113: 5-chloro-2-(difluoromethoxy)-N-(3-fluorobenzyl)-N-methylpyridine-3-sulfonamide

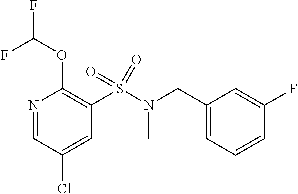 [SMILES: CN(Cc1cccc(F)c1)S(=O)(=O)c1cc(Cl)cnc1OC(F)F]

Following the procedure employed for Example 106 using 5-chloro-2-(difluoromethoxy)pyridine-3-sulfonyl chloride (Intermediate 58) (45 mg, crude) and 1-(3-fluorophenyl)-N-methyl-methanamine (Intermediate 55) gave 5-chloro-2-(difluoromethoxy)-N-[(3-fluorophenyl)methyl]-N-methyl-pyridine-3-sulfonamide (6.54 mg, 0.017 mmol, 10.48% yield) as a pink solid.

¹H NMR (400 MHz, DMSO-d₆): 8.64 (d, J=2.4 Hz, 1H), 8.37 (d, J=2.4 Hz, 1H), 8.06-7.57 (m, 1H), 7.48-7.34 (m, 1H), 7.23-7.03 (m, 3H), 4.40 (s, 2H), 2.81 (s, 3H); MS ES⁺: 381.2

### Example 114: 4-((2-(difluoromethoxy)-5-fluoropyridin-3-yl)sulfonyl)-7-fluoro-2,3,4,5-tetrahydrobenzo[f][1,4]oxazepine

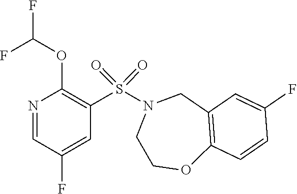 [SMILES: O=S(=O)(c1cc(F)cnc1OC(F)F)N1CCOc2ccc(F)cc2C1]

Following the procedure employed for Example 106 using 2-(difluoromethoxy)-5-fluoropyridine-3-sulfonyl chloride (Intermediate 57) (85 mg, crude) and 7-Fluoro-2,3,4,5-tetrahydrobenzo[f][1,4]oxazepine (Intermediate 44) (54.3 mg, 0.325 mmol) gave 4-[[2-(difluoromethoxy)-5-fluoro-3-pyridyl]sulfonyl]-7-fluoro-3,5-dihydro-2H-1,4-benzoxazepine (35-53 mg, 0.0905 mmol, 27.84% yield) as a white solid.

¹H NMR (400 MHz, DMSO-d₆): 8.53 (d, J=2.5 Hz, 1H), 8.21 (dd, J=2.6, 7.3 Hz, 1H), 7.71 (t, J=71.4 Hz, 1H), 7.11 (dd, J=2.8, 8.8 Hz, 1H), 7.06-6.96 (m, 1H), 6.96-6.87 (m, 1H), 4.52 (s, 2H), 4.17-4.08 (m, 2H), 3.82 (d, J=4.3 Hz, 2H); MS ES⁺: 393.3

### Example 115: 4-((5-chloro-2-(difluoromethoxy)pyridin-3-yl)sulfonyl)-7-fluoro-2,3,4,5-tetrahydrobenzo[f][1,4]oxazepine

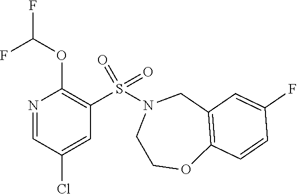 [SMILES: O=S(=O)(c1cc(Cl)cnc1OC(F)F)N1CCOc2ccc(F)cc2C1]

Following the procedure employed for Example 106 using 5-chloro-2-(difluoromethoxy)pyridine-3-sulfonyl chloride (Intermediate 58) (0 mg, crude) and 7-Fluoro-2,3,4,5-tetrahydrobenzo[f][1,4]oxazepine (Intermediate 44) (36.1 mg, 0.216 mmol) gave 4-[[5-chloro-2-(difluoromethoxy)-3-pyridyl]sulfonyl]-7-fluoro-3,5-dihydro-2H-1,4-benzoxazepine (2.68 mg, 0.007 mmol, 3.62% yield, 99.2% purity) as a white solid.

¹H NMR (400 MHz, MeOD-d₄): 8.36 (d, J=2.0 Hz, 1H), 8.29-8.20 (m, 1H), 7.83-7.43 (m, 1H), 7.01-6.84 (m, 3H), 4.56 (s, 2H), 4.17-4.12 (m, 2H), 3.88 (d, J=4.4 Hz, 2H); MS ES⁺: 409.2

### Example 116: 2-((5-chloro-2-(difluoromethoxy)pyridin-3-yl)sulfonyl)-6,8-difluoro-1,2,3,4-tetrahydroisoquinoline

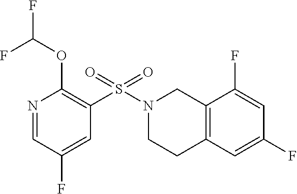 [SMILES: O=S(=O)(c1cc(F)cnc1OC(F)F)N1CCc2cc(F)cc(F)c2C1]

Following the procedure employed for Example 106 using 2-(difluoromethoxy)-5-fluoropyridine-3-sulfonyl chloride (Intermediate 57) (54.1 mg, crude) and 6,8-difluoro-1,2,3,4-tetrahydroisoquinoline (Intermediate 59) (35 mg, crude) gave 2-((5-chloro-2-(difluoromethoxy)pyridin-3-yl)sulfonyl)-6,8-difluoro-1,2,3,4-tetrahydroisoquinoline (2.45 mg, 0.006 mmol, 2.96% yield) as a white solid.

¹H NMR (400 MHz, MeOD-d₄): 8.35 (d, J=2.9 Hz, 1H), 8.27 (dd, J=2.9, 7.3 Hz, 1H), 7.56 (t, J=71.6 Hz, 1H), 6.86-6.73 (m, 2H), 4.54 (s, 2H), 3.68 (t, J=5.9 Hz, 2H), 2.87 (t, J=5.8 Hz, 2H); MS ES⁺: 394.9

### Example 117: 5-chloro-N-(2,6-difluorobenzyl)-2-methoxy-N-methylnicotinamide

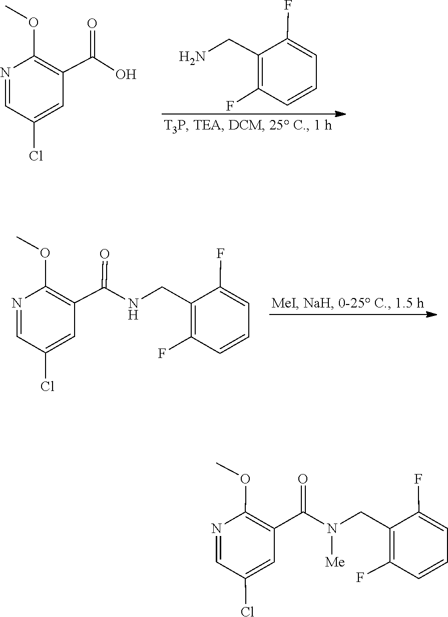 [SMILES: COc1ncc(Cl)cc1C(=O)O ; NCc1c(F)cccc1F ; COc1ncc(Cl)cc1C(=O)NCc1c(F)cccc1F ; COc1ncc(Cl)cc1C(=O)N(C)Cc1c(F)cccc1F]

Step 1: To a mixture of 5-chloro-2-methoxy-pyridine-3-carboxylic acid (500 mg, 2.67 mmol), (2,6-difluorophenyl)methanamine (381.5 mg, 2.67 mmol) and triethylamine (809.2 mg, 8.00 mmol) in dichloromethane (5 mL) was added T₃P (2.54 g, 4.00 mmol, 50% purity in ethyl acetate) in one portion at 25° C. The mixture was stirred at 25° C. for 1 hour. The reaction mixture was diluted with H₂O (30 mL) and extracted with ethyl acetate (3×50 mL). The combined organic layers were dried over Na₂SO₄, filtered and concentrated under reduced pressure to give a residue which was purified by flash silica gel chromatography (SiO₂, Petroleum ether:Ethyl acetate=1:0 to 2:1) to give 5-chloro-N-(2,6-difluorobenzyl)-2-methoxynicotinamide (750 mg, 2.24 mmol, 84.22% yield) as a white solid. ¹H NMR (400 MHz, DMSO-d₆): 8.78-8.68 (m, 1H), 8.38-8.29 (m, 1H), 8.07-7.96 (m, 1H), 7.45-7.34 (m, 1H), 7.14-7.05 (m, 2H), 4.58-4.49 (m, 2H), 3.93 (s, 3H); MS ES⁺: 313.0

Step 2: To a solution of 5-chloro-N-(2,6-difluorobenzyl)-2-methoxynicotinamide (100 mg, 0.320 mmol) in THF (2 mL) was added NaH (19.2 mg, 0.480 mmol, 60% purity) at 0° C. under N₂. The mixture was stirred at 0° C. for 0.5 hour. Mel (227 mg, 1.60 mmol) was added to the mixture and the reaction was stirred at 25° C. for 1 hour. The reaction mixture was quenched by saturated NH₄Cl (aq.) (30 mL) at 0° C., and then diluted with H₂O (10 mL) and extracted with ethyl acetate (3×10 mL). The combined organic layers were washed with brine (20 mL), dried over Na₂SO₄, filtered and concentrated under reduced pressure to dryness which was purified by prep. HPLC (column: Phenomenex luna 30*30 mm*10 μm+YMC AQ 100*30*10 μm; mobile phase A: water (0.05% NH₃H₂O), mobile phase B: acetonitrile; Flow rate: 25 mL/min, gradient condition from 20% B to 70%). The pure fractions were collected and the volatiles were removed under vacuum. The residue was partitioned between acetonitrile (2 mL) and water (10 mL). The solution was lyophilized to dryness to give the product 5-chloro-N-(2,6-difluorobenzyl)-2-methoxy-N-methylnicotinamide (10.02 mg, 0.0307 mmol, 9.59% yield, 100% purity) as a white solid.

¹H NMR (400 MHz, DMSO-d₆): 8.27-8.21 (m, 1H), 7.71-7.63 (m, 1H), 7.52-7.35 (m, 1H), 7.15-7.00 (m, 2H), 4.81-4.40 (m, 2H), 3.87 (s, 3H), 2.98-2.68 (m, 3H); MS ES⁺: 327.3

### Example 118: 5-chloro-N-(2,6-dichlorobenzyl)-2-methoxy-N-methylnicotinamide

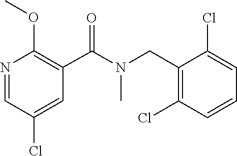 [SMILES: COc1ncc(Cl)cc1C(=O)N(C)Cc1c(Cl)cccc1Cl]

Following the procedure employed for Example 117 using 5-chloro-2-methoxy-pyridine-3-carboxylic acid (500 mg, 2.67 mmol) and (2,6-dichlorophenyl)methanamine (470 mg, 2.67 mmol) and triethylamine (809 mg, 8.00 mmol) gave 5-chloro-N-(2,6-dichlorobenzyl)-2-methoxy-N-methylnicotinamide (123.25 mg, 0.343 mmol, 59.22% yield) as a white solid.

¹H NMR (400 MHz, DMSO-d₆): 8.35-8.17 (m, 1H), 7.80-7.66 (m, 1H), 7.55-7.42 (m, 2H), 7.42-7.35 (m, 1H), 5.01-4.59 (m, 2H), 3.91 (s, 3H), 2.85-2.55 (m, 3H); MS ES⁺: 359.

### Example 119: 5-chloro-2-(difluoromethoxy)-N-(1,1-dioxido-2,3-dihydrobenzo[b]thiophen-3-yl)-N-methylnicotinamide

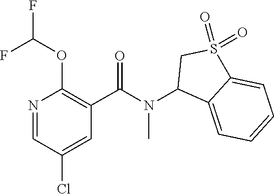 [SMILES: CN(C(=O)c1cc(Cl)cnc1OC(F)F)C1CS(=O)(=O)c2ccccc21]

Following the procedure employed for Example 74 using 1,1-dioxo-2,3-dihydrobenzothiophen-3-amine hydrochloride (Intermediate 60) (136 mg, 0.744 mmol) and 5-chloro-2-(difluoromethoxy)pyridine-3-carbonyl chloride (Intermediate 47) (180 mg, crude) gave 5-chloro-2-(difluoromethoxy)-N-(1,1-dioxo-2,3-dihydrobenzothiophen-3-yl)-N-methyl-pyridine-3-carboxamide (30 mg, 0.075 mmol, 82.73% yield) as a white solid.

¹H NMR (400 MHz, DMSO-d₆): 8.54-8.42 (m, 1H), 8.35-8.16 (m, 1H), 7.97-7.43 (m, 5H), 6.58-6.39 (m, 0.5H), 5.82-5.52 (m, 0.5H), 4.11 (dd, J=8.8, 13.8 Hz, 2H), 2.78-2.60 (m, 3H); MS ES⁺: 402.8

### Example 120: 5-fluoro-2-((7-fluoro-2,3-dihydrobenzo[f][1,4]oxazepin-4(5H)-yl)sulfonyl)benzonitrile

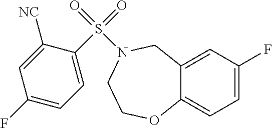 [SMILES: N#Cc1cc(F)ccc1S(=O)(=O)N1CCOc2ccc(F)cc2C1]

By following the procedure employed in Example 51 using 7-fluoro-2,3,4,5-tetrahydrobenzo[f][1,4]oxazepine (Intermediate 44) (0.04 g, 0.4 mmol) and 2-cyano-4-fluorobenzenesulfonyl chloride (0.04 g, 0.2 mmol), gave 5-fluoro-2-((7-fluoro-2,3-dihydrobenzo[f][1,4]oxazepin-4(5H)-yl)sulfonyl)benzonitrile (45 mg, 53%) as a white solid.

¹H NMR (400 MHz, DMSO-d₆): δ 8.13 (dd, J=2.40 Hz, 8.60 Hz, 1H), 8.06-8.03 (m, 1H), 7.70 (dt, J=2.80 Hz, 6.67 Hz, 1H), 7.09 (dd, J=2.80 Hz, 8.4 Hz, 1H), 6.99 (dt, J=3.20 Hz, 12.53 Hz, 1H), 6.96-6.82 (m, 1H), 4.59 (s, 2H), 4.14 (t, J=2.00 Hz, 2H), 3.83 (t, J=4.40 Hz, 2H); MS m/z: 351.26 (M+H).

### 3. Inflammasome Assay in Primary Mouse Postnatal Microglia Using LPS/0 mM Extracellular K⁺

Newly shaken mouse microglia cells were added to a 96-well plate and left to adhere overnight. After this time, 100 ng/mL LPS was added to each well and incubated at 37° C. for 3.5 hours, at which point compound addition was undertaken and plates incubated at 37° C. for an additional 30 minutes. After this time, the medium in each well was removed and replaced with K⁺ free buffer and the plates then incubated for an additional 2 hours at 37° C. Measurement of IL-1β levels in the sample wells was undertaken using MesoScale Discovery™ MESO QuickPlex SQ 120 and IL-1β antibodies from mouse IL-1β DuoSet ELISA kit (R&D System, DY401). The results are summarised in table 2.

TABLE 2

| Example | Mouse primary microglia IL-1β, IC₅₀ (μM) |
|---|---|
| 16 | + |
| 24 | +++ |
| 39 | ++ |

IC₅₀ (≤0.60 μM = ‘+’; ≤0.30 μM = ‘++’; ≤0.15 μM = ‘+++’)

### 4. Biological Efficacy of Compounds of the Invention

KCNK13 antagonist activity was determined by measuring changes in intracellular Thallium (Tl⁺) concentrations using a Tl⁺ sensitive fluorescent dye. The changes in fluorescent signal were monitored by Fluorescent Imaging Plate Reader (FLIPR™) technology available from Molecular Devices, LLC, US. KCNK13 mediated increases in intracellular Tl⁺ concentration were readily detected by addition of a thallium sulfate Stimulus. 24 hours prior to the assay, human embryonic kidney 293 cells (HEK 293 cells) stably expressing human KCNK13 were seeded in cell culture medium in PDL coated black, clear-bottom 384-well plates (commercially available from Corning Inc., 356663) and grown overnight at 37° C., 500 CO₂. On the day of the assay, cell culture media was removed and cells were loaded with potassium dye (commercially sold by Molecular Devices, LLC, US, R8222) for 1 hour at room temperature in the dark. Test compounds (at 10 point half log concentration response curves from 100 M) were added to cells for 15 minutes prior to the addition of thallium sulfate to all wells. The IC₅₀ values were determined from ten point concentration response curves. Curves were generated using the average of two wells for each data point. The results are summarised in table 3.

TABLE 3

| Example | hKCNK13 IC₅₀ (μM) |
|---|---|
| 1 | +++++ |
| 2 | ++ |
| 3 | +++ |
| 4 | ++ |
| 5 | +++ |
| 6 | +++ |
| 7 | ++++ |
| 8 | ++++ |
| 9 | ++ |
| 10 | +++ |
| 11 | ++ |
| 12 | ++ |
| 13 | +++ |
| 14 | +++ |
| 15 | +++ |
| 16 | +++ |
| 17 | +++ |
| 18 | +++ |
| 19 | +++ |
| 20 | +++ |
| 21 | ++++ |
| 22 | +++ |
| 23 | +++ |
| 24 | ++++++ |
| 25 | +++ |
| 26 | +++ |
| 27 | +++ |
| 28 | +++ |
| 29 | +++ |
| 30 | +++ |
| 31 | ++ |
| 32 | ++ |
| 33 | +++ |
| 34 | +++ |
| 35 | +++ |
| 36 | +++ |
| 37 | ++++ |
| 38 | +++ |
| 39 | ++++ |
| 40 | ++++ |
| 41 | ++++ |
| 42 | ++ |
| 43 | ++ |
| 44 | ++++ |
| 45 | + |
| 46 | ++ |
| 47 | ++ |
| 48 | +++ |
| 49 | ++ |
| 50 | ++++ |
| 51 | ++++ |
| 52 | ++++ |
| 53 | +++ |
| 54 | +++ |
| 55 | +++ |
| 56 | +++ |
| 57 | ++ |
| 58 | ++++ |
| 59 | +++ |
| 60 | + |
| 61 | ++ |
| 62 | ++ |
| 63 | ++ |
| 64 | + |
| 65 | ++ |
| 66 | + |
| 67 | +++ |
| 68 | + |
| 69 | ++++ |
| 70 | ++++ |
| 71 | +++++ |
| 72 | ++++ |
| 73 | +++ |
| 74 | +++ |
| 75 | +++ |
| 76 | ++++ |
| 77 | +++ |
| 78 | ++++++ |
| 79 | ++++ |
| 80 | +++++ |
| 81 | ++++ |
| 82 | +++ |
| 83 | +++++ |
| 84 | +++++ |
| 85 | ++++ |
| 86 | +++++ |
| 87 | ++++ |
| 88 | +++++ |
| 89 | ++++ |
| 90 | +++ |
| 91 | +++ |
| 92 | +++ |
| 93 | +++++ |
| 94 | +++++ |
| 95 | +++++ |
| 96 | +++ |
| 97 | ++ |
| 98 | ++++ |
| 99 | ++ |
| 100 | +++++ |
| 101 | +++++ |
| 102 | +++++ |
| 103 | ++++ |
| 104 | ++ |
| 105 | ++ |
| 106 | +++ |
| 107 | +++++ |
| 108 | ++++ |
| 109 | ++ |
| 110 | +++++ |
| 111 | ++++ |
| 112 | +++++ |
| 113 | +++ |
| 114 | ++++ |
| 115 | +++ |
| 116 | +++ |
| 117 | ++++ |
| 118 | ++++ |
| 119 | +++ |
| 120 | ++++ |

IC₅₀ (≤5 μM = ‘+’; ≤1.0 μM = ‘++’; ≤0.5 μM = ‘+++’; ≤0.2 μM = ‘++++’; ≤0.1 μM = ‘+++++’; ≤0.05 μM = ‘++++++’)

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I): 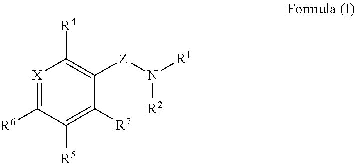
or a pharmaceutically acceptable salt, N-oxide, solvate or prodrug thereof, wherein:
X is N or CH;
Z is —C(O)— or —SO₂—;
R¹ is -L-R³;
R² is hydrogen or C₁-C₃ alkyl;
L is a bond, —CH₂—, —CHMe-, or —C(C₂H₄)—;
R³ is a 5- to 11-membered cyclic group, wherein the cyclic group is optionally substituted with one or more substituents independently selected from halo, cyano, C₁-C₃ alkyl, C₁-C₃ haloalkyl, —O—(C₁-C₃ alkyl), —O—(C₁-C₃ haloalkyl), oxo (═O), or —C(O)O(C₁-C₃ alkyl);
or R¹ and R² and the nitrogen atom to which they are attached together form a 5- to 11-membered heterocyclic group, wherein the heterocyclic group is optionally substituted with one or more substituents independently selected from halo, cyano, C₁-C₃ alkyl, C₁-C₃ haloalkyl, —O—(C₁-C₃ alkyl), —O—(C₁-C₃ haloalkyl), or halophenyl;
R⁴ is cyano, —O—(C₁-C₃ alkyl), —O—(C₁-C₃ haloalkyl), or —N—(C₁-C₃ alkyl)₂;
or R⁴ and X together with the carbon atom to which they are attached form a 5-membered heteroaryl group;
R⁵ is hydrogen, halo, C₁-C₃ alkyl, C₁-C₃ haloalkyl, or C₃-C₅ cycloalkyl;
R⁶ is hydrogen, methyl or halo; and
R⁷ is hydrogen, methyl or halo.

2. The compound, salt, N-oxide, solvate or prodrug as claimed in claim 1, wherein the compound is of formula (Ia): 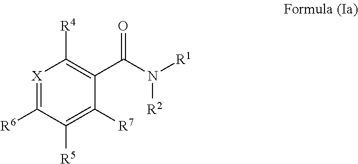
or a pharmaceutically acceptable salt, N-oxide, solvate or prodrug thereof, wherein X, R¹, R², R⁴, R⁵, R⁶ and R⁷ are as defined in claim 1.

3. The compound, salt, N-oxide, solvate or prodrug as claimed in claim 1 or 2, wherein:
X is N;
Z is —C(O)—;
R¹ is -L-R³;
R² is C₁-C₃ alkyl;
L is a bond, —CH₂—, —CHMe-, or —C(C₂H₄)—;
R³ is a 6- to 11-membered cyclic group, wherein the cyclic group is substituted with one or more substituents independently selected from halo, cyano, C₁-C₃ alkyl, C₁-C₃ haloalkyl, —O—(C₁-C₃ alkyl), —O—(C₁-C₃ haloalkyl), oxo (═O), or —C(O)O(C₁-C₃ alkyl);
R⁴ is cyano, —O—(C₁-C₃ alkyl), —O—(C₁-C₃ haloalkyl), or —N—(C₁-C₃ alkyl)₂;
R⁵ is hydrogen, halo, C₁-C₃ alkyl, C₁-C₃ haloalkyl, or C₃-C₅ cycloalkyl;
R⁶ is hydrogen, methyl or halo;
R⁷ is hydrogen, methyl or halo;
provided that at least one of R⁵, R⁶ and R⁷ is not hydrogen; and
provided that the compound is not:
i) 5-chloro-N-(4-fluorophenyl)-2-methoxy-N-methylnicotinamide;
ii) 5-chloro-2-methoxy-N-methyl-N-(1-methylpiperidin-4-yl)nicotinamide;
iii) 5-chloro-N-ethyl-2-methoxy-N-(m-tolyl)nicotinamide;
iv) 5-chloro-N-ethyl-2-methoxy-N-(o-tolyl)nicotinamide;
v) 5-bromo-N-(4-fluorophenyl)-2-methoxy-N-methylnicotinamide;
vi) 5-bromo-2-methoxy-N-methyl-N-(1-methylpiperidin-4-yl)nicotinamide;
vii) 5-bromo-N-ethyl-2-methoxy-N-(m-tolyl)nicotinamide;
viii) 5-bromo-N-ethyl-2-methoxy-N-(o-tolyl)nicotinamide;
ix) 5-bromo-N-(2,4-difluorophenyl)-2-methoxy-N-methylnicotinamide;
x) 5-bromo-N-(2,4-difluorophenyl)-2-(dimethylamino)-N-methylnicotinamide;
xi) 5-bromo-2-(dimethylamino)-N-(3-isopropylphenyl)-N-methylnicotinamide;
xii) 5-bromo-2-(dimethylamino)-N-((2-ethylbenzofuran-3-yl)methyl)-N-methylnicotinamide;
xiii) N-(4-fluorophenyl)-2-methoxy-N,4,5,6-tetramethylnicotinamide;
xiv) 2-methoxy-N,4,5,6-tetramethyl-N-(1-methylpiperidin-4-yl)nicotinamide;
xv) N-ethyl-2-methoxy-4,5,6-trimethyl-N-(m-tolyl)nicotinamide;
xvi) N-ethyl-2-methoxy-4,5,6-trimethyl-N-(o-tolyl)nicotinamide;
xvii) 2-methoxy-N,6-dimethyl-N-(1-methyl-4,5,6,7-tetrahydro-1H-indazol-4-yl)nicotinamide;
xviii) N-(5-chloro-2-cyanophenyl)-2-methoxy-N,6-dimethylnicotinamide;
xix) 5-chloro-N-(4-fluorophenyl)-2-methoxy-N,4,6-trimethylnicotinamide;
xx) N-(4-cyanobenzyl)-2-methoxy-6-methyl-N-propylnicotinamide;
xxi) 5-chloro-2-methoxy-N,4,6-trimethyl-N-(1-methylpiperidin-4-yl)nicotinamide;
xxii) N-(2-(dimethylamino)pyridin-3-yl)-N,6-dimethyl-2-propoxynicotinamide;
xxiii) 2-methoxy-N,6-dimethyl-N-(2-methyl-4,5,6,7-tetrahydrobenzo[d]thiazol-7-yl)nicotinamide;
xxiv) 5-chloro-N-ethyl-2-methoxy-4,6-dimethyl-N-(m-tolyl)nicotinamide;
xxv) 5-chloro-N-ethyl-2-methoxy-4,6-dimethyl-N-(o-tolyl)nicotinamide;
xxvi) 2-ethoxy-N-((5-methoxy-6-methyl-4-oxo-1,4-dihydropyridin-3-yl)methyl)-N,4-dimethylnicotinamide;
xxvii) 2-ethoxy-N-((5-methoxy-6-methyl-4-oxo-1,4-dihydropyridin-3-yl)methyl)-N,6-dimethylnicotinamide;
xxviii) N-(2-ethyl-4,5,6,7-tetrahydrobenzo[d]thiazol-7-yl)-2-methoxy-N,6-dimethylnicotinamide;
xxix) 2-ethoxy-N,6-dimethyl-N-(2-methyl-4,5,6,7-tetrahydrobenzo[d]thiazol-7-yl)nicotinamide;
xxx) 2-ethoxy-N-(2-ethyl-4,5,6,7-tetrahydrobenzo[d]thiazol-7-yl)-N,6-dimethylnicotinamide;
xxxi) 5-bromo-N-(4-fluorophenyl)-2-methoxy-N,4,6-trimethylnicotinamide;
xxxii) 5-bromo-2-methoxy-N,4,6-trimethyl-N-(1-methylpiperidin-4-yl)nicotinamide;
xxxiii) 5-bromo-N-ethyl-2-methoxy-4,6-dimethyl-N-(m-tolyl)nicotinamide;
xxxiv) 5-bromo-N-ethyl-2-methoxy-4,6-dimethyl-N-(o-tolyl)nicotinamide; or
xxxv) N-(6-bromochroman-4-yl)-2-methoxy-N,6-dimethylnicotinamide;
or a stereoisomer of any of the above compounds.

4. The compound, salt, N-oxide, solvate or prodrug as claimed in claim 1, wherein the compound is of formula (Ib): 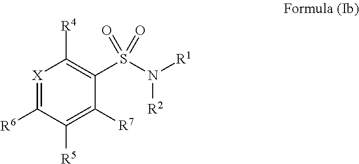
or a pharmaceutically acceptable salt, N-oxide, solvate or prodrug thereof, wherein X, R¹, R², R⁴, R⁵, R⁶ and R⁷ are as defined in claim 1.

5. The compound, salt, N-oxide, solvate or prodrug as claimed in any one of claims 1 to 4, wherein X is N.

6. The compound, salt, N-oxide, solvate or prodrug as claimed in any one of claim 1, 2 or 4, wherein X is CH.

7. The compound, salt, N-oxide, solvate or prodrug as claimed in any one of claim 1, 2, 4, 5 or 6, wherein:
R¹ is -L-R³;
R² is methyl or ethyl;
L is a bond, —CH₂—, —CHMe-, or —C(C₂H₄)—; and
R³ is a 6- to 10-membered cyclic group selected from phenyl, indanyl, dihydrobenzo[b]thiophenyl, chromanyl, and 2,3-dihydrobenzofuranyl, wherein the cyclic group is optionally substituted with one, two or three substituents independently selected from fluoro, chloro, cyano, methyl, fluoromethyl, methoxy, fluoromethoxy, oxo (═O), or —CO₂Me.

8. The compound, salt, N-oxide, solvate or prodrug as claimed in any one of claim 1, 2, 4, 5 or 6, wherein R¹ and R² and the nitrogen atom to which they are attached together form a heterocyclic group selected from piperidin-1-yl, tetrahydroisoquinolin-2-yl, or 2,3,4,5-tetrahydrobenzo[f][1,4]oxazepin-4-yl, wherein the heterocyclic group is optionally substituted with one or two substituents independently selected from fluoro or fluorophenyl.

9. The compound, salt, N-oxide, solvate or prodrug as claimed in any one of the preceding claims, wherein:
(i) R⁴ is cyano, methoxy, ethoxy, fluoromethoxy, or fluoroethoxy; and/or
(ii) R⁵ is hydrogen, fluoro, chloro, methyl, fluoromethyl, cyclopropyl, or cyclobutyl; and/or
(iii) R⁶ is hydrogen, methyl, or fluoro; and/or
(iv) R⁷ is hydrogen or methyl.

10. The compound, salt, N-oxide, solvate or prodrug as claimed in any one of the preceding claims, wherein the compound is selected from:
N-benzyl-5-chloro-2-methoxy-N-methylnicotinamide;
5-chloro-N-(6-cyano-2,3-dihydro-1H-inden-1-yl)-2-methoxy-N-methylnicotinamide;
5-chloro-N-(7-cyano-2,3-dihydro-1H-inden-1-yl)-2-methoxy-N-methylnicotinamide;
(5-fluoro-2-methoxypyridin-3-yl)(2-(3-fluorophenyl)piperidin-1-yl)methanone;
5-fluoro-N-(1-(2-fluorophenyl)ethyl)-2-methoxy-N-methylnicotinamide;
5-fluoro-N-(1-(3-fluorophenyl)ethyl)-2-methoxy-N-methylnicotinamide;
5-fluoro-N-(6-fluoro-2,3-dihydro-1H-inden-1-yl)-2-methoxy-N-methylnicotinamide;
N-(2,4-difluorobenzyl)-2-methoxy-N,6-dimethylnicotinamide;
5-chloro-N-(2,3-dihydro-1H-inden-1-yl)-2-ethoxy-N-methylnicotinamide;
N-(chroman-4-yl)-5-fluoro-2-methoxy-N-methylnicotinamide;
N-(2, 3-dihydro-1H-inden-1-yl)-5-fluoro-2-methoxy-N-methylnicotinamide;
5-chloro-N-(chroman-4-yl)-2-methoxy-N-methylnicotinamide;
5-chloro-N-(6-fluoro-2,3-dihydro-1H-inden-1-yl)-2-methoxy-N-methylnicotinamide;
5-chloro-N-(2,3-dihydro-1H-inden-1-yl)-2-methoxy-N-methylnicotinamide;
5-chloro-N-(3-fluorobenzyl)-2-methoxy-N-methylnicotinamide;
5-chloro-N-(2,3-dihydrobenzofuran-3-yl)-2-methoxy-N-methylnicotinamide;
5-chloro-N-(8-fluorochroman-4-yl)-2-methoxy-N-methylnicotinamide;
5-fluoro-N-(8-fluorochroman-4-yl)-2-methoxy-N-methylnicotinamide;
N-(3-fluorobenzyl)-2-methoxy-N-methylnicotinamide;
N-(2-fluorobenzyl)-2-methoxy-N-methylnicotinamide;
5-fluoro-N-(1-(3-fluorophenyl)cyclopropyl)-2-methoxy-N-methylnicotinamide;
5-fluoro-N-(5-fluoro-2,3-dihydrobenzofuran-3-yl)-2-methoxy-N-methylnicotinamide;
N-(2-cyanobenzyl)-5-fluoro-2-methoxy-N-methylnicotinamide;
5-fluoro-N-(2-fluorobenzyl)-2-methoxy-N-methylnicotinamide;
N-(2,3-difluorobenzyl)-5-fluoro-2-methoxy-N-methylnicotinamide;
N-(2,4-difluorobenzyl)-5-fluoro-2-methoxy-N-methylnicotinamide;
5-chloro-N-(2,3-difluorobenzyl)-2-methoxy-N-methylnicotinamide;
5-chloro-N-(3,5-difluorobenzyl)-2-methoxy-N-methylnicotinamide;
5-fluoro-N-(6-fluoro-2,3-dihydrobenzofuran-3-yl)-2-methoxy-N-methylnicotinamide;
5-chloro-N-(7-fluorochroman-4-yl)-2-methoxy-N-methylnicotinamide;
5-chloro-N-(3-chloro-5-fluorobenzyl)-2-methoxy-N-methylnicotinamide;
5-chloro-N-(6-fluoro-2,3-dihydrobenzofuran-3-yl)-2-methoxy-N-methylnicotinamide;
N-benzyl-5-chloro-N-ethyl-2-methoxynicotinamide;
5-chloro-N-ethyl-N-(2-fluorobenzyl)-2-methoxynicotinamide;
5-chloro-N-ethyl-N-(3-fluorobenzyl)-2-methoxynicotinamide;
5-fluoro-N-(3-fluorobenzyl)-2-methoxy-N-methylnicotinamide;
2-((2-methoxy-5-methylpyridin-3-yl)sulfonyl)-1,2,3,4-tetrahydroisoquinoline;
2-((5-chloro-2-methoxypyridin-3-yl)sulfonyl)-1,2,3,4-tetrahydroisoquinoline;
7-fluoro-4-((2-methoxy-5-methylpyridin-3-yl)sulfonyl)-2,3,4,5-tetrahydrobenzo[f][1,4]oxazepine;
4-((5-chloro-2-methoxypyridin-3-yl)sulfonyl)-7-fluoro-2,3,4,5-tetrahydrobenzo[f][1,4]oxazepine;
7-fluoro-4-((2-methoxypyridin-3-yl)sulfonyl)-2,3,4,5-tetrahydrobenzo[f][1,4]oxazepine;
methyl 4-fluoro-2-((5-fluoro-2-methoxy-N-methylnicotinamido)methyl)benzoate;
5-chloro-N-(4-fluoro-2-(trifluoromethyl)benzyl)-2-methoxy-N-methylnicotinamide;
5-chloro-N-(2-cyano-4-fluorobenzyl)-2-methoxy-N-methylnicotinamide;
5-chloro-N-(1-(2-cyano-5-fluorophenyl)ethyl)-2-methoxy-N-methylnicotinamide;
N-(2-cyano-4,6-difluorobenzyl)-5-fluoro-2-methoxy-N-methylnicotinamide;
N-(1-(2-cyano-5-fluorophenyl)ethyl)-5-fluoro-2-methoxy-N-methylnicotinamide;
N-(1-(2-cyano-4-fluorophenyl)ethyl)-5-fluoro-2-methoxy-N-methylnicotinamide;
5-chloro-N-(1-(2-cyano-4-fluorophenyl)ethyl)-2-methoxy-N-methylnicotinamide;
N-(1-(2,4-difluorophenyl)ethyl)-5-fluoro-2-methoxy-N-methylnicotinamide;
2-(difluoromethoxy)-5-fluoro-N-(1-(2-fluorophenyl)ethyl)-N-methylnicotinamide;
N-(2,4-difluorobenzyl)-2-methoxy-N,5-dimethylnicotinamide;
N-(2,4-difluorobenzyl)-2-methoxy-N,4-dimethylnicotinamide;
2-(difluoromethoxy)-5-fluoro-N-(6-fluoro-2,3-dihydrobenzofuran-3-yl)-N-methylnicotinamide;
2-(difluoromethoxy)-5-fluoro-N-(7-fluoro-2,3-dihydrobenzofuran-3-yl)-N-methylnicotinamide;
5-chloro-2-(difluoromethoxy)-N-(3-fluoro-5-methylbenzyl)-N-methylnicotinamide;
5-chloro-2-(difluoromethoxy)-N-(6-fluoro-2,3-dihydrobenzofuran-3-yl)-N-methylnicotinamide;
5-chloro-N-(3,5-difluorobenzyl)-2-(difluoromethoxy)-N-methylnicotinamide;
5-chloro-N-(3-chloro-5-fluorobenzyl)-2-(difluoromethoxy)-N-methylnicotinamide;
5-chloro-2-(difluoromethoxy)-N-methyl-N-(3-methylbenzyl)nicotinamide;
5-chloro-2-(difluoromethoxy)-N-(3-methoxybenzyl)-N-methylnicotinamide;
5-chloro-2-(difluoromethoxy)-N-methyl-N-(3-(trifluoromethyl)benzyl) nicotinamide;
2-(difluoromethoxy)-5-fluoro-N-(1-(3-fluorophenyl)ethyl)-N-methylnicotinamide;
5-chloro-N-(3-chlorobenzyl)-2-(difluoromethoxy)-N-methylnicotinamide;
2-(difluoromethoxy)-N-(2,3-dihydrobenzofuran-3-yl)-5-fluoro-N-methylnicotinamide;
5-chloro-2-(difluoromethoxy)-N-(1-(3-fluorophenyl)ethyl)-N-methylnicotinamide;
2-(difluoromethoxy)-5-fluoro-N-(1-(3-fluorophenyl)ethyl)-N-methylnicotinamide;
5-chloro-N-(3-cyanobenzyl)-2-(difluoromethoxy)-N-methylnicotinamide;
5-chloro-2-(difluoromethoxy)-N-(2,3-dihydrobenzofuran-3-yl)-N-methylnicotinamide;
5-chloro-2-(difluoromethoxy)-N-(7-fluoro-2,3-dihydrobenzofuran-3-yl)-N-methylnicotinamide;
N-(2,3-difluorobenzyl)-2-(difluoromethoxy)-5-fluoro-N-methylnicotinamide;
N-(2,4-difluorobenzyl)-2-(difluoromethoxy)-5-fluoro-N-methylnicotinamide;
2-(difluoromethoxy)-5-fluoro-N-(3-fluorobenzyl)-N-methylnicotinamide;
2-(difluoromethoxy)-N-(3-fluorobenzyl)-N-methyl-5-(trifluoromethyl) nicotinamide;
5-cyclobutyl-2-(difluoromethoxy)-N-(3-fluorobenzyl)-N-methylnicotinamide;
5-chloro-2-(difluoromethoxy)-N-(3-(difluoromethoxy)benzyl)-N-methylnicotinamide;
5-chloro-2-(difluoromethoxy)-N-methyl-N-(3-(trifluoromethoxy)benzyl) nicotinamide;
5-chloro-N-(2,4-difluorobenzyl)-2-(difluoromethoxy)-N-methylnicotinamide;
5-chloro-2-(difluoromethoxy)-N-(3-fluorobenzyl)-N-methylnicotinamide;
5-chloro-N-(2, 3-difluorobenzyl)-2-(difluoromethoxy)-N-methylnicotinamide;
N-(3,5-difluorobenzyl)-2-(difluoromethoxy)-5-fluoro-N-methylpyridine-3-sulfonamide;
(R)-2-(difluoromethoxy)-5-fluoro-N-(1-(3-fluorophenyl)ethyl)-N-methylpyridine-3-sulfonamide;
(S)-2-(difluoromethoxy)-5-fluoro-N-(1-(3-fluorophenyl)ethyl)-N-methylpyridine-3-sulfonamide;
N-(3-cyanobenzyl)-2-(difluoromethoxy)-5-fluoro-N-methylpyridine-3-sulfonamide;
N-(2,3-difluorobenzyl)-2-(difluoromethoxy)-5-fluoro-N-methylpyridine-3-sulfonamide;
N-(3-chlorobenzyl)-2-(difluoromethoxy)-5-fluoro-N-methylpyridine-3-sulfonamide;
2-(difluoromethoxy)-N-ethyl-5-fluoro-N-(3-fluorobenzyl)pyridine-3-sulfonamide;
2-((2-(difluoromethoxy)-5-fluoropyridin-3-yl)sulfonyl)-1,2,3,4-tetrahydroisoquinoline;
2-((2-(difluoromethoxy)-5-fluoropyridin-3-yl)sulfonyl)-7-fluoro-1,2,3,4-tetrahydroisoquinoline;
2-(difluoromethoxy)-5-fluoro-N-(3-fluorobenzyl)-N-methylpyridine-3-sulfonamide;
5-chloro-2-(difluoromethoxy)-N-(3-fluorobenzyl)-N-methylpyridine-3-sulfonamide;
4-((2-(difluoromethoxy)-5-fluoropyridin-3-yl)sulfonyl)-7-fluoro-2,3,4,5-tetrahydrobenzo[f][1,4]oxazepine;
4-((5-chloro-2-(difluoromethoxy)pyridin-3-yl)sulfonyl)-7-fluoro-2,3,4,5-tetrahydrobenzo[f][1,4]oxazepine;
2-((5-chloro-2-(difluoromethoxy)pyridin-3-yl)sulfonyl)-6,8-difluoro-1,2,3,4-tetrahydroisoquinoline;
5-chloro-N-(2,6-difluorobenzyl)-2-methoxy-N-methylnicotinamide;
5-chloro-N-(2,6-dichlorobenzyl)-2-methoxy-N-methylnicotinamide;
5-chloro-2-(difluoromethoxy)-N-(1,1-dioxido-2,3-dihydrobenzo[b]thiophen-3-yl)-N-methylnicotinamide;
5-fluoro-2-((7-fluoro-2,3-dihydrobenzo[f][1,4]oxazepin-4(5H)-yl)sulfonyl)benzonitrile;
or an enantiomer of any of the foregoing;
or a pharmaceutically acceptable salt, solvate or prodrug of any of the foregoing.

11. A process for the preparation of a compound of formula (I) or a pharmaceutically acceptable salt, N-oxide, solvate or prodrug thereof, as claimed in any one of claims 1 to 10, wherein the process comprises:
(a) reacting a compound of formula (II), or a salt thereof, with an amine of formula (III), or a salt thereof: 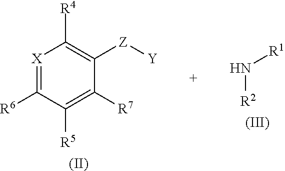
wherein X, Z, R¹, R², R⁴, R⁵, R⁶ and R⁷ are as defined in any one of claims 1 to 10; Y is —OH, —OR⁸, or —Cl; and R⁸ is C₁-C₃ alkyl; or
(b) reacting a compound of formula (IV), or a salt thereof, with a compound of formula (V), or a salt thereof: 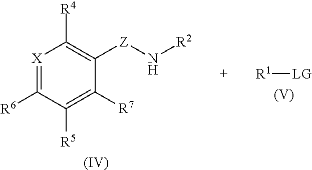
wherein X, Z, R¹, R², R⁴, R⁵, R⁶ and R⁷ are as defined in any one of claims 1 to 10, and LG is a leaving group such as halo (such as fluoro, chloro, bromo, or iodo), a sulfate group (such as methyl sulfate), or a sulfonate group (such as mesylate, triflate, or tosylate);
and optionally thereafter carrying out one or more of the following procedures:
converting a compound of formula (I) into another compound of formula (I);
removing any protecting groups;
forming a pharmaceutically acceptable salt or N-oxide.

12. A pharmaceutical composition comprising the compound, salt, N-oxide, solvate or prodrug as claimed in any one of claims 1 to 10, in association with a pharmaceutically acceptable adjuvant, diluent or carrier, and optionally one or more other therapeutic agents.

13. The compound, salt, N-oxide, solvate or prodrug as claimed in any one of claims 1 to 10 for use in therapy.

14. The compound, salt, N-oxide, solvate or prodrug as claimed in any one of claims 1 to 10, for use in treating or preventing a disease, disorder or condition associated with KCNK13 activity.

15. The compound, salt, N-oxide, solvate or prodrug as claimed in any one of claims 1 to 10, for use in treating or preventing a neurodegenerative disease, a psychiatric disease, a genetic disease, hearing loss, an ocular or retinal disease, a cardiovascular disease, an inflammatory disease, an autoimmune disease, or a metabolic disease.

16. The compound, salt, N-oxide, solvate or prodrug as claimed in any one of claims 1 to 10, for use in treating or preventing Alzheimer's disease, Parkinson's disease, frontal temporal dementia, progressive supranuclear palsy (PSP) and related tauopathies, amyotrophic lateral sclerosis (ALS)/motor neuron disease (MND), traumatic brain injury, multiple sclerosis, stroke, ischemic insult, depression, stress, anxiety related disorder (including social and generalised anxiety), post-traumatic stress disorder (PTSD), schizophrenia, bipolar disorder, cryopyrin-associated periodic syndrome (CAPS) (including Muckle-Wells syndrome (MWS), familial cold autoinflammatory syndrome (FCAS), chronic infantile neurological cutaneous and articular (CINCA) syndrome, and neonatal onset multisystem inflammatory disease (NOMID)), age related hearing loss, genetic related hearing loss (including NLRP3 mutation related hearing loss), autoimmune related hearing loss, macular degeneration, age related macular degeneration, diabetic retinopathy, atherosclerosis, myocardial infarction, ischemia, rheumatoid arthritis, gout, Lupus, asthma, respiratory inflammation, inflammatory or autoimmune skin disease, psoriasis, inflammatory bowel disease, colitis, metabolic syndrome, non-alcoholic fatty liver disease (NAFLD), non-alcoholic steatohepatitis (NASH), fibrosis, or diabetes.
